SEQUENCE LISTING (12 sequences)

SEQ ID NO: 1 - protein, 8 aa, synthetic construct
SIINFEKL

SEQ ID NO: 2 - RNA, 930 nt, synthetic construct
  source 1..930: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgtactccatgcagctggcctcctgcgtgaccctgaccctggtgctgctggtgaactccgcccccacctcctcctccacctcctcctccaccgccgaggcccagcagcagcagcagcagcagcagcagcagcagcagcacctggagcagctgctgatggacctgcaggagctgctgtcccgcatggagaactaccgcaacctgaagctgccccgcatgctgaccttcaagttctacctgcccaagcaggccaccgagctgaaggacctgcagtgcctggaggacgagctgggccccctgcgccacgtgctggacctgacccagtccaagtccttccagctggaggacgccgagaacttcatctccaacatccgcgtgaccgtggtgaagctgaagggctccgacaacaccttcgagtgccagttcgacgacgagtccgccaccgtggtggacttcctgcgccgctggatcgccttctgccagtccatcatctccacctccccccagtaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - RNA, 1056 nt, synthetic construct
  source 1..1056: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgaagttcctgtccgcccgcgacttccaccccgtggccttcctgggcctgatgctggtgaccaccaccgccttccccacctcccaggtgcgccgcggcgacttcaccgaggacaccacccccaaccgccccgtgtacaccacctcccaggtgggcggcctgatcacccacgtgctgtgggagatcgtggagatgcgcaaggagctgtgcaacggcaactccgactgcatgaacaacgacgacgccctggccgagaacaacctgaagctgcccgagatccagcgcaacgacggctgctaccagaccggctacaaccaggagatctgcctgctgaagatctcctccggcctgctggagtaccactcctacctggagtacatgaagaacaacctgaaggacaacaagaaggacaaggcccgcgtgctgcagcgcgacaccgagaccctgatccacatcttcaaccaggaggtgaaggacctgcacaagatcgtgctgcccacccccatctccaacgccctgctgaccgacaagctggagtcccagaaggagtggctgcgcaccaagaccatccagttcatcctgaagtccctggaggagttcctgaaggtgaccctgcgctccacccgccagacctaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - RNA, 2181 nt, synthetic construct
  source 1..2181: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgtgcccccagaagctgaccatctcctggttcgccatcgtgctgctggtgtcccccctgatggccatgtgggagctggagaaggacgtgtacgtggtggaggtggactggacccccgacgcccccggcgagaccgtgaacctgacctgcgacacccccgaggaggacgacatcacctggacctccgaccagcgccacggcgtgatcggctccggcaagaccctgaccatcaccgtgaaggagttcctggacgccggccagtacacctgccacaagggcggcgagaccctgtcccactcccacctgctgctgcacaagaaggagaacggcatctggtccaccgagatcctgaagaacttcaagaacaagaccttcctgaagtgcgaggcccccaactactccggccgcttcacctgctcctggctggtgcagcgcaacatggacctgaagttcaacatcaagtcctcctcctcctcccccgactcccgcgccgtgacctgcggcatggcctccctgtccgccgagaaggtgaccctggaccagcgcgactacgagaagtactccgtgtcctgccaggaggacgtgacctgccccaccgccgaggagaccctgcccatcgagctggccctggaggcccgccagcagaacaagtacgagaactactccacctccttcttcatccgcgacatcatcaagcccgacccccccaagaacctgcagatgaagcccctgaagaactcccaggtggaggtgtcctgggagtaccccgactcctggtccaccccccactcctacttctccctgaagttcttcgtgcgcatccagcgcaagaaggagaagatgaaggagaccgaggagggctgcaaccagaagggcgccttcctggtggagaagacctccaccgaggtgcagtgcaagggcggcaacgtgtgcgtgcaggcccaggaccgctactacaactcctcctgctccaagtgggcctgcgtgccctgccgcgtgcgctcctccggcggcggcggctccggcggcggcggctccggcggcggcggctcccccgtgtccgtgcccaccgcctccccctccgcctcctcctcctcctcccagtgccgctcctccatgtgccagtcccgctacctgctgttcctggccaccctggccctgctgaaccacctgtccctggcccgcgtgatccccgtgtccggccccgcccgctgcctgtcccagtcccgcaacctgctgaagaccaccgacgacatggtgaagaccgcccgcgagaagctgaagcactactcctgcaccgccgaggacatcgaccacgaggacatcacccgcgaccagacctccaccctgaagacctgcctgcccctggagctgcacaagaacgagtcctgcctggccacccgcgagacctcctccaccacccgcggctcctgcctgcccccccagaagacctccctgatgatgaccctgtgcctgggctccatctacgaggacctgaagatgtaccagaccgagttccaggccatcaacgccgccctgcagaaccacaaccaccagcagatcatcctggacaagggcatgctggtggccatcgacgagctgatgcagtccctgaaccacaacggcgagaccctgcgccagaagccccccgtgggcgaggccgacccctaccgcgtgaagatgaagctgtgcatcctgctgcacgccttctccacccgcgtggtgaccatcaaccgcgtgatgggctacctgtcctccgcctgaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 5 - RNA, 2172 nt, synthetic construct
  source 1..2172: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcgcatgcagctgctgctgctgatcgccctgtccctggccctggtgaccaactccatgtgggagctggagaaggacgtgtacgtggtggaggtggactggacccccgacgcccccggcgagaccgtgaacctgacctgcgacacccccgaggaggacgacatcacctggacctccgaccagcgccacggcgtgatcggctccggcaagaccctgaccatcaccgtgaaggagttcctggacgccggccagtacacctgccacaagggcggcgagaccctgtcccactcccacctgctgctgcacaagaaggagaacggcatctggtccaccgagatcctgaagaacttcaagaacaagaccttcctgaagtgcgaggcccccaactactccggccgcttcacctgctcctggctggtgcagcgcaacatggacctgaagttcaacatcaagtcctcctcctcctcccccgactcccgcgccgtgacctgcggcatggcctccctgtccgccgagaaggtgaccctggaccagcgcgactacgagaagtactccgtgtcctgccaggaggacgtgacctgccccaccgccgaggagaccctgcccatcgagctggccctggaggcccgccagcagaacaagtacgagaactactccacctccttcttcatccgcgacatcatcaagcccgacccccccaagaacctgcagatgaagcccctgaagaactcccaggtggaggtgtcctgggagtaccccgactcctggtccaccccccactcctacttctccctgaagttcttcgtgcgcatccagcgcaagaaggagaagatgaaggagaccgaggagggctgcaaccagaagggcgccttcctggtggagaagacctccaccgaggtgcagtgcaagggcggcaacgtgtgcgtgcaggcccaggaccgctactacaactcctcctgctccaagtgggcctgcgtgccctgccgcgtgcgctcctccggcggcggcggctccggcggcggcggctccggcggcggcggctcccccgtgtccgtgcccaccgcctccccctccgcctcctcctcctcctcccagtgccgctcctccatgtgccagtcccgctacctgctgttcctggccaccctggccctgctgaaccacctgtccctggcccgcgtgatccccgtgtccggccccgcccgctgcctgtcccagtcccgcaacctgctgaagaccaccgacgacatggtgaagaccgcccgcgagaagctgaagcactactcctgcaccgccgaggacatcgaccacgaggacatcacccgcgaccagacctccaccctgaagacctgcctgcccctggagctgcacaagaacgagtcctgcctggccacccgcgagacctcctccaccacccgcggctcctgcctgcccccccagaagacctccctgatgatgaccctgtgcctgggctccatctacgaggacctgaagatgtaccagaccgagttccaggccatcaacgccgccctgcagaaccacaaccaccagcagatcatcctggacaagggcatgctggtggccatcgacgagctgatgcagtccctgaaccacaacggcgagaccctgcgccagaagccccccgtgggcgaggccgacccctaccgcgtgaagatgaagctgtgcatcctgctgcacgccttctccacccgcgtggtgaccatcaaccgcgtgatgggctacctgtcctccgcctgaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - RNA, 1005 nt, synthetic construct
  source 1..1005: mol_type = other RNA
atgtgcccccagaagctgaccatctcctggttcgccatcgtgctgctggtgtcccccctgatggccatgtgggagctggagaaggacgtgtacgtggtggaggtggactggacccccgacgcccccggcgagaccgtgaacctgacctgcgacacccccgaggaggacgacatcacctggacctccgaccagcgccacggcgtgatcggctccggcaagaccctgaccatcaccgtgaaggccgccctggacgccggccagtacacctgccacaagggcggcgagaccctgtcccactcccacctgctgctgcacaagaaggagaacggcatctggtccaccgagatcctgaagaacttcaagaacaagaccttcctgaagtgcgaggcccccaactactccggccgcttcacctgctcctggctggtgcagcgcaacatggacctgaagttcaacatcaagtcctcctcctcctcccccgactcccgcgccgtgacctgcggcatggcctccctgtccgccgagaaggtgaccctggaccagcgcgactacgagaagtactccgtgtcctgccaggaggacgtgacctgccccaccgccgaggagaccctgcccatcgagctggccctggaggcccgccagcagaacaagtacgagaactactccacctccttcttcatccgcgacatcatcaagcccgacccccccaagaacctgcagatgaagcccctgaagaactcccaggtggaggtgtcctgggagtaccccgactcctggtccaccccccactcctacttctccctgaagttcttcgtgcgcatccagcgcaagaaggagaagatgaaggagaccgaggagggctgcaaccagaagggcgccttcctggtggagaagacctccaccgaggtgcagtgcaagggcggcaacgtgtgcgtgcaggcccaggaccgctactacaactcctcctgctccaagtgggcctgcgtgccctgccgcgtgcgctcc

SEQ ID NO: 7 - RNA, 1005 nt, synthetic construct
  source 1..1005: mol_type = other RNA
atgtgcccccagaagctgaccatctcctggttcgccatcgtgctgctggtgtcccccctgatggccatgtgggagctggagaaggacgtgtacgtggtggaggtggactggacccccgacgcccccggcgagaccgtgaacctgacctgcgacacccccgaggaggacgacatcacctggacctccgaccagcgccacggcgtgatcggctccggcaagaccctgaccatcaccgtgaaggccgccctggacgccggccagtacacctgccacaagggcggcgagaccctgtcccactcccacctgctgctgcacgccaaggagaacggcatctggtccaccgagatcctgaagaacttcaagaacaagaccttcctgaagtgcgaggcccccaactactccggccgcttcacctgctcctggctggtgcagcgcaacatggacctgaagttcaacatcaagtcctcctcctcctcccccgactcccgcgccgtgacctgcggcatggcctccctgtccgccgagaaggtgaccctggaccagcgcgactacgagaagtactccgtgtcctgccaggaggacgtgacctgccccaccgccgaggagaccctgcccatcgagctggccctggaggcccgccagcagaacaagtacgagaactactccacctccttcttcatccgcgacatcatcaagcccgacccccccaagaacctgcagatgaagcccctgaagaactcccaggtggaggtgtcctgggagtaccccgactcctggtccaccccccactcctacttctccctgaagttcttcgtgcgcatccagcgcaagaaggagaagatgaaggagaccgaggagggctgcaaccagaagggcgccttcctggtggagaagacctccaccgaggtgcagtgcaagggcggcaacgtgtgcgtgcaggcccaggaccgctactacaactcctcctgctccaagtgggcctgcgtgccctgccgcgtgcgctcc

SEQ ID NO: 8 - RNA, 1005 nt, synthetic construct
  source 1..1005: mol_type = other RNA
atgtgcccccagaagctgaccatctcctggttcgccatcgtgctgctggtgtcccccctgatggccatgtgggagctggagaaggacgtgtacgtggtggaggtggactggacccccgacgcccccggcgagaccgtgaacctgacctgcgacacccccgaggaggacgacatcacctggacctccgaccagcgccacggcgtgatcggctccggcaagaccctgaccatcaccgtgaaggccgccctggacgccggccagtacacctgccacaagggcggcgagaccctgtcccactcccacctgctgctgcacgccaaggagaacggcatctggtccaccgagatcctgaagaacttcaagaacaagaccttcctgaagtgcgaggcccccaactactccggccgcttcacctgctcctggctggtgcagcgcaacatggacctgaagttcaacatcaagtcctcctcctcctcccccgactcccgcgccgtgacctgcggcatggcctccctgtccgccgagaaggtgaccctggaccagcgcgactacgagaagtactccgtgtcctgccaggaggacgtgacctgccccaccgccgaggagaccctgcccatcgagctggccctggaggcccgccagcagaacgcctacgagaactactccacctccttcttcatccgcgacatcatcaagcccgacccccccaagaacctgcagatgaagcccctgaagaactcccaggtggaggtgtcctgggagtaccccgactcctggtccaccccccactcctacttctccctgaagttcttcgtgcgcatccagcgcaagaaggagaagatgaaggagaccgaggagggctgcaaccagaagggcgccttcctggtggagaagacctccaccgaggtgcagtgcaagggcggcaacgtgtgcgtgcaggcccaggaccgctactacaactcctcctgctccaagtgggcctgcgtgccctgccgcgtgcgctcc

SEQ ID NO: 9 - RNA, 1795 nt, synthetic construct
  source 1..1795: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgtccgccctgctgatcctggccctggtgggcgccgccgtggccgactacaaggacgacgacgacaagctgggctacaccgagaccgccctggtggccctgtcccagccccgcgtgcagtgccacgcctcccgctaccccgtggccgtggactgctcctggacccccctgcaggcccccaactccacccgctccacctccttcatcgccacctaccgcctgggcgtggccacccagcagcagtcccagccctgcctgcagcgctccccccaggcctcccgctgcaccatccccgacgtgcacctgttctccaccgtgccctacatgctgaacgtgaccgccgtgcaccccggcggcgcctcctcctccctgctggccttcgtggccgagcgcatcatcaagcccgacccccccgagggcgtgcgcctgcgcaccgccggccagcgcctgcaggtgctgtggcacccccccgcctcctggcccttccccgacatcttctccctgaagtaccgcctgcgctaccgccgccgcggcgcctcccacttccgccaggtgggccccatcgaggccaccaccttcaccctgcgcaactccaagccccacgccaagtactgcatccaggtgtccgcccaggacctgaccgactacggcaagccctccgactggtccctgcccggccaggtggagtccgccccccacaagccctcccgcggctccggctccggcggctccggcggctccggctccggcaagctgcccaccgaccccctgtccctgcaggagctgcgccgcgagttcaccgtgtccctgtacctggcccgcaagctgctgtccgaggtgcagggctacgtgcactccttcgccgagtcccgcctgcccggcgtgaacctggacctgctgcccctgggctaccacctgcccaacgtgtccctgaccttccaggcctggcaccacctgtccgactccgagcgcctgtgcttcctggccaccaccctgcgccccttccccgccatgctgggcggcctgggcacccagggcacctggacctcctccgagcgcgagcagctgtgggccatgcgcctggacctgcgcgacctgcaccgccacctgcgcttccaggtgctggccgccggcttcaagtgctccaaggaggaggaggacaaggaggaggaggaggaggaggaggaggaggagaagaagctgcccctgggcgccctgggcggccccaaccaggtgtcctcccaggtgtcctggccccagctgctgtacacctaccagctgctgcactccctggagctggtgctgtcccgcgccgtgcgcgacctgctgctgctgtccctgccccgccgccccggctccgcctgggactccttaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 10 - protein, 335 aa, synthetic construct
MCPQKLTISWFAIVLLVSPLMAMWELEKDVYVVEVDWTPDAPGETVNLTCDTPEEDDITWTSDQRHGVIGSGKTLTITVKAALDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 11 - protein, 335 aa, synthetic construct
MCPQKLTISWFAIVLLVSPLMAMWELEKDVYVVEVDWTPDAPGETVNLTCDTPEEDDITWTSDQRHGVIGSGKTLTITVKAALDAGQYTCHKGGETLSHSHLLLHAKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 12 - protein, 335 aa, synthetic construct
MCPQKLTISWFAIVLLVSPLMAMWELEKDVYVVEVDWTPDAPGETVNLTCDTPEEDDITWTSDQRHGVIGSGKTLTITVKAALDAGQYTCHKGGETLSHSHLLLHAKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNAYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

(19) United States
(12) Patent Application Publication
(10) Pub. No.: US 2026/0076905 A1
(43) Pub. Date: Mar. 19, 2026

(54) COMPOSITIONS AND METHODS COMPRISING LIPID NANOPARTICLE VACCINES THAT ELICIT A MODULATED IMMUNE RESPONSE

(71) Applicant: THE TRUSTEES OF THE UNIVERSITY OF PENNSYLVANIA, Philadelphia, PA (US)

(72) Inventors: Christopher A. Hunter, Philadelphia, PA (US); Anthony T. Phan, Philadelphia, PA (US); Mohamad-Gabriel Alameh, Philadelphia, PA (US); Drew Weissman, Philadelphia, PA (US); Emily Anne Aunins, Philadelphia, PA (US)

(21) Appl. No.: 19/109,495

(22) PCT Filed: Sep. 8, 2023
(86) PCT No.: PCT/US2023/073782

§ 371 Date: Mar. 6, 2025

Related U.S. Application Data
(60) Provisional application No. 63/375,098, filed on Sep. 9, 2022.

(51) Int. Cl. A61K 9/1272; A61K 9/00; A61K 9/51; A61K 39/00; A61K 39/215; A61P 31/04; A61P 35/00; A61P 37/04; C12N 7/00

(52) U.S. Cl. CPC A61K 9/1272; A61K 9/0019; A61K 9/5123; A61K 39/00114; A61K 39/215; A61P 31/04; A61P 35/00; A61P 37/04; C12N 7/00; A61K 2039/53; A61K 2039/54; A61K 2039/55555; A61K 2039/572; C12N 2770/20034

(57) ABSTRACT

The invention includes lipid nanoparticles (LNP) capable of eliciting a modulated immune response against an antigen in a subject. The LNPs comprise: (a) at least one first nucleoside-modified ribonucleic acid (RNA) encoding an antigen; (b) at least one second nucleoside-modified RNA encoding a cytokine or immune receptor (such as but not limited to a cytokine receptor); and (c) at least one ionizable lipid. The invention also includes pharmaceutical compositions comprising the LNP of the invention, as well as a method of eliciting a modulated immune response against an antigen in a subject, the method comprising administering an effective amount of a pharmaceutical compositions comprising the LNP of the invention.

### CROSS-REFERENCE TO RELATED APPLICATIONS

This application claims priority under 35 U.S.C. § 119(e) to U.S. Provisional Patent Application No. 63/375,098 filed Sep. 9, 2022, which is incorporated herein by reference in its entirety.

### STATEMENT REGARDING FEDERALLY SPONSORED RESEARCH OR DEVELOPMENT

This invention was made with government support under AI160664 awarded by the National Institutes of Health. The government has certain rights in the invention.

### BACKGROUND OF THE INVENTION

Lipid nanoparticle (LNP)-mRNA vaccines against severe acute respiratory syndrome coronavirus 2 (SARS-CoV-2) have been shown to promote robust protective antibody and T cell responses to the virus (Polack, et al., N Engl J Med, 2020, 383:2603-2615; Baden, et al., N Engl J Med, 2021, 384:403-416; Tarke, et al., Cell, 2022, 185, 847-859.e11; Urbanowicz, et al., Sci Trans Med, 2021, eabj0847; Yokoyama, et al., Ann Rev Immun, 2021, 39, v-vii) and significantly reduce the risk of severe illness, hospitalization, and death from coronavirus disease 2019 (COVID-19) (Chung, et al., BMJ, 2021, 374:n1943; Abu-Radded, et al., N Engl J Med, 2021 May 5: NEJMc210497; Chemaitelly, et al., Nat. Med., 2021, 27(9):1614-1621; Hall, et al., Lancet, 2021, 397(10286): 1725-1735). Clinical studies of patients immunized with these vaccines (i.e., mRNA-1273 and BNT162b2) have shown that they induce significant populations of SARS-CoV-2-specific T cells that respond to additional doses of vaccine or encounter with SARS-CoV-2.

The success of LNP-mRNA SARS-CoV-2 vaccines and the flexibility of the modular LNP-mRNA vaccine platform provide a solid foundation for the development and use of nucleic acid-based LNP vaccines for prevention of illness and death caused by other pathogens. That said, despite their widespread use against SARS-CoV-2, there remain significant knowledge gaps in how LNP-mRNA vaccines induce protective immunity.

Additionally, it is noteworthy that SARS-CoV-2 specific CD8⁺ T cell responses were observed to be reduced in mRNA-1273 vaccinated patients during clinical trials in comparison to patients immunized with BNT162b2. The differential induction of SARS-CoV-2-specific CD8⁺ T cell responses by these two LNP-mRNA vaccines begs the question whether fundamental immunological differences underly the differential clinical outcomes. Further, the level of protection that might be achieved for an LNP vaccine against any given pathogen is largely unknown and could be lower than that observed for the SARS-CoV-2 vaccines.

There is an urgent need in the art for tools to augment the immune response induced by nucleic acid-based LNP vaccines and to drive the appropriate immune response for protection against a given pathogen, as well as optimize for reducing vaccine dose and/or number of doses required. The present invention addresses this need.

### SUMMARY OF THE INVENTION

The present invention includes lipid nanoparticles (LNP) capable of eliciting a modulated immune response against an antigen in a subject. The invention also includes pharmaceutical compositions comprising the LNP of the invention, as well as a method of eliciting a modulated immune response against an antigen in a subject, the method comprising administering an effective amount of a pharmaceutical compositions comprising the LNP of the invention.

As such, in one aspect, the invention provides a lipid nanoparticle (LNP), wherein the LNP comprises:
(a) at least one first nucleoside-modified ribonucleic acid (RNA), wherein the at least one first nucleoside-modified RNA encodes an antigen;
(b) at least one second nucleoside-modified RNA, wherein the at least one second nucleoside-modified RNA encodes a cytokine or immune receptor (such as but not limited to a cytokine receptor); and
(c) at least one ionizable lipid;
wherein the LNP is capable of eliciting a modulated immune response against the antigen in a subject.

In certain embodiments, the modulated immune response comprises an enhanced immune response and/or a decreased immune response.

In certain embodiments, the modulated immune response is tissue-specific.

In certain embodiments, the first nucleoside-modified RNA, the second nucleoside-modified RNA, or both, is/are messenger RNA (mRNA).

In certain embodiments, the first nucleoside-modified RNA, the second nucleoside-modified RNA, or both, independently comprise pseudouridines and/or 1-methyl-pseudouridines.

In certain embodiments, the first nucleoside-modified RNA, the second nucleoside-modified RNA, or both, is/are in vitro transcribed (IVT) RNA.

In certain embodiments, the cytokine is selected from the group consisting of a chemokine, an interleukin (IL), an interferon (IFN), a tumor necrosis factor (TNF) family member, a transforming growth factor (TGF), and any combination thereof.

In certain embodiments, the cytokine is selected from the group consisting of IL-2, IL-6, IL-12, IL-15, IL-27, TGF-β, and any combination thereof.

In certain embodiments, the cytokine comprises IL-27.

In certain embodiments, the second nucleoside-modified RNA encodes an Epstein-Barr virus-induced gene 3 (Ebi3) subunit of IL-27 linked to an IL-27p28 subunit of IL-27 via a flexible protein linker.

In certain embodiments, the cytokine comprises IL-12.

In certain embodiments, the second nucleoside-modified RNA encodes a p40 subunit of IL-12 linked to a p35 subunit of IL-12 via a flexible protein linker.

In certain embodiments, the immune receptor comprises a soluble immune receptor or immune receptor decoy.

In certain embodiments, the cytokine receptor comprises a soluble cytokine receptor or a cytokine receptor decoy.

In certain embodiments, the ionizable lipid encapsulates the first nucleoside-modified RNA and the second nucleoside-modified RNA.

In certain embodiments, the ionizable lipid is a cationic lipid.

In certain embodiments, the ionizable lipid is selected from those similar to that formulated in the BNT162b2 vaccine and SM-102.

In certain embodiments, the antigen is derived from a pathogen, and wherein the pathogen is selected from a virus, a bacterium, a fungus, or a parasite.

In certain embodiments, the pathogen is a severe acute respiratory syndrome coronavirus 2 (SARS-CoV-2) virus.

In certain embodiments, the SARS-CoV-2 is wild-type SARS-CoV-2 or a variant SARS-CoV-2.

In certain embodiments, the antigen is a SARS-CoV-2 antigen.

In certain embodiments, the antigen is a tumor antigen.

In certain embodiments:
(i) the enhanced immune response in the subject comprises an augmented CD8⁺ T cell response in the subject compared to a CD8⁺ T cell response in a subject administered a control LNP lacking the second nucleoside-modified RNA encoding a cytokine or immune receptor (such as but not limited to a cytokine receptor); or
(ii) the reduced immune response in the subject comprises a decreased CD8⁺ T cell response in the subject compared to a CD8⁺ T cell response in a subject administered a control LNP lacking the second nucleoside-modified RNA encoding a cytokine or immune receptor (such as but not limited to a cytokine receptor).

In one aspect, the invention provides a pharmaceutical composition comprising the LNP disclosed herein and at least one pharmaceutically acceptable carrier, diluent, and/or excipient.

In one aspect, the invention provides the LNP disclosed herein, for use in a method of eliciting a modulated immune response against the antigen in the subject.

In one aspect, the invention provides a method of eliciting a modulated immune response against an antigen in a subject, the method comprising administering to the subject an effective amount of a pharmaceutical composition comprising a lipid nanoparticle (LNP) and at least one pharmaceutically acceptable carrier, diluent, and/or excipient, wherein the LNP comprises:
(a) at least one first nucleoside-modified ribonucleic acid (RNA) encoding the antigen;
(b) at least one second nucleoside-modified RNA encoding a cytokine or immune receptor (such as but not limited to a cytokine receptor); and
(c) at least one ionizable lipid;
wherein the LNP elicits a modulated immune response against the antigen in the subject.

In certain embodiments, the modulated immune response comprises an enhanced immune response and/or a decreased immune response.

In certain embodiments, the modulated immune response is tissue-specific.

In certain embodiments, the first nucleoside-modified RNA, the second nucleoside-modified RNA, or both, is/are messenger RNA (mRNA).

In certain embodiments, the first nucleoside-modified RNA, the second nucleoside-modified RNA, or both, independently comprise pseudouridine and/or 1-methyl-pseudouridine.

In certain embodiments, the first nucleoside-modified RNA, the second nucleoside-modified RNA, or both, is/are in vitro transcribed (IVT) RNA.

In certain embodiments, the cytokine is selected from the group consisting of a chemokine, an interleukin (IL), an interferon (IFN), a tumor necrosis factor (TNF) family member, a transforming growth factor (TGF), and any combination thereof.

In certain embodiments, the cytokine is selected from the group consisting of IL-2, IL-6, IL-12, IL-15, IL-27, TGF-β, and any combination thereof.

In certain embodiments, the cytokine comprises IL-27.

In certain embodiments, the second nucleoside-modified RNA encodes an Epstein-Barr virus-induced gene 3 (Ebi3) subunit of IL-27 linked to an IL-27p28 subunit of IL-27 via a flexible protein linker.

In certain embodiments, the cytokine comprises IL-12.

In certain embodiments, the second nucleoside-modified RNA encodes a p40 subunit of IL-12 linked to a p35 subunit of IL-12 via a flexible protein linker.

In certain embodiments, the immune receptor comprises a soluble immune receptor or immune receptor decoy.

In certain embodiments, the cytokine receptor comprises a soluble cytokine receptor or a cytokine receptor decoy.

In certain embodiments, the ionizable lipid encapsulates the first nucleoside-modified RNA and the second nucleoside-modified RNA.

In certain embodiments, the ionizable lipid is a cationic lipid.

In certain embodiments, the ionizable lipid is selected from those similar to that formulated in the BNT162b2 vaccine and SM-102.

In certain embodiments, the antigen is derived from a pathogen, and wherein the pathogen is selected from a virus, a bacterium, a fungus, or a parasite.

In certain embodiments, the pathogen is a severe acute respiratory syndrome coronavirus 2 (SARS-CoV-2) virus.

In certain embodiments, the SARS-CoV-2 is wild-type SARS-CoV-2 or a variant SARS-CoV-2.

In certain embodiments, the antigen is a SARS-CoV-2 antigen.

In certain embodiments, the antigen is a tumor antigen.

In certain embodiments:
(i) the enhanced immune response in the subject comprises an augmented CD8⁺ T cell response in the subject compared to a CD8⁺ T cell response in a subject administered a control composition lacking the second nucleoside-modified RNA encoding a cytokine or immune receptor (such as but not limited to a cytokine receptor); or
(ii) the reduced immune response in the subject comprises a decreased CD8⁺ T cell response in the subject compared to a CD8⁺ T cell response in a subject administered a control LNP lacking the second nucleoside-modified RNA encoding a cytokine or immune receptor (such as but not limited to a cytokine receptor).

In certain embodiments, the subject is a human.

In certain embodiments, the administering comprises intramuscular injection.

In certain embodiments, the administering comprises administering a first dose.

In certain embodiments, the administering further comprises administering at least one booster dose.

### BRIEF DESCRIPTION OF THE DRAWINGS

The following detailed description of preferred embodiments of the invention will be better understood when read in conjunction with the appended drawings. For the purpose of illustrating the invention, non-limiting illustrative embodiments are shown in the drawings. It should be understood, however, that the invention is not limited to the precise arrangements and instrumentalities of the embodiments shown in the drawings.

FIGS. 1A-1B illustrate the finding that the cytokine IL-27 is induced following LNP-mRNA immunization in vivo. FIG. 1A is a schematic illustrating the experimental design. FIG. 1B is a plot of the quantified ELISA assay results.

FIGS. 2A-2E provide data from an experiment used to determine the cellular source of IL-27 following LNP-mRNA immunization. FIG. 2A is a schematic illustrating the experimental design. FIG. 2B shows flow cytometry plots for cells of inguinal and popliteal draining lymph nodes 6 hours post-immunization. FIG. 2C is a graph showing quantification of the number of IL-27p28GFP+ CD45+ cells. FIG. 2D shows additional flow cytometry plots for determining the cellular source of IL-27. FIG. 2E shows quantification of the IL-27 expressing cells (i.e., IL-27p28GFP+ cells).

FIGS. 3A-3B illustrate the finding that endogenous IL-27 regulates CD8⁺ T cell responses following LNP-mRNA immunization in vivo. FIG. 3A is a schematic illustrating the experimental design. FIG. 3B is a plot of the quantified results showing that loss of IL-27 negatively impacts CD8⁺ T cell expansion following LNP-OVA immunization.

FIGS. 4A-4C illustrate the finding that CD8⁺ T cell-intrinsic IL-27 signaling is necessary for maximal expansion. FIG. 4A is a schematic illustrating the experimental design. FIG. 4B is a flow cytometry plot of the mixed splenocytes prior to immunization. FIG. 4C provides two plots of the quantified data from this experiment, one showing the percent of donor cells vs. days post immunization and the other showing the % of CD8a+ cells vs. days post immunization.

FIGS. 5A-5C relate to an experiment used to test the effect of a booster dose of LNP-OVA. FIG. 5A is a schematic illustrating the experimental design. FIG. 5B provides two plots of the quantified data from this experiment, one showing the percent of donor cells vs. days post immunization and the other showing the % of CD8a+ cells vs. days post immunization. FIG. 5C is a chart of the quantified data showing fold expansion 8 days post-boost.

FIGS. 6A-6C relate to an experiment used to determine whether loss of IL-27 signaling impacts distribution of memory cells or tissue residency. FIG. 6A is a schematic illustrating the experimental design. FIG. 6B is a chart of quantified data showing the % of donor cell type across various tissues. FIG. 6C is a chart of the quantified data showing there is limited generation of tissue resident memory cells in lung and liver.

FIGS. 7A-7B relate to the N1-methyl-pseudouridine-modified codon-optimized mRNA that encodes both subunits of IL-27, Ebi3 and IL27p28, linked by a flexible glycine serine linker. FIG. 7A is a diagram of the Ebi3p28 mRNA construct. FIG. 7B is a vector map of the pUC vector used to express the Ebi3p28 mRNA construct.

FIGS. 8A-8B relate to a dose response titration for LNP particles comprising the Ebi3p28 mRNA construct (LNP-Ebi3p28). FIG. 8A is a schematic illustrating the experimental design. FIG. 8B is a chart of the quantified ELISA data.

FIGS. 9A-9B illustrate the finding that cytokine produced via LNP-Ebi3p28 immunization is biologically active and can induce alterations in the immune response in vivo. FIG. 9A is a schematic illustrating the experimental design. FIG. 9B shows the flow cytometry plots and quantification of the flow cytometry data.

FIGS. 10A-10C illustrate the finding that LNPs that contain mRNA encoding IL-27 enhance the generation of CD8⁺ T cell immunity of LNP vaccines. FIG. 10A is a schematic illustrating the experimental design. FIG. 10B shows the flow cytometry plots. FIG. 10C shows quantification of the flow cytometry data.

FIGS. 11A-11B illustrate the finding that different LNP-mRNA vaccines possess differential capability to induce CD8⁺ T cell responses that is dependent on the specific ionizable lipid of the LNP. Mice were immunized with LNP-OVA particles formulated with the ionizable lipid from either mRNA-1273 (SM-102) or similar to that in BNT162b2. FIG. 11A shows flow cytometry plots for this experiment. FIG. 11B shows quantification of the flow cytometry data.

FIGS. 12A-12C illustrate the finding that IL-6 or IL-12 deficiency does not replicate the impact of loss of IL-27 on CD8⁺ T cell responses. FIG. 12A shows flow cytometry analysis of the proportion of adoptively transferred OT-I cells in the spleens of WT, IL-6, or IL-12-deficient hosts 8 days post a 1 mg dose of LNP-OVA administered i.m. FIG. 12B shows charts summarizing proportions of donor OT-Is of host CD8⁺ T cells and number of OT-Is from FIG. 12A. FIG. 12C shows charts summarizing proportions of donor OT-Is of host CD8⁺ T cells and number of OT-Is in the spleen of IL-27-deficient mice (p28 KO and Ebi3 KO).

FIGS. 13A-13D illustrate the finding that addition of IL-27 mRNA improves efficacy of SM-102 formulated LNPs to induce CD8⁺ T cell response. FIG. 13A is a graph of donor OT-I CD8⁺ T cells tracked in peripheral blood via flow cytometry analysis comparing mice immunized with BNT-OVA vs SM102-OVA. FIG. 13B is chart of the number of cells observed in blood at day 6 and day 28 post-immunization. FIG. 13C is a graph of donor OT-I CD8⁺ T cells tracked in peripheral blood via flow cytometry analysis comparing mice immunized with SM102-OVA vs SM102-OVA+IL-27. FIG. 13D is chart of the number of cells observed in blood at day 6 and day 28 post-immunization.

FIGS. 14A-14B illustrate the finding that co-administration of IL-27 mRNA with antigen mRNA results in improved protection to LM-OVA challenge. FIG. 14A shows an experimental schema for mice immunized with BNT-OVA formulations with or without IL-27 mRNA. Wildtype mice received no immunization, BNT-OVA, or BNT-OVA+IL-27. All mice received a 1 μg dose i.m. FIG. 14B is a chart showing mean CFU/g LM-OVA isolated from homogenized spleens of immunized mice challenged with LM-OVA 60 hrs prior to harvest. Data from 4 independent challenge studies.

FIGS. 15A-15B provide flow cytometry data illustrating the importance of IL-27 in supporting CD4⁺ T follicular helper responses. FIG. 15A shows flow cytometry analysis of activated polyclonal CD4⁺ T cells from the spleen of mice with T cell-specific deletion of the IL-27 receptor 10 days post-immunization with 1 g of BNT-OVA. FIG. 15B shows charts of the proportion and number of CD4⁺Bcl-6⁺CXCR5^{hi} T cells (CD4⁺ T_{FH}) observed.

FIG. 16 illustrates the finding that alternative cytokines, but not all, can modulate CD8⁺ T cell response to vaccine antigens. The charts show proportion and number, respectively, of observed donor CD8⁺ T cells in peripheral blood of mice 8 days post-immunization with a mixture of LNPs that encode for the antigen Ovalbumin and either IL-6 (top), IL-12 (center), or IL-2 (bottom).

FIGS. 17A-17B illustrate the finding that therapeutic administration of LNP-Ebi3IL27p28 along with LNP-OVA improves the inhibition of melanoma tumor growth. FIG. 17A is an experimental design schema for therapeutic vaccine treatment of subcutaneous B16F0-OVA tumors. FIG. 17B is a graph of tumor volume by days post-implantation. Primary and secondary dose of vaccine indicated at day 4 and day 12 post-implantation. N=10 mice per group for all groups except IL-27 only where N=5. By Two-way ANOVA Modified OVA+IL-27 and Unmodified OVA treated groups exhibited significantly different tumor volumes in comparison to IL-27 alone or PBS beginning day 14 post-implantation.

FIGS. 18A-18C provide data related to production of IL-12 protein in response to LNP-p40p35 particles. FIG. 18A is a schematic of the IL-12 construct used to create LNP-p40p35 particles. FIG. 18B is a graph of IL-12 production in vitro. WT and IL-12 deficient bone marrow derived macrophages were incubated with LNP-p40p35 particles overnight and assessed for IL12p40 production by ELISA. FIG. 18C is a graph of IL-12 production in vivo. Draining lymph nodes from WT mice immunized 24 hours prior to sacrifice were lysed and assessed for IL12p40 production by ELISA.

FIGS. 19A-19B illustrate the finding that LNP-p40p35 particles enhance the magnitude of OT-I expansion. FIG. 19A is an experimental design schema. FIG. 19B is a graph showing percentage of donor OT-I CD8b⁺ T cells in peripheral blood at indicated days post immunization. Mann-Whitney test: *=p<0.05.

FIGS. 20A-20C illustrate the finding that the expanded OT-I population induced by LNP-p40p35 augmented immunization persists to day 30 across tissues. FIG. 20A is an experimental design schema. FIG. 20B shows charts of the frequency and total number of OT-Is in the spleen at day 30 post-immunization. FIG. 20C shows charts of the frequency and total number of OT-Is in the spleen at day 30 post-immunization. Normality and log-normality tests were used for each data set. Unpaired t tests were performed for normally distributed data, Mann-Whitney was performed for non-normally distributed data (Lung-donor OT-I #of cells).

FIGS. 21A-21B illustrate the finding that LNP-p40p35 particles alter CD8⁺ T cell differentiation. FIG. 21A shows representative flow cytometry plots from the spleen 31 days post immunization. FIG. 21B shows charts of the frequency of KLRG1^{hi}CD127^{low} and CD127^{hi}KLRG1^{low} OT-Is generated by immunization.

FIGS. 22A-22B illustrate the finding that the ability of OT-Is to produce IFNγ at day 30 post immunization is augmented by LNP-p40p35 treatment. FIG. 22A shows representative flow cytometry plots. FIG. 22B shows charts of the frequency and total number of IFNγ producing OT-Is in spleen.

FIG. 23 is a chart showing antibody production is enhanced in aged mice that receive LNP-p40p35 particles.

FIGS. 24A-24B illustrate the finding that incorporation of IL-12 into mRNA vaccination enhances protection against Listeria monocytogenes. FIG. 24A is an experimental design schema. FIG. 24B is a chart showing correlation of bacteria in spleen and number of antigen-specific T cells in blood prior to challenge.

FIGS. 25A-25B illustrate the finding that LNP-OVA/LNP-p40p35 enhances clearance of tumors. FIG. 25A is an experimental design schema. FIG. 25B is a chart showing tumor volume over time. Two-way ANOVA with Tukey's post-test for multiple comparisons correction *<0.05, **<0.01 for day 16.

### DETAILED DESCRIPTION

The present invention relates generally to compositions and methods for eliciting a modulated immune response against an antigen in a subject. The invention includes improved lipid nanoparticle (LNP) vaccines and is based, in part, on the surprising discovery disclosed herein that LNP vaccines harboring nucleoside-modified RNA encoding a cytokine (e.g., IL-27) improve the efficacy of LNP-mRNA vaccine-induced CD8+ T cell responses and provide a proof of concept that the LNP-mRNA vaccine platform can modulate the functional phenotype and magnitude of immune responses to antigens.

In one aspect, the invention includes a lipid nanoparticle (LNP) comprising: (a) a first nucleoside-modified ribonucleic acid (RNA) encoding an antigen; (b) a second nucleoside-modified RNA encoding a cytokine or immune receptor (such as but not limited to a cytokine receptor); and (c) an ionizable lipid; wherein the LNP is capable of eliciting a modulated immune response against the antigen in a subject. In some embodiments, the modulated immune response comprises an enhanced immune response. In some embodiments, the modulated immune response comprises a decreased immune response. In certain embodiments, the modulated immune response is tissue-specific. In some embodiments, the cytokine or immune receptor (such as but not limited to cytokine receptor) is capable of modulating the magnitude and/or functional phenotype of the immune response.

In one aspect, the invention includes a method of eliciting a modulated immune response against an antigen in a subject, the method comprising administering to the subject a pharmaceutical composition comprising a lipid nanoparticle (LNP) and at least one pharmaceutically acceptable carrier, diluent, and/or excipient, wherein the LNP comprises: (a) a first nucleoside-modified ribonucleic acid (RNA) encoding the antigen; (b) a second nucleoside-modified RNA encoding a cytokine or immune receptor (such as but not limited to a cytokine receptor); and (c) an ionizable lipid; wherein the LNP elicits a modulated immune response against the antigen in the subject. In some embodiments, the modulated immune response comprises an enhanced immune response. In some embodiments, the modulated immune response comprises a decreased immune response. In certain embodiments, the modulated immune response is tissue-specific. In some embodiments, the cytokine or immune receptor (such as but not limited to cytokine receptor) modulates the magnitude and/or functional phenotype of the immune response.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice of and/or for the testing of the present invention, the preferred materials and methods are described herein. In describing and claiming the present invention, the following terminology will be used according to how it is defined, where a definition is provided.

It is to be understood that the terminology used herein is for the purpose of describing particular embodiments only, and is not intended to be limiting.

It is also to be understood that the methods described in this disclosure are not limited to particular methods and experimental conditions disclosed herein as such methods and conditions may vary.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook, and Harlow et al., Antibodies: A Laboratory Manual, Chapter 14, Cold Spring Harbor Laboratory, Cold Spring Harbor (2013, 2nd edition).

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

So that the disclosure may be more readily understood, select terms are defined below.

The articles “a” and “an” are used herein to refer to one or to more than one (i.e., to at least one) of the grammatical object of the article. By way of example, “an element” means one element or more than one element.

“About,” as used herein, when referring to a measurable value such as an amount, a temporal duration, and the like, is meant to encompass variations of ±20% or ±10%, in some instances ±5%, in some instances ±1%, and in some instance ±0.1% from the specified value, as such variations are appropriate to perform the disclosed methods.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et ak, 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations, κ and λ light chains refer to the two major antibody light chain isotypes.

By the term “synthetic antibody” as used herein, is meant an antibody, which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned) or other technology, which is available and well known in the art.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequence or a partial nucleotide sequence encoding a protein that elicits an adaptive immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated or synthesized or can be derived from a biological sample. Such a biological sample can include, but is not limited to a tissue sample, a tumor sample, a cell or a biological fluid.

The term “adjuvant” as used herein is defined as any molecule to enhance an antigen-specific adaptive immune response.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) RNA, and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Immunogen,” as the term is used herein, is synonymous with “antigen” and refers to any substance introduced into the body in order to generate an immune response. That substance can be a biologic molecule, such as a polypeptide or protein, and can be encoded by a vector, such as DNA, mRNA, or a virus.

“Immune response,” as the term is used herein, means a process involving the activation and/or induction of an effector function in, by way of non-limiting examples, a T cell, B cell, natural killer (NK) cell, and/or antigen-presenting cells (APC). Thus, an immune response, as would be understood by the skilled artisan, includes, but is not limited to, any detectable antigen-specific activation and/or induction of a helper T cell or cytotoxic T cell activity or response, production of antibodies, antigen presenting cell activity or infiltration, macrophage activity or infiltration, neutrophil activity or infiltration, and the like.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleosides (nucleobase bound to ribose or deoxyribose sugar via N-glycosidic linkage) are used. “A” refers to adenosine, “C” refers to cytidine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, such as, a human.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. In addition, the nucleotide sequence may contain modified nucleosides that are capable of being translated by translational machinery in a cell. For example, an mRNA where all of the uridines have been replaced with pseudouridine, 1-methyl psuedouridine, or another modified nucleoside.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

The terms “patient,” “subject,” “individual,” and the like are used interchangeably herein, and refer to any animal, or cells thereof whether in vitro or in situ, amenable to the methods described herein. In certain non-limiting embodiments, the patient, subject, or individual is a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In certain instances, the polynucleotide or nucleic acid of the invention is a “nucleoside-modified nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27: 196-197).

The term “pseudouridine” refers to the natural product which is a C-glycosyl pyrimidine that consists of uracil having a beta-D-ribofuranosyl residue attached at position 5 (i.e., 5-(beta-D-Ribofuranosyl)uracil). In some embodiments, the term refers to m¹acp³ψ (l-methyl-3-(3-amino-3-carboxypropyl) pseudouridine. In another embodiment, the term refers to m¹Ψ (1-methylpseudouridine). In another embodiment, the term refers to ψm (2′-O-methylpseudouridine. In another embodiment, the term refers to m⁵D (5-methyldihydrouridine). In another embodiment, the term refers to m³ψ (3-methylpseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the present invention.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

Cytokines include, but are not limited to, chemokines, interleukins (such as, IL-1, IL-2, IL-6, IL-7, IL-10, IL-12, IL-15, IL-18, IL-21, IL-23, and IL-27), interferons (IFN-α, IFN-β, and IFN-γ), tumor necrosis factors (TNF-α and TNF-β) and their family members, and transforming growth factor (TGF-β), natural variants thereof, engineered cytokine variants (e.g., super/hyper IL-15 and decoy-resistant IL-18 (DR-18)), and novel cytokine mimetics.

The term “promoter” as used herein is defined as a DNA sequence recognized by the synthetic machinery of the cell, or introduced synthetic machinery, required to initiate the specific transcription of a polynucleotide sequence. For example, the promoter that is recognized by bacteriophage RNA polymerase and is used to generate the mRNA by in vitro transcription.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “therapeutic” as used herein means a treatment and/or prophylaxis. A therapeutic effect is obtained by suppression, diminution, remission, or eradication of at least one sign or symptom of a disease or disorder.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

### DESCRIPTION

The present invention relates to compositions and methods for eliciting a modulated immune response against an antigen in a subject. The invention is not limited to any particular antigen and one skilled in the art will understand that the invention pertains to any antigen. In certain embodiments, the antigen is derived from a pathogen.

In one aspect, the invention provides a lipid nanoparticle (LNP). In certain embodiments, the LNP comprises a first nucleoside-modified ribonucleic acid (RNA) encoding an antigen. In certain embodiments, the LNP comprises a second nucleoside-modified RNA encoding a cytokine or immune receptor (such as but not limited to a cytokine receptor). In certain embodiments, the LNP comprises an ionizable lipid. In certain embodiments, the LNP is capable of eliciting a modulated immune response against the antigen in a subject. In certain embodiments, the LNP is capable of eliciting an enhanced immune response against the antigen in a subject. In some embodiments, the enhanced immune response in a subject comprises an augmented CD8⁺ T cell response in the subject compared to a CD8⁺ T cell response in a subject administered a control LNP lacking the second nucleoside-modified RNA encoding a cytokine or immune receptor (such as but not limited to a cytokine receptor). In certain embodiments, the LNP is capable of eliciting a reduced immune response against the antigen in a subject. In some embodiments, the reduced immune response in a subject comprises a decreased CD8⁺ T cell response in the subject compared to a CD8⁺ T cell response in a subject administered a control LNP lacking the second nucleoside-modified RNA encoding a cytokine or immune receptor (such as but not limited to a cytokine receptor). In certain embodiments, the decreased CD8⁺ T cell response is tissue-specific. In some embodiments, the modulated immune response comprises reduced memory CD8⁺ T cells in spleen and peripheral blood and increased CD8⁺ T cells in mucosal sites (i.e., gut and lung). In some embodiments, the cytokine or immune receptor (such as but not limited to cytokine receptor) is capable of modulating the magnitude and/or functional phenotype of the immune response.

In another aspect, the invention provides a method of eliciting a modulated immune response against an antigen in a subject. In certain embodiments, the method comprises administering to the subject a pharmaceutical composition comprising a lipid nanoparticle (LNP) and at least one pharmaceutically acceptable carrier, diluent, and/or excipient. In certain embodiments, the LNP comprises a first nucleoside-modified ribonucleic acid (RNA) encoding the antigen. In certain embodiments, the LNP comprises a second nucleoside-modified RNA encoding a cytokine or immune receptor (such as but not limited to a cytokine receptor). In certain embodiments, the LNP comprises an ionizable lipid. In certain embodiments, the LNP elicits an enhanced immune response against the antigen in the subject. In some embodiments, the enhanced immune response in the subject comprises an augmented CD8⁺ T cell response in the subject compared to a CD8⁺ T cell response in a subject administered a control composition lacking the second nucleoside-modified RNA encoding a cytokine or immune receptor (such as but not limited to a cytokine receptor). In some embodiments, the LNP elicits a reduced immune response against the antigen in the subject. In some embodiments, the reduced immune response in the subject comprises a decreased CD8⁺ T cell response in the subject compared to a CD8⁺ T cell response in a subject administered a control composition lacking the second nucleoside-modified RNA encoding a cytokine or immune receptor (such as but not limited to a cytokine receptor). In certain embodiments, the decreased CD8⁺ T cell response is tissue-specific. In some embodiments, the modulated immune response comprises reduced memory CD8⁺ T cells in spleen and peripheral blood and increased CD8⁺ T cells in mucosal sites (i.e., gut and lung). In some embodiments, the cytokine or immune receptor (such as but not limited to cytokine receptor) modulates the magnitude and/or functional phenotype of the immune response.

Modulation of the magnitude and functional phenotype of the immune response can be achieved by selecting the appropriate cytokine or immune receptor (such as but not limited to a cytokine receptor) for a given LNP vaccine, depending on the desired effect. CD8⁺ T cell subsets can be defined by the localization of the cells to particular tissues (i.e. skin/gut vs. lymph nodes/spleen), as well as by cell surface markers which correlate with the propensity of those cells to become long-lived protective memory cells. Thus, in certain embodiments, manipulating the mixture of CD8⁺ T cells that are generated from the immunization (i.e., by inclusion of mRNA encoding one or more appropriate cytokines or immune receptors (such as but not limited to cytokine receptors) drives increased retention of the CD8⁺ T cells in specific tissues for localized protection against future challenge. In other embodiments, the cytokine or immune receptor (such as but not limited to a cytokine receptor) improves the number of memory cells present within a host following vaccination.

In certain embodiments, the cytokine or immune receptor (such as but not limited to a cytokine receptor) suppresses or bypasses certain inflammatory responses that are not beneficial to the patient, such as inflammatory responses which result in side effects that are not important for generating protective immunity. In some embodiments, the cytokine or immune receptor (such as but not limited to a cytokine receptor) prevents certain immune responses from being utilized in order to skew the antigen-specific response towards localizing to at least one particular tissue (e.g., a barrier tissue) rather than systemically to increase the number of cells that reside in said tissue(s).

Additionally, it is contemplated herein that therapeutic administration of the vaccines described herein would boost the number of circulating effector T cells to generate a very robust response. Although such a robust T cell response may be significantly shorter lived (compared to cases of preventative vaccine administration), the primary goal of therapeutic administration would be to eliminate the disease as quickly as possible, and not necessarily to enhance memory T cell populations.

### Vaccines

In some embodiments, the present invention provides a pharmaceutical composition (e.g., an immunogenic composition) for inducing a modulated immune response against an antigen in a subject. For example, in some embodiments, the immunogenic composition is a vaccine. For a composition to be useful as a vaccine, the composition must induce an immune response to the antigen in a cell, a tissue, or a mammal (e.g., a human). In certain instances, the vaccine induces a protective immune response in the mammal. The pharmaceutical compositions (e.g., vaccines) of the invention comprise an LNP which elicits a modulated immune response against an antigen in a subject. In some embodiments, the LNP elicits an enhanced immune response against the antigen in the subject. In some embodiments, the LNP elicits a reduced immune response against the antigen in the subject. In some embodiments, the LNP modulates the magnitude and/or functional phenotype of the immune response as described herein.

In the context of the present invention, the term “vaccine” refers to a composition that induces an immune response upon inoculation into animals, e.g., vertebrate animals. In some embodiments, the induced immune response provides protective immunity.

In certain aspects, a vaccine of the present invention is an LNP-nucleic acid vaccine which may vary in its composition of nucleic acid and/or lipid components. In a non-limiting example, the LNP of the invention comprises an ionizable lipid. In some embodiments, the LNP comprises an ionizable lipid and one or more other lipid(s). In some embodiments, the LNP comprises a first nucleoside-modified RNA encoding an antigen. In some embodiments, the first nucleoside-modified RNA encodes more than one antigen. In some embodiments, each antigen is encoded by a corresponding nucleoside-modified RNA that is comprised within the LNP and that encodes the antigen. In some embodiments, the LNP comprises a second nucleoside-modified RNA encoding a cytokine or immune receptor (such as but not limited to a cytokine receptor). In certain embodiments, the second nucleoside-modified RNA encodes more than one cytokine or immune receptor (such as but not limited to a cytokine receptor). In some embodiments, each cytokine or immune receptor (such as but not limited to a cytokine receptor) is encoded by a corresponding nucleoside-modified RNA that is comprised within the LNP and that encodes the cytokine or immune receptor (such as but not limited to a cytokine receptor).

An LNP vaccine of the present invention, and its various components, may be prepared and/or administered by any method disclosed herein or as would be known to one of ordinary skill in the art, in light of the present disclosure.

The induction of the immunity by the expression of the antigen can be detected by observing in vivo or in vitro the response of all or any part of the immune system in the host against the antigen.

For example, a method for detecting the induction of cytotoxic T lymphocytes is well known. A foreign substance that enters the living body is presented to T cells and B cells by the action of APCs. CD8⁺ T cells that respond to the antigen presented by APC in an antigen specific manner proliferate. As the CD8⁺ T cells proliferate they differentiate into cytotoxic T cells (also referred to as cytotoxic T lymphocytes or CTLs) due to stimulation by the antigen. This process is referred to herein as “activation” of T cells. Therefore, CTL induction by an epitope of a polypeptide or peptide or combinations thereof can be evaluated by presenting an epitope of a polypeptide or peptide or combinations thereof to a T cell by APC, and detecting the induction of cytotoxicity. Furthermore, APCs have the effect of activating B cells, CD4⁺ T cells, CD8⁺ T cells, macrophages, eosinophils, and NK cells, as well as producing cytokines that can influence the functionality of the aforementioned cells.

A method for evaluating the inducing action of CTL using dendritic cells (DCs) as APC is well known in the art. DC is a representative APC having a robust CTL inducing action among APCs. In the methods of the invention, the epitope of a polypeptide or peptide or combinations thereof is initially expressed by the DC and then this DC contacts T cells. Detection of T cells having cytotoxic effects against the cells of interest after the contact with DC shows that the epitope of a polypeptide or peptide or combinations thereof has an activity of inducing the differentiation of cytotoxic T cells.

Furthermore, the induced immune response can be also examined by measuring IFN-gamma produced and released by CTL in the presence of antigen-presenting cells that carry immobilized peptide or combination of peptides by visualizing using anti-IFN-gamma antibodies, such as an ELISPOT assay, or via flow cytometry.

Apart from DC, peripheral blood mononuclear cells (PBMCs) may also be used as the APC. The induction of CTL is reported to be enhanced by culturing PBMC in the presence of GM-CSF and IL-4. Similarly, CTL activity has been shown to be induced by culturing PBMC in the presence of keyhole limpet hemocyanin (KLH) and IL-7.

The antigens confirmed to possess CTL-inducing activity by these methods are antigens that can be presented by DCs to T cells and subsequently induce CTL activity. Furthermore, CTLs that have acquired cytotoxicity due to presentation of the antigen by APC can be also used as vaccines against antigen-associated disorders.

The induction of immunity by expression of the antigen can be further confirmed by observing the induction of antibody production against the antigen. For example, when antibodies against an antigen are induced in a laboratory animal immunized with the composition encoding the antigen, and when antigen-associated pathology is suppressed by those antibodies, the composition is determined to induce immunity.

The induction of immunity by expression of the antigen can be further confirmed by observing the induction of CD4⁺ T cells. CD4⁺ T cells can also lyse target cells, but mainly supply help in the induction of other types of immune responses, including CTL and antibody generation. The type of CD4⁺ T cell help can be characterized, as Th1, Th2, Th9, Th17, T regulatory, or T follicular helper (T_{fh}) cells. Each subtype of CD4⁺ T cell supplies help to certain types of immune responses. In some embodiments, the composition selectively induces T follicular helper cells, which drive potent antibody responses.

The therapeutic compounds or compositions of the invention may be administered prophylactically (i.e., to prevent a disease or disorder) or therapeutically (i.e., to treat a disease or disorder) to subjects suffering from, or at risk of (or susceptible to) developing the disease or disorder. Such subjects may be identified using standard clinical methods. In the context of the present invention, prophylactic administration occurs prior to the manifestation of overt clinical symptoms of disease, such that a disease or disorder is prevented or alternatively delayed in its progression. In the context of the field of medicine, the term “prevent” encompasses any activity, which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

### Cytokines and Immune Receptors (Such as but not Limited to Cytokine Receptors)

The present invention is based, in part, on the unexpected discovery that LNP vaccines comprising a nucleoside-modified RNA encoding an antigen and a nucleoside-modified RNA encoding a cytokine (i.e., IL-27 or IL-12) enhance the immune response of LNP-mRNA vaccines against the antigen by augmenting the CD8⁺ T cell response in the subject compared to a CD8⁺ T cell response in a subject administered a control composition lacking the nucleoside-modified RNA encoding a cytokine.

The invention includes an LNP and compositions comprising the LNP (e.g. pharmaceutical compositions and/or vaccines), wherein the LNP is capable of eliciting a modulated immune response against an antigen in a subject. In some embodiments, the LNP comprises a first nucleoside-modified RNA encoding an antigen and a second nucleoside-modified RNA encoding a cytokine or immune receptor (such as but not limited to a cytokine receptor). In some embodiments, the LNP does not comprise a further nucleoside-modified RNA encoding an additional agent. In some embodiments, the LNP comprises a further nucleoside-modified RNA encoding an additional agent.

In some embodiments, the additional agent comprises a cytokine. In some embodiments, the additional agent comprises a cytokine which is not encoded by the second nucleoside-modified RNA. In some embodiments, the additional agent comprises an immune receptor (such as but not limited to a cytokine receptor). In some embodiments, the additional agent comprises an immune receptor (such as but not limited to a cytokine receptor) which is not encoded by the second nucleoside-modified RNA. In some embodiments, the additional agent comprises an adjuvant. In some embodiments, the additional agent comprises an adjuvant which is not encoded by the second nucleoside-modified RNA.

In some embodiments, the LNP further comprises at least one ionizable lipid. In some embodiments, the modulated immune response is an enhanced immune response. In some embodiments, the modulated immune response is a decreased immune response. In certain embodiments, the modulated immune response is tissue-specific. In some embodiments, the cytokine or immune receptor (such as but not limited to a cytokine receptor) is capable of modulating the magnitude and/or functional phenotype of the immune response.

In some embodiments, the cytokine is selected from the group consisting of a chemokine, an interleukin (IL), an interferon (IFN), any tumor necrosis factor (TNF) family member, a transforming growth factor (TGF), and any combination thereof.

In some embodiments, the second nucleoside-modified RNA encodes a chemokine. In some embodiments, the second nucleoside-modified RNA encodes an interleukin (IL). In some embodiments, the second nucleoside-modified RNA encodes an interferon (IFN). In some embodiments, the second nucleoside-modified RNA encodes a tumor necrosis factor (TNF). In some embodiments, the second nucleoside-modified RNA encodes a transforming growth factor (TGF).

In some embodiments, the cytokine is selected from the group consisting of IL-1, IL-2, IL-6, IL-7, IL-10, IL-12, IL-15, IL-18, IL-21, IL-23, IL-27, IL-33, Type-I IFNs (IFN-α, IFN-β), IFN-γ, TNF-α, TNF-β, TGF-β, super/hyper IL-15, DR-18, and any combination thereof.

In some embodiments, the second nucleoside-modified RNA encodes IL-1. In some embodiments, the second nucleoside-modified RNA encodes IL-2. In some embodiments, the second nucleoside-modified RNA encodes IL-6. In some embodiments, the second nucleoside-modified RNA encodes IL-7. In some embodiments, the second nucleoside-modified RNA encodes IL-10. In some embodiments, the second nucleoside-modified RNA encodes IL-12. In some embodiments, the second nucleoside-modified RNA encodes IL-15. In some embodiments, the second nucleoside-modified RNA encodes IL-18. In some embodiments, the second nucleoside-modified RNA encodes IL-21. In some embodiments, the second nucleoside-modified RNA encodes IL-23. In some embodiments, the second nucleoside-modified RNA encodes IL-27. In some embodiments, the second nucleoside-modified RNA encodes IL-33. In some embodiments, the second nucleoside-modified RNA encodes a Type-I IFN. In some embodiments, the second nucleoside-modified RNA encodes IFN-α. In some embodiments, the second nucleoside-modified RNA encodes IFN-β. In some embodiments, the second nucleoside-modified RNA encodes IFN-γ. In some embodiments, the second nucleoside-modified RNA encodes TNF-α. In some embodiments, the second nucleoside-modified RNA encodes TNF-β. In some embodiments, the second nucleoside-modified RNA encodes TGF-β.

In some embodiments, the cytokine comprises an engineered cytokine variant. In certain embodiments, the engineered cytokine variant comprises a super/hyper IL-15 (i.e., a fusion protein comprising IL-15 connected to IL-15Rα-sushi domain via a flexible linker, non-limiting examples of which are described in Mortier, et al., J. Biol. Chem. 2006, 281(3):1612-1619). In certain embodiments, the engineered cytokine variant comprises a mutant IL-12 with alterations that impact its affinity to receptors (i.e., IL-12 muteins examples of which are described in Glassman, et al., Cell 2021, 184(4):983-999.e24). In certain embodiments, the engineered cytokine variant comprises decoy-resistant IL-18 (DR-18). DR-18 maintains signaling potential but is impervious to inhibition by IL-18BP, a high-affinity IL-18 decoy receptor (Zhou, et al., Nature, 2020, 583:609-614).

In some embodiments, the second nucleoside-modified RNA encodes an engineered cytokine variant. In some embodiments, the second nucleoside-modified RNA encodes super/hyper IL-15. In some embodiments, the second nucleoside-modified RNA encodes IL-12 variants. In some embodiments, the second nucleoside-modified RNA encodes DR-18.

In certain embodiments, the immune receptor (such as but not limited to a cytokine receptor) comprises a soluble immune receptor/cytokine receptor (i.e., an immune receptor and/or cytokine receptor that is engineered to be soluble). In some embodiments, the soluble cytokine receptor comprises soluble IL-15Rα. In some embodiments, the soluble cytokine receptor comprises soluble IL-1Rα. Soluble versions of cytokine receptors, such as soluble IL-15Rα and soluble IL-1Rα, consist essentially of the extracellular domain of the native cytokine receptor. Soluble cytokine receptors are known in the art and can be readily generated by those of skill in the art (see, e.g., Wei, et al., J. Immunol., 2001, 167(1):277-282).

In some embodiments, the second nucleoside-modified RNA encodes soluble IL-15Rα. In some embodiments, the second nucleoside-modified RNA encodes soluble IL-1Rα.

In certain embodiments, the immune receptor/cytokine receptor comprises an immune receptor/cytokine receptor decoy. In some embodiments, the cytokine receptor decoy comprises IL-18 binding protein (IL-18BP).

In some embodiments, the second nucleoside-modified RNA encodes an immune receptor/cytokine receptor decoy. In some embodiments, the second nucleoside-modified RNA encodes IL-18 binding protein (IL-18BP).

In some embodiments, the cytokine or immune receptor (such as but not limited to a cytokine receptor) comprises more than one domain or subunit (e.g., two or more subunits) of the cytokine or immune receptor (such as but not limited to a cytokine receptor). In certain embodiments, the two or more subunits of the cytokine or immune receptor (such as but not limited to a cytokine receptor) can be expressed as a fusion protein. In some embodiments, the second nucleoside-modified RNA encodes a first subunit of the cytokine or immune receptor (such as but not limited to a cytokine receptor) and a second subunit of the cytokine or immune receptor (such as but not limited to a cytokine receptor), wherein the first subunit and the second subunit are linked to each other via a flexible linker, such as a glycine serine linker. In other embodiments, the LNP comprises a distinct nucleoside-modified RNA for encoding each individual subunit of the cytokine or immune receptor (such as but not limited to a cytokine receptor).

The nucleotide sequence(s) encoding the cytokine or immune receptor (such as but not limited to a cytokine receptor) may be derived from any animal which expresses the cytokine or immune receptor (such as but not limited to a cytokine receptor). Non-limiting examples include a mouse, a rat, a pig, a simian, and a human. In some embodiments, the LNP comprises a nucleoside modified RNA encoding a cytokine or immune receptor (such as but not limited to a cytokine receptor) from a mammal (e.g., a human), and said LNP is used in a method of modulating an immune response against an antigen in the same mammal (e.g., a human). In other embodiments, the LNP comprises a nucleoside modified RNA encoding a cytokine or immune receptor (such as but not limited to a cytokine receptor) from a mammal (e.g., a human), and said LNP is used in a method of modulating an immune response against an antigen in the same mammal (e.g., a mouse). In certain embodiments, the cytokine or immune receptor (such as but not limited to a cytokine receptor) is an engineered and/or variant version of a naturally-occurring cytokine or immune receptor (such as but not limited to a cytokine receptor). Such engineered cytokines and immune receptors (such as but not limited to a cytokine receptors) include recombinant, edited, tagged, and/or fusion cytokines and immune receptors (such as but not limited to a cytokine receptors).

In certain embodiments, the cytokine or immune receptor (such as but not limited to a cytokine receptor) is a variant of a naturally occurring cytokine or immune receptor (such as but not limited to a cytokine receptor). Tolerable variations of the nucleotide and amino acid sequences of the cytokine or immune receptor (such as but not limited to a cytokine receptor) (e.g., cytokine subunit sequences) will be known to those of skill in the art. For example, in certain embodiments the cytokine or immune receptor (such as but not limited to a cytokine receptor) or a subunit thereof comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any naturally-occurring or known reference amino acid sequence of the cytokine or immune receptor (such as but not limited to a cytokine receptor) or subunit thereof.

In other embodiments, the cytokine or immune receptor (such as but not limited to a cytokine receptor) or a subunit thereof is encoded by a nucleotide sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any naturally-occurring or known reference nucleotide sequence encoding the cytokine or immune receptor (such as but not limited to a cytokine receptor) or subunit thereof.

In some embodiments, the cytokine comprises IL-27. IL-27 comprises an Epstein-Barr virus-induced gene 3 (Ebi3) subunit and an IL-27p28 subunit.

In some embodiments, the LNP comprises a nucleoside-modified RNA encoding an antigen, a nucleoside-modified RNA encoding an Epstein-Barr virus-induced gene 3 (Ebi3) subunit of IL-27, and a nucleoside-modified RNA encoding an IL-27p28 subunit of IL-27.

In some embodiments, the cytokine comprises an IL-27 fusion protein comprising an Epstein-Barr virus-induced gene 3 (Ebi3) subunit of IL-27 linked to an IL-27p28 subunit of IL-27 via a flexible linker (e.g., as described in Pflanz, et al. (2002) Immunity, 16, 779-790.).

In some embodiments, the second nucleoside-modified RNA encoding a cytokine or cytokine receptor encodes an Epstein-Barr virus-induced gene 3 (Ebi3) subunit of IL-27 linked to an IL-27p28 subunit of IL-27 via a flexible linker.

In some embodiments, the second nucleoside-modified RNA does not encode alpha-interferon. In some embodiments, the second nucleoside-modified RNA does not encode IFN-γ. In some embodiments, the second nucleoside-modified RNA does not encode platelet derived growth factor (PDGF). In some embodiments, the second nucleoside-modified RNA does not encode TNF-α. In some embodiments, the second nucleoside-modified RNA does not encode TNF-β. In some embodiments, the second nucleoside-modified RNA does not encode GM-CSF. In some embodiments, the second nucleoside-modified RNA does not encode epidermal growth factor (EGF). In some embodiments, the second nucleoside-modified RNA does not encode cutaneous T cell-attracting chemokine (CTACK). In some embodiments, the second nucleoside-modified RNA does not encode epithelial thymus-expressed chemokine (TECK). In some embodiments, the second nucleoside-modified RNA does not encode mucosae-associated epithelial chemokine (MEC). In some embodiments, the second nucleoside-modified RNA does not encode IL-12. In some embodiments, the second nucleoside-modified RNA does not encode IL-15. In some embodiments, the second nucleoside-modified RNA does not encode MHC. In some embodiments, the second nucleoside-modified RNA does not encode CD80. In some embodiments, the second nucleoside-modified RNA does not encode CD86. In some embodiments, the second nucleoside-modified RNA does not encode MCP-1. In some embodiments, the second nucleoside-modified RNA does not encode MIP-1α. In some embodiments, the second nucleoside-modified RNA does not encode MIP-1β. In some embodiments, the second nucleoside-modified RNA does not encode IL-8. In some embodiments, the second nucleoside-modified RNA does not encode RANTES. In some embodiments, the second nucleoside-modified RNA does not encode L-selectin. In some embodiments, the second nucleoside-modified RNA does not encode P-selectin. In some embodiments, the second nucleoside-modified RNA does not encode E-selectin. In some embodiments, the second nucleoside-modified RNA does not encode CD34. In some embodiments, the second nucleoside-modified RNA does not encode GlyCAM-1. In some embodiments, the second nucleoside-modified RNA does not encode MadCAM-1. In some embodiments, the second nucleoside-modified RNA does not encode LFA-I. In some embodiments, the second nucleoside-modified RNA does not encode VLA-I. In some embodiments, the second nucleoside-modified RNA does not encode Mac-1. In some embodiments, the second nucleoside-modified RNA does not encode pl50.95. In some embodiments, the second nucleoside-modified RNA does not encode PEC AM. In some embodiments, the second nucleoside-modified RNA does not encode ICAM-1. In some embodiments, the second nucleoside-modified RNA does not encode ICAM-2. In some embodiments, the second nucleoside-modified RNA does not encode ICAM-3. In some embodiments, the second nucleoside-modified RNA does not encode CD2. In some embodiments, the second nucleoside-modified RNA does not encode LFA-3. In some embodiments, the second nucleoside-modified RNA does not encode M-CSF. In some embodiments, the second nucleoside-modified RNA does not encode G-CSF. In some embodiments, the second nucleoside-modified RNA does not encode IL-4. In some embodiments, the second nucleoside-modified RNA does not encode mutant forms of IL-18. In some embodiments, the second nucleoside-modified RNA does not encode CD40. In some embodiments, the second nucleoside-modified RNA does not encode CD40L. In some embodiments, the second nucleoside-modified RNA does not encode vascular growth factor. In some embodiments, the second nucleoside-modified RNA does not encode fibroblast growth factor. In some embodiments, the second nucleoside-modified RNA does not encode IL-7. In some embodiments, the second nucleoside-modified RNA does not encode nerve growth factor. In some embodiments, the second nucleoside-modified RNA does not encode vascular endothelial growth factor. In some embodiments, the second nucleoside-modified RNA does not encode Fas. In some embodiments, the second nucleoside-modified RNA does not encode TNF receptor. In some embodiments, the second nucleoside-modified RNA does not encode Fit. In some embodiments, the second nucleoside-modified RNA does not encode Apo-1. In some embodiments, the second nucleoside-modified RNA does not encode p55. In some embodiments, the second nucleoside-modified RNA does not encode WSL-I. In some embodiments, the second nucleoside-modified RNA does not encode DR3. In some embodiments, the second nucleoside-modified RNA does not encode TRAMP. In some embodiments, the second nucleoside-modified RNA does not encode Apo-3. In some embodiments, the second nucleoside-modified RNA does not encode AIR. In some embodiments, the second nucleoside-modified RNA does not encode LARD. In some embodiments, the second nucleoside-modified RNA does not encode NGRF. In some embodiments, the second nucleoside-modified RNA does not encode DR4. In some embodiments, the second nucleoside-modified RNA does not encode DR5. In some embodiments, the second nucleoside-modified RNA does not encode KILLER. In some embodiments, the second nucleoside-modified RNA does not encode TRAIL-R2. In some embodiments, the second nucleoside-modified RNA does not encode TRICK2. In some embodiments, the second nucleoside-modified RNA does not encode DR6. In some embodiments, the second nucleoside-modified RNA does not encode Caspase ICE. In some embodiments, the second nucleoside-modified RNA does not encode Fos. In some embodiments, the second nucleoside-modified RNA does not encode c-jun. In some embodiments, the second nucleoside-modified RNA does not encode Sp-I. In some embodiments, the second nucleoside-modified RNA does not encode Ap-I. In some embodiments, the second nucleoside-modified RNA does not encode Ap-2. In some embodiments, the second nucleoside-modified RNA does not encode p38. In some embodiments, the second nucleoside-modified RNA does not encode p65Rel. In some embodiments, the second nucleoside-modified RNA does not encode MyD88. In some embodiments, the second nucleoside-modified RNA does not encode IRAK. In some embodiments, the second nucleoside-modified RNA does not encode TRAF6. In some embodiments, the second nucleoside-modified RNA does not encode IkB. In some embodiments, the second nucleoside-modified RNA does not encode Inactive NIK. In some embodiments, the second nucleoside-modified RNA does not encode SAP K. In some embodiments, the second nucleoside-modified RNA does not encode SAP-I. In some embodiments, the second nucleoside-modified RNA does not encode JNK. In some embodiments, the second nucleoside-modified RNA does not encode interferon response genes. In some embodiments, the second nucleoside-modified RNA does not encode NFkB. In some embodiments, the second nucleoside-modified RNA does not encode ax. In some embodiments, the second nucleoside-modified RNA does not encode TRAIL. In some embodiments, the second nucleoside-modified RNA does not encode TRAILrec. In some embodiments, the second nucleoside-modified RNA does not encode TRAILrecDRC 5. In some embodiments, the second nucleoside-modified RNA does not encode TRAIL-R3. In some embodiments, the second nucleoside-modified RNA does not encode TRAIL-R4. In some embodiments, the second nucleoside-modified RNA does not encode RANK. In some embodiments, the second nucleoside-modified RNA does not encode RANK ligand. In some embodiments, the second nucleoside-modified RNA does not encode OX40. In some embodiments, the second nucleoside-modified RNA does not encode OX40 ligand. In some embodiments, the second nucleoside-modified RNA does not encode NKG2D. In some embodiments, the second nucleoside-modified RNA does not encode MICA. In some embodiments, the second nucleoside-modified RNA does not encode MICB. In some embodiments, the second nucleoside-modified RNA does not encode NKG2A. In some embodiments, the second nucleoside-modified RNA does not encode NKG2B. In some embodiments, the second nucleoside-modified RNA does not encode NKG2C. In some embodiments, the second nucleoside-modified RNA does not encode NKG2E. In some embodiments, the second nucleoside-modified RNA does not encode NKG2F. In some embodiments, the second nucleoside-modified RNA does not encode TAP 1. In some embodiments, the second nucleoside-modified RNA does not encode TAP2. In some embodiments, the second nucleoside-modified RNA does not encode anti-CTLA4-sc. In some embodiments, the second nucleoside-modified RNA does not encode anti-LAG3-Ig. In some embodiments, the second nucleoside-modified RNA does not encode anti-TIM3-Ig. In some embodiments, the second nucleoside-modified RNA encodes IFN-α. In some embodiments, the second nucleoside-modified RNA encodes IFN-γ. In some embodiments, the second nucleoside-modified RNA encodes platelet derived growth factor (PDGF). In some embodiments, the second nucleoside-modified RNA encodes TNFα. In some embodiments, the second nucleoside-modified RNA encodes TNFβ. In some embodiments, the second nucleoside-modified RNA encodes GM-CSF. In some embodiments, the second nucleoside-modified RNA encodes epidermal growth factor (EGF). In some embodiments, the second nucleoside-modified RNA encodes cutaneous T cell-attracting chemokine (CTACK). In some embodiments, the second nucleoside-modified RNA encodes epithelial thymus-expressed chemokine (TECK). In some embodiments, the second nucleoside-modified RNA encodes mucosae-associated epithelial chemokine (MEC). In some embodiments, the second nucleoside-modified RNA encodes IL-12. In some embodiments, the second nucleoside-modified RNA encodes IL-15. In some embodiments, the second nucleoside-modified RNA encodes MHC. In some embodiments, the second nucleoside-modified RNA encodes CD80. In some embodiments, the second nucleoside-modified RNA encodes CD86. In some embodiments, the second nucleoside-modified RNA encodes MCP-1. In some embodiments, the second nucleoside-modified RNA encodes MIP-1α. In some embodiments, the second nucleoside-modified RNA encodes MIP-1β. In some embodiments, the second nucleoside-modified RNA encodes IL-8. In some embodiments, the second nucleoside-modified RNA encodes RANTES. In some embodiments, the second nucleoside-modified RNA encodes L-selectin. In some embodiments, the second nucleoside-modified RNA encodes P-selectin. In some embodiments, the second nucleoside-modified RNA encodes E-selectin. In some embodiments, the second nucleoside-modified RNA encodes CD34. In some embodiments, the second nucleoside-modified RNA encodes GlyCAM-1. In some embodiments, the second nucleoside-modified RNA encodes MadCAM-1. In some embodiments, the second nucleoside-modified RNA encodes LFA-1. In some embodiments, the second nucleoside-modified RNA encodes VLA-1. In some embodiments, the second nucleoside-modified RNA encodes Mac-1. In some embodiments, the second nucleoside-modified RNA encodes p150.95. In some embodiments, the second nucleoside-modified RNA encodes PECAM. In some embodiments, the second nucleoside-modified RNA encodes ICAM-1. In some embodiments, the second nucleoside-modified RNA encodes ICAM-2. In some embodiments, the second nucleoside-modified RNA encodes ICAM-3. In some embodiments, the second nucleoside-modified RNA encodes CD2. In some embodiments, the second nucleoside-modified RNA encodes LFA-3. In some embodiments, the second nucleoside-modified RNA encodes M-CSF. In some embodiments, the second nucleoside-modified RNA encodes G-CSF. In some embodiments, the second nucleoside-modified RNA encodes IL-4. In some embodiments, the second nucleoside-modified RNA encodes mutant forms of IL-18. In some embodiments, the second nucleoside-modified RNA encodes CD40. In some embodiments, the second nucleoside-modified RNA encodes CD40L. In some embodiments, the second nucleoside-modified RNA encodes vascular growth factor. In some embodiments, the second nucleoside-modified RNA encodes fibroblast growth factor. In some embodiments, the second nucleoside-modified RNA encodes IL-7. In some embodiments, the second nucleoside-modified RNA encodes nerve growth factor. In some embodiments, the second nucleoside-modified RNA encodes vascular endothelial growth factor. In some embodiments, the second nucleoside-modified RNA encodes Fas. In some embodiments, the second nucleoside-modified RNA encodes TNF receptor. In some embodiments, the second nucleoside-modified RNA encodes Fit. In some embodiments, the second nucleoside-modified RNA encodes Apo-1. In some embodiments, the second nucleoside-modified RNA encodes p55. In some embodiments, the second nucleoside-modified RNA encodes WSL-I. In some embodiments, the second nucleoside-modified RNA encodes DR3. In some embodiments, the second nucleoside-modified RNA encodes TRAMP. In some embodiments, the second nucleoside-modified RNA encodes Apo-3. In some embodiments, the second nucleoside-modified RNA encodes AIR. In some embodiments, the second nucleoside-modified RNA encodes LARD. In some embodiments, the second nucleoside-modified RNA encodes NGRF. In some embodiments, the second nucleoside-modified RNA encodes DR4. In some embodiments, the second nucleoside-modified RNA encodes DR5. In some embodiments, the second nucleoside-modified RNA encodes KILLER. In some embodiments, the second nucleoside-modified RNA encodes TRAIL-R2. In some embodiments, the second nucleoside-modified RNA encodes TRICK2. In some embodiments, the second nucleoside-modified RNA encodes DR6. In some embodiments, the second nucleoside-modified RNA encodes Caspase ICE. In some embodiments, the second nucleoside-modified RNA encodes Fos. In some embodiments, the second nucleoside-modified RNA encodes c-jun. In some embodiments, the second nucleoside-modified RNA encodes Sp-I. In some embodiments, the second nucleoside-modified RNA encodes Ap-I. In some embodiments, the second nucleoside-modified RNA encodes Ap-2. In some embodiments, the second nucleoside-modified RNA encodes p38. In some embodiments, the second nucleoside-modified RNA encodes p65Rel. In some embodiments, the second nucleoside-modified RNA encodes MyD88. In some embodiments, the second nucleoside-modified RNA encodes IRAK. In some embodiments, the second nucleoside-modified RNA encodes TRAF6. In some embodiments, the second nucleoside-modified RNA encodes IkB. In some embodiments, the second nucleoside-modified RNA encodes Inactive NIK. In some embodiments, the second nucleoside-modified RNA encodes SAP K. In some embodiments, the second nucleoside-modified RNA encodes SAP-I. In some embodiments, the second nucleoside-modified RNA encodes JNK. In some embodiments, the second nucleoside-modified RNA encodes interferon response genes. In some embodiments, the second nucleoside-modified RNA encodes NFkB. In some embodiments, the second nucleoside-modified RNA encodes ax. In some embodiments, the second nucleoside-modified RNA encodes TRAIL. In some embodiments, the second nucleoside-modified RNA encodes TRAILrec. In some embodiments, the second nucleoside-modified RNA encodes TRAILrecDRC 5. In some embodiments, the second nucleoside-modified RNA encodes TRAIL-R3. In some embodiments, the second nucleoside-modified RNA encodes TRAIL-R4. In some embodiments, the second nucleoside-modified RNA encodes RANK. In some embodiments, the second nucleoside-modified RNA encodes RANK ligand. In some embodiments, the second nucleoside-modified RNA encodes OX40. In some embodiments, the second nucleoside-modified RNA encodes OX40 ligand. In some embodiments, the second nucleoside-modified RNA encodes NKG2D. In some embodiments, the second nucleoside-modified RNA encodes MICA. In some embodiments, the second nucleoside-modified RNA encodes MICB. In some embodiments, the second nucleoside-modified RNA encodes NKG2A. In some embodiments, the second nucleoside-modified RNA encodes NKG2B. In some embodiments, the second nucleoside-modified RNA encodes NKG2C. In some embodiments, the second nucleoside-modified RNA encodes NKG2E. In some embodiments, the second nucleoside-modified RNA encodes NKG2F. In some embodiments, the second nucleoside-modified RNA encodes TAP 1. In some embodiments, the second nucleoside-modified RNA encodes TAP2. In some embodiments, the second nucleoside-modified RNA encodes anti-CTLA4-sc. In some embodiments, the second nucleoside-modified RNA encodes anti-LAG3-Ig. In some embodiments, the second nucleoside-modified RNA encodes anti-TIM3-Ig.

In some embodiments, the additional agent does not comprise alpha-interferon. In some embodiments, the additional agent does not comprise gamma-interferon. In some embodiments, the additional agent does not comprise platelet derived growth factor (PDGF). In some embodiments, the additional agent does not comprise TNFa. In some embodiments, the additional agent does not comprise TNFP. In some embodiments, the additional agent does not comprise GM-CSF. In some embodiments, the additional agent does not comprise epidermal growth factor (EGF). In some embodiments, the additional agent does not comprise cutaneous T cell-attracting chemokine (CTACK). In some embodiments, the additional agent does not comprise epithelial thymus-expressed chemokine (TECK). In some embodiments, the additional agent does not comprise mucosae-associated epithelial chemokine (MEC). In some embodiments, the additional agent does not comprise IL-12. In some embodiments, the additional agent does not comprise IL-15. In some embodiments, the additional agent does not comprise MHC. In some embodiments, the additional agent does not comprise CD80. In some embodiments, the additional agent does not comprise CD86. In some embodiments, the additional agent does not comprise MCP-I. In some embodiments, the additional agent does not comprise MIP-1α. In some embodiments, the additional agent does not comprise MIP-1p. In some embodiments, the additional agent does not comprise IL-8. In some embodiments, the additional agent does not comprise RANTES. In some embodiments, the additional agent does not comprise L-selectin. In some embodiments, the additional agent does not comprise P-selectin. In some embodiments, the additional agent does not comprise E-selectin. In some embodiments, the additional agent does not comprise CD34. In some embodiments, the additional agent does not comprise GlyCAM-1. In some embodiments, the additional agent does not comprise MadCAM-1. In some embodiments, the additional agent does not comprise LFA-1. In some embodiments, the additional agent does not comprise VLA-I. In some embodiments, the additional agent does not comprise Mac-1. In some embodiments, the additional agent does not comprise p150.95. In some embodiments, the additional agent does not comprise PECAM. In some embodiments, the additional agent does not comprise ICAM-1. In some embodiments, the additional agent does not comprise ICAM-2. In some embodiments, the additional agent does not comprise ICAM-3. In some embodiments, the additional agent does not comprise CD2. In some embodiments, the additional agent does not comprise LFA-3. In some embodiments, the additional agent does not comprise M-CSF. In some embodiments, the additional agent does not comprise G-CSF. In some embodiments, the additional agent does not comprise IL-4. In some embodiments, the additional agent does not comprise mutant forms of IL-18. In some embodiments, the additional agent does not comprise CD40. In some embodiments, the additional agent does not comprise CD40L. In some embodiments, the additional agent does not comprise vascular growth factor. In some embodiments, the additional agent does not comprise fibroblast growth factor. In some embodiments, the additional agent does not comprise IL-7. In some embodiments, the additional agent does not comprise nerve growth factor. In some embodiments, the additional agent does not comprise vascular endothelial growth factor. In some embodiments, the additional agent does not comprise Fas. In some embodiments, the additional agent does not comprise TNF receptor. In some embodiments, the additional agent does not comprise Fit. In some embodiments, the additional agent does not comprise Apo-1. In some embodiments, the additional agent does not comprise p55. In some embodiments, the additional agent does not comprise WSL-I. In some embodiments, the additional agent does not comprise DR3. In some embodiments, the additional agent does not comprise TRAMP. In some embodiments, the additional agent does not comprise Apo-3. In some embodiments, the additional agent does not comprise AIR. In some embodiments, the additional agent does not comprise LARD. In some embodiments, the additional agent does not comprise NGRF. In some embodiments, the additional agent does not comprise DR4. In some embodiments, the additional agent does not comprise DR5. In some embodiments, the additional agent does not comprise KILLER. In some embodiments, the additional agent does not comprise TRAIL-R2. In some embodiments, the additional agent does not comprise TRICK2. In some embodiments, the additional agent does not comprise DR6. In some embodiments, the additional agent does not comprise Caspase ICE. In some embodiments, the additional agent does not comprise Fos. In some embodiments, the additional agent does not comprise c-jun. In some embodiments, the additional agent does not comprise Sp-I. In some embodiments, the additional agent does not comprise Ap-I. In some embodiments, the additional agent does not comprise Ap-2. In some embodiments, the additional agent does not comprise p38. In some embodiments, the additional agent does not comprise p65Rel. In some embodiments, the additional agent does not comprise MyD88. In some embodiments, the additional agent does not comprise IRAK. In some embodiments, the additional agent does not comprise TRAF6. In some embodiments, the additional agent does not comprise IkB. In some embodiments, the additional agent does not comprise Inactive NIK. In some embodiments, the additional agent does not comprise SAP K. In some embodiments, the additional agent does not comprise SAP-I. In some embodiments, the additional agent does not comprise JNK. In some embodiments, the additional agent does not comprise interferon response genes. In some embodiments, the additional agent does not comprise NFkB. In some embodiments, the additional agent does not comprise ax. In some embodiments, the additional agent does not comprise TRAIL. In some embodiments, the additional agent does not comprise TRAILrec. In some embodiments, the additional agent does not comprise TRAILrecDRC 5. In some embodiments, the additional agent does not comprise TRAIL-R3. In some embodiments, the additional agent does not comprise TRAIL-R4. In some embodiments, the additional agent does not comprise RANK. In some embodiments, the additional agent does not comprise RANK ligand. In some embodiments, the additional agent does not comprise OX40. In some embodiments, the additional agent does not comprise OX40 ligand. In some embodiments, the additional agent does not comprise NKG2D. In some embodiments, the additional agent does not comprise MICA. In some embodiments, the additional agent does not comprise MICB. In some embodiments, the additional agent does not comprise NKG2A. In some embodiments, the additional agent does not comprise NKG2B. In some embodiments, the additional agent does not comprise NKG2C. In some embodiments, the additional agent does not comprise NKG2E. In some embodiments, the additional agent does not comprise NKG2F. In some embodiments, the additional agent does not comprise TAP 1. In some embodiments, the additional agent does not comprise TAP2. In some embodiments, the additional agent does not comprise anti-CTLA4-sc. In some embodiments, the additional agent does not comprise anti-LAG3-Ig. In some embodiments, the additional agent does not comprise anti-TIM3-Ig.

In some embodiments, the additional agent comprises alpha-interferon. In some embodiments, the additional agent comprises gamma-interferon. In some embodiments, the additional agent comprises platelet derived growth factor (PDGF). In some embodiments, the additional agent comprises TNFa. In some embodiments, the additional agent comprises TNFP. In some embodiments, the additional agent comprises GM-CSF. In some embodiments, the additional agent comprises epidermal growth factor (EGF). In some embodiments, the additional agent comprises cutaneous T cell-attracting chemokine (CTACK). In some embodiments, the additional agent comprises epithelial thymus-expressed chemokine (TECK). In some embodiments, the additional agent comprises mucosae-associated epithelial chemokine (MEC). In some embodiments, the additional agent comprises IL-12. In some embodiments, the additional agent comprises IL-15. In some embodiments, the additional agent comprises MHC. In some embodiments, the additional agent comprises CD80. In some embodiments, the additional agent comprises CD86. In some embodiments, the additional agent comprises MCP-I. In some embodiments, the additional agent comprises MIP-1a. In some embodiments, the additional agent comprises MIP-Ip. In some embodiments, the additional agent comprises IL-8. In some embodiments, the additional agent comprises RANTES. In some embodiments, the additional agent comprises L-selectin. In some embodiments, the additional agent comprises P-selectin. In some embodiments, the additional agent comprises E-selectin. In some embodiments, the additional agent comprises CD34. In some embodiments, the additional agent comprises GlyCAM-1. In some embodiments, the additional agent comprises MadCAM-1. In some embodiments, the additional agent comprises LFA-1. In some embodiments, the additional agent comprises VLA-I. In some embodiments, the additional agent comprises Mac-1. In some embodiments, the additional agent comprises p150.95. In some embodiments, the additional agent comprises PEC AM. In some embodiments, the additional agent comprises ICAM-I. In some embodiments, the additional agent comprises ICAM-2. In some embodiments, the additional agent comprises ICAM-3. In some embodiments, the additional agent comprises CD2. In some embodiments, the additional agent comprises LFA-3. In some embodiments, the additional agent comprises M-CSF. In some embodiments, the additional agent comprises G-CSF. In some embodiments, the additional agent comprises IL-4. In some embodiments, the additional agent comprises mutant forms of IL-18. In some embodiments, the additional agent comprises CD40. In some embodiments, the additional agent comprises CD40L. In some embodiments, the additional agent comprises vascular growth factor. In some embodiments, the additional agent comprises fibroblast growth factor. In some embodiments, the additional agent comprises IL-7. In some embodiments, the additional agent comprises nerve growth factor. In some embodiments, the additional agent comprises vascular endothelial growth factor. In some embodiments, the additional agent comprises Fas. In some embodiments, the additional agent comprises TNF receptor. In some embodiments, the additional agent comprises Fit. In some embodiments, the additional agent comprises Apo-1. In some embodiments, the additional agent comprises p55. In some embodiments, the additional agent comprises WSL-I. In some embodiments, the additional agent comprises DR3. In some embodiments, the additional agent comprises TRAMP. In some embodiments, the additional agent comprises Apo-3. In some embodiments, the additional agent comprises AIR. In some embodiments, the additional agent comprises LARD. In some embodiments, the additional agent comprises NGRF. In some embodiments, the additional agent comprises DR4. In some embodiments, the additional agent comprises DR5. In some embodiments, the additional agent comprises KILLER. In some embodiments, the additional agent comprises TRAIL-R2. In some embodiments, the additional agent comprises TRICK2. In some embodiments, the additional agent comprises DR6. In some embodiments, the additional agent comprises Caspase ICE. In some embodiments, the additional agent comprises Fos. In some embodiments, the additional agent comprises c-jun. In some embodiments, the additional agent comprises Sp-I. In some embodiments, the additional agent comprises Ap-I. In some embodiments, the additional agent comprises Ap-2. In some embodiments, the additional agent comprises p38. In some embodiments, the additional agent comprises p65Rel. In some embodiments, the additional agent comprises MyD88. In some embodiments, the additional agent comprises IRAK. In some embodiments, the additional agent comprises TRAF6. In some embodiments, the additional agent comprises IkB. In some embodiments, the additional agent comprises Inactive NIK. In some embodiments, the additional agent comprises SAP K. In some embodiments, the additional agent comprises SAP-I. In some embodiments, the additional agent comprises JNK. In some embodiments, the additional agent comprises interferon response genes. In some embodiments, the additional agent comprises NFkB. In some embodiments, the additional agent comprises ax. In some embodiments, the additional agent comprises TRAIL. In some embodiments, the additional agent comprises TRAILrec. In some embodiments, the additional agent comprises TRAILrecDRC 5. In some embodiments, the additional agent comprises TRAIL-R3. In some embodiments, the additional agent comprises TRAIL-R4. In some embodiments, the additional agent comprises RANK. In some embodiments, the additional agent comprises RANK ligand. In some embodiments, the additional agent comprises OX40. In some embodiments, the additional agent comprises OX40 ligand. In some embodiments, the additional agent comprises NKG2D. In some embodiments, the additional agent comprises MICA. In some embodiments, the additional agent comprises MICB. In some embodiments, the additional agent comprises NKG2A. In some embodiments, the additional agent comprises NKG2B. In some embodiments, the additional agent comprises NKG2C. In some embodiments, the additional agent comprises NKG2E. In some embodiments, the additional agent comprises NKG2F. In some embodiments, the additional agent comprises TAP 1. In some embodiments, the additional agent comprises TAP2. In some embodiments, the additional agent comprises anti-CTLA4-sc. In some embodiments, the additional agent comprises anti-LAG3-Ig. In some embodiments, the additional agent comprises anti-TIM3-Ig.

### Lipid Nanoparticles

Lipid nanoparticles (LNPs) enhance the efficacy of mRNA and protein subunit vaccines by inducing robust T follicular helper cell and humoral responses, where IL-6 induction and the ionizable lipid component are critical for the adjuvant activity of LNPs (Alameh, et al., 2021, Immunity, 54, 2877-2892).

In some embodiments, the invention provides a lipid nanoparticle (LNP), compositions comprising the LNP and their use in methods of eliciting a modulated immune response against an antigen in a subject. In some embodiments, the LNP comprises one or more nucleic acid molecules described herein. For example, in some embodiments, the LNP comprises one or more nucleoside-modified RNA molecules encoding one or more antigens, one or more nucleoside-modified RNA molecules encoding one or more cytokines or immune receptors (such as but not limited to a cytokine receptors), and an ionizable lipid.

In certain embodiments, the ionizable lipid is selected from those similar to that formulated in the BNT162b2 vaccine and SM-102.

The term “lipid nanoparticle” refers to a particle having at least one dimension on the order of nanometers (e.g., 1-1,000 nm), which includes one or more lipids, for example a lipid of Formula (I), (II) or (III). In some embodiments, lipid nanoparticles are included in a formulation comprising a nucleoside-modified RNA as described herein. In some embodiments, such lipid nanoparticles comprise a cationic lipid (e.g., a lipid of Formula (I), (II) or (III)) and one or more excipient selected from neutral lipids, charged lipids, steroids and polymer conjugated lipids (e.g., a pegylated lipid such as a pegylated lipid of structure (IV), such as compound IVa). In some embodiments, the nucleoside-modified RNA is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells, e.g., an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In certain embodiments, the nucleoside-modified RNA, when present in the lipid nanoparticles, is resistant in aqueous solution to degradation with a nuclease.

The LNP may comprise any lipid capable of forming a particle to which the one or more nucleic acid molecules are attached, or in which the one or more nucleic acid molecules are encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

In some embodiments, the LNP comprises at least one ionizable lipid. In some embodiments, the ionizable lipid is a cationic lipid.

In some embodiments, the LNP comprises one or more cationic lipids, and one or more stabilizing lipids. Stabilizing lipids include neutral lipids and pegylated lipids.

In some embodiments, the LNP comprises a cationic lipid. As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In certain embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxy spermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In some embodiments, the cationic lipid is an amino lipid. Suitable amino lipids useful in the invention include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

Suitable amino lipids include those having the formula:

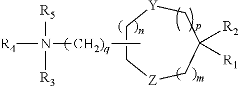

wherein R₁ and R₂ are either the same or different and independently optionally substituted C₁₀-C₂₄ alkyl, optionally substituted C₁₀-C₂₄ alkenyl, optionally substituted C₁₀-C₂₄ alkynyl, or optionally substituted C₁₀-C₂₄ acyl;
R₃ and R₄ are either the same or different and independently optionally substituted C₁-C₆ alkyl, optionally substituted C₂-C₆ alkenyl, or optionally substituted C₂-C₆ alkynyl or R₃ and R₄ may join to form an optionally substituted heterocyclic ring of 4 to 6 carbon atoms and 1 or 2 heteroatoms chosen from nitrogen and oxygen;
R₅ is either absent or present and when present is hydrogen or C₁-C₆ alkyl; m, n, and p are either the same or different and independently either 0 or 1 with the proviso that m, n, and p are not simultaneously 0;
q is 0, 1, 2, 3, or 4; and
Y and Z are either the same or different and independently O, S, or NH.

In some embodiments, R₁ and R₂ are each linoleyl, and the amino lipid is a dilinoleyl amino lipid. In some embodiments, the amino lipid is a dilinoleyl amino lipid.

A representative useful dilinoleyl amino lipid has the formula:

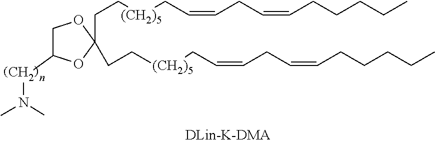

wherein n is 0, 1, 2, 3, or 4.

In some embodiments, the cationic lipid is a DLin-K-DMA. In some embodiments, the cationic lipid is DLin-KC2-DMA (DLin-K-DMA above, wherein n is 2)

In some embodiments, the cationic lipid component of the LNPs has the structure of Formula (I):

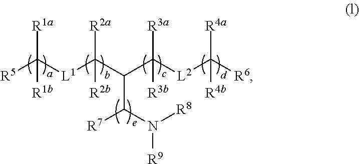

or a pharmaceutically acceptable salt, tautomer, prodrug or stereoisomer thereof, wherein:
L¹ and L² are each independently —O(C═O)—, —(C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent RAF and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl;
R⁸ and R⁹ are each independently C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24;
b and c are each independently an integer from 1 to 24; and
e is 1 or 2.

In certain embodiments of Formula (I), at least one of R^{1a}, R^{2a}, R^{3a} or R^{4a} is C₁-C₁₂ alkyl, or at least one of L¹ or L² is —O(C═O)— or —(C═O)O—. In other embodiments, R^{1a} and R^{1b} are not isopropyl when a is 6 or n-butyl when a is 8.

In still further embodiments of Formula (I), at least one of R^{1a}, R^{2a}, R^{3a} or R^{4a} is C₁-C₁₂ alkyl, or at least one of L¹ or L² is —O(C═O)— or —(C═O)O—; and
R^{1a} and R^{1b} are not isopropyl when a is 6 or n-butyl when a is 8.

In other embodiments of Formula (I), R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;

In certain embodiments of Formula (I), any one of L¹ or L² may be —O(C═O)— or a carbon-carbon double bond. L¹ and L² may each be —O(C═O)— or may each be a carbon-carbon double bond.

In some embodiments of Formula (I), one of L¹ or L² is —O(C═O)—. In other embodiments, both L¹ and L² are —O(C═O)—.

In some embodiments of Formula (I), one of L¹ or L² is —O(C═O)—. In other embodiments, both L¹ and L² are —O(C═O)—. In some other embodiments of Formula (I), one of L¹ or L² is a carbon-carbon double bond. In other embodiments, both L¹ and L² are a carbon-carbon double bond.

In still other embodiments of Formula (I), one of L¹ or L² is —O(C═O)— and the other of L¹ or L² is —(C═O)O—. In more embodiments, one of L¹ or L² is —O(C═O)— and the other of L¹ or L² is a carbon-carbon double bond. In yet more embodiments, one of L¹ or L² is —(C═O)O— and the other of L¹ or L² is a carbon-carbon double bond.

It is understood that “carbon-carbon” double bond, as used throughout the specification, refers to one of the following structures:

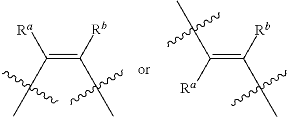

wherein R^{a} and R^{b} are, at each occurrence, independently H or a substituent. For example, in some embodiments R^{a} and R^{b} are, at each occurrence, independently H, C₁-C₁₂ alkyl or cycloalkyl, for example H or C₁-C₁₂ alkyl.

In other embodiments, the lipid compounds of Formula (I) have the following structure (Ia):

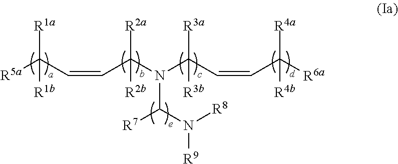

In other embodiments, the lipid compounds of Formula (I) have the following structure (Ib):

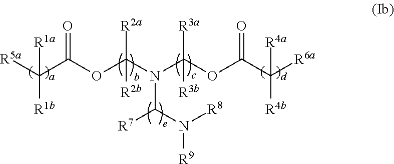

In yet other embodiments, the lipid compounds of Formula (I) have the following structure (Ic):

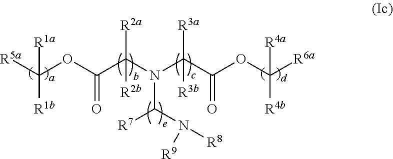

In certain embodiments of the lipid compound of Formula (I), a, b, c and d are each independently an integer from 2 to 12 or an integer from 4 to 12. In other embodiments, a, b, c and d are each independently an integer from 8 to 12 or 5 to 9. In some certain embodiments, a is 0. In some embodiments, a is 1. In other embodiments, a is 2. In more embodiments, a is 3. In yet other embodiments, a is 4. In some embodiments, a is 5. In other embodiments, a is 6. In more embodiments, a is 7. In yet other embodiments, a is 8. In some embodiments, a is 9. In other embodiments, a is 10. In more embodiments, a is 11. In yet other embodiments, a is 12. In some embodiments, a is 13. In other embodiments, a is 14. In more embodiments, a is 15. In yet other embodiments, a is 16.

In some other embodiments of Formula (I), b is 1. In other embodiments, b is 2. In more embodiments, b is 3. In yet other embodiments, b is 4. In some embodiments, b is 5. In other embodiments, b is 6. In more embodiments, b is 7. In yet other embodiments, b is 8. In some embodiments, b is 9. In other embodiments, b is 10. In more embodiments, b is 11. In yet other embodiments, b is 12. In some embodiments, b is 13. In other embodiments, b is 14. In more embodiments, b is 15. In yet other embodiments, b is 16.

In some more embodiments of Formula (I), c is 1. In other embodiments, c is 2. In more embodiments, c is 3. In yet other embodiments, c is 4. In some embodiments, c is 5. In other embodiments, c is 6. In more embodiments, c is 7. In yet other embodiments, c is 8. In some embodiments, c is 9. In other embodiments, c is 10. In more embodiments, c is 11. In yet other embodiments, c is 12. In some embodiments, c is 13. In other embodiments, c is 14. In more embodiments, c is 15. In yet other embodiments, c is 16.

In some certain other embodiments of Formula (I), d is 0. In some embodiments, d is 1. In other embodiments, d is 2. In more embodiments, d is 3. In yet other embodiments, d is 4. In some embodiments, d is 5. In other embodiments, d is 6.

In more embodiments, d is 7. In yet other embodiments, d is 8. In some embodiments, d is 9. In other embodiments, d is 10. In more embodiments, d is 11. In yet other embodiments, d is 12. In some embodiments, d is 13. In other embodiments, d is 14. In more embodiments, d is 15. In yet other embodiments, d is 16.

In some other various embodiments of Formula (I), a and d are the same.

In some other embodiments, b and c are the same. In some other specific embodiments, a and d are the same and b and c are the same.

The sum of a and b and the sum of c and d in Formula (I) are factors which may be varied to obtain a lipid of Formula (I) having the desired properties. In some embodiments, a and b are chosen such that their sum is an integer ranging from 14 to 24.

In other embodiments, c and d are chosen such that their sum is an integer ranging from 14 to 24. In further embodiment, the sum of a and b and the sum of c and d are the same. For example, in some embodiments the sum of a and b and the sum of c and d are both the same integer which may range from 14 to 24. In still more embodiments, a, b, c and d are selected such the sum of a and b and the sum of c and d is 12 or greater.

In some embodiments of Formula (I), e is 1. In other embodiments, e is 2.

The substituents at R^{1a}, R^{2a}, R^{3a} and R^{4a} of Formula (I) are not particularly limited. In certain embodiments R^{1a}, R^{2a}, R^{3a} and R^{4a} are H at each occurrence. In certain other embodiments, at least one of R^{1a}, R^{2a}, R^{3a} and R^{4a} is C₁-C₁₂ alkyl. In certain other embodiments, at least one of R^{1a}, R^{2a}, R^{3a} and R^{4a} is C₁-C₈ alkyl. In certain other embodiments, at least one of R^{1a}, R^{2a}, R^{3a} and R^{4a} is C₁-C₆ alkyl. In some of the foregoing embodiments, the C₁-C₈ alkyl is methyl, ethyl, n-propyl, iso-propyl, n-butyl, iso-butyl, tert-butyl, n-hexyl or n-octyl.

In certain embodiments of Formula (I), R^{1a}, R^{1b}, R^{4a} and R^{4b} are C₁-C₁₂ alkyl at each occurrence.

In further embodiments of Formula (I), at least one of R^{1a}, R^{1b}, R^{4a} and R^{4b} is H or R^{1a}, R^{1b}, R^{4a} and R^{4b} are H at each occurrence.

In certain embodiments of Formula (I), R^{1b} together with the carbon atom to which it is bound is taken together with an adjacent R^{1b} and the carbon atom to which it is bound to form a carbon-carbon double bond. In other embodiments of the foregoing R^{4b} together with the carbon atom to which it is bound is taken together with an adjacent R^{4b} and the carbon atom to which it is bound to form a carbon-carbon double bond.

The substituents at R⁵ and R⁶ of Formula (I) are not particularly limited in the foregoing embodiments. In certain embodiments one or both of R⁵ or R⁶ is methyl.

In certain other embodiments one or both of R⁵ or R⁶ is cycloalkyl for example cyclohexyl. In these embodiments, the cycloalkyl may be substituted or not substituted. In certain other embodiments, the cycloalkyl is substituted with C₁-C₁₂ alkyl, for example tert-butyl.

The substituents at R⁷ are not particularly limited in the foregoing embodiments of Formula (I). In certain embodiments, at least one R⁷ is H. In some other embodiments, R⁷ is H at each occurrence. In certain other embodiments R⁷ is C₁-C₁₂ alkyl.

In certain other of the foregoing embodiments of Formula (I), one of R⁸ or R⁹ is methyl. In other embodiments, both and cu are methyl.

In some different embodiments of Formula (I), R⁸ and R⁹, together with the nitrogen atom to which they are attached, form a 5, 6 or 7-membered heterocyclic ring. In some embodiments of the foregoing, R⁸ and R⁹, together with the nitrogen atom to which they are attached, form a 5-membered heterocyclic ring, for example a pyrrolidinyl ring.

In various different embodiments, the lipid of Formula (I) has one of the structures set forth in Table 1 below.

TABLE 1
Representative Lipids of Formula (I)

| | | Prep. |
|---|---|---|
| No. | Structure | Method |
| I-1 | 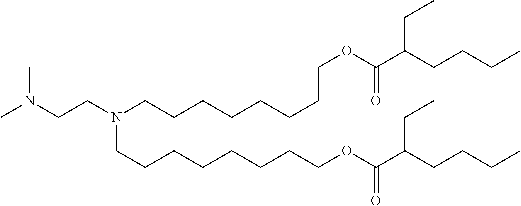 [SMILES: CCCCC(CC)C(=O)OCCCCCCCCN(CCCCCCCCOC(=O)C(CC)CCCC)CCN(C)C] | B |
| I-2 | 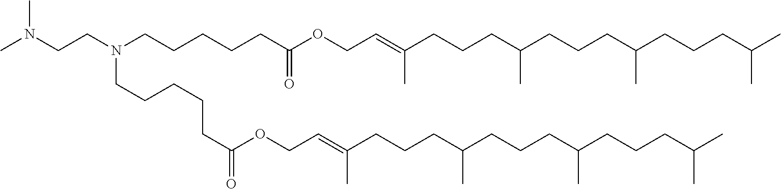 [SMILES: C/C(=C\COC(=O)CCCCCN(CCCCCC(=O)OC/C=C(\C)CCCC(C)CCCC(C)CCCC(C)C)CCN(C)C)CCCC(C)CCCC(C)CCCC(C)C] | A |
| I-3 | 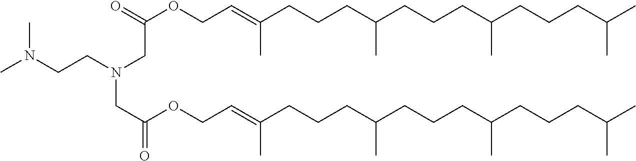 [SMILES: C/C(=C\COC(=O)CN(CCN(C)C)CC(=O)OC/C=C(\C)CCCC(C)CCCC(C)CCCC(C)C)CCCC(C)CCCC(C)CCCC(C)C] | A |
| I-4 | 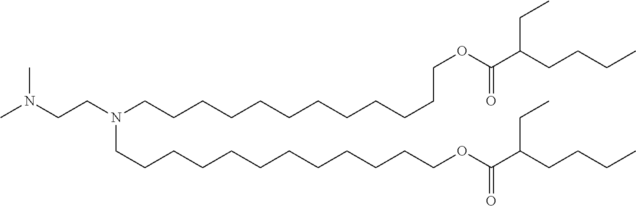 [SMILES: CCCCC(CC)C(=O)OCCCCCCCCCCCCN(CCCCCCCCCCCCOC(=O)C(CC)CCCC)CCN(C)C] | B |
| I-5 | 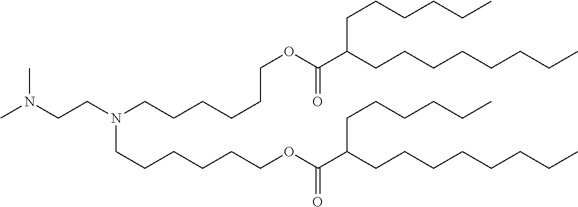 [SMILES: CCCCCCCCC(CCCCCC)C(=O)OCCCCCCN(CCCCCCOC(=O)C(CCCCCC)CCCCCCCC)CCN(C)C] | B |
| I-6 | 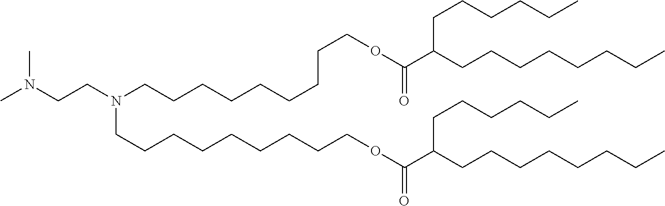 [SMILES: CCCCCCCCC(CCCCCC)C(=O)OCCCCCCCCCN(CCCCCCCCCOC(=O)C(CCCCCC)CCCCCCCC)CCN(C)C] | B |
| I-7 | 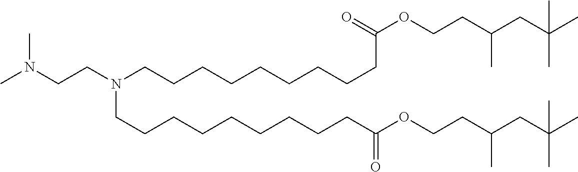 [SMILES: CC(CCOC(=O)CCCCCCCCCN(CCCCCCCCCC(=O)OCCC(C)CC(C)(C)C)CCN(C)C)CC(C)(C)C] | A |
| I-8 | 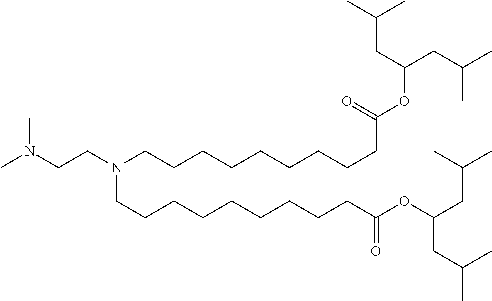 [SMILES: CC(C)CC(CC(C)C)OC(=O)CCCCCCCCCN(CCCCCCCCCC(=O)OC(CC(C)C)CC(C)C)CCN(C)C] | A |
| I-9 | 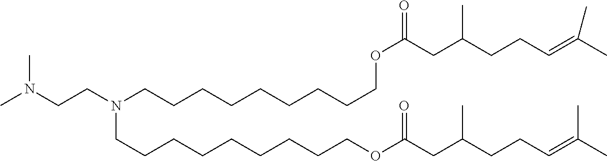 [SMILES: CC(C)=CCCC(C)CC(=O)OCCCCCCCCCN(CCCCCCCCCOC(=O)CC(C)CCC=C(C)C)CCN(C)C] | B |
| I-10 | 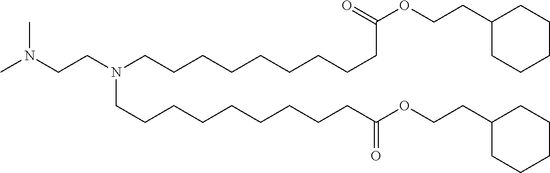 [SMILES: CN(C)CCN(CCCCCCCCCC(=O)OCCC1CCCCC1)CCCCCCCCCC(=O)OCCC1CCCCC1] | A |
| I-11 | 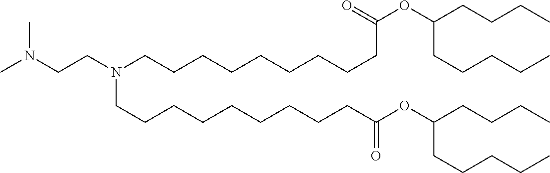 [SMILES: CCCCCC(CCCC)OC(=O)CCCCCCCCCN(CCCCCCCCCC(=O)OC(CCCC)CCCCC)CCN(C)C] | A |
| I-12 | 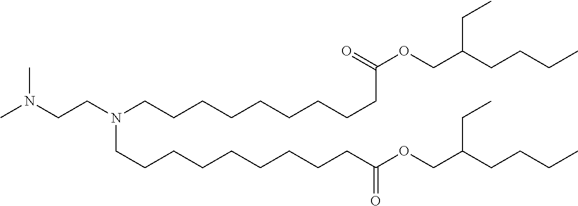 [SMILES: CCCCC(CC)COC(=O)CCCCCCCCCN(CCCCCCCCCC(=O)OCC(CC)CCCC)CCN(C)C] | A |
| I-13 | 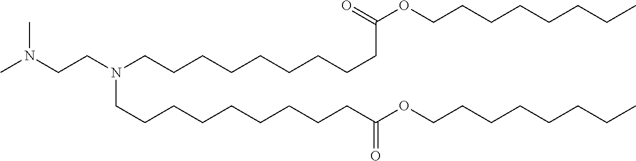 [SMILES: CCCCCCCCOC(=O)CCCCCCCCCN(CCCCCCCCCC(=O)OCCCCCCCC)CCN(C)C] | A |
| I-14 | 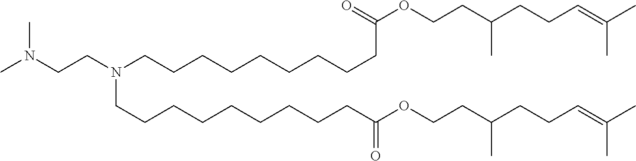 [SMILES: CC(C)=CCCC(C)CCOC(=O)CCCCCCCCCN(CCCCCCCCCC(=O)OCCC(C)CCC=C(C)C)CCN(C)C] | A |
| I-15 | 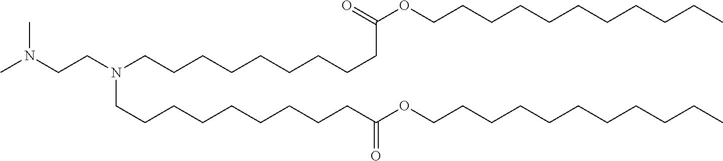 [SMILES: CCCCCCCCCCCOC(=O)CCCCCCCCCN(CCCCCCCCCC(=O)OCCCCCCCCCCC)CCN(C)C] | A |
| I-16 | 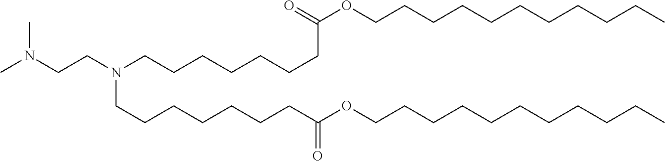 [SMILES: CCCCCCCCCCCOC(=O)CCCCCCCN(CCCCCCCC(=O)OCCCCCCCCCCC)CCN(C)C] | A |
| I-17 | 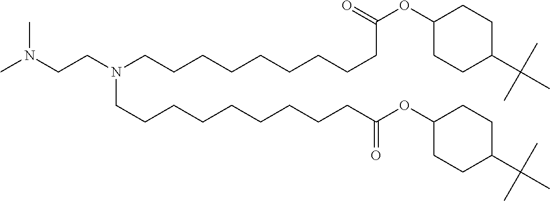 [SMILES: CN(C)CCN(CCCCCCCCCC(=O)OC1CCC(C(C)(C)C)CC1)CCCCCCCCCC(=O)OC1CCC(C(C)(C)C)CC1] | A |
| I-18 | 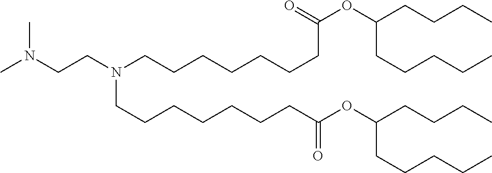 [SMILES: CCCCCC(CCCC)OC(=O)CCCCCCCN(CCCCCCCC(=O)OC(CCCC)CCCCC)CCN(C)C] | A |
| I-19 | 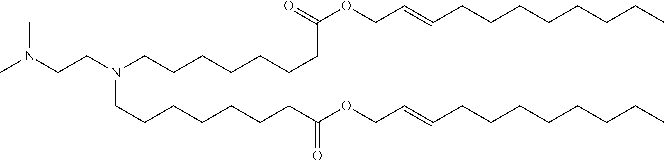 [SMILES: CCCCCCCC/C=C/COC(=O)CCCCCCCN(CCCCCCCC(=O)OC/C=C/CCCCCCCC)CCN(C)C] | A |
| I-20 | 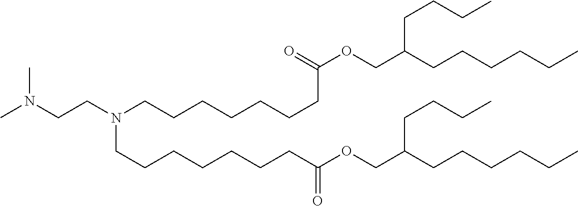 [SMILES: CCCCCCC(CCCC)COC(=O)CCCCCCCN(CCCCCCCC(=O)OCC(CCCC)CCCCCC)CCN(C)C] | A |
| I-21 | 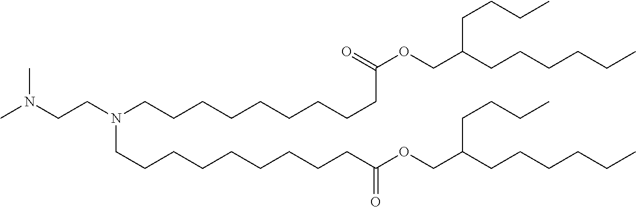 [SMILES: CCCCCCC(CCCC)COC(=O)CCCCCCCCCN(CCCCCCCCCC(=O)OCC(CCCC)CCCCCC)CCN(C)C] | A |
| I-22 | 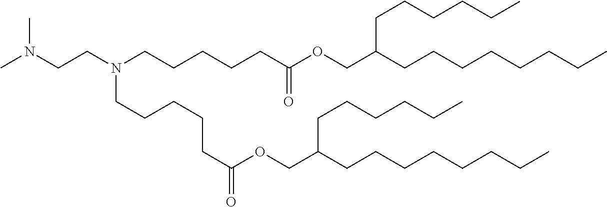 [SMILES: CCCCCCCCC(CCCCCC)COC(=O)CCCCCN(CCCCCC(=O)OCC(CCCCCC)CCCCCCCC)CCN(C)C] | A |
| I-23 | 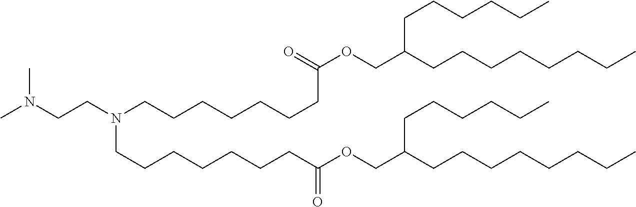 [SMILES: CCCCCCCCC(CCCCCC)COC(=O)CCCCCCCN(CCCCCCCC(=O)OCC(CCCCCC)CCCCCCCC)CCN(C)C] | A |
| I-24 | 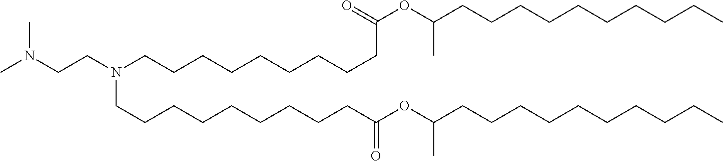 [SMILES: CCCCCCCCCCC(C)OC(=O)CCCCCCCCCN(CCCCCCCCCC(=O)OC(C)CCCCCCCCCC)CCN(C)C] | A |
| I-25 | 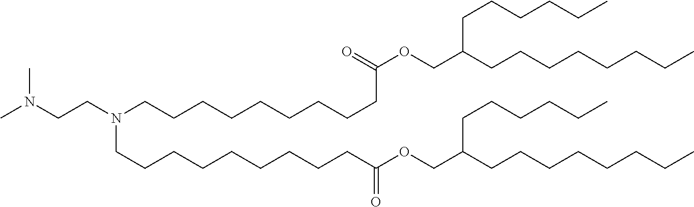 [SMILES: CCCCCCCCC(CCCCCC)COC(=O)CCCCCCCCCN(CCCCCCCCCC(=O)OCC(CCCCCC)CCCCCCCC)CCN(C)C] | A |
| I-26 | 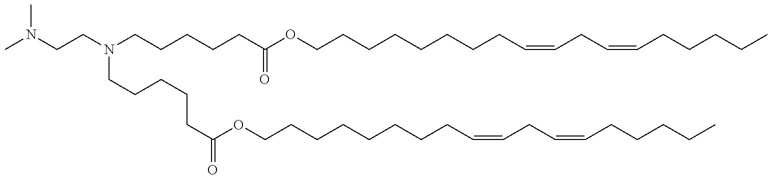 [SMILES: CCCCC/C=C\C/C=C\CCCCCCCCOC(=O)CCCCCN(CCCCCC(=O)OCCCCCCCC/C=C\C/C=C\CCCCC)CCN(C)C] | A |
| I-27 | 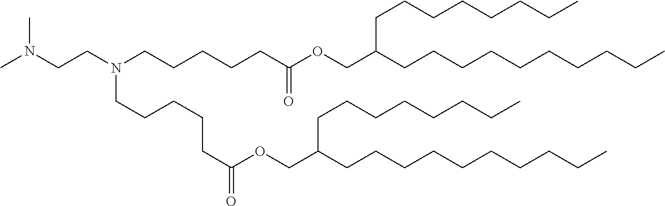 [SMILES: CCCCCCCCCCC(CCCCCCCC)COC(=O)CCCCCN(CCCCCC(=O)OCC(CCCCCCCC)CCCCCCCCCC)CCN(C)C] | A |
| I-28 | 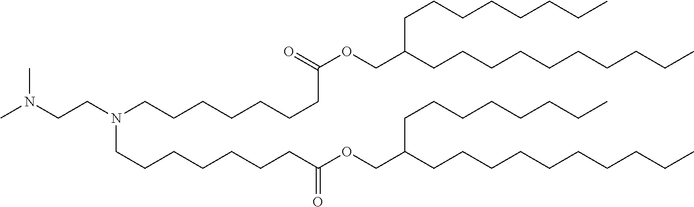 [SMILES: CCCCCCCCCCC(CCCCCCCC)COC(=O)CCCCCCCN(CCCCCCCC(=O)OCC(CCCCCCCC)CCCCCCCCCC)CCN(C)C] | A |
| I-29 | 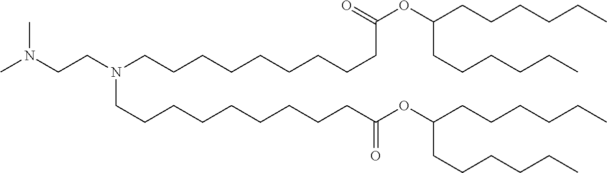 [SMILES: CCCCCCC(CCCCCC)OC(=O)CCCCCCCCCN(CCCCCCCCCC(=O)OC(CCCCCC)CCCCCC)CCN(C)C] | A |
| I-30 | 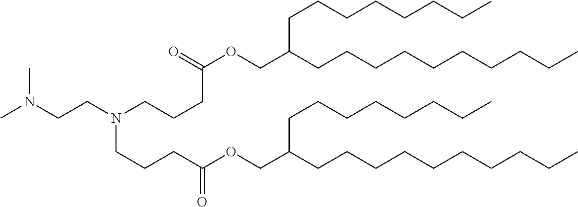 [SMILES: CCCCCCCCCCC(CCCCCCCC)COC(=O)CCCN(CCCC(=O)OCC(CCCCCCCC)CCCCCCCCCC)CCN(C)C] | A |
| I-31 | 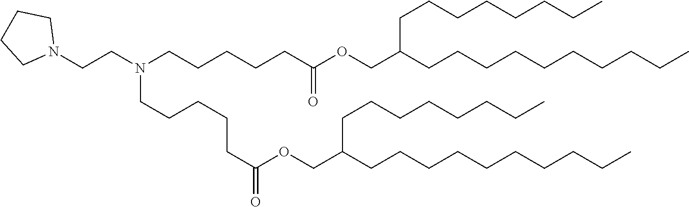 [SMILES: CCCCCCCCCCC(CCCCCCCC)COC(=O)CCCCCN(CCCCCC(=O)OCC(CCCCCCCC)CCCCCCCCCC)CCN1CCCC1] | C |
| I-32 | 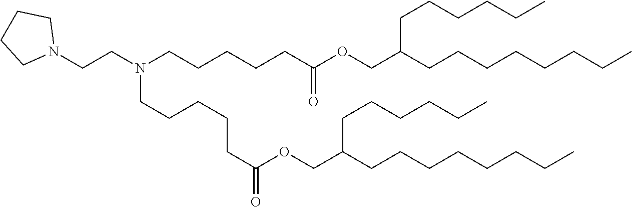 [SMILES: CCCCCCCCC(CCCCCC)COC(=O)CCCCCN(CCCCCC(=O)OCC(CCCCCC)CCCCCCCC)CCN1CCCC1] | C |
| I-33 | 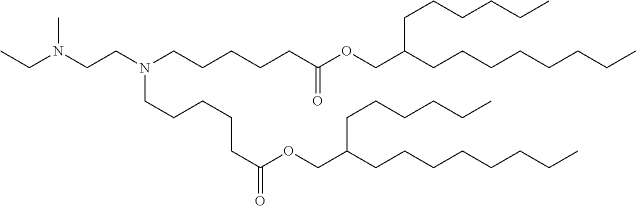 [SMILES: CCCCCCCCC(CCCCCC)COC(=O)CCCCCN(CCCCCC(=O)OCC(CCCCCC)CCCCCCCC)CCN(C)CC] | C |
| I-34 | 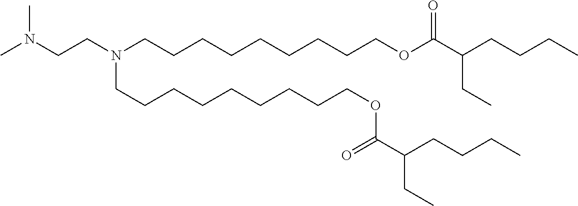 [SMILES: CCCCC(CC)C(=O)OCCCCCCCCCN(CCCCCCCCCOC(=O)C(CC)CCCC)CCN(C)C] | B |
| I-35 | 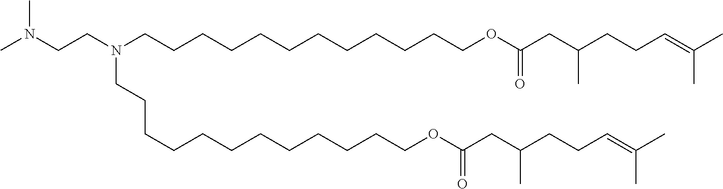 [SMILES: CC(C)=CCCC(C)CC(=O)OCCCCCCCCCCCCN(CCCCCCCCCCCCOC(=O)CC(C)CCC=C(C)C)CCN(C)C] | B |
| I-36 | 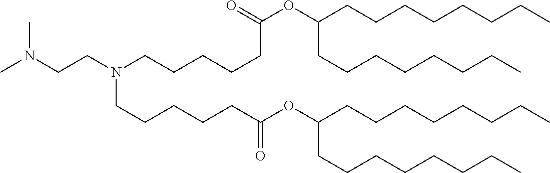 [SMILES: CCCCCCCCC(CCCCCCCC)OC(=O)CCCCCN(CCCCCC(=O)OC(CCCCCCCC)CCCCCCCC)CCN(C)C] | C |
| I-37 | 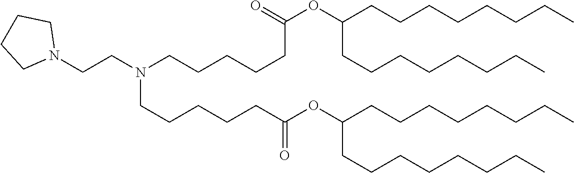 [SMILES: CCCCCCCCC(CCCCCCCC)OC(=O)CCCCCN(CCCCCC(=O)OC(CCCCCCCC)CCCCCCCC)CCN1CCCC1] | C |
| I-38 | 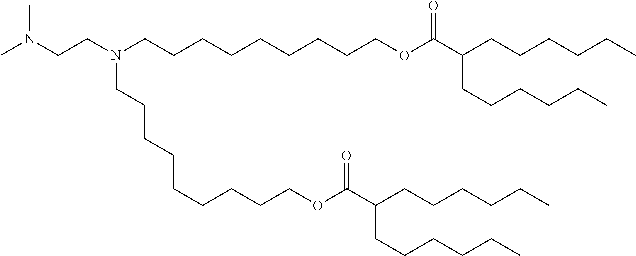 [SMILES: CCCCCCC(CCCCCC)C(=O)OCCCCCCCCCN(CCCCCCCCCOC(=O)C(CCCCCC)CCCCCC)CCN(C)C] | B |
| I-39 | 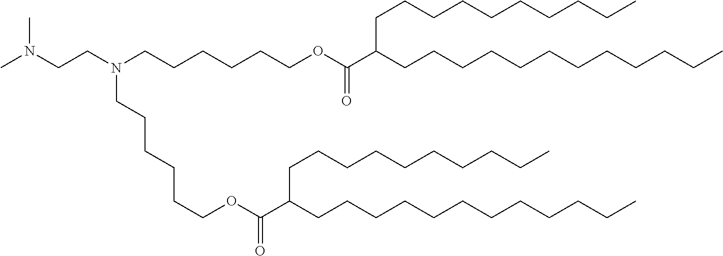 [SMILES: CCCCCCCCCCCCC(CCCCCCCCCC)C(=O)OCCCCCCN(CCCCCCOC(=O)C(CCCCCCCCCC)CCCCCCCCCCCC)CCN(C)C] | B |
| I-40 | 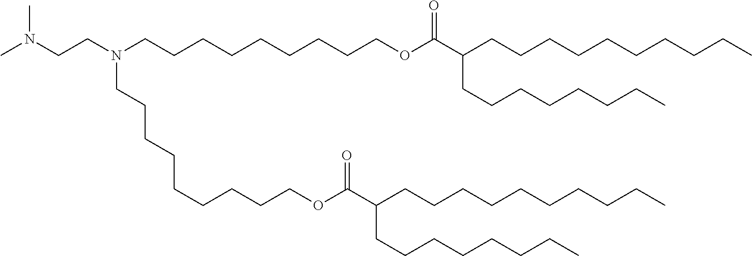 [SMILES: CCCCCCCCCCC(CCCCCCCC)C(=O)OCCCCCCCCCN(CCCCCCCCCOC(=O)C(CCCCCCCC)CCCCCCCCCC)CCN(C)C] | B |
| I-41 | 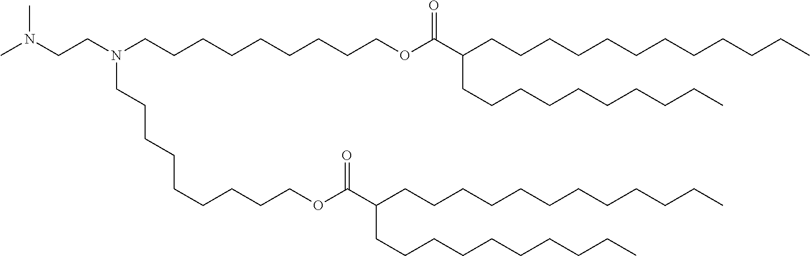 [SMILES: CCCCCCCCCCCCC(CCCCCCCCCC)C(=O)OCCCCCCCCCN(CCCCCCCCCOC(=O)C(CCCCCCCCCC)CCCCCCCCCCCC)CCN(C)C] | B |

In some embodiments, the LNPs comprise a lipid of Formula (I), a first nucleoside-modified RNA encoding an antigen, a second nucleoside-modified RNA encoding a cytokine or immune receptor (such as but not limited to a cytokine receptor), and one or more excipients selected from neutral lipids, steroids and pegylated lipids. In some embodiments the lipid of Formula (I) is compound I-5. In some embodiments the lipid of Formula (I) is compound I-6.

In some other embodiments, the cationic lipid component of the LNPs has the structure of Formula (II):

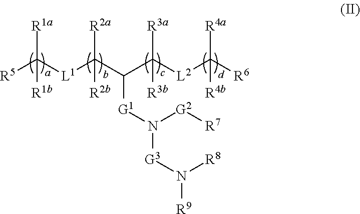

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}, —OC(═O)NR^{a}—, —NR^{a}C(═O)O—, or a direct bond;
G¹ is C₁-C₂ alkylene, —(C═O)—, —O(C═O)—, —SC(═O)—, —NR^{a}C(═O)— or a direct bond;
G² is —C(═O)—, —(C═O)O—, —C(═O)S—, —C(═O)NR^{a} or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either: (a) H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and
x is 0, 1 or 2.

In some embodiments of Formula (II), L¹ and L² are each independently —O(C═O)—, —(C═O)O— or a direct bond. In other embodiments, G¹ and G² are each independently —(C═O)— or a direct bond. In some different embodiments, L¹ and L² are each independently —O(C═O)—, —(C═O)O— or a direct bond; and G¹ and G² are each independently —(C═O)— or a direct bond.

In some different embodiments of Formula (II), L¹ and L² are each independently —C(═O)—, —O—, —S(O)ₓ—, —S—S—, —C(═O)S—, —SC(═O)—, —NR^{a}—, —NR^{a}C(═O)—, —C(═O)NR^{a}—, —NR^{a}C(═O)NR^{a}, —OC(═O)NR^{a}—, —NR^{a}C(═O)O—, —NR^{a}S(O)ₓNR^{a}—, —NR^{a}S(O)ₓ— or —S(O)ₓNR^{a}—.

In other of the foregoing embodiments of Formula (II), the lipid compound has one of the following structures (IIA) or (IIB):

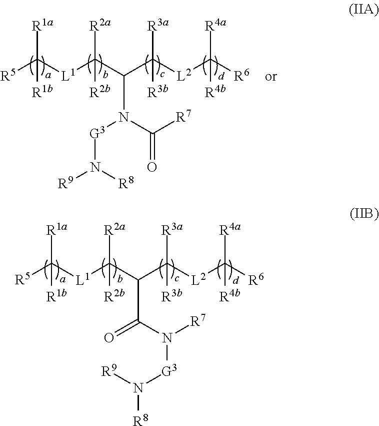

In some embodiments of Formula (II), the lipid compound has structure (IIA). In other embodiments, the lipid compound has structure (IIB).

In any of the foregoing embodiments of Formula (II), one of L¹ or L² is —O(C═O)—. For example, in some embodiments each of L¹ and L² are —O(C═O)—.

In some different embodiments of Formula (II), one of L¹ or L² is —(C═O)O—. For example, in some embodiments each of L¹ and L² is —(C═O)O—.

In different embodiments of Formula (II), one of L¹ or L² is a direct bond. As used herein, a “direct bond” means the group (e.g., L¹ or L²) is absent. For example, in some embodiments each of L¹ and L² is a direct bond.

In other different embodiments of Formula (II), for at least one occurrence of R^{1a} and R^{1b}, R^{1a} is H or C₁-C₁₂ alkyl, and R^{1b} together with the carbon atom to which it is bound is taken together with an adjacent R^{1b} and the carbon atom to which it is bound to form a carbon-carbon double bond.

In still other different embodiments of Formula (II), for at least one occurrence of R^{4a} and R^{4b}, R^{4a} is H or C₁-C₁₂ alkyl, and R^{4b} together with the carbon atom to which it is bound is taken together with an adjacent R^{4b} and the carbon atom to which it is bound to form a carbon-carbon double bond.

In more embodiments of Formula (II), for at least one occurrence of R^{2a} and R^{2b}, R^{2a} is H or C₁-C₁₂ alkyl, and R^{2b} together with the carbon atom to which it is bound is taken together with an adjacent R^{2b} and the carbon atom to which it is bound to form a carbon-carbon double bond.

In other different embodiments of Formula (II), for at least one occurrence of R^{3a} and R^{3b}, R^{3a} is H or C₁-C₁₂ alkyl, and R^{3b} together with the carbon atom to which it is bound is taken together with an adjacent R^{3b} and the carbon atom to which it is bound to form a carbon-carbon double bond.

In various other embodiments of Formula (II), the lipid compound has one of the following structures (IIC) or (IID):

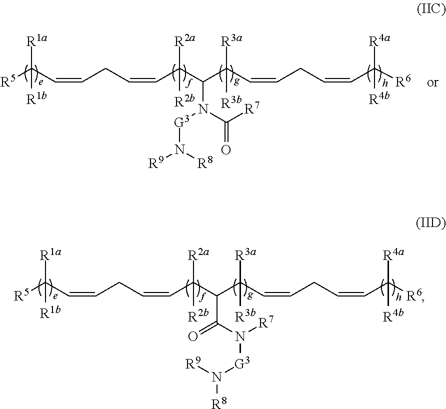

wherein e, f, g and h are each independently an integer from 1 to 12.

In some embodiments of Formula (II), the lipid compound has structure (IIC). In other embodiments, the lipid compound has structure (IID).

In various embodiments of structures (IIC) or (IID), e, f, g and h are each independently an integer from 4 to 10.

In certain embodiments of Formula (II), a, b, c and d are each independently an integer from 2 to 12 or an integer from 4 to 12. In other embodiments, a, b, c and d are each independently an integer from 8 to 12 or 5 to 9. In some certain embodiments, a is 0. In some embodiments, a is 1. In other embodiments, a is 2. In more embodiments, a is 3. In yet other embodiments, a is 4. In some embodiments, a is 5. In other embodiments, a is 6. In more embodiments, a is 7. In yet other embodiments, a is 8. In some embodiments, a is 9. In other embodiments, a is 10. In more embodiments, a is 11. In yet other embodiments, a is 12. In some embodiments, a is 13. In other embodiments, a is 14. In more embodiments, a is 15. In yet other embodiments, a is 16.

In some embodiments of Formula (II), b is 1. In other embodiments, b is 2. In more embodiments, b is 3. In yet other embodiments, b is 4. In some embodiments, b is 5. In other embodiments, b is 6. In more embodiments, b is 7. In yet other embodiments, b is 8. In some embodiments, b is 9. In other embodiments, b is 10. In more embodiments, b is 11. In yet other embodiments, b is 12. In some embodiments, b is 13. In other embodiments, b is 14. In more embodiments, b is 15. In yet other embodiments, b is 16.

In some embodiments of Formula (II), c is 1. In other embodiments, c is 2. In more embodiments, c is 3. In yet other embodiments, c is 4. In some embodiments, c is 5. In other embodiments, c is 6. In more embodiments, c is 7. In yet other embodiments, c is 8. In some embodiments, c is 9. In other embodiments, c is 10. In more embodiments, c is 11. In yet other embodiments, c is 12. In some embodiments, c is 13. In other embodiments, c is 14. In more embodiments, c is 15. In yet other embodiments, c is 16.

In some certain embodiments of Formula (II), d is 0. In some embodiments, d is 1. In other embodiments, d is 2. In more embodiments, d is 3. In yet other embodiments, d is 4. In some embodiments, d is 5. In other embodiments, d is 6.

In more embodiments, d is 7. In yet other embodiments, d is 8. In some embodiments, d is 9. In other embodiments, d is 10. In more embodiments, d is 11. In yet other embodiments, d is 12. In some embodiments, d is 13. In other embodiments, d is 14. In more embodiments, d is 15. In yet other embodiments, d is 16.

In some embodiments of Formula (II), e is 1. In other embodiments, e is 2. In more embodiments, e is 3. In yet other embodiments, e is 4. In some embodiments, e is 5. In other embodiments, e is 6. In more embodiments, e is 7. In yet other embodiments, e is 8. In some embodiments, e is 9. In other embodiments, e is 10. In more embodiments, e is 11. In yet other embodiments, e is 12.

In some embodiments of Formula (II), f is 1. In other embodiments, f is 2. In more embodiments, f is 3. In yet other embodiments, f is 4. In some embodiments, f is 5. In other embodiments, f is 6. In more embodiments, f is 7. In yet other embodiments, f is 8. In some embodiments, f is 9. In other embodiments, f is 10. In more embodiments, f is 11. In yet other embodiments, f is 12.

In some embodiments of Formula (II), g is 1. In other embodiments, g is 2. In more embodiments, g is 3. In yet other embodiments, g is 4. In some embodiments, g is 5. In other embodiments, g is 6. In more embodiments, g is 7. In yet other embodiments, g is 8. In some embodiments, g is 9. In other embodiments, g is 10. In more embodiments, g is 11. In yet other embodiments, g is 12.

In some embodiments of Formula (II), h is 1. In other embodiments, e is 2. In more embodiments, h is 3. In yet other embodiments, h is 4. In some embodiments, e is 5. In other embodiments, h is 6. In more embodiments, h is 7. In yet other embodiments, h is 8. In some embodiments, h is 9. In other embodiments, h is 10. In more embodiments, h is 11. In yet other embodiments, h is 12.

In some other various embodiments of Formula (II), a and d are the same. In some other embodiments, b and c are the same. In some other specific embodiments and a and d are the same and b and c are the same.

The sum of a and b and the sum of c and d of Formula (II) are factors which may be varied to obtain a lipid having the desired properties. In some embodiments, a and b are chosen such that their sum is an integer ranging from 14 to 24. In other embodiments, c and d are chosen such that their sum is an integer ranging from 14 to 24. In further embodiment, the sum of a and b and the sum of c and d are the same. For example, in some embodiments the sum of a and b and the sum of c and d are both the same integer which may range from 14 to 24. In still more embodiments, a. b, c and d are selected such that the sum of a and b and the sum of c and d is 12 or greater.

The substituents at R^{1a}, R^{2a}, R^{3a} and R^{4a} of Formula (II) are not particularly limited. In some embodiments, at least one of R^{1a}, R^{2a}, R^{3a} and R^{4a} is H. In certain embodiments R^{1a}, R^{2a}, R^{3a} and R^{4a} are H at each occurrence. In certain other embodiments, at least one of R^{1a}, R^{2a}, R^{3a} and R^{4a} is C₁-C₁₂ alkyl. In certain other embodiments, at least one of R^{1a}, R^{2a}, R^{3a} and R^{4a} is C₁-C₈ alkyl. In certain other embodiments, at least one of R^{1a}, R^{2a}, R^{3a} and R^{4a} is C₁-C₆ alkyl. In some of the foregoing embodiments, the C₁-C₈ alkyl is methyl, ethyl, n-propyl, iso-propyl, n-butyl, iso-butyl, tert-butyl, n-hexyl or n-octyl.

In certain embodiments of Formula (II), R^{1a}, R^{1b}, R^{4a} and R^{4b} are C₁-C₁₂ alkyl at each occurrence.

In further embodiments of Formula (II), at least one of R^{1a}, R^{2b}, R^{3b} and R^{4b} is H or R^{1b}, R^{2b}, R^{3b} and R^{4b} are H at each occurrence.

In certain embodiments of Formula (II), R^{1b} together with the carbon atom to which it is bound is taken together with an adjacent R^{1a} and the carbon atom to which it is bound to form a carbon-carbon double bond. In other embodiments of the foregoing R^{4b} together with the carbon atom to which it is bound is taken together with an adjacent R^{4b} and the carbon atom to which it is bound to form a carbon-carbon double bond.

The substituents at R⁵ and R⁶ of Formula (II) are not particularly limited in the foregoing embodiments. In certain embodiments one of R⁵ or R⁶ is methyl. In other embodiments, each of R⁵ or R⁶ is methyl.

The substituents at R⁷ of Formula (II) are not particularly limited in the foregoing embodiments. In certain embodiments R⁷ is C₆-C₁₆ alkyl. In some other embodiments, R⁷ is C₆-C₉ alkyl. In some of these embodiments, R⁷ is substituted with —(C═O)OR^{b}, —O(C═O)R^{b}, —C(═O)R^{b}, —OR^{b}, —S(O)ₓR^{b}, —S—SR^{b}, —C(═O)SR^{b}, —SC(═O)R^{b}, —NR^{a}R^{b}, —NR^{a}C(═O)R^{b}, —C(═O)NR^{a}R^{b}, —NR^{a}C(═O)NR^{a}R^{b}, —OC(═O)NR^{a}R^{b}, —NR^{a}C(═O)OR^{b}, —NR^{a}S(O)ₓNR^{a}R^{b}, —NR^{a}S(O)ₓR^{b} or —S(O)ₓNR^{a}R^{b}, wherein: R^{a} is H or C₁-C₁₂ alkyl; R^{b} is C₁-C₁₅ alkyl; and x is 0, 1 or 2. For example, in some embodiments R⁷ is substituted with —(C═O)OR^{b} or —O(C═O)R.

In various of the foregoing embodiments of Formula (II), R^{b} is branched C₁-C₁₅ alkyl. For example, in some embodiments R^{b} has one of the following structures:

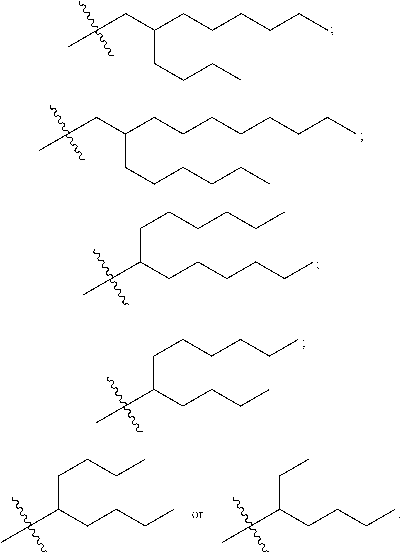

In certain other of the foregoing embodiments of Formula (II), one of R⁸ or R⁹ is methyl. In other embodiments, both R⁸ and R⁹ are methyl.

In some different embodiments of Formula (II), R⁸ and R⁹, together with the nitrogen atom to which they are attached, form a 5, 6 or 7-membered heterocyclic ring. In some embodiments of the foregoing, R⁸ and R⁹, together with the nitrogen atom to which they are attached, form a 5-membered heterocyclic ring, for example a pyrrolidinyl ring. In some different embodiments of the foregoing, R⁸ and R⁹, together with the nitrogen atom to which they are attached, form a 6-membered heterocyclic ring, for example a piperazinyl ring.

In still other embodiments of the foregoing lipids of Formula (II), G³ is C₂-C₄ alkylene, for example C₃ alkylene.

In various different embodiments, the lipid compound has one of the structures set forth in Table 2 below.

TABLE 2
Representative Lipids of Formula (II)

| | | Prep. |
|---|---|---|
| No. | Structure | Method |
| II-1 | 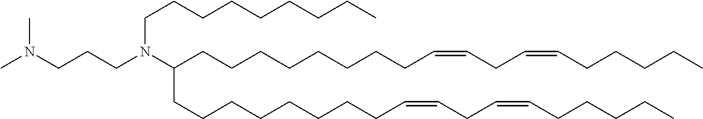 [SMILES: CCCCC/C=C\C/C=C\CCCCCCCCC(CCCCCCCC/C=C\C/C=C\CCCCC)N(CCCCCCCCC)CCCN(C)C] | D |
| II-2 | 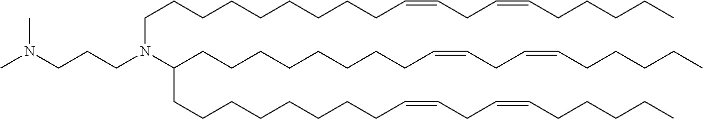 [SMILES: CCCCC/C=C\C/C=C\CCCCCCCCCN(CCCN(C)C)C(CCCCCCCC/C=C\C/C=C\CCCCC)CCCCCCCC/C=C\C/C=C\CCCCC] | D |
| II-3 | 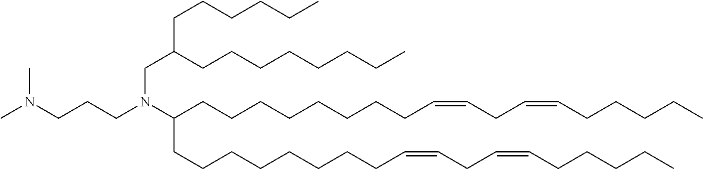 [SMILES: CCCCC/C=C\C/C=C\CCCCCCCCC(CCCCCCCC/C=C\C/C=C\CCCCC)N(CCCN(C)C)CC(CCCCCC)CCCCCCCC] | D |
| II-4 | 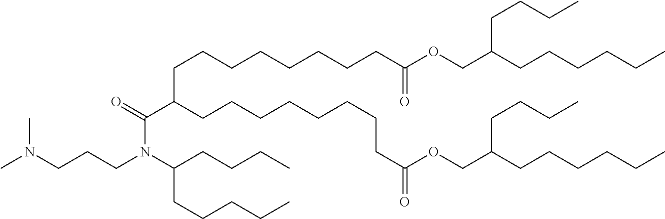 [SMILES: CCCCCCC(CCCC)COC(=O)CCCCCCCCC(CCCCCCCCC(=O)OCC(CCCC)CCCCCC)C(=O)N(CCCN(C)C)C(CCCC)CCCCC] | E |
| II-5 | 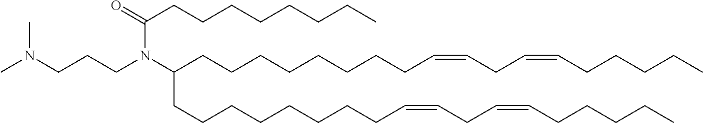 [SMILES: CCCCC/C=C\C/C=C\CCCCCCCCC(CCCCCCCC/C=C\C/C=C\CCCCC)N(CCCN(C)C)C(=O)CCCCCCCC] | D |
| II-6 | 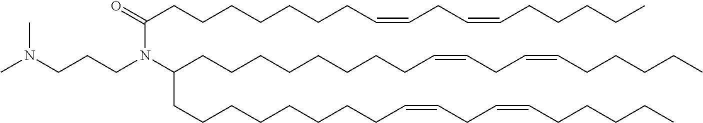 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC] | D |
| II-7 | 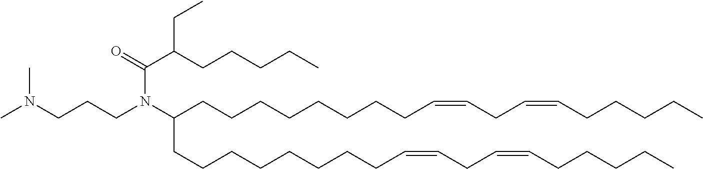 [SMILES: CCCCC/C=C\C/C=C\CCCCCCCCC(CCCCCCCC/C=C\C/C=C\CCCCC)N(CCCN(C)C)C(=O)C(CC)CCCCC] | D |
| II-8 | 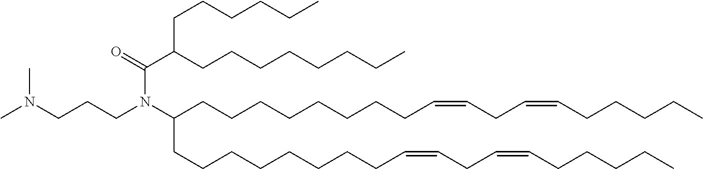 [SMILES: CCCCC/C=C\C/C=C\CCCCCCCCC(CCCCCCCC/C=C\C/C=C\CCCCC)N(CCCN(C)C)C(=O)C(CCCCCC)CCCCCCCC] | D |
| II-9 | 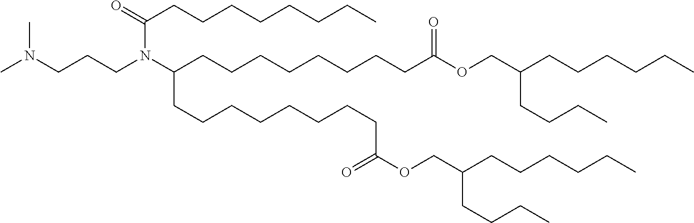 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC] | D |
| II-10 | 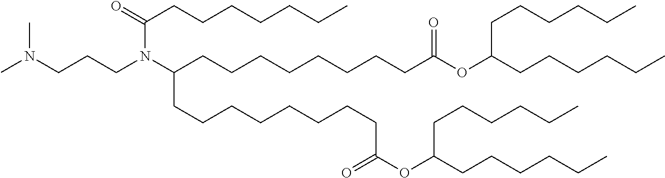 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC] | D |
| II-11 | 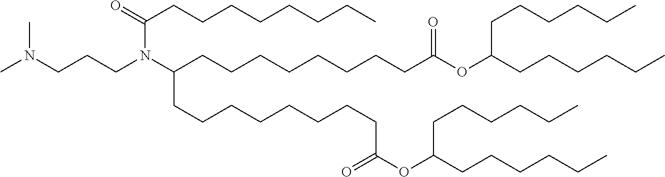 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC] | D |
| II-12 | 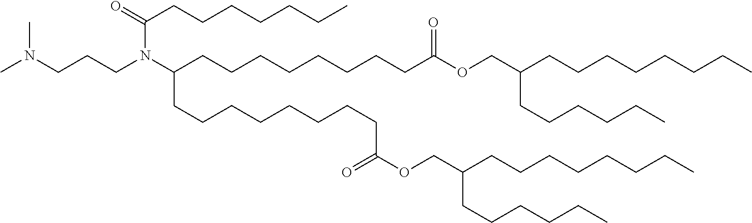 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC] | D |
| II-13 | 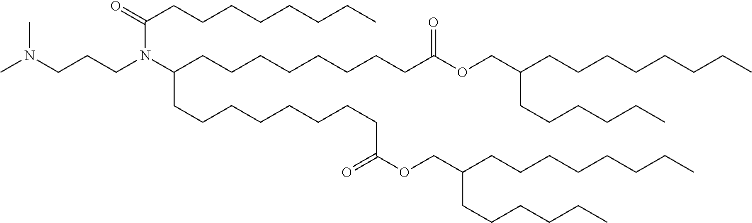 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC] | D |
| II-14 | 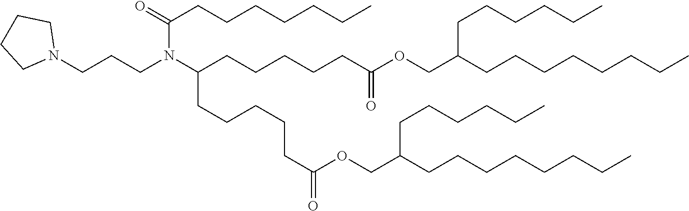 [SMILES: CCCCCCCCC(CCCCCC)COC(=O)CCCCCC(CCCCCC(=O)OCC(CCCCCC)CCCCCCCC)N(CCCN1CCCC1)C(=O)CCCCCCC] | D |
| II-15 | 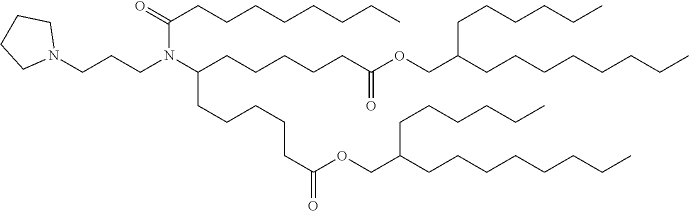 [SMILES: CCCCCCCCC(=O)N(CCCN1CCCC1)C(CCCCCC(=O)OCC(CCCCCC)CCCCCCCC)CCCCCC(=O)OCC(CCCCCC)CCCCCCCC] | D |
| II-16 | 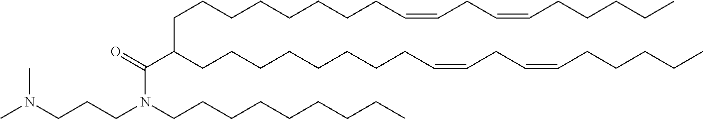 [SMILES: CCCCC/C=C\C/C=C\CCCCCCCCC(CCCCCCCC/C=C\C/C=C\CCCCC)C(=O)N(CCCCCCCCC)CCCN(C)C] | E |
| II-17 | 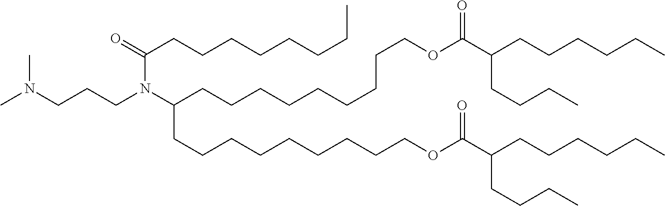 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC] | D |
| II-18 | 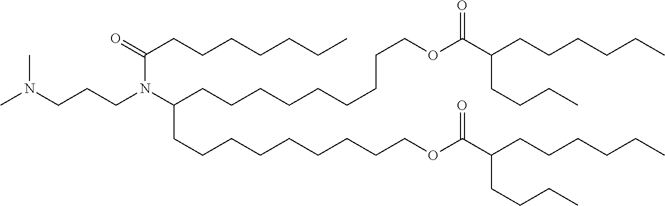 [SMILES: CCCCCCCC(=O)N(CCCN(C)C)C(CCCCCCCCCOC(=O)C(CCCC)CCCCCC)CCCCCCCCCOC(=O)C(CCCC)CCCCCC] | D |
| II-19 | 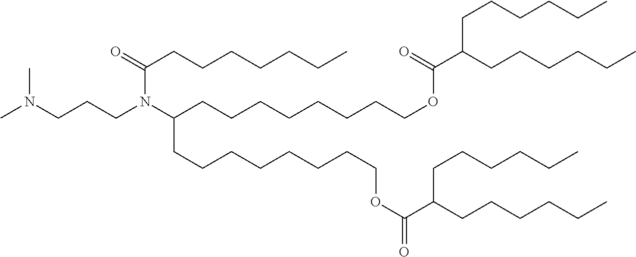 [SMILES: CCCCCCCC(=O)N(CCCN(C)C)C(CCCCCCCCOC(=O)C(CCCCCC)CCCCCC)CCCCCCCCOC(=O)C(CCCCCC)CCCCCC] | D |
| II-20 | 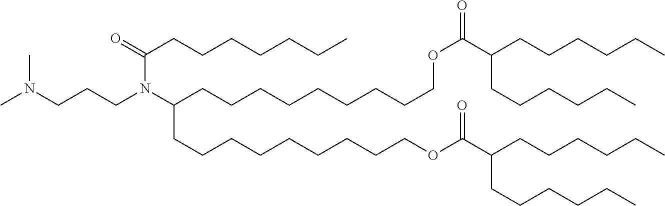 [SMILES: CCCCCCCC(=O)N(CCCN(C)C)C(CCCCCCCCCOC(=O)C(CCCCCC)CCCCCC)CCCCCCCCCOC(=O)C(CCCCCC)CCCCCC] | D |
| II-21 | 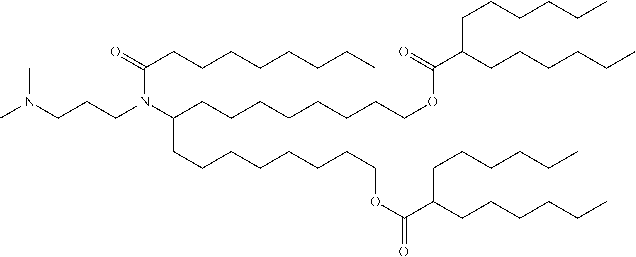 [SMILES: CCCCCCCCC(=O)N(CCCN(C)C)C(CCCCCCCCOC(=O)C(CCCCCC)CCCCCC)CCCCCCCCOC(=O)C(CCCCCC)CCCCCC] | D |
| II-22 | 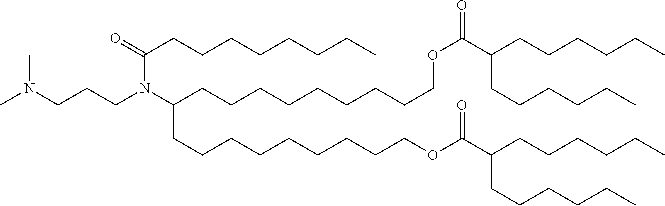 [SMILES: CCCCCCCCC(=O)N(CCCN(C)C)C(CCCCCCCCCOC(=O)C(CCCCCC)CCCCCC)CCCCCCCCCOC(=O)C(CCCCCC)CCCCCC] | D |
| II-23 | 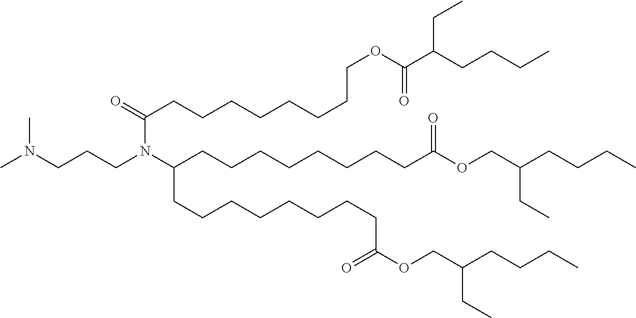 [SMILES: CCCCC(CC)COC(=O)CCCCCCCCC(CCCCCCCCC(=O)OCC(CC)CCCC)N(CCCN(C)C)C(=O)CCCCCCCCOC(=O)C(CC)CCCC] | D |
| II-24 | 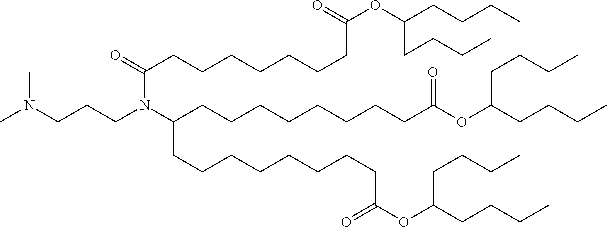 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC] | D |
| II-25 | 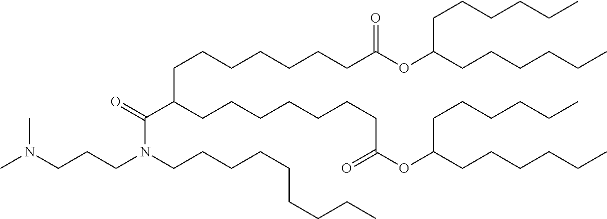 [SMILES: CCCCCCCCCN(CCCN(C)C)C(=O)C(CCCCCCCC(=O)OC(CCCCCC)CCCCCC)CCCCCCCC(=O)OC(CCCCCC)CCCCCC] | E |
| II-26 | 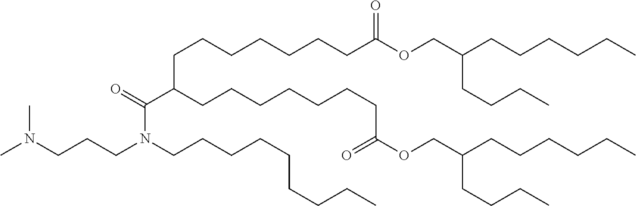 [SMILES: CCCCCCCCCN(CCCN(C)C)C(=O)C(CCCCCCCC(=O)OCC(CCCC)CCCCCC)CCCCCCCC(=O)OCC(CCCC)CCCCCC] | E |
| II-27 | 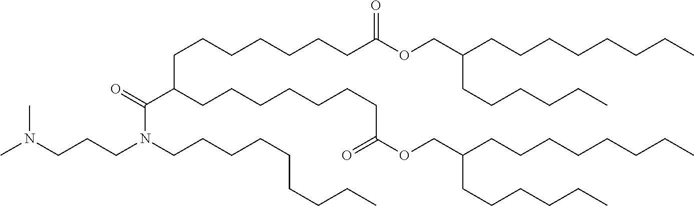 [SMILES: CCCCCCCCCN(CCCN(C)C)C(=O)C(CCCCCCCC(=O)OCC(CCCCCC)CCCCCCCC)CCCCCCCC(=O)OCC(CCCCCC)CCCCCCCC] | E |
| II-28 | 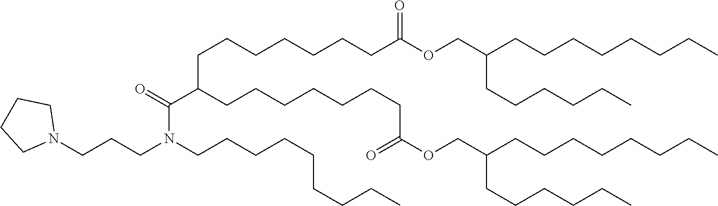 [SMILES: CCCCCCCCCN(CCCN1CCCC1)C(=O)C(CCCCCCCC(=O)OCC(CCCCCC)CCCCCCCC)CCCCCCCC(=O)OCC(CCCCCC)CCCCCCCC] | E |
| II-29 | 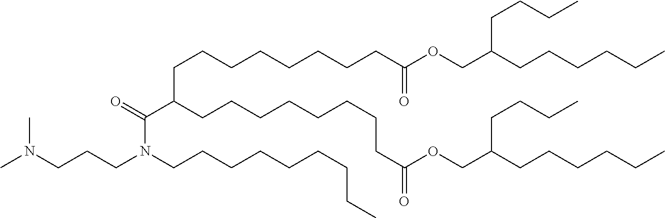 [SMILES: CCCCCCCCCN(CCCN(C)C)C(=O)C(CCCCCCCCC(=O)OCC(CCCC)CCCCCC)CCCCCCCCC(=O)OCC(CCCC)CCCCCC] | E |
| II-30 | 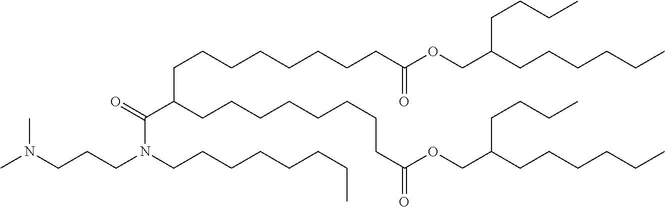 [SMILES: CCCCCCCCN(CCCN(C)C)C(=O)C(CCCCCCCCC(=O)OCC(CCCC)CCCCCC)CCCCCCCCC(=O)OCC(CCCC)CCCCCC] | E |
| II-31 | 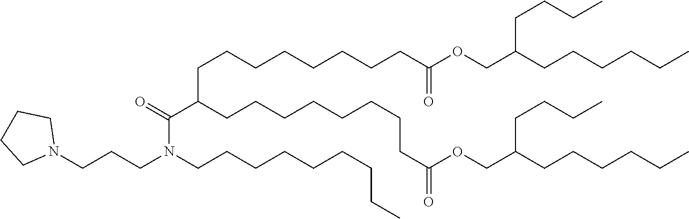 [SMILES: CCCCCCCCCN(CCCN1CCCC1)C(=O)C(CCCCCCCCC(=O)OCC(CCCC)CCCCCC)CCCCCCCCC(=O)OCC(CCCC)CCCCCC] | E |
| II-32 | 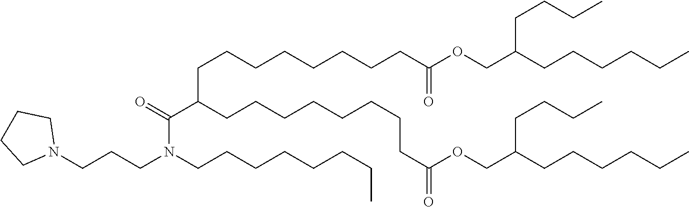 [SMILES: CCCCCCCCN(CCCN1CCCC1)C(=O)C(CCCCCCCCC(=O)OCC(CCCC)CCCCCC)CCCCCCCCC(=O)OCC(CCCC)CCCCCC] | E |
| II-33 | 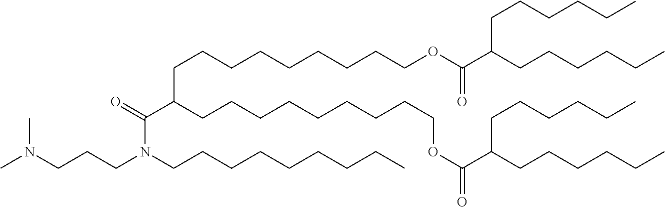 [SMILES: CCCCCCCCCN(CCCN(C)C)C(=O)C(CCCCCCCCCOC(=O)C(CCCCCC)CCCCCC)CCCCCCCCCOC(=O)C(CCCCCC)CCCCCC] | E |
| II-34 | 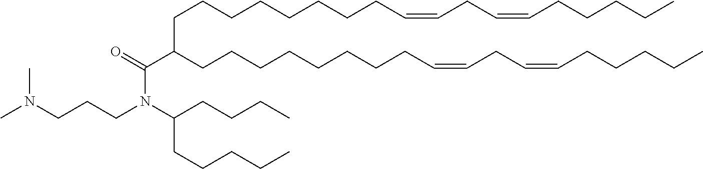 [SMILES: CCCCC/C=C\C/C=C\CCCCCCCCC(CCCCCCCC/C=C\C/C=C\CCCCC)C(=O)N(CCCN(C)C)C(CCCC)CCCCC] | E |

In some embodiments, the LNPs comprise a lipid of Formula (II), a first nucleoside-modified RNA encoding an antigen, a second nucleoside-modified RNA encoding a cytokine or immune receptor (such as but not limited to a cytokine receptor), and one or more excipient selected from neutral lipids, steroids and pegylated lipids. In some embodiments, the lipid of Formula (II) is compound II-9. In some embodiments, the lipid of Formula (II) is compound II-10. In some embodiments, the lipid of Formula (II) is compound II-11. In some embodiments, the lipid of Formula (II) is compound 11-12. In some embodiments, the lipid of Formula (II) is compound II-32.

In some other embodiments, the cationic lipid component of the LNPs has the structure of Formula (III):

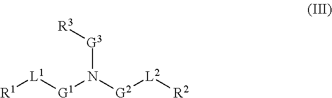

or a pharmaceutically acceptable salt, tautomer, prodrug or stereoisomer thereof, wherein:
one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

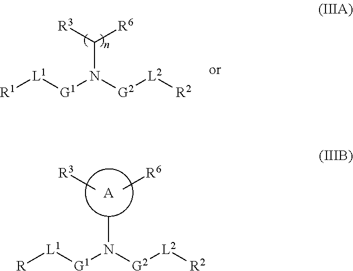

wherein:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (HID):

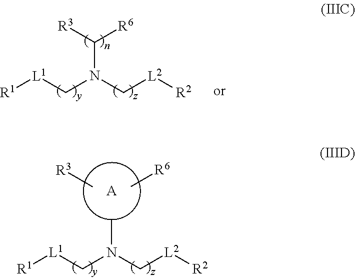

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (III), the lipid has one of the following structures (HIE) or (IIIF):

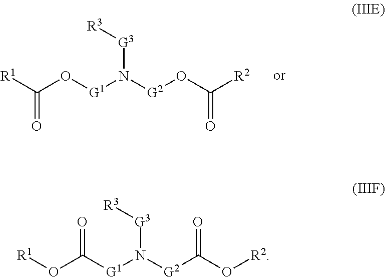

In some of the foregoing embodiments of Formula (III), the lipid has one of the following structures (IIIG), (IIIH), (IIII), or (IIIJ):

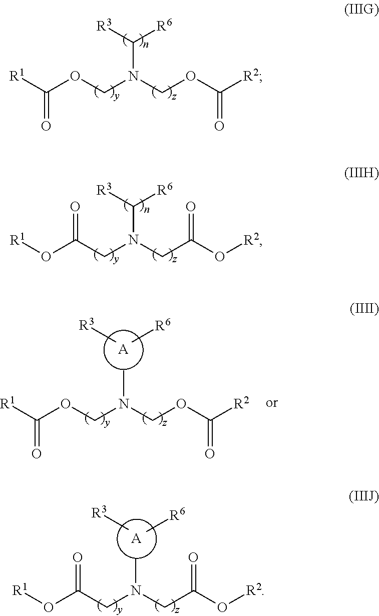

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G³ is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

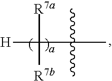

wherein:
R^{7a} and R^{7b} are, at each occurrence, independently H or C₁-C₁₂ alkyl; and a is an integer from 2 to 12,
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R⁷¹¹ is C₁-C₈ alkyl. For example, in some embodiments, C₁-C₈ alkyl is methyl, ethyl, n-propyl, isopropyl, n-butyl, iso-butyl, tert-butyl, n-hexyl or n-octyl.

In different embodiments of Formula (III), R¹ or R², or both, has one of the following structures:

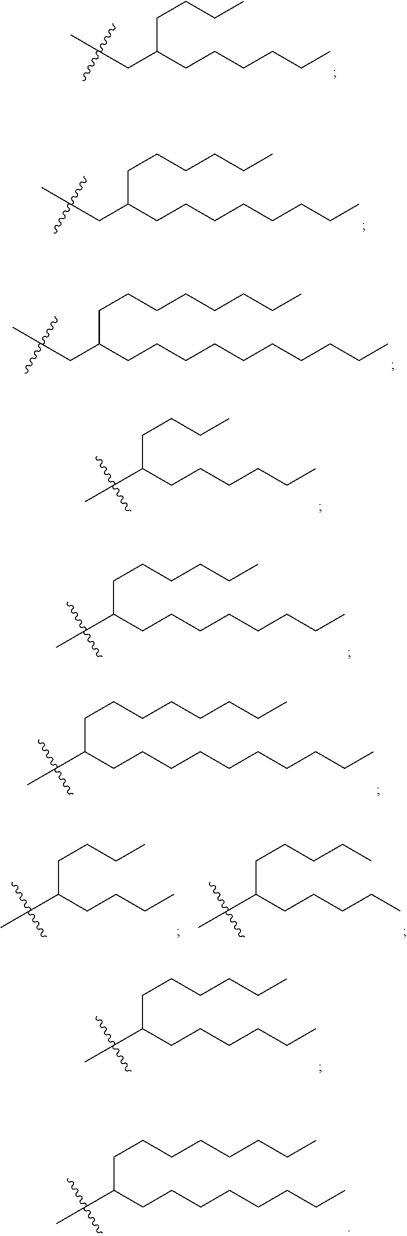

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the structures set forth in Table 3 below.

TABLE 3
Representative Compounds of Formula (III)

| | | Prep. |
|---|---|---|
| No. | Structure | Method |
| III-1 | 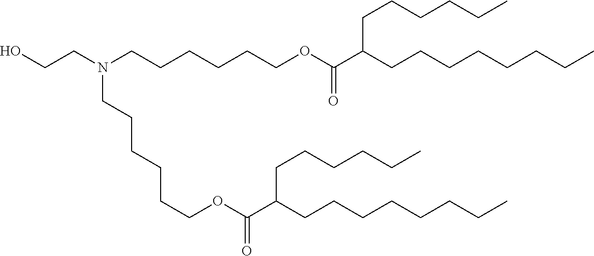 [SMILES: CCCCCCCCC(CCCCCC)C(=O)OCCCCCCN(CCO)CCCCCCOC(=O)C(CCCCCC)CCCCCCCC] | F |
| III-2 | 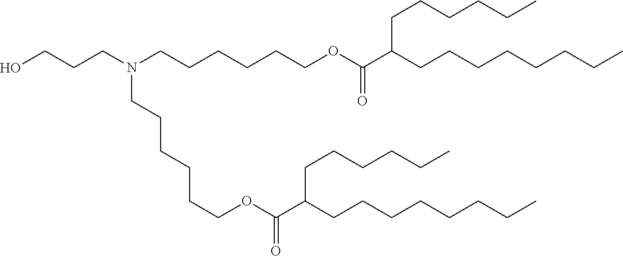 [SMILES: CCCCCCCCC(CCCCCC)C(=O)OCCCCCCN(CCCO)CCCCCCOC(=O)C(CCCCCC)CCCCCCCC] | F |
| III-3 | 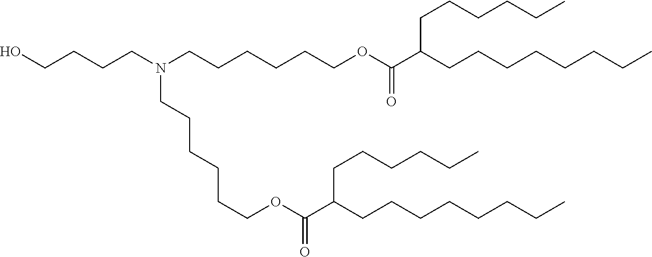 [SMILES: CCCCCCCCC(CCCCCC)C(=O)OCCCCCCN(CCCCO)CCCCCCOC(=O)C(CCCCCC)CCCCCCCC] | F |
| III-4 | 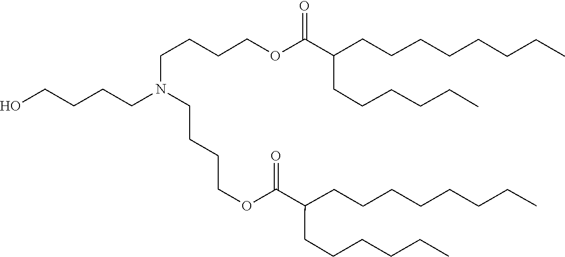 [SMILES: CCCCCCCCC(CCCCCC)C(=O)OCCCCN(CCCCO)CCCCOC(=O)C(CCCCCC)CCCCCCCC] | F |
| III-5 | 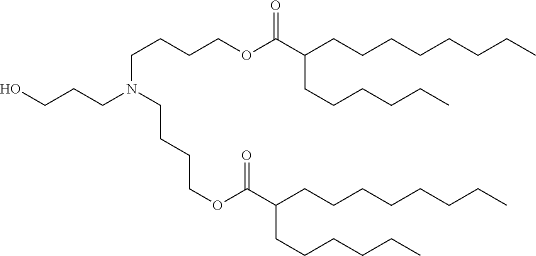 [SMILES: CCCCCCCCC(CCCCCC)C(=O)OCCCCN(CCCO)CCCCOC(=O)C(CCCCCC)CCCCCCCC] | F |
| III-6 | 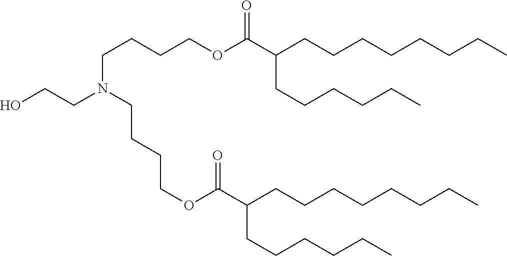 [SMILES: CCCCCCCCC(CCCCCC)C(=O)OCCCCN(CCO)CCCCOC(=O)C(CCCCCC)CCCCCCCC] | F |
| III-7 | 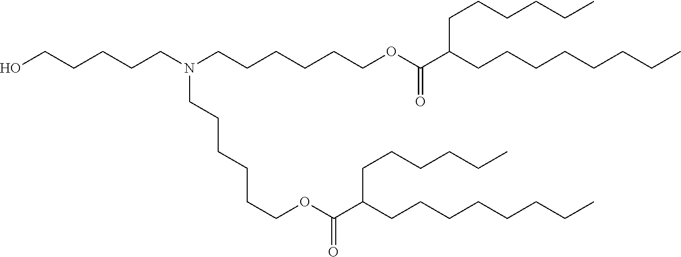 [SMILES: CCCCCCCCC(CCCCCC)C(=O)OCCCCCCN(CCCCCO)CCCCCCOC(=O)C(CCCCCC)CCCCCCCC] | F |
| III-8 | 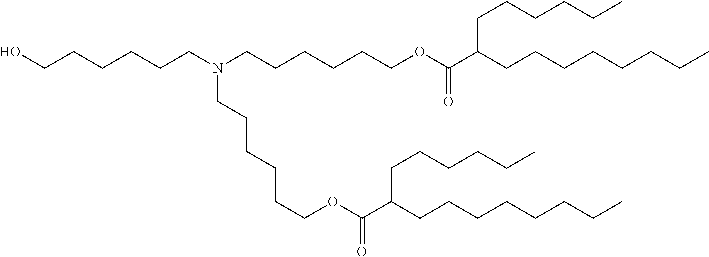 [SMILES: CCCCCCCCC(CCCCCC)C(=O)OCCCCCCN(CCCCCCO)CCCCCCOC(=O)C(CCCCCC)CCCCCCCC] | F |
| III-9 | 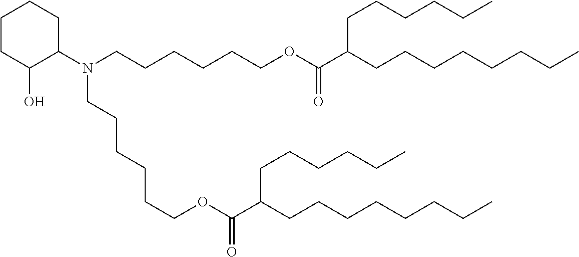 [SMILES: CCCCCCCCC(CCCCCC)C(=O)OCCCCCCN(CCCCCCOC(=O)C(CCCCCC)CCCCCCCC)C1CCCCC1O] | F |
| III-10 | 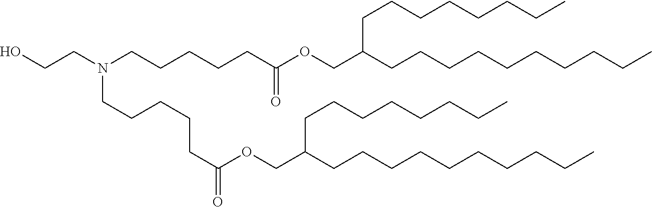 [SMILES: CCCCCCCCCCC(CCCCCCCC)COC(=O)CCCCCN(CCO)CCCCCC(=O)OCC(CCCCCCCC)CCCCCCCCCC] | F |
| III-11 | 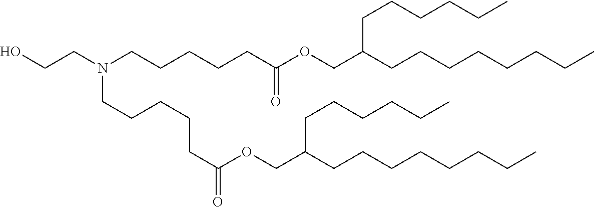 [SMILES: CCCCCCCCC(CCCCCC)COC(=O)CCCCCN(CCO)CCCCCC(=O)OCC(CCCCCC)CCCCCCCC] | F |
| III-12 | 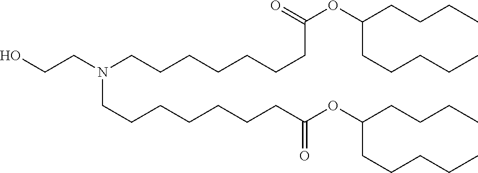 [SMILES: CCCCCC(CCCC)OC(=O)CCCCCCCN(CCO)CCCCCCCC(=O)OC(CCCC)CCCCC] | F |
| III-13 | 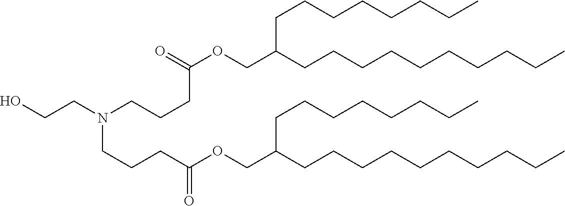 [SMILES: CCCCCCCCCCC(CCCCCCCC)COC(=O)CCCN(CCO)CCCC(=O)OCC(CCCCCCCC)CCCCCCCCCC] | F |
| III-14 | 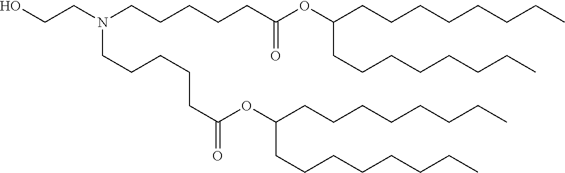 [SMILES: CCCCCCCCC(CCCCCCCC)OC(=O)CCCCCN(CCO)CCCCCC(=O)OC(CCCCCCCC)CCCCCCCC] | F |
| III-15 | 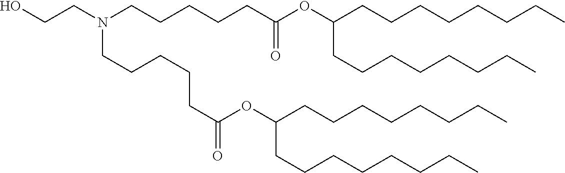 [SMILES: CCCCCCCCC(CCCCCCCC)OC(=O)CCCCCN(CCO)CCCCCC(=O)OC(CCCCCCCC)CCCCCCCC] | F |
| III-16 | 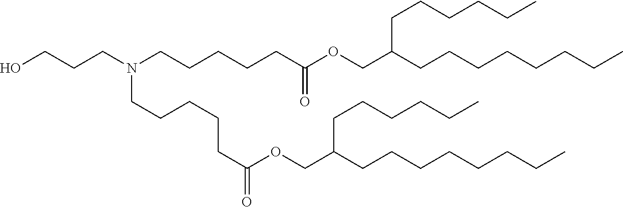 [SMILES: CCCCCCCCC(CCCCCC)COC(=O)CCCCCN(CCCO)CCCCCC(=O)OCC(CCCCCC)CCCCCCCC] | F |
| III-17 | 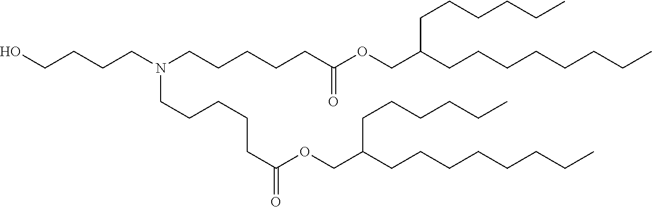 [SMILES: CCCCCCCCC(CCCCCC)COC(=O)CCCCCN(CCCCO)CCCCCC(=O)OCC(CCCCCC)CCCCCCCC] | F |
| III-18 | 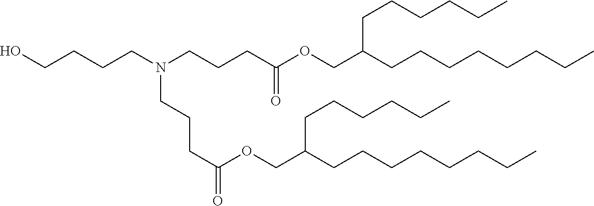 [SMILES: CCCCCCCCC(CCCCCC)COC(=O)CCCN(CCCCO)CCCC(=O)OCC(CCCCCC)CCCCCCCC] | F |
| III-19 | 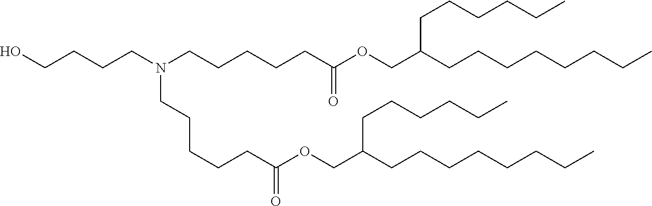 [SMILES: CCCCCCCCC(CCCCCC)COC(=O)CCCCCN(CCCCO)CCCCCC(=O)OCC(CCCCCC)CCCCCCCC] | F |
| III-20 | 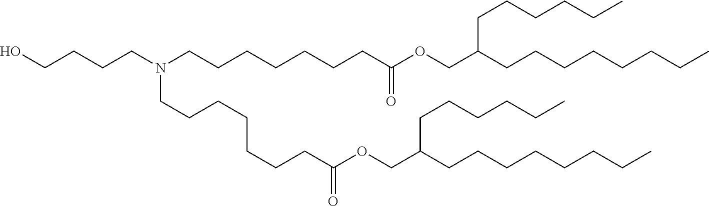 [SMILES: CCCCCCCCC(CCCCCC)COC(=O)CCCCCCCN(CCCCO)CCCCCCCC(=O)OCC(CCCCCC)CCCCCCCC] | F |
| III-21 | 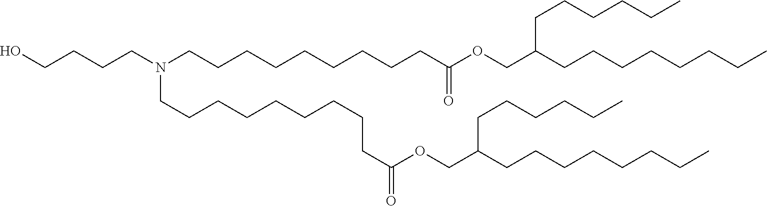 [SMILES: CCCCCCCCC(CCCCCC)COC(=O)CCCCCCCCCN(CCCCO)CCCCCCCCCC(=O)OCC(CCCCCC)CCCCCCCC] | F |
| III-22 | 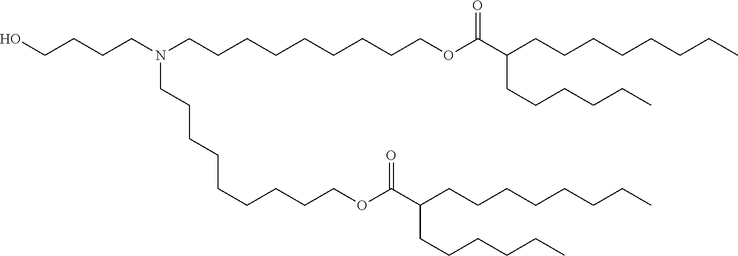 [SMILES: CCCCCCCCC(CCCCCC)C(=O)OCCCCCCCCCN(CCCCO)CCCCCCCCCOC(=O)C(CCCCCC)CCCCCCCC] | F |
| III-23 | 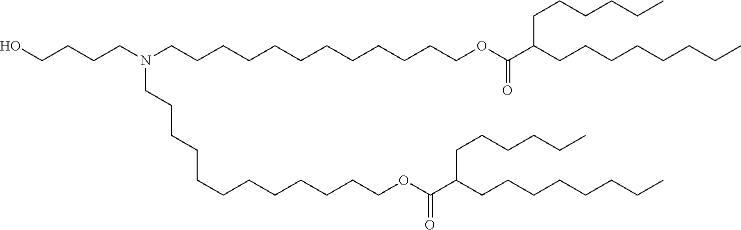 [SMILES: CCCCCCCCC(CCCCCC)C(=O)OCCCCCCCCCCCCN(CCCCO)CCCCCCCCCCCCOC(=O)C(CCCCCC)CCCCCCCC] | F |
| III-24 | 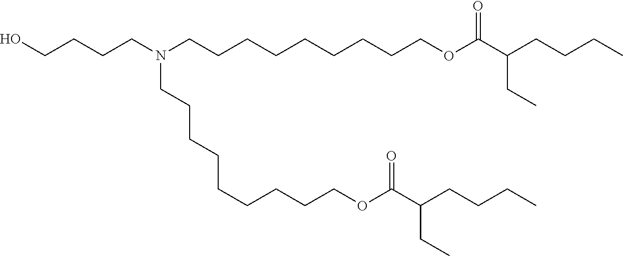 [SMILES: CCCCC(CC)C(=O)OCCCCCCCCCN(CCCCO)CCCCCCCCCOC(=O)C(CC)CCCC] | F |
| III-25 | 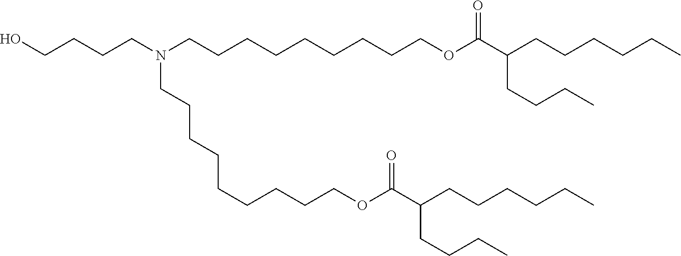 [SMILES: CCCCCCC(CCCC)C(=O)OCCCCCCCCCN(CCCCO)CCCCCCCCCOC(=O)C(CCCC)CCCCCC] | F |
| III-26 | 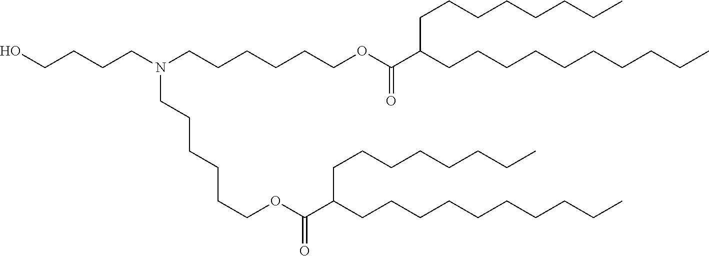 [SMILES: CCCCCCCCCCC(CCCCCCCC)C(=O)OCCCCCCN(CCCCO)CCCCCCOC(=O)C(CCCCCCCC)CCCCCCCCCC] | F |
| III-27 | 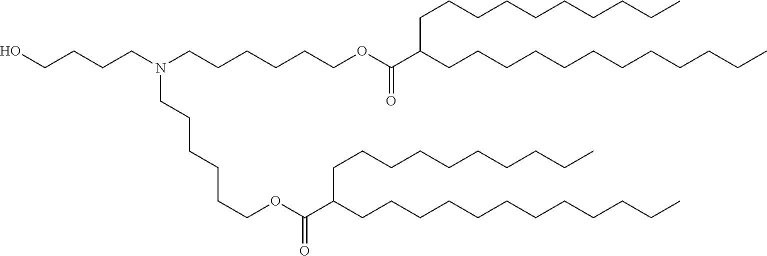 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] | F |
| III-28 | 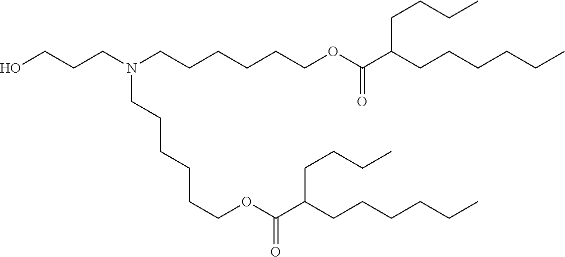 [SMILES: CCCCCCC(CCCC)C(=O)OCCCCCCN(CCCO)CCCCCCOC(=O)C(CCCC)CCCCCC] | F |
| III-29 | 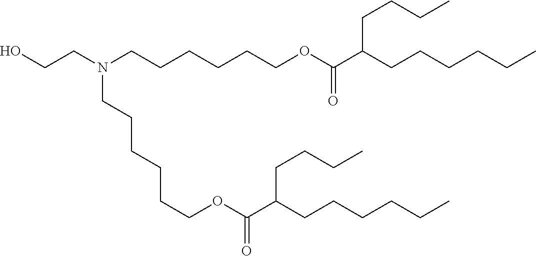 [SMILES: CCCCCCC(CCCC)C(=O)OCCCCCCN(CCO)CCCCCCOC(=O)C(CCCC)CCCCCC] | F |
| III-30 | 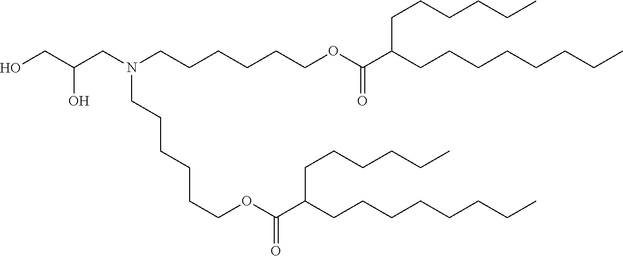 [SMILES: CCCCCCCCC(CCCCCC)C(=O)OCCCCCCN(CCCCCCOC(=O)C(CCCCCC)CCCCCCCC)CC(O)CO] | F |
| III-31 | 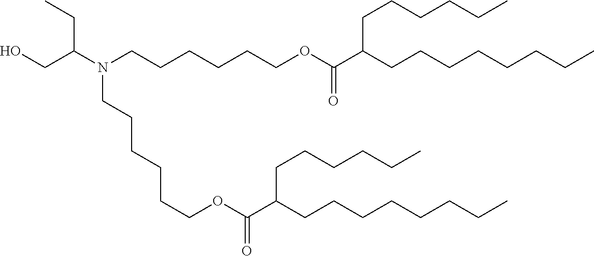 [SMILES: CCCCCCCCC(CCCCCC)C(=O)OCCCCCCN(CCCCCCOC(=O)C(CCCCCC)CCCCCCCC)C(CC)CO] | F |
| III-32 | 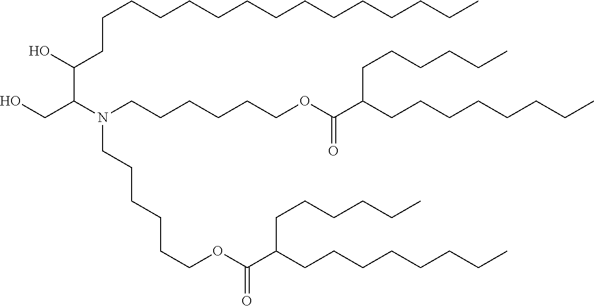 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] | F |
| III-33 | 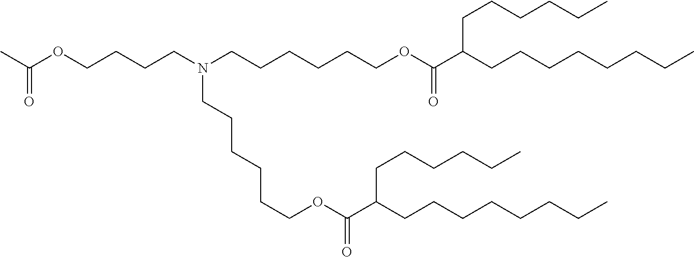 [SMILES: CCCCCCCCC(CCCCCC)C(=O)OCCCCCCN(CCCCCCOC(=O)C(CCCCCC)CCCCCCCC)CCCCOC(C)=O] | F |
| III-34 | 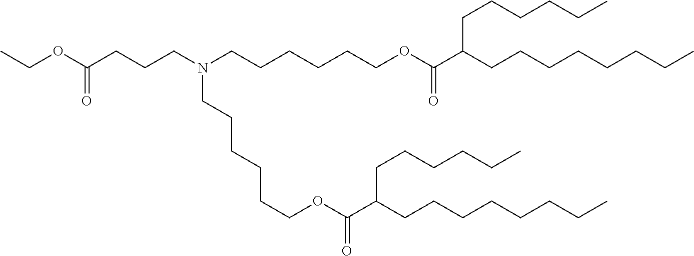 [SMILES: CCCCCCCCC(CCCCCC)C(=O)OCCCCCCN(CCCCCCOC(=O)C(CCCCCC)CCCCCCCC)CCCC(=O)OCC] | F |
| III-35 | 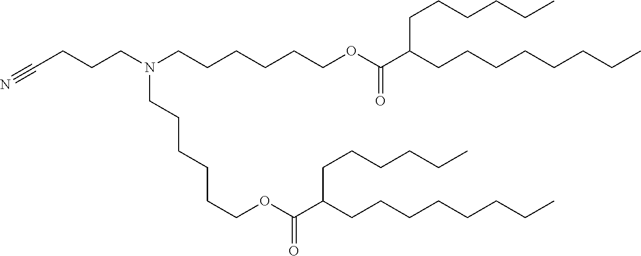 [SMILES: CCCCCCCCC(CCCCCC)C(=O)OCCCCCCN(CCCC#N)CCCCCCOC(=O)C(CCCCCC)CCCCCCCC] | F |
| III-36 | 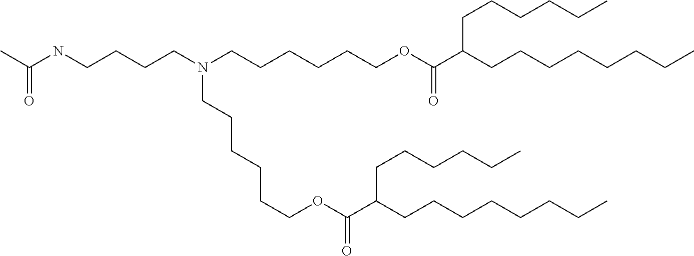 [SMILES: CCCCCCCCC(CCCCCC)C(=O)OCCCCCCN(CCCCCCOC(=O)C(CCCCCC)CCCCCCCC)CCCCNC(C)=O] | F |

In some embodiments, the LNPs comprise a lipid of Formula (III), a first nucleoside-modified RNA encoding an antigen, a second nucleoside-modified RNA encoding a cytokine or immune receptor (such as but not limited to a cytokine receptor), and one or more excipient selected from neutral lipids, steroids and pegylated lipids. In some embodiments, the lipid of Formula (III) is compound III-3. In some embodiments, the lipid of Formula (III) is compound III-7.

In certain embodiments, the cationic lipid is present in the LNP in an amount from about 30 to about 95 mole percent. In some embodiments, the cationic lipid is present in the LNP in an amount from about 30 to about 70 mole percent. In some embodiments, the cationic lipid is present in the LNP in an amount from about 40 to about 60 mole percent. In some embodiments, the cationic lipid is present in the LNP in an amount of about 50 mole percent. In some embodiments, the LNP comprises only cationic lipids.

In certain embodiments, the LNP comprises one or more additional lipids which stabilize the formation of particles during their formation.

Suitable stabilizing lipids include neutral lipids and anionic lipids.

The term “neutral lipid” refers to any one of a number of lipid species that exist in either an uncharged or neutral zwitterionic form at physiological pH.

Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE). In some embodiments, the neutral lipid is 1,2-distearoyl-sn-glycero-3-phosphocholine (DSPC).

In some embodiments, the LNPs comprise a neutral lipid selected from DSPC, DPPC, DMPC, DOPC, POPC, DOPE and SM. In various embodiments, the molar ratio of the cationic lipid (e.g., lipid of Formula (I)) to the neutral lipid ranges from about 2:1 to about 8:1.

In various embodiments, the LNPs further comprise a steroid or steroid analogue. A “steroid” is a compound comprising the following carbon skeleton:

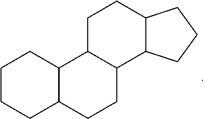 [SMILES: C1CCC2C(C1)CCC1C3CCCC3CCC21]

In certain embodiments, the steroid or steroid analogue is cholesterol. In some of these embodiments, the molar ratio of the cationic lipid (e.g., lipid of Formula (I)) to cholesterol ranges from about 2:1 to 1:1.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitovloleyolphosphatidylglycerol (POPG), and other anionic modifying groups joined to neutral lipids.

In certain embodiments, the LNP comprises glycolipids (e.g., monosialoganglioside GM₁). In certain embodiments, the LNP comprises a sterol, such as cholesterol.

In some embodiments, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

In certain embodiments, the LNP comprises an additional, stabilizing-lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In some embodiments, the polyethylene glycol-lipid is N-[(methoxy poly(ethylene glycol)2ooo)carbamyl]-1,2-dimyristyloxlpropyl-3-amine (PEG-c-DMA). In some embodiments, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-l-O—(O-methoxy(polyethoxy)ethyl)butanedioate (PEG-S-DMG), a pegylated ceramide (PEG-cer), or a PEG dialkoxypropylcarbamate such as co-methoxy(polyethoxy)ethyl-N-(2,3-di(tetradecanoxy)propyl)carbamate or 2,3-di(tetradecanoxy)propyl-N-(co-methoxy(polyethoxy)ethyl)carbamate. In various embodiments, the molar ratio of the cationic lipid to the pegylated lipid ranges from about 100:1 to about 25:1.

In some embodiments, the LNPs comprise a pegylated lipid having the following structure (IV):

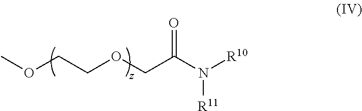

or a pharmaceutically acceptable salt, tautomer or stereoisomer thereof wherein:
R¹⁰ and R¹¹ are each independently a straight or branched, saturated or unsaturated alkyl chain containing from 10 to 30 carbon atoms, wherein the alkyl chain is optionally interrupted by one or more ester bonds; and
z has mean value ranging from 30 to 60.

In some of the foregoing embodiments of the pegylated lipid (IV), R¹⁰ and R¹¹ are not both n-octadecyl when z is 42. In some other embodiments, R¹⁰ and R¹¹ are each independently a straight or branched, saturated or unsaturated alkyl chain containing from 10 to 18 carbon atoms. In some embodiments, R¹⁰ and R¹¹ are each independently a straight or branched, saturated or unsaturated alkyl chain containing from 12 to 16 carbon atoms. In some embodiments, R¹⁰ and R¹¹ are each independently a straight or branched, saturated or unsaturated alkyl chain containing 12 carbon atoms. In some embodiments, R¹⁰ and R¹¹ are each independently a straight or branched, saturated or unsaturated alkyl chain containing 14 carbon atoms. In other embodiments, R¹⁰ and R¹¹ are each independently a straight or branched, saturated or unsaturated alkyl chain containing 16 carbon atoms. In still more embodiments, R¹⁰ and R¹¹ are each independently a straight or branched, saturated or unsaturated alkyl chain containing 18 carbon atoms. In still other embodiments, R¹⁰ is a straight or branched, saturated or unsaturated alkyl chain containing 12 carbon atoms and R¹¹ is a straight or branched, saturated or unsaturated alkyl chain containing 14 carbon atoms.

In various embodiments, z spans a range that is selected such that the PEG portion of (II) has an average molecular weight of about 400 to about 6000 g/mol. In some embodiments, the average z is about 45.

In other embodiments, the pegylated lipid has one of the following structures:

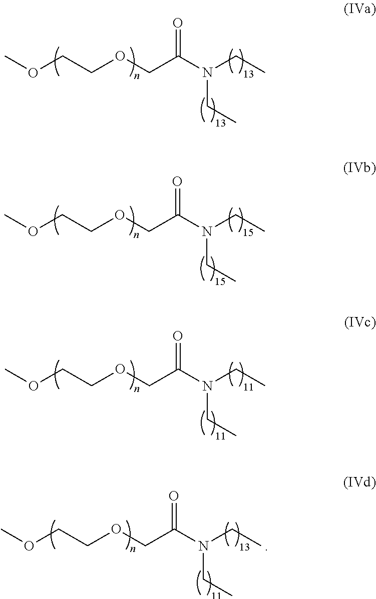

wherein n is an integer selected such that the average molecular weight of the pegylated lipid is about 2500 g/mol.

In certain embodiments, the additional lipid is present in the LNP in an amount from about 1 to about 10 mole percent. In some embodiments, the additional lipid is present in the LNP in an amount from about 1 to about 5 mole percent. In some embodiments, the additional lipid is present in the LNP in about 1 mole percent or about 1.5 mole percent.

In some embodiments, the LNPs comprise a lipid of Formula (I), a first nucleoside-modified RNA encoding an antigen, a second nucleoside-modified RNA encoding a cytokine or immune receptor (such as but not limited to a cytokine receptor), a neutral lipid, a steroid and a pegylated lipid. In some embodiments the lipid of Formula (I) is compound I-6. In certain embodiments, the neutral lipid is DSPC. In some embodiments, the steroid is cholesterol. In certain embodiments, the pegylated lipid is compound IVa.

In certain embodiments, the LNP comprises one or more targeting moieties, which are capable of targeting the LNP to a cell or cell population. For example, in some embodiments, the targeting moiety is a ligand, which directs the LNP to a receptor found on a cell surface.

In certain embodiments, the LNP comprises one or more internalization domains. For example, in some embodiments, the LNP comprises one or more domains, which bind to a cell to induce the internalization of the LNP. For example, in some embodiments, the one or more internalization domains bind to a receptor found on a cell surface to induce receptor-mediated uptake of the LNP. In certain embodiments, the LNP is capable of binding a biomolecule in vivo, where the LNP-bound biomolecule can then be recognized by a cell-surface receptor to induce internalization. For example, in some embodiments, the LNP binds systemic ApoE, which leads to the uptake of the LNP and associated cargo.

Other exemplary LNPs and their manufacture are described in the art, for example in U.S. Patent Application Publication No. US20120276209, Semple et ak, 2010, Nat Biotechnok, 28(2): 172-176; Akinc et ak, 2010, Mol Then, 18(7): 1357-1364; Basha et ak, 2011, Mol Ther, 19(12): 2186-2200; Leung et ak, 2012, J Phys Chem C Nanomater Interfaces, 116(34): 18440-18450; Lee et ak, 2012, Int J Cancer., 131(5): E781-90; Belliveau et ak, 2012, Mol Ther nucleic Acids, 1: e37; Jayaraman et ak, 2012, Angew Chem Int Ed Engl., 51(34): 8529-8533; Mui et ak, 2013, Mol Ther Nucleic Acids. 2, e139; Maier et ak, 2013, Mol Then, 21(8): 1570-1578; and Tam et ak, 2013, Nanomedicine, 9(5): 665-74, each of which is incorporated by reference in its entirety. Additionally, WO 2022/081752, incorporated herein by reference in its entirety, describes LNP manufacturing techniques for increasing the potency of nucleic acid loaded lipid nanoparticles.

The following Reaction Schemes illustrate methods to make lipids of Formula (I), (II) or (III).

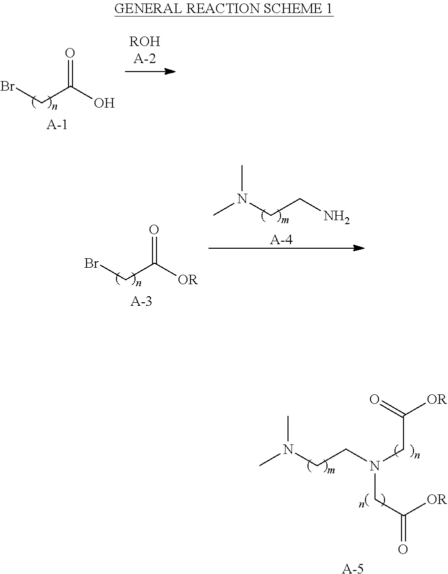

Embodiments of the lipid of Formula (I) (e.g., compound A-5) can be prepared according to General Reaction Scheme 1 (“Method A”), wherein R is a saturated or unsaturated C₁-C₂₄ alkyl or saturated or unsaturated cycloalkyl, m is 0 or 1 and n is an integer from 1 to 24. Referring to General Reaction Scheme 1, compounds of structure A-1 can be purchased from commercial sources or prepared according to methods familiar to one of ordinary skill in the art. A mixture of A-1, A-2 and DMAP is treated with DCC to give the bromide A-3. A mixture of the bromide A-3, a base (e.g., N,N-diisopropylethylamine) and the N,N-dimethyldiamine A-4 is heated at a temperature and time sufficient to produce A-5 after any necessary workup and or purification step.

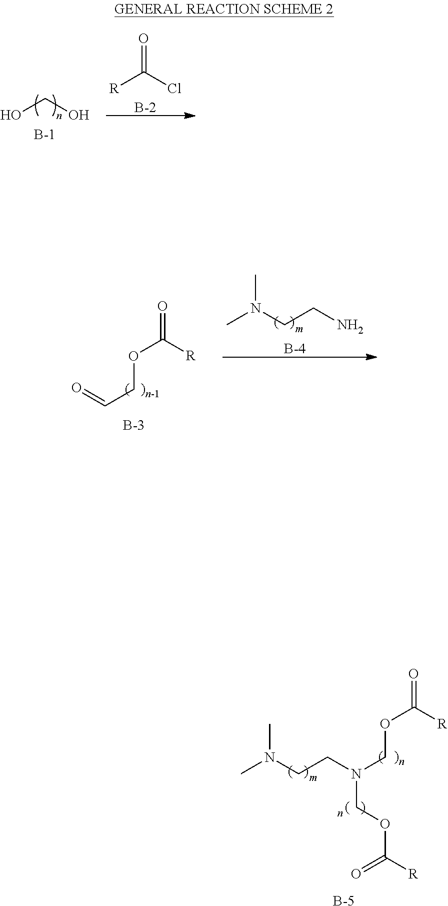

Other embodiments of the compound of Formula (I) (e.g., compound B-5) can be prepared according to General Reaction Scheme 2 (“Method B”), wherein R is a saturated or unsaturated C₁-C₂₄ alkyl or saturated or unsaturated cycloalkyl, m is 0 or 1 and n is an integer from 1 to 24. As shown in General Reaction Scheme 2, compounds of structure B-1 can be purchased from commercial sources or prepared according to methods familiar to one of ordinary skill in the art. A solution of B-1 (1 equivalent) is treated with acid chloride B-2 (1 equivalent) and a base (e.g., triethylamine). The crude product is treated with an oxidizing agent (e.g., pyridinum chlorochromate) and intermediate product B-3 is recovered. A solution of crude B-3, an acid (e.g., acetic acid), and N,N-dimethylaminoamine B-4 is then treated with a reducing agent (e.g., sodium triacetoxyborohydride) to obtain B-5 after any necessary work up and/or purification.

It should be noted that although starting materials A-1 and B-1 are depicted above as including only saturated methylene carbons, starting materials which include carbon-carbon double bonds may also be employed for preparation of compounds which include carbon-carbon double bonds.

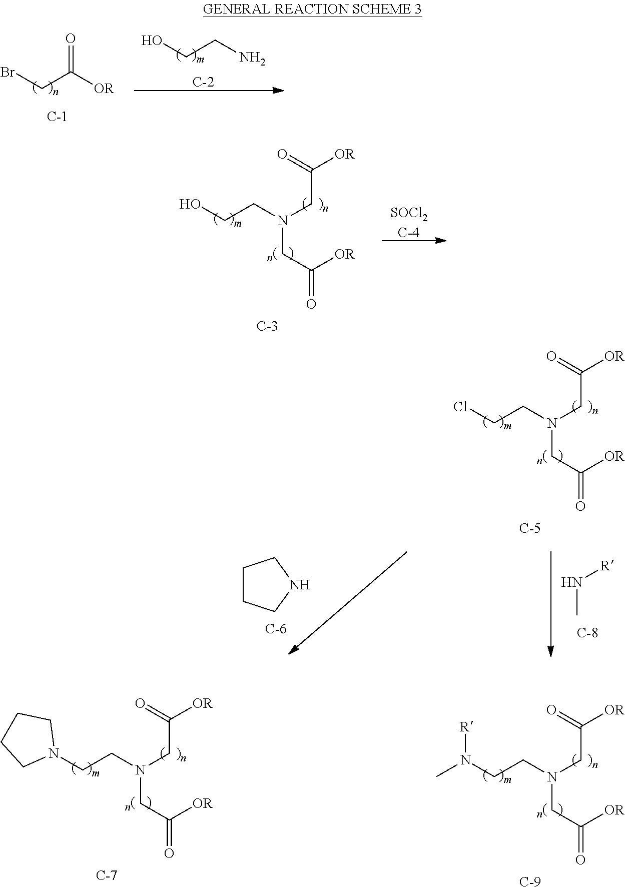

Different embodiments of the lipid of Formula (I) (e.g., compound C-7 or C9) can be prepared according to General Reaction Scheme 3 (“Method C”), wherein R is a saturated or unsaturated C₁-C₂₄ alkyl or saturated or unsaturated cycloalkyl, m is 0 or 1 and n is an integer from 1 to 24. Referring to General Reaction Scheme 3, compounds of structure C-1 can be purchased from commercial sources or prepared according to methods familiar to one of ordinary skill in the art.

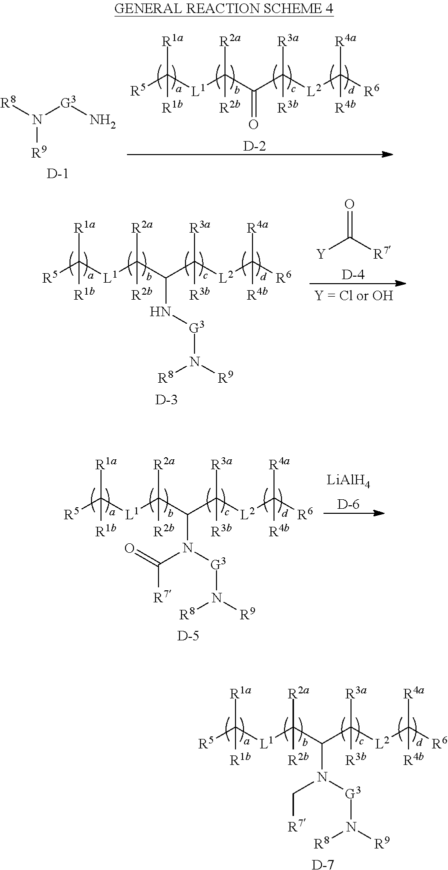

Embodiments of the compound of Formula (II) (e.g., compounds D-5 and D-7) can be prepared according to General Reaction Scheme 4 (“Method D”), wherein R^{1a}, R^{1b}, R^{2a}, R^{2b}, R^{3a}, R^{3b}, R^{4a}, R^{4b}, R⁵, R⁶, R⁸, R⁹, L¹, L², G¹, G², G³, a, b, c and d are as defined herein, and R⁷ represents R⁷ or a C₃-C₁₉ alkyl. Referring to General Reaction Scheme 1, compounds of structure D-1 and D-2 can be purchased from commercial sources or prepared according to methods familiar to one of ordinary skill in the art. A solution of D-1 and D-2 is treated with a reducing agent (e.g., sodium triacetoxyborohydride) to obtain D-3 after any necessary work up. A solution of D-3 and a base (e.g. trimethylamine, DMAP) is treated with acyl chloride D-4 (or carboxylic acid and DCC) to obtain D-5 after any necessary work up and/or purification. D-5 can be reduced with LiAlH₄ D-6 to give D-7 after any necessary work up and/or purification.

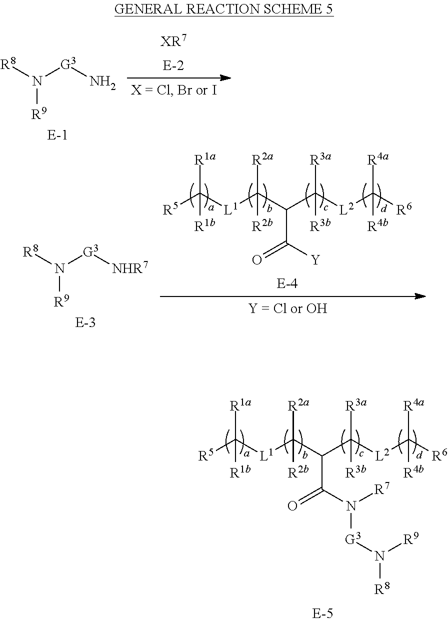

Embodiments of the lipid of Formula (II) (e.g., compound E-5) can be prepared according to General Reaction Scheme 5 (“Method E”), wherein R^{1a}, R^{1b}, R^{2a}, R^{2b}, R^{3a}, R^{3b}R^{4a}, R^{4b}, R⁵, R⁶, R⁷, R⁸, R⁹, L¹, L², G³, a, b, c and d are as defined herein. Referring to General Reaction Scheme 5, compounds of structure E-1 and E-2 can be purchased from commercial sources or prepared according to methods familiar to one of ordinary skill in the art. A mixture of E-1 (in excess), E-2 and a base (e.g., potassium carbonate) is heated to obtain E-3 after any necessary work up. A solution of E-3 and a base (e.g. trimethylamine, DMAP) is treated with acyl chloride E-4 (or carboxylic acid and DCC) to obtain E-5 after any necessary work up and/or purification.

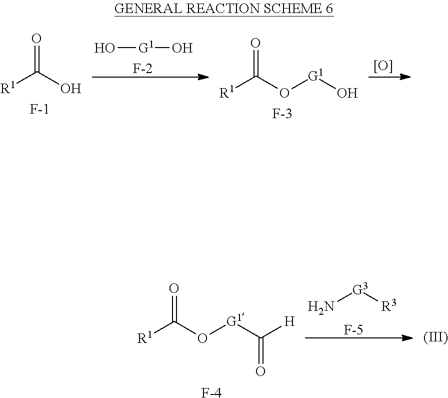

General Reaction Scheme 6 provides an exemplary method (Method F) for preparation of Lipids of Formula (III). G¹, G³, R¹ and R³ in General Reaction Scheme 6 are as defined herein for Formula (III), and G1′ refers to a one-carbon shorter homologue of G1. Compounds of structure F-1 are purchased or prepared according to methods known in the art. Reaction of F-1 with diol F-2 under appropriate condensation conditions (e.g., DCC) yields ester/alcohol F-3, which can then be oxidized (e.g., PCC) to aldehyde F-4. Reaction of F-4 with amine F-5 under reductive amination conditions yields a lipid of Formula (III).

It should be noted that various alternative strategies for preparation of lipids of Formula (III) are available to those of ordinary skill in the art. For example, other lipids of Formula (III) wherein L¹ and L² are other than ester can be prepared according to analogous methods using the appropriate starting material. Further, General Reaction Scheme 6 depicts preparation of a lipids of Formula (III), wherein G¹ and G² are the same; however, this is not a required aspect of the invention and modifications to the above reaction scheme are possible to yield compounds wherein G¹ and G² are different.

It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethyl silyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

### Antigens

The present invention provides an LNP and composition comprising the LNP that elicit a modulated immune response against an antigen in a subject. In some embodiments, the LNP comprises a nucleic acid sequence which encodes an antigen. For example, in certain embodiments, the LNP comprises a nucleoside-modified RNA encoding an antigen. In certain embodiments, the LNP comprises a purified, nucleoside-modified RNA encoding an antigen. The antigen may include, but is not limited to, a polypeptide, peptide, or protein that induces an immune response in a subject. As one skilled in the art would understand, any polypeptide that induces an immune response in a subject is an antigen, regardless of whether that polypeptide is a full-length protein or a fragment thereof. As used herein, the term “immunogen” is synonymous with “antigen.”

In some embodiments, the antigen is encoded by a nucleic acid sequence of a nucleic acid molecule. In certain embodiments, the nucleic acid sequence comprises DNA, RNA, cDNA, a variant thereof, a fragment thereof, or a combination thereof. In some embodiments, the nucleic acid sequence comprises a modified nucleic acid sequence. For example, in one embodiment the antigen-encoding nucleic acid sequence comprises nucleoside-modified RNA, as described in detail elsewhere herein. In certain instances, the nucleic acid sequence comprises additional sequences that encode linker or tag sequences that are linked to the encoded antigen by a peptide bond.

RNA molecules used with the invention for immunization purposes, in some embodiments, encode a polypeptide immunogen. In these embodiments, after administration, the RNA is translated in vivo and the immunogen can elicit an immune response in the recipient. The immunogen may elicit an immune response against a pathogen (e.g. a bacterium, a virus, a fungus or a parasite) but, in some embodiments, it elicits an immune response against an allergen or a tumor antigen. The immune response may comprise an antibody response (usually including IgG) and/or a cell mediated immune response. The polypeptide immunogen will typically elicit an immune response which recognizes the corresponding pathogen (or allergen or tumor) polypeptide, but in some embodiments the polypeptide may act as a mimotope to elicit an immune response which recognizes a saccharide. The immunogen will typically be a surface polypeptide e.g. an adhesin, a hemagglutinin, an envelope glycoprotein, a spike glycoprotein, etc.

The RNA molecule can encode a single polypeptide immunogen or multiple polypeptides. Multiple immunogens can be presented as a single polypeptide immunogen (fusion polypeptide) or as separate polypeptides. If immunogens are expressed as separate polypeptides from an mRNA, then one or more of these may be provided with an upstream IRES or an additional viral promoter element. Alternatively, multiple immunogens may be expressed from a polyprotein that encodes individual immunogens fused to a short autocatalytic protease (e.g. foot-and-mouth disease virus 2A protein), or as inteins.

In certain embodiments, polypeptide immunogens (e.g., 1, 2, 3, 4, 5, 6, 7, 8, 9, 10, or more immunogens) may be used, either alone or together with a RNA molecule, such as a self-replicating RNA, encoding one or more immunogens (either the same or different as the polypeptide immunogens).

Tolerable variations of the nucleotide and amino acid sequences of the antigen will be known to those of skill in the art. For example, in certain embodiments the antigen comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any naturally-occurring or known reference amino acid sequence of the antigen.

In other embodiments, the antigen is encoded by a nucleotide sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any naturally-occurring or known reference nucleotide sequence encoding the antigen.

In some embodiments, the antigen is derived from a pathogen. Pathogens include viruses, bacteria, fungi, and parasites.

In some embodiments the immunogen (i.e., the antigen) is derived from and elicits an immune response against one of these bacteria:

Neisseria meningitidis: useful immunogens include, but are not limited to, membrane proteins such as adhesins, autotransporters, toxins, iron acquisition proteins, and factor H binding protein. A combination of three useful polypeptides is disclosed in Giuliani et al. (2006) Proc Natl Acad Sci USA 103(29): 10834-9.

Streptococcus pneumoniae: useful polypeptide immunogens are disclosed in WO2009/016515. These include, but are not limited to, the RrgB pilus subunit, the beta-N-acetyl-hexosaminidase precursor (spr0057), spr0096, General stress protein GSP-781 (spr2021, SP2216), serine/threonine kinase StkP (SP1732), and pneumococcal surface adhesin PsaA.

Streptococcus pyogenes: useful immunogens include, but are not limited to, the polypeptides disclosed in WO02/34771 and WO2005/032582.
Moraxella catarrhalis.

Bordetella pertussis: Useful pertussis immunogens include, but are not limited to, pertussis toxin or toxoid (PT), filamentous haemagglutinin (FHA), pertactin, and agglutinogens 2 and 3.

Staphylococcus aureus: Useful immunogens include, but are not limited to, the polypeptides disclosed in WO2010/119343, such as a hemolysin, esxA, esxB, ferrichrome-binding protein (sta006) and/or the sta011 lipoprotein.

Clostridium tetani: the typical immunogen is tetanus toxoid.

Cornynebacterium diphtheriae: the typical immunogen is diphtheria toxoid.

Haemophilus influenzae: Useful immunogens include, but are not limited to, the polypeptides disclosed in WO2006/110413 and WO2005/111066.
Pseudomonas aeruginosa

Streptococcus agalactiae: useful immunogens include, but are not limited to, the polypeptides disclosed in WO02/34771.

Chlamydia trachomatis: Useful immunogens include, but are not limited to, PepA, LcrE, ArtJ, DnaK, CT398, OmpH-like, L7/L12, OmcA, AtoS, CT547, Eno, HtrA and MurG (e.g. as disclosed in WO2005/002619). LcrE (WO2006/138004) and HtrA (WO2009/109860) are two preferred immunogens.

Chlamydia pneumoniae: Useful immunogens include, but are not limited to, the polypeptides disclosed in WO02/02606.

Helicobacter pylori: Useful immunogens include, but are not limited to, CagA, VacA, NAP, and/or urease (WO03/018054).

Escherichia coli: Useful immunogens include, but are not limited to, immunogens derived from enterotoxigenic E. coli (ETEC), enteroaggregative E. coli (EAggEC), diffusely adhering E. coli (DAEC), enteropathogenic E. coli (EPEC), extraintestinal pathogenic E. coli (ExPEC) and/or enterohemorrhagic E. coli (EHEC). ExPEC strains include uropathogenic E. coli (UPEC) and meningitis/sepsis-associated E. coli (MNEC). Useful UPEC immunogens are disclosed in WO2006/091517 and WO2008/020330. Useful MNEC immunogens are disclosed in WO2006/089264. A useful immunogen for several E. coli types is AcfD (WO2009/104092).
Bacillus anthracis

Yersinia pestis: Useful immunogens include, but are not limited to, those disclosed in WO2007/049155 and WO2009/031043.
Staphylococcus epidermis
Clostridium perfringens or Clostridium botulinums
Legionella pneumophila
Coxiella burnetiid
Brucella, such as B. abortus, B. canis, B. melitensis, B. neotomae, B. ovis, B. suis, B. pinnipediae.
Francisella, such as F. novicida, F. philomiragia, F. tularensis
Neisseria gonorrhoeae
Treponema pallidum
Haemophilus ducreyi
Enterococcus faecalis or Enterococcus faecium
Staphylococcus saprophyticus
Yersinia enterocolitica
Mycobacterium tuberculosis
Rickettsia
Listeria monocytogenes
Vibrio cholerae
Salmonella typhi
Borrelia burgdorferi
Porphyromonas gingivalis
Klebsiella

In some embodiments the immunogen elicits an immune response against one of these viruses:
Orthomyxovirus: Useful immunogens can be from an influenza A, B or C virus, such as the hemagglutinin, neuraminidase or matrix M2 proteins. Where the immunogen is an influenza A virus hemagglutinin it may be from any subtype e.g. H1, H2, H3, H4, H5, H6, H7, H8, H9, H10, H11, H12, H13, H14, H15 or H16. Useful influenza immunogens are described in WO21/202734, which is incorporated herein by reference in its entirety.

Paramyxoviridae viruses: immunogens include, but are not limited to, those derived from Pneumoviruses (e.g. respiratory syncytial virus, RSV), Rubulaviruses (e.g. mumps virus), Paramyxoviruses (e.g. parainfluenza virus), Metapneumoviruses and Morbilliviruses (e.g. measles virus).

Poxviridae: immunogens include, but are not limited to, those derived from Orthopoxvirus such as Variola vera, including but not limited to, Variola major and Variola minor.

Picornavirus: immunogens include, but are not limited to, those derived from Picornaviruses, such as Enteroviruses, Rhinoviruses, Heparnavirus, Cardioviruses and Aphthoviruses. In some embodiments, the enterovirus is a poliovirus e.g. a type 1, type 2 and/or type 3 poliovirus. In another embodiment, the enterovirus is an EV71 enterovirus. In another embodiment, the enterovirus is a coxsackie A or B virus.

Bunyavirus: immunogens include, but are not limited to, those derived from an Orthobunyavirus, such as California encephalitis virus, a Phlebovirus, such as Rift Valley Fever virus, or a Nairovirus, such as Crimean-Congo hemorrhagic fever virus.

Heparnavirus: immunogens include, but are not limited to, those derived from a Heparnavirus, such as hepatitis A virus (HAV).

Filovirus: immunogens include, but are not limited to, those derived from a filovirus, such as an Ebola virus (including a Zaire, Ivory Coast, Reston or Sudan ebolavirus) or a Marburg virus.

Togavirus: immunogens include, but are not limited to, those derived from a Togavirus, such as a Rubivirus, an Alphavirus, or an Arterivirus. This includes rubella virus.

Flavivirus: immunogens include, but are not limited to, those derived from a Flavivirus, such as Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus, Powassan encephalitis virus.

Pestivirus: immunogens include, but are not limited to, those derived from a Pestivirus, such as Bovine viral diarrhea (BVDV), Classical swine fever (CSFV) or Border disease (BDV).

Hepadnavirus: immunogens include, but are not limited to, those derived from a Hepadnavirus, such as Hepatitis B virus. A composition can include hepatitis B virus surface antigen (HBsAg).

Other hepatitis viruses: A composition can include an immunogen from a hepatitis C virus, delta hepatitis virus, hepatitis E virus, or hepatitis G virus.

Rhabdovirus: immunogens include, but are not limited to, those derived from a Rhabdovirus, such as a Lyssavirus (e.g. a Rabies virus) and Vesiculovirus (VSV).

Caliciviridae: immunogens include, but are not limited to, those derived from Caliciviridae, such as Norwalk virus (Norovirus), and Norwalk-like Viruses, such as Hawaii Virus and Snow Mountain Virus.

Coronavirus: immunogens include, but are not limited to, those derived from SARS-CoV-2, a SARS coronavirus, avian infectious bronchitis (IBV), Mouse hepatitis virus (MHV), severe acute respiratory syndrome (SARS) virus, Middle East respiratory syndrome (MERS), and Porcine transmissible gastroenteritis virus (TGEV). In addition, immunogens from bat and pangolin coronaviruses with pandemic potential can be used. The coronavirus immunogen may be a spike polypeptide or other viral protein. Specific Coronavirus epitopes are comprehensively analyzed and described in Shrock et al, Science, Sep. 29, 2020 which is incorporated herein by reference.

The SARS-CoV-2 antigen may be of any type or strain of SARS-CoV-2. For example, in some embodiments, the SARS-CoV-2 antigen is a protein, or fragment thereof, of a SARS-CoV-2 strain including, but not limited to, Wuhan-Hu-1 (GenBank: MN908947.3) which was initially identified in Wuhan, China in December, 2019. This initially-identified SARS-CoV-2 virus is referred to herein as “wild-type” or “WT” SARS-CoV-2. Numerous genetic variants of SARS-CoV-2 have since emerged and have been identified through sequence-based surveillance, laboratory studies, and epidemiological investigations. These SARS-CoV-2 genetic variants include, but are not limited to, the B.1.1.7 variant (also termed the “UK variant” or “Alpha variant”), the B.1.351 variant (also termed the “South African variant” or “Beta variant”), the P.1 variant (also termed “Gamma variant”), the B.1.427 variant, the B.1.429 variant, the B.1.526 variant, the B.1.526.1 variant, the B.1.525 variant, the P.2 variant, the B.1.617 variant (“Indian variant”), the cluster 5/ΔFVI-spike variant (“Denmark Mink variant”), the B.1.526 variant (“New York variant”), the B.1.427 and B.1.429 variants (“California variants”), the BV-1 variant (“Texas variant”), the B.1.617.2 variant (“Delta variant”), B.1.1.529 (“Omicron variant”) and sublineages thereof (e.g., BA.1, BA.1.1, BA.2, BA.3, BA.4, and BA.5). SARS-CoV-2 variants contain mutations relative to wild-type SARS-CoV-2. For example, the L452R spike protein mutation is present in the B.1.526.1, B.1.427, and B.1.429 variants and the E484K spike protein mutation is present in B.1.525, P.2, P.1, and B.1.351 variants, but only in some strains of B.1.526 and B.1.1.7 variants. SARS-CoV-2 antigens that are useful in the present invention are described, e.g., in WO 2022/011092, which is incorporated herein by reference in its entirety.

Retrovirus: immunogens include, but are not limited to, those derived from an Oncovirus, a Lentivirus (e.g. HIV-1 or HIV-2) or a Spumavirus.

Reovirus: immunogens include, but are not limited to, those derived from an Orthoreovirus, a Rotavirus, an Orbivirus, or a Coltivirus.

Parvovirus: immunogens include, but are not limited to, those derived from Parvovirus B19.

Herpesvirus: immunogens include, but are not limited to, those derived from a human herpesvirus, such as, by way of example only, Herpes Simplex Viruses (HSV) (e.g. HSV types 1 and 2), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8).

Papovaviruses: immunogens include, but are not limited to, those derived from Papillomaviruses and Polyomaviruses. The (human) papillomavirus may be of serotype 1, 2, 4, 5, 6, 8, 11, 13, 16, 18, 31, 33, 35, 39, 41, 42, 47, 51, 57, 58, 63 or 65 e.g. from one or more of serotypes 6, 11, 16 and/or 18.

Adenovirus: immunogens include those derived from serotype 36 (Ad-36).

In some embodiments, the immunogen elicits an immune response against a virus which infects fish, such as: infectious salmon anemia virus (ISAV), salmon pancreatic disease virus (SPDV), infectious pancreatic necrosis virus (IPNV), channel catfish virus (CCV), fish lymphocystis disease virus (FLDV), infectious hematopoietic necrosis virus (H4NV), koi herpesvirus, salmon picoma-like virus (also known as picoma-like virus of Atlantic salmon), landlocked salmon virus (LSV), Atlantic salmon rotavirus (ASR), trout strawberry disease virus (TSD), coho salmon tumor virus (CSTV), or viral hemorrhagic septicemia virus (VHSV).

Fungal immunogens may be derived from Dermatophytres, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme; or from Aspergillus fumigatus, Aspergillus flavus, Aspergillus niger, Aspergillus nidulans, Aspergillus terreus, Aspergillus sydowii, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi; the less common are Brachiola spp, Microsporidium spp., Nosema spp., Pleistophora spp., Trachipleistophora spp., Vittaforma spp Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Altemaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

In some embodiments the immunogen elicits an immune response against a parasite. There are three main classes of parasites that can cause disease in humans: protozoa, helminths, and ectoparasites.

The protozoa that are infectious to humans can be classified into four groups based on their mode of movement, including: Sarcodina (the ameba, e.g., Entamoeba), Mastigophora (the flagellates, e.g., Giardia, Leishmania), Ciliophora (the ciliates, e.g., Balantidium), and Sporozoa (organisms whose adult stage is not motile e.g., Plasmodium, Cryptosporidium, Toxoplasma).

There are three main groups of helminths (derived from the Greek word for worms) that are human parasites: Flatworms (platyhelminths—these include the trematodes (flukes) and cestodes (tapeworms)). Thomy-headed worms (acanthocephalins—the adult forms of these worms reside in the gastrointestinal tract). Roundworms (nematodes—the adult forms of these worms can reside in the gastrointestinal tract, blood, lymphatic system or subcutaneous tissues. Alternatively, the immature (larval) states can cause disease through their infection of various body tissues).

In some embodiments the immunogen elicits an immune response against a parasite from the Plasmodium genus, such as P. falciparum, P. vivax, P. malariae or P. ovale. Thus the invention may be used for immunizing against malaria. In some embodiments the immunogen elicits an immune response against a parasite from the Caligidae family, particularly those from the Lepeophtheirus and Caligus genera e.g. sea lice such as Lepeophtheirus salmonis or Caligus rogercresseyi.

In some embodiments the immunogen elicits an immune response against: pollen allergens (tree-, herb, weed-, and grass pollen allergens); insect or arachnid allergens (inhalant, saliva and venom allergens, e.g. mite allergens, cockroach and midges allergens, hymenopthera venom allergens); animal hair and dandruff allergens (from e.g. dog, cat, horse, rat, mouse, etc.); and food allergens (e.g. a gliadin). Important pollen allergens from trees, grasses and herbs are such originating from the taxonomic orders of Fagales, Oleales, Pinales and platanaceae including, but not limited to, birch (Betula), alder (Alnus), hazel (Corylus), hornbeam (Carpinus) and olive (Olea), cedar (Cryptomeria and Juniperus), plane tree (Platanus), the order of Poales including grasses of the genera Lolium, Phleum, Poa, Cynodon, Dactylis, Holcus, Phalaris, Secale, and Sorghum, the orders of Asterales and Urticales including herbs of the genera Ambrosia, Artemisia, and Parietaria. Other important inhalation allergens are those from house dust mites of the genus Dermatophagoides and Euroglyphus, storage mite e.g. Lepidoglyphys, Glycyphagus and Tyrophagus, those from cockroaches, midges and fleas e.g. Blatella, Periplaneta, Chironomus and Ctenocepphalides, and those from mammals such as cat, dog and horse, venom allergens including such originating from stinging or biting insects such as those from the taxonomic order of Hymenoptera including bees (Apidae), wasps (Vespidea), and ants (Formicoidae).

In some embodiments the immunogen is a tumor antigen selected from: (a) cancer testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors; (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUM1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT; (c) overexpressed antigens, for example, Galectin 4 (associated with, e.g., colorectal cancer), Galectin 9 (associated with, e.g., Hodgkin's disease), proteinase 3 (associated with, e.g., chronic myelogenous leukemia), VVT 1 (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), mammaglobin, alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer); (d) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, e.g., melanoma); (e) prostate associated antigens such as PAP, PSA, PSMA, PSH-P1, PSM-P1, PSM-P2, associated with e.g., prostate cancer; (f) immunoglobulin idiotypes (associated with myeloma and B cell lymphomas, for example). In certain embodiments, tumor immunogens include, but are not limited to, pl 5, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, pl85erbB2, pl80erbB-3, c-met, mn-23Hl, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29BCAA), CA 195, CA 242, CA-50, CAM43, CD68KP1, CO-029, FGF-5, Ga733 (EpCAM), HTgp-175, M344, MA-50, MG7-Ag, M0V18, NB/70K, NY-CO-1, RCAS1, SDCCAG16, TA-90 (Mac-2 binding protein/cyclophilin C-associated protein), TAAL6, TAG72, TLP, TPS, and the like.

### Nucleic Acids

In some embodiments, the invention includes a nucleoside-modified nucleic acid molecule (e.g., a nucleoside-modified RNA). In some embodiments, the nucleoside-modified RNA encodes an antigen. In some embodiments, the nucleoside-modified RNA encodes a cytokine or immune receptor (such as but not limited to a cytokine receptor). In certain embodiments, the nucleoside-modified RNA encodes an antigen that induces an adaptive immune response against the antigen. In certain embodiments, the nucleoside-modified RNA encodes a cytokine or immune receptor (such as but not limited to a cytokine receptor) that elicits a modified immune response against the antigen. In some embodiments, the modified immune response is an enhanced immune response. In some embodiments, the modified immune response is a decreased immune response. In certain embodiments, the modulated immune response is tissue-specific. In some embodiments, the cytokine or immune receptor (such as but not limited to a cytokine receptor) is capable of modulating the magnitude and/or functional phenotype of the immune response.

The nucleotide sequences encoding an antigen, a cytokine or immune receptor (such as but not limited to a cytokine receptor), as described herein, can alternatively comprise sequence variations with respect to the original nucleotide sequences, for example, substitutions, insertions and/or deletions of one or more nucleotides, with the condition that the resulting polynucleotide encodes a polypeptide according to the invention. Therefore, the scope of the present invention includes nucleotide sequences that are substantially homologous to the known and/or reference nucleotide sequences which encode an antigen or a cytokine or immune receptor (such as but not limited to a cytokine receptor) of interest.

As used herein, a nucleotide sequence is “substantially homologous” to any of the nucleotide sequences described herein when its nucleotide sequence has a degree of identity with respect to the nucleotide sequence of at least 60%, of at least 65%, of at least 70%, of at least 65%, of at least 80%, of at least 85%, of at least 90%, of at least 95%, of at least 96%, of at least 97%, of at least 98%, or of at least 99%. A nucleotide sequence that is substantially homologous to a nucleotide sequence encoding an antigen or a cytokine or immune receptor (such as but not limited to a cytokine receptor) can typically be isolated from a producer organism of the antigen or cytokine or immune receptor (such as but not limited to a cytokine receptor) based on the information contained in the nucleotide sequence by means of introducing conservative or non-conservative substitutions, for example. Other examples of possible modifications include the insertion of one or more nucleotides within the sequence, the addition of one or more nucleotides at the 3′ and/or 5′ end of the sequence, or the deletion of one or more nucleotides at the 3′ and/or 5′ end or from within the sequence. The degree of identity between two polynucleotides is determined using computer algorithms and methods that are widely known for the persons skilled in the art.

Further, the scope of the invention includes nucleotide sequences that encode amino acid sequences of the antigen that preserve the immunogenic function of the antigen as well as nucleotide sequences that encode amino acid sequences of the cytokine or immune receptor (such as but not limited to a cytokine receptor) that preserve the immunogenic function of the cytokine or immune receptor (such as but not limited to a cytokine receptor).

As used herein, an amino acid sequence is “substantially homologous” to a known or reference amino acid sequence when its amino acid sequence has a degree of identity with respect to the amino acid sequence of at least 60%, of at least 65%, of at least 70%, of at least 65%, of at least 80%, of at least 85%, of at least 90%, of at least 95%, of at least 96%, of at least 97%, of at least 98%, or of at least 99%. The identity between two amino acid sequences can be determined by using the BLASTP algorithm (BLAST Manual, Altschul, S., et ah, NCBI NLM NIH Bethesda, Md. 20894, Altschul, S., et ah, J. Mol. Biol. 215: 403-410 (1990)).

### In Vitro Transcribed RNA

In some embodiments, the LNP and/or composition of the invention comprises in vitro transcribed (IVT) RNA encoding an antigen and IVT RNA encoding a cytokine or immune receptor (such as but not limited to a cytokine receptor). In some embodiments, the LNP and/or composition of the invention comprises IVT RNA encoding one or more antigens and one or more cytokines or immune receptors (such as but not limited to a cytokine receptors).

In some embodiments, an IVT RNA can be introduced to a cell as a form of transient transfection. The RNA is produced by in vitro transcription using a plasmid DNA template generated synthetically. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. In some embodiments, the desired template for in vitro transcription is an antigen capable of inducing an adaptive immune response. In some embodiments, the desired template for in vitro transcription is a cytokine or immune receptor (such as but not limited to a cytokine receptor) capable of modulating the magnitude and/or functional phenotype of an adaptive immune response.

In some embodiments, the DNA to be used for PCR contains an open reading frame. The DNA can be from a naturally occurring DNA sequence from the genome of an organism. In some embodiments, the DNA is a full length gene of interest of a portion of a gene. The gene can include some or all of the 5′ and/or 3′ untranslated regions (UTRs). The gene can include exons and introns. In some embodiments, the DNA to be used for PCR is a human gene. In another embodiment, the DNA to be used for PCR is a human gene including the 5′ and 3′ UTRs. In another embodiment, the DNA to be used for PCR is a gene from a pathogenic or commensal organism, including bacteria, viruses, parasites, and fungi. In another embodiment, the DNA to be used for PCR is from a pathogenic or commensal organism, including bacteria, viruses, parasites, and fungi, including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

Genes that can be used as sources of DNA for PCR include genes that encode polypeptides that induce an enhanced or a decreased adaptive immune response in an organism. In certain instances, the genes are useful for a short term treatment. In certain instances, the genes have limited safety concerns regarding dosage of the expressed gene.

In various embodiments, a plasmid is used to generate a template for in vitro transcription of mRNA, which is used for transfection.

Chemical structures with the ability to promote stability and/or translation efficiency may also be used. In certain embodiments, the RNA has 5′ and 3′ UTRs. In some embodiments, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In some embodiments, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In some embodiments, the promoter is a T7 RNA polymerase promoter, as described elsewhere herein. Other useful promoters include, but are not limited to, T3 and SP6 RNA polymerase promoters. Consensus nucleotide sequences for T7, T3 and SP6 promoters are known in the art.

In some embodiments, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product, which is not suitable for expression in eukaryotic cells. The transcription of plasmid DNA linearized at the end of the 3′ UTR results in normal sized mRNA, which is effective in eukaryotic transfection when it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However, polyA/T sequence integrated into plasmid DNA can cause plasmid instability, which can be ameliorated through the use of recombination incompetent bacterial cells for plasmid propagation.

Poly(A) tails of RNAs can be further extended following in vitro transcription with the use of a poly(A) polymerase, such as E. coli polyA polymerase (E-PAP) or yeast polyA polymerase. In some embodiments, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to mRNA molecules. In some embodiments, RNAs produced by the methods to include a 5′ cap structure. Such cap structure can be generated using Vaccinia capping enzyme and T-O-methyl transferase enzymes (CellScript, Madison, WI). Alternatively, 5′ cap is provided using techniques known in the art and described herein (Cougot, et ah, Trends in Biochem. Sci., 29:436-444 (2001); Stepinski, et ah, RNA, 7:1468-95 (2001); Elango, et ah, Biochim. Biophys. Res. Commun., 330:958-966 (2005)).

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001). In certain embodiments RNA of the invention is introduced to a cell with a method comprising the use of TransIT®-mRNA transfection Kit (Mirus, Madison WI), which, in some instances, provides high efficiency, low toxicity, transfection. In certain embodiments RNA of the invention is introduced into a cell (e.g., a cell of a subject) via encapsulation within an LNP as described herein.

### Nucleoside-Modified RNA

In some embodiments, the LNP and/or composition of the present invention comprises a nucleoside-modified nucleic acid encoding an antigen or a cytokine or immune receptor (such as but not limited to a cytokine receptor) as described herein.

In some embodiments, the LNP and/or composition of the present invention comprises a nucleoside-modified nucleic acid encoding a plurality of antigens. In some embodiments, the composition of the present invention comprises a nucleoside-modified nucleic acid encoding a cytokine or immune receptor (such as but not limited to a cytokine receptor) as described herein. In some embodiments, the composition of the present invention comprises a nucleoside-modified nucleic acid encoding one or more cytokines or immune receptors (such as but not limited to a cytokine receptors).

For example, in some embodiments, the LNP and/or composition comprises a nucleoside-modified RNA. In some embodiments, the LNP and/or composition comprises a nucleoside-modified mRNA. Nucleoside-modified mRNA have particular advantages over non-modified mRNA, including for example, increased stability, low or absent innate immunogenicity, and enhanced translation. Nucleoside-modified mRNA useful in the present invention is further described in U.S. Pat. Nos. 8,278,036, 8,691,966, and 8,835,108, each of which is incorporated by reference herein in its entirety.

In certain embodiments, nucleoside-modified mRNA does not activate any pathophysiologic pathways, translates very efficiently and almost immediately following delivery, and serve as templates for continuous protein production in vivo lasting for several days (Kariko et al., 2008, Mol Ther 16:1833-1840; Kariko et al., 2012, Mol Ther 20:948-953). The amount of mRNA required to exert a physiological effect is small and that makes it applicable for human therapy. For example, as described herein, nucleoside-modified mRNA encoding a SARS-CoV-2 antigen has demonstrated the ability to elicit antigen-specific antibody production. For example, in certain instances, antigen encoded by nucleoside-modified mRNA induces greater production of antigen-specific antibody production as compared to antigen encoded by non-modified mRNA.

In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Moreover, purification of proteins is problematic and they can contain aggregates and other impurities that cause adverse effects (Kromminga and Schellekens, 2005, Ann NY Acad Sci 1050:257-265).

In certain embodiments, the nucleoside-modified RNA comprises the naturally occurring modified-nucleoside pseudouridine. In certain embodiments, inclusion of pseudouridine makes the mRNA more stable, non-immunogenic, and highly translatable (Kariko et ah, 2008, Mol Ther 16:1833-1840; Anderson et ah, 2010, Nucleic Acids Res 38:5884-5892; Anderson et al., 2011, Nucleic Acids Research 39:9329-9338; Kariko et al., 2011, Nucleic Acids Research 39:e142; Kariko et al., 2012, Mol Ther 20:948-953; Kariko et al., 2005, Immunity 23:165-175).

It has been demonstrated that the presence of modified nucleosides, including pseudouridines, in RNA suppress their innate immunogenicity (Kariko et al., 2005, Immunity 23:165-175). Further, protein-encoding, in vitro-transcribed RNA containing pseudouridine can be translated more efficiently than RNA containing no or other modified nucleosides (Kariko et al., 2008, Mol Ther 16:1833-1840). Subsequently, it is shown that the presence of pseudouridine improves the stability of RNA (Anderson et al., 2011, Nucleic Acids Research 39:9329-9338) and abates both activation of PKR and inhibition of translation (Anderson et al., 2010, Nucleic Acids Res 38:5884-5892).

In certain embodiments, the nucleoside-modified nucleic acid molecule is a purified nucleoside-modified nucleic acid molecule. For example, in certain embodiments, the composition is purified to remove double-stranded contaminants. In certain instances, a preparative high performance liquid chromatography (HPLC) purification procedure is used to obtain pseudouridine-containing RNA that has superior translational potential and no innate immunogenicity (Kariko et al., 2011, Nucleic Acids Research 39:el42). Administering HPLC-purified, pseudourine-containing RNA coding for erythropoietin into mice and macaques resulted in a significant increase of serum EPO levels (Kariko et al., 2012, Mol Ther 20:948-953), thus confirming that pseudouridine-containing mRNA is suitable for in vivo protein therapy. In certain embodiments, the nucleoside-modified nucleic acid molecule is purified using non-HPLC methods. In certain instances, the nucleoside-modified nucleic acid molecule is purified using chromatography methods, including but not limited to HPLC and fast protein liquid chromatography (FPLC). An exemplary FPLC-based purification procedure is described in Weissman et al., 2013, Methods Mol Biol, 969: 43-54. Exemplary purification procedures are also described in U.S. Patent Application Publication No. US2016/0032316, which is hereby incorporated by reference in its entirety.

The present invention encompasses RNA, oligoribonucleotide, and polyribonucleotide molecules comprising pseudouridine or a modified nucleoside. In certain embodiments, the composition comprises an isolated nucleic acid encoding an antigen, wherein the nucleic acid comprises a pseudouridine or a modified nucleoside. In certain embodiments, the composition comprises a vector, comprising an isolated nucleic acid encoding an antigen, adjuvant, or combination thereof, wherein the nucleic acid comprises a pseudouridine or a modified nucleoside.

In some embodiments, the nucleoside-modified RNA of the invention is IVT RNA, as described elsewhere herein. For example, in certain embodiments, the nucleoside-modified RNA is synthesized by T7 phage RNA polymerase. In another embodiment, the nucleoside-modified mRNA is synthesized by SP6 phage RNA polymerase. In another embodiment, the nucleoside-modified RNA is synthesized by T3 phage RNA polymerase.

In some embodiments, the modified nucleoside is m¹acp³Ψ (1-methyl-3-(3-amino-3-carboxypropyl) pseudouridine. In another embodiment, the modified nucleoside is m¹Ψ (1-methylpseudouridine). In another embodiment, the modified nucleoside is Ψm (2′-O-methylpseudouridine). In another embodiment, the modified nucleoside is m⁵D (5-methyldihydrouridine). In another embodiment, the modified nucleoside is m³Ψ (3-methylpseudouridine). In another embodiment, the modified nucleoside is a pseudouridine moiety that is not further modified (Ψ). In another embodiment, the modified nucleoside is a monophosphate, diphosphate, or triphosphate of any of the above pseudouridines. In another embodiment, the modified nucleoside is any other pseudouridine-like nucleoside known in the art.

In another embodiment, the nucleoside that is modified in the nucleoside-modified RNA the present invention is a modified uridine (U). In another embodiment, the modified nucleoside is a modified cytidine (C). In another embodiment, the modified nucleoside is a modified adenosine (A). In another embodiment, the modified nucleoside is a modified guanosine (G).

In another embodiment, the modified nucleoside of the present invention is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is Ψ (pseudouridine). In another embodiment, the modified nucleoside is Um (2′-O-methyluridine).

In other embodiments, the modified nucleoside is m¹A (1-methyladenosine); m²A (2-methyladenosine); Am (2′-0-methyladenosine); ms²m⁶A (2-methylthio-N⁶-methyladenosine); i⁶A (N⁶-isopentenyladenosine); ms²io⁶A (2-methylthio-N⁶-isopentenyladenosine); io⁶A (N⁶-(cis-hydroxyisopentenyl)adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A (N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²2G (N²,N²-dimethylguanosine); m²Gm (N²,2′-O-dimethylguanosine); m²2Gm (N²,N²,2′-O-trimethylguanosine); Gr(p) (2′-0-ribosylguanosine (phosphate)); yW (wybutosine); O₂yW (peroxywybutosine); OHyW (hydroxywybutosine); OHyW* (undermodified hydroxvwybutosine); imG (wyosine); mimG (methylwxyosine); Q (queuosine); oQ (epoxyqueuosine); galQ (galactosyl-queuosine); manQ (mannosyl-queuosine); preQo (7-cyano-7-deazaguanosine); preQi (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵s²U (5-aminomethyl-2-thiouridine); mnm⁵U (5-methylaminomethyluridine); mnmVU (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶₂A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶2Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine).

In another embodiment, a nucleoside-modified RNA of the present invention comprises a combination of 2 or more of the above modifications. In another embodiment, the nucleoside-modified RNA comprises a combination of 3 or more of the above modifications. In another embodiment, the nucleoside-modified RNA comprises a combination of more than 3 of the above modifications.

In various embodiments, between 0.10% and 100% of the residues in the nucleoside-modified of the present invention are modified (e.g., either by the presence of pseudouridine or another modified nucleoside base). In some embodiments, the fraction of modified residues is 0.1%. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.7%. In another embodiment, the fraction is 0.8%. In another embodiment, the fraction is 0.9%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 7%. In another embodiment, the fraction is 8%. In another embodiment, the fraction is 9%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 55%. In another embodiment, the fraction is 60%. In another embodiment, the fraction is 65%. In another embodiment, the fraction is 70%. In another embodiment, the fraction is 75%. In another embodiment, the fraction is 80%. In another embodiment, the fraction is 85%. In another embodiment, the fraction is 90%. In another embodiment, the fraction is 91%. In another embodiment, the fraction is 92%. In another embodiment, the fraction is 93%. In another embodiment, the fraction is 94%. In another embodiment, the fraction is 95%. In another embodiment, the fraction is 96%. In another embodiment, the fraction is 97%. In another embodiment, the fraction is 98%. In another embodiment, the fraction is 99%. In another embodiment, the fraction is 100%.

In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 8%. In another embodiment, the fraction is less than 10%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, 0.1% of the residues of a given nucleoside (i.e., uridine, cytidine, guanosine, or adenosine) are modified. In another embodiment, the fraction of modified residues is 0.2%. In another embodiment, the fraction is 0.3%. In another embodiment, the fraction is 0.4%. In another embodiment, the fraction is 0.5%.

In another embodiment, the fraction is 0.6%. In another embodiment, the fraction is 0.7%. In another embodiment, the fraction is 0.8%. In another embodiment, the fraction is 0.9%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 7%. In another embodiment, the fraction is 8%. In another embodiment, the fraction is 9%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 55%. In another embodiment, the fraction is 60%. In another embodiment, the fraction is 65%. In another embodiment, the fraction is 70%. In another embodiment, the fraction is 75%. In another embodiment, the fraction is 80%. In another embodiment, the fraction is 85%. In another embodiment, the fraction is 90%. In another embodiment, the fraction is 91%. In another embodiment, the fraction is 92%. In another embodiment, the fraction is 93%. In another embodiment, the fraction is 94%. In another embodiment, the fraction is 95%. In another embodiment, the fraction is 96%. In another embodiment, the fraction is 97%. In another embodiment, the fraction is 98%. In another embodiment, the fraction is 99%. In another embodiment, the fraction is 100%. In another embodiment, the fraction of the given nucleotide that is modified is less than 8%. In another embodiment, the fraction is less than 10%. In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, a nucleoside-modified RNA of the present invention is translated in the cell more efficiently than an unmodified RNA molecule with the same sequence. In another embodiment, the nucleoside-modified RNA exhibits enhanced ability to be translated by a target cell. In another embodiment, translation is enhanced by a factor of 2-fold relative to its unmodified counterpart. In another embodiment, translation is enhanced by a 3-fold factor. In another embodiment, translation is enhanced by a 4-fold factor. In another embodiment, translation is enhanced by a 5-fold factor. In another embodiment, translation is enhanced by a 6-fold factor. In another embodiment, translation is enhanced by a 7-fold factor. In another embodiment, translation is enhanced by a 8-fold factor. In another embodiment, translation is enhanced by a 9-fold factor. In another embodiment, translation is enhanced by a 10-fold factor. In another embodiment, translation is enhanced by a 15-fold factor. In another embodiment, translation is enhanced by a 20-fold factor. In another embodiment, translation is enhanced by a 50-fold factor. In another embodiment, translation is enhanced by a 100-fold factor. In another embodiment, translation is enhanced by a 200-fold factor. In another embodiment, translation is enhanced by a 500-fold factor. In another embodiment, translation is enhanced by a 1000-fold factor. In another embodiment, translation is enhanced by a 2000-fold factor. In another embodiment, the factor is 10-1000-fold. In another embodiment, the factor is 10-100-fold. In another embodiment, the factor is 10-200-fold. In another embodiment, the factor is 10-300-fold. In another embodiment, the factor is 10-500-fold. In another embodiment, the factor is 20-1000-fold. In another embodiment, the factor is 30-1000-fold. In another embodiment, the factor is 50-1000-fold. In another embodiment, the factor is 100-1000-fold. In another embodiment, the factor is 200-1000-fold. In another embodiment, translation is enhanced by any other significant amount or range of amounts.

In another embodiment, the nucleoside-modified antigen-encoding RNA of the present invention induces a significantly more robust adaptive immune response as compared with an unmodified in vitro-synthesized RNA molecule of the same sequence. In another embodiment, the modified RNA molecule induces an adaptive immune response that is 2-fold greater than its unmodified counterpart. In another embodiment, the adaptive immune response is increased by a 3-fold factor. In another embodiment, the adaptive immune response is increased by a 4-fold factor. In another embodiment the adaptive immune response is increased by a 5-fold factor. In another embodiment, the adaptive immune response is increased by a 6-fold factor. In another embodiment, the adaptive immune response is increased by a 7-fold factor. In another embodiment, the adaptive immune response is increased by a 8-fold factor. In another embodiment, the adaptive immune response is increased by a 9-fold factor. In another embodiment, the adaptive immune response is increased by a 10-fold factor. In another embodiment, the adaptive immune response is increased by a 15-fold factor. In another embodiment, the adaptive immune response is increased by a 20-fold factor. In another embodiment, the adaptive immune response is increased by a 50-fold factor. In another embodiment, the adaptive immune response is increased by a 100-fold factor. In another embodiment, the adaptive immune response is increased by a 200-fold factor. In another embodiment, the adaptive immune response is increased by a 500-fold factor. In another embodiment, the adaptive immune response is increased by a 1000-fold factor. In another embodiment, the adaptive immune response is increased by a 2000-fold factor. In another embodiment, the adaptive immune response is increased by another fold difference.

In another embodiment, “induces significantly more robust adaptive immune response” refers to a detectable increase in an adaptive immune response. In another embodiment, the term refers to a fold increase in the adaptive immune response (e.g., 1 of the fold increases enumerated above). In another embodiment, the term refers to an increase such that the nucleoside-modified RNA can be administered at a lower dose or frequency than an unmodified RNA molecule while still inducing a similarly effective adaptive immune response. In another embodiment, the increase is such that the nucleoside-modified RNA can be administered using a single dose to induce an effective adaptive immune response.

In another embodiment, the nucleoside-modified RNA of the present invention exhibits significantly less innate immunogenicity than an unmodified in vitro-synthesized RNA molecule of the same sequence. In another embodiment, the modified RNA molecule exhibits an innate immune response that is 2-fold less than its unmodified counterpart. In another embodiment, innate immunogenicity is reduced by a 3-fold factor. In another embodiment, innate immunogenicity is reduced by a 4-fold factor. In another embodiment, innate immunogenicity is reduced by a 5-fold factor. In another embodiment, innate immunogenicity is reduced by a 6-fold factor. In another embodiment, innate immunogenicity is reduced by a 7-fold factor. In another embodiment, innate immunogenicity is reduced by a 8-fold factor. In another embodiment, innate immunogenicity is reduced by a 9-fold factor. In another embodiment, innate immunogenicity is reduced by a 10-fold factor. In another embodiment, innate immunogenicity is reduced by a 15-fold factor. In another embodiment, innate immunogenicity is reduced by a 20-fold factor. In another embodiment, innate immunogenicity is reduced by a 50-fold factor. In another embodiment, innate immunogenicity is reduced by a 100-fold factor. In another embodiment, innate immunogenicity is reduced by a 200-fold factor. In another embodiment, innate immunogenicity is reduced by a 500-fold factor. In another embodiment, innate immunogenicity is reduced by a 1000-fold factor. In another embodiment, innate immunogenicity is reduced by a 2000-fold factor. In another embodiment, innate immunogenicity is reduced by another fold difference.

In another embodiment, “exhibits significantly less innate immunogenicity” refers to a detectable decrease in innate immunogenicity. In another embodiment, the term refers to a fold decrease in innate immunogenicity (e.g., 1 of the fold decreases enumerated above). In another embodiment, the term refers to a decrease such that an effective amount of the nucleoside-modified RNA can be administered without triggering a detectable innate immune response. In another embodiment, the term refers to a decrease such that the nucleoside-modified RNA can be repeatedly administered without eliciting an innate immune response sufficient to detectably reduce production of the protein encoded by the modified RNA. In another embodiment, the decrease is such that the nucleoside-modified RNA can be repeatedly administered without eliciting an innate immune response sufficient to eliminate detectable production of the protein encoded by the modified RNA.

In some embodiments, the vaccine does not elicit antibodies with antibody-dependent enhancement (ADE) activity. Vaccine-induced enhancement of susceptibility to virus infection or of aberrant viral pathogenesis have been documented for infections by members of different virus families including, but not limited to Dengue virus, Zika virus and feline coronavirus. In some embodiments, “antibodies with ADE activity” refers to antibodies that enhance the entry of virus, and in some cases the replication of virus, into monocytes/macrophages and granulocytic cells through interaction with Fc and/or complement receptors. Therefore, in some embodiments, the vaccine elicits antibodies that do not enhance or cause ADE of disease associated with the antigen, but still neutralize the antigen.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts.

Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient, which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems.

Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, In certain embodiments, the formulation may comprise dry particles which comprise the active ingredient and which have a diameter in the range from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. In certain embodiments, such powders comprise particles wherein at least 98% of the particles by weight have a diameter greater than 0.5 nanometers and at least 95% of the particles by number have a diameter less than 7 nanometers. In certain embodiments, at least 95% of the particles by weight have a diameter greater than 1 nanometer and at least 90% of the particles by number have a diameter less than 6 nanometers. In certain embodiments, dry powder compositions include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in certain instances having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system.

Compositions for sustained release or implantation may comprise pharmaceutically acceptable polymeric or hydrophobic materials such as an emulsion, an ion exchange resin, a sparingly soluble polymer, or a sparingly soluble salt.

As used herein. “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

### Methods of Eliciting a Modulated Immune Response

The present invention provides a method of eliciting a modulated immune response against an antigen in a subject. In some embodiments, the method comprises administering to the subject an effective amount of a composition comprising a lipid nanoparticle (LNP) and at least one pharmaceutically acceptable carrier, diluent, and/or excipient, wherein the LNP comprises: (a) at least one first nucleoside-modified ribonucleic acid (RNA) encoding the antigen; (b) at least one second nucleoside-modified RNA encoding a cytokine or cytokine receptor; and (c) at least one ionizable lipid; wherein the LNP elicits a modified immune response against the antigen in the subject. In some embodiments, the modulated immune response is an enhanced immune response against the antigen in the subject. In certain embodiments, the enhanced immune response in the subject comprises an augmented CD8⁺ T cell response in the subject compared to a CD8⁺ T cell response in a subject administered a control composition lacking the second nucleoside-modified RNA encoding a cytokine or immune receptor (such as but not limited to a cytokine receptor). In some embodiments, the modulated immune response is a reduced immune response against the antigen in the subject. In some embodiments, the reduced immune response in the subject comprises a decreased CD8⁺ T cell response in the subject compared to a CD8⁺ T cell response in a subject administered a control composition lacking the second nucleoside-modified RNA encoding a cytokine or immune receptor (such as but not limited to a cytokine receptor). In certain embodiments, the decreased CD8⁺ T cell response is tissue-specific. In some embodiments, the modulated immune response comprises reduced memory CD8⁺ T cells in spleen and peripheral blood and increased CD8⁺ T cells in mucosal sites (i.e., gut and lung). In some embodiments, the cytokine or immune receptor (such as but not limited to a cytokine receptor) modulates the magnitude and/or functional phenotype of the immune response as described herein.

In some embodiments, the method provides immunity in the subject to the antigen, or to an infection, a disease, or a disorder associated with the antigen. The present invention thus provides a method of treating or preventing the infection, disease, or disorder associated with the antigen.

In some embodiments, the antigen is a SARS-CoV-2 antigen and the disease or disorder associated with SARS-CoV-2 is COVID-19 or a comorbidity of COVID-19.

In some embodiments, the composition is administered to a subject having an infection, disease, or disorder associated with the antigen. In some embodiments, the composition is administered to a subject at risk for developing the infection, disease, or disorder associated with the antigen. For example, the composition may be administered to a subject who is at risk for being in contact with the antigen. In some embodiments, the composition is administered to a subject who lives in, traveled to, or is expected to travel to a geographic region in which the antigen is prevalent. In some embodiments, the composition is administered to a subject who is in contact with or expected to be in contact with another person who lives in, traveled to, or is expected to travel to a geographic region in which the antigen is prevalent. In some embodiments, the composition is administered to a subject who has knowingly been exposed to the antigen through their occupation or contact.

In some embodiments, the method comprises administering a composition comprising an LNP comprising one or more nucleoside-modified nucleic acid molecules encoding one or more antigens and one or more cytokines or immune receptors (such as but not limited to a cytokine receptors). In some embodiments, the method comprises administering a composition comprising an LNP comprising a first nucleoside-modified nucleic acid molecule encoding one or more antigens and a second nucleoside-modified nucleic acid molecule encoding one or more cytokines or immune receptors (such as but not limited to a cytokine receptors).

In certain embodiments, the method of the invention allows for sustained expression of the antigen and/or cytokine or immune receptor (such as but not limited to a cytokine receptor) described herein, for at least several days following administration. In certain embodiments, the method of the invention allows for sustained expression of the antigen and/or cytokine or immune receptor (such as but not limited to a cytokine receptor) described herein for at least 2 weeks following administration. In certain embodiments, the method of the invention allows for sustained expression of the antigen and/or cytokine or immune receptor (such as but not limited to a cytokine receptor) described herein, for at least 1 month following administration. However, the method, in certain embodiments, also provides for transient expression, as in certain embodiments, the nucleic acid is not integrated into the subject genome.

In certain embodiments, the method comprises administering nucleoside-modified RNA, which provides stable expression of the antigen and/or cytokine or immune receptor (such as but not limited to a cytokine receptor) described herein. In some embodiments, administration of nucleoside-modified RNA results in little to no innate immune response, while inducing an effective adaptive immune response.

In certain embodiments, the method provides sustained protection against the antigen and the causative agent from which the antigen is derived (e.g., a pathogen, a tumor, or an allergen). For example, in certain embodiments, the method provides sustained protection against the antigen for more than 2 weeks. In certain embodiments, the method provides sustained protection against the antigen for 1 month or more. In certain embodiments, the method provides sustained protection against the antigen for 2 months or more. In certain embodiments, the method provides sustained protection against the antigen for 3 months or more. In certain embodiments, the method provides sustained protection against the antigen for 4 months or more. In certain embodiments, the method provides sustained protection against the antigen for 5 months or more. In certain embodiments, the method provides sustained protection against the antigen for 6 months or more. In certain embodiments, the method provides sustained protection against the antigen for 1 year or more.

In some embodiments, a single immunization of the composition induces a sustained protection against the antigen for 1 month or more, 2 months or more, 3 months or more, 4 months or more, 5 months or more, 6 months or more, or 1 year or more.

Administration of the compositions of the invention in a method of treatment can be achieved in a number of different ways, using methods known in the art. In some embodiments, the method of the invention comprises systemic administration of the subject, including for example enteral or parenteral administration. In certain embodiments, the method comprises intradermal delivery of the composition. In another embodiment, the method comprises intravenous delivery of the composition. In some embodiments, the method comprises intramuscular delivery of the composition. In some embodiments, the method comprises subcutaneous delivery of the composition. In some embodiments, the method comprises inhalation of the composition. In some embodiments, the method comprises intranasal delivery of the composition.

It will be appreciated that the composition of the invention may be administered to a subject either alone, or in conjunction with another agent.

The therapeutic and prophylactic methods of the invention thus encompass the use of pharmaceutical compositions encoding an antigen and a cytokine or immune receptor (such as but not limited to a cytokine receptor) described herein to practice the methods of the invention. The pharmaceutical compositions useful for practicing the invention may be administered to deliver a dose of from 1 ng/kg/day and 100 mg/kg/day. In some embodiments, the invention envisions administration of a dose, which results in a concentration of the compound of the present invention from 10 nM and 10 mM in a mammal.

Typically, dosages which may be administered in a method of the invention to a mammal, such as a human, range in amount from 0.01 pg to about 50 mg per kilogram of body weight of the mammal, while the precise dosage administered will vary depending upon any number of factors, including but not limited to, the type of mammal and type of disease state being treated, the age of the mammal and the route of administration. In certain embodiments, the dosage of the compound will vary from about 0.1 pg to about 10 mg per kilogram of body weight of the mammal. In certain embodiments, the dosage will vary from about 1 pg to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In certain embodiments, administration of an immunogenic composition or vaccine of the present invention may be performed by single administration or boosted by multiple administrations.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein. Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Lipid Nanoparticle Induced IL-27 Promotes Circulating Memory CD8⁺ T Cells

### IL-27 is Induced Following LNP-mRNA Immunization

The cytokine IL-6 is critical for the development of T follicular helper cells and a robust antibody response following LNP-mRNA immunization, but how it and related cytokine family members impact the induction of CD8+ T cell responses following immunization have remained unexplored. IL-27 is a member of the IL-6 family and has been shown to be critical for the CD8+ T cell response to protein subunit immunizations.

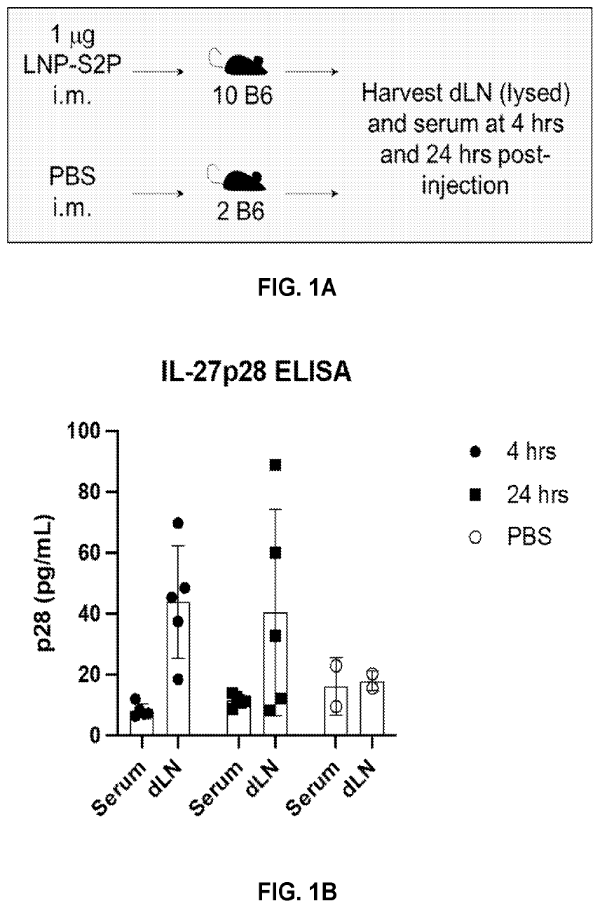

To test whether IL-27 is induced following LNP-mRNA immunization, an LNP-mRNA vaccine against the SARS-CoV-2 spike protein (LNP-S2P) was used. B6 mice were immunized intramuscularly with 1 μg LNP-S2P, whereas control B6 mice received intramuscular injection of phosphate buffered saline (PBS) (FIG. 1A). Draining lymph node (dLN) was harvested, placed in 600 μl of EZ Lyse solution with HALT protease inhibitor cocktail added (Thermo Scientific, cat: 78429) then lysed with a Qiagen Tissue Lyser at 4 h and 24 h post-injection. Serum was also harvested at both time points. Expression of IL-27 was assessed by IL-27p28 ELISA assay (R&D Systems, cat: M2728) which showed production of IL-27 following LNP immunization in draining lymph node at both time points (FIG. 1B).

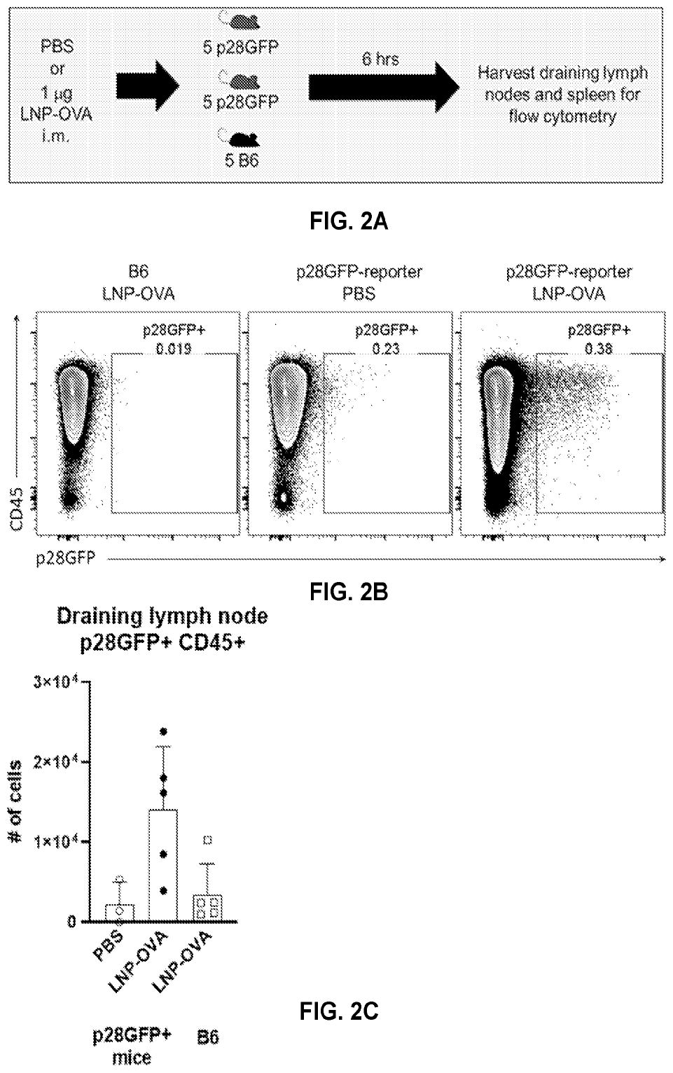

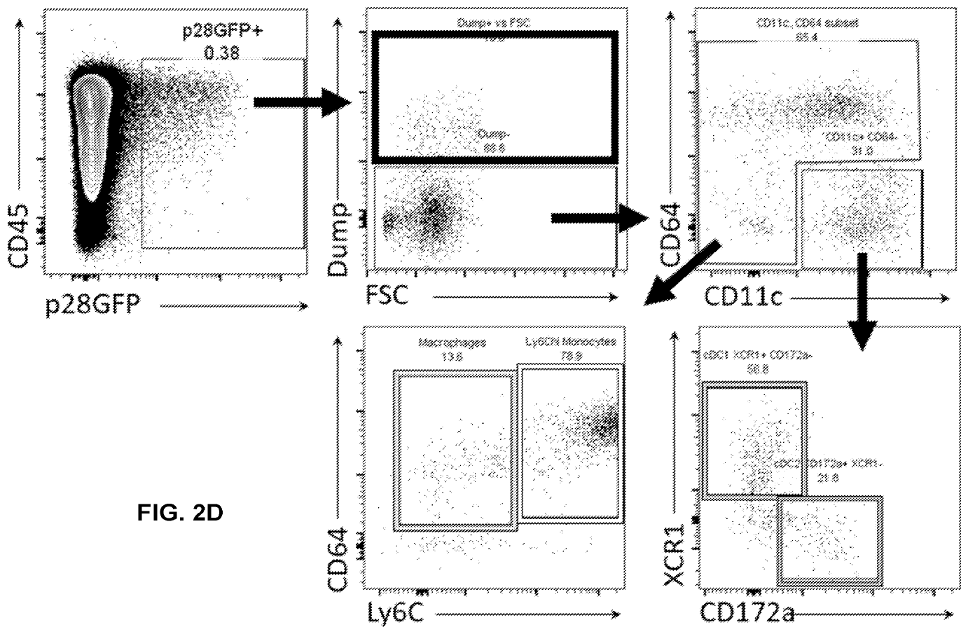

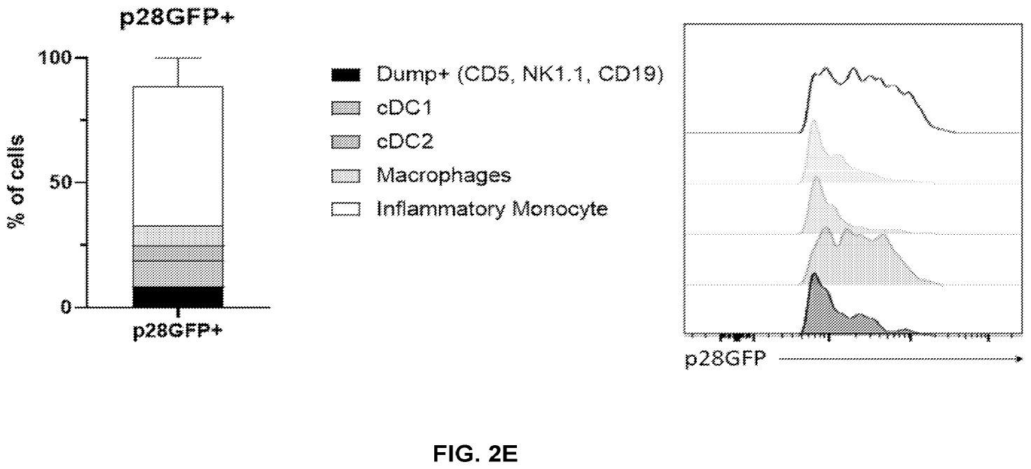

In order to determine the cellular source of IL-27 following LNP-mRNA immunization, B6 mice genetically engineered to express a GFP when IL-27p28 mRNA is expressed (p28GFP) were immunized with 1 g LNPs formulated with modified mRNA encoding the model antigen Ovalbumin (LNP-OVA), whereas control B6 mice received intramuscular injection of PBS (FIG. 2A). Inguinal and popliteal draining lymph nodes were harvested 6 h post i.m. injection and analyzed via flow cytometry. The results show that LNP-OVA immunization rapidly induces IL27p28 expression in draining lymph nodes (FIGS. 2B-2C). IL-27p28 expressing cells are primarily inflammatory monocytes early after immunization, while cDC1s produce at equal levels/cell (FIGS. 2D-2E).

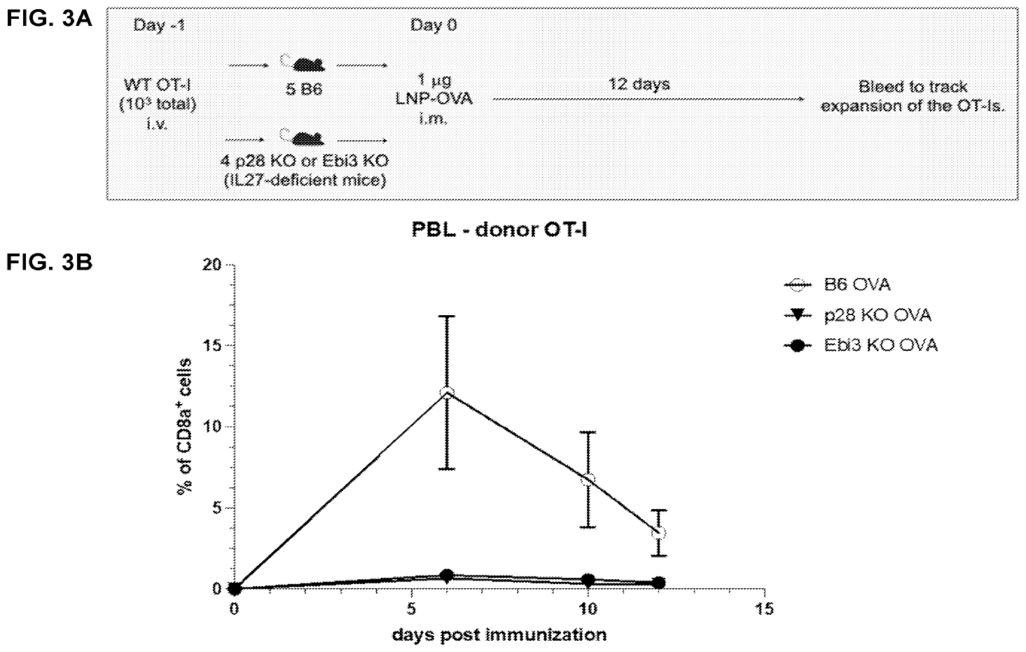

Next, whether IL-27 regulates CD8⁺ T cell responses following LNP-mRNA immunization was investigated using IL-27 knock out (KO) mice. Both p28 KO and Ebi3 KO mice were used (described in Tait Wojno, et al., J Immunol. 2011 Jul. 1; 187(1): 266-273), as well as B6 control mice. The mice received I.V. infusion of 10³ wildtype (WT) OT-I cells on Day −1 and 1 μg i.m. injection of LNP-OVA on Day 0 (FIG. 3A). OT-I cells express transgenic TCR that recognizes the peptide SIINFEKL (SEQ ID NO: 1) bound to MHC class I (K^{b}). The mice were bled 12 days later to track the expansion of the OT-I cells, which showed that loss of IL-27 negatively impacts CD8⁺ T cell expansion following LNP-OVA immunization (FIG. 3B).

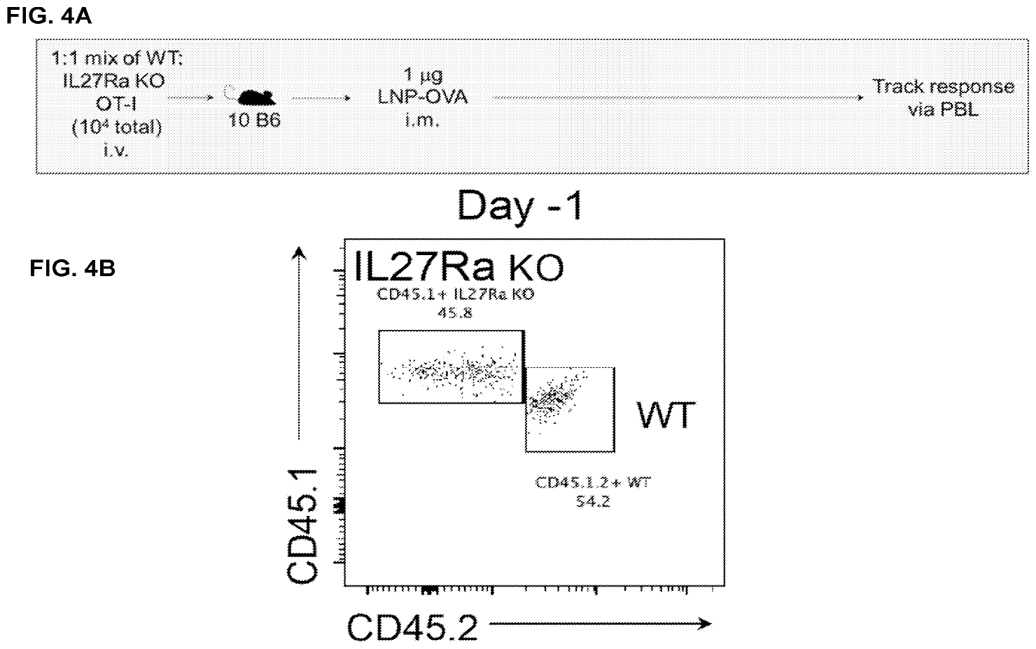

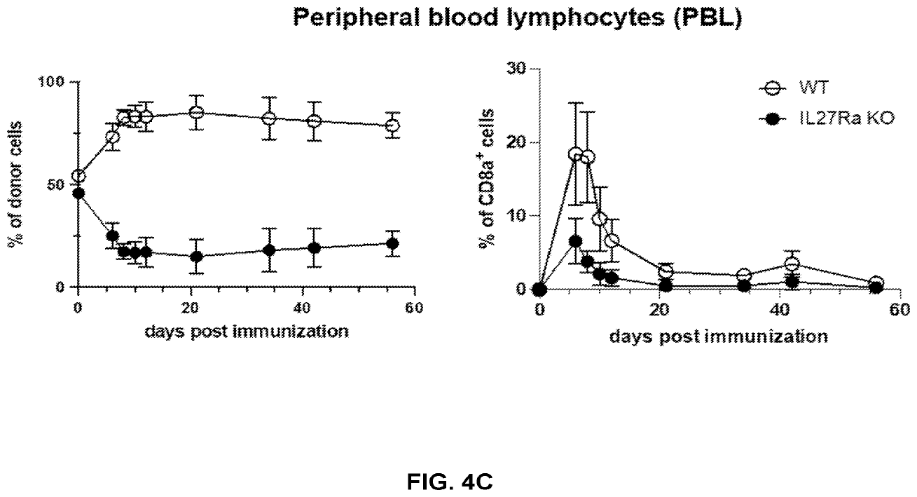

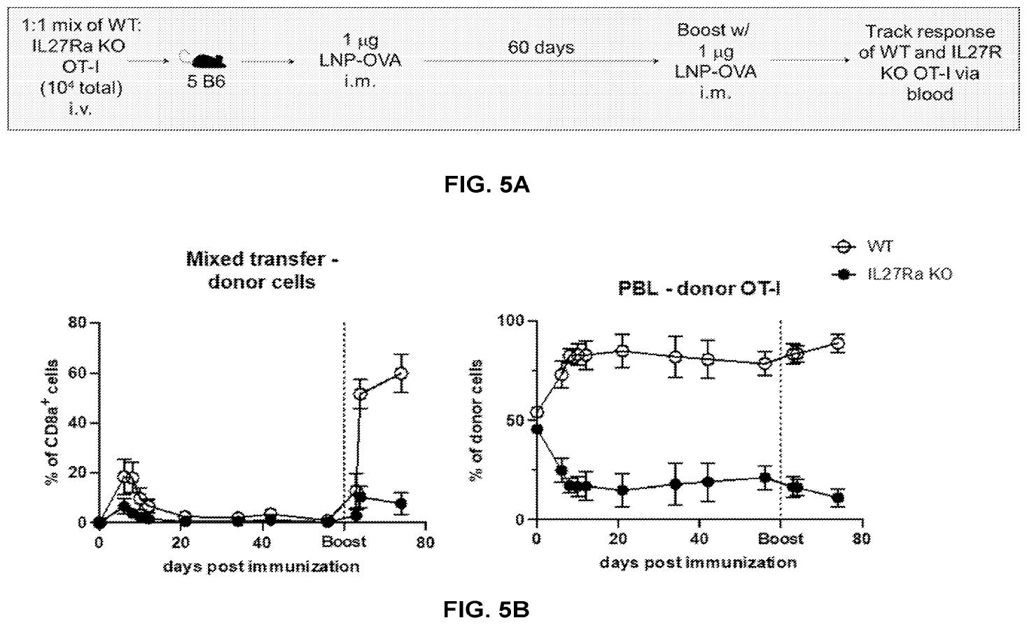

To determine whether the requirement for IL-27 is CD8⁺ T cell-intrinsic, B6 mice received I.V. infusion of 10⁴ total cells which consisted of a 1:1 mix of WT OT-I cells: IL27Ra KO OT-I cells on Day −1 and 1 g i.m. injection of LNP-OVA on Day 0 (FIG. 4A). The response of WT and IL27Ra KO OT-I cells was tracked via peripheral blood lymphocytes (PBLs), which showed that CD8⁺ T cell-intrinsic IL-27 signaling is necessary for maximal expansion (FIGS. 4B-4C). Differences at all time points are statistically significant via paired t test, p<0.0004.

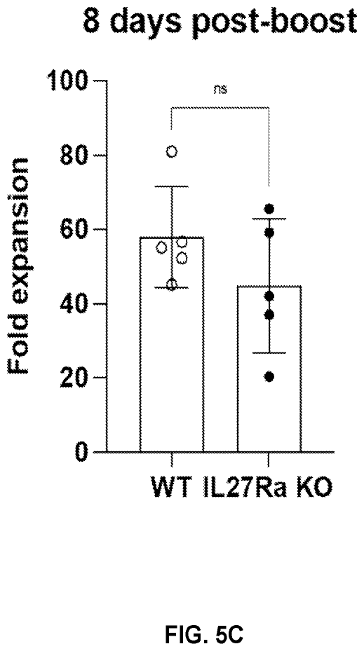

Next, an experiment was performed to determine whether IL-27 signaling is necessary for response to a booster dose of LNP-OVA, and whether long-lived IL-27R-deficient cells respond. B6 mice received I.V. infusion of 10⁴ total cells which was a 1:1 mix of WT OT-I cells: IL27Ra KO OT-T cells on Day −1 and 1 μg i.m. injection of LNP-OVA on Day 0 and Day 60 (FIG. 5A). The response of WT and IL27Ra KO OT-I cells was tracked via PBLs, which showed that the limited number of IL-27R-deficient OT-I cells are still responsive to boost, suggesting that IL-27 may not be as necessary upon boost with LNP-OVA (FIGS. 5B-5C).

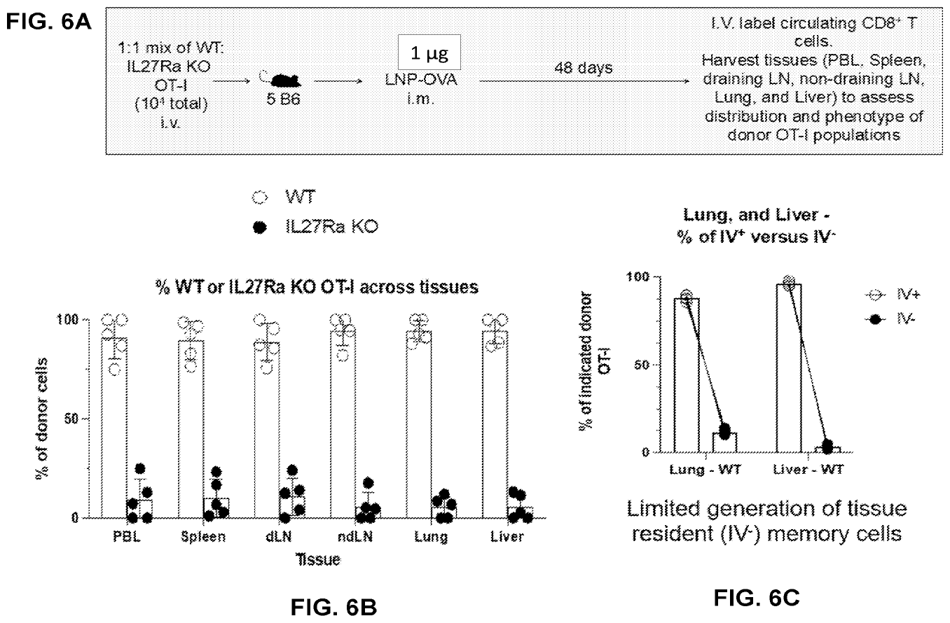

Whether loss of IL-27 signaling impacts distribution of memory cells or tissue residency was also tested. B6 mice received I.V. infusion of 10⁴ total cells which was a 1:1 mix of WT OT-I cells: IL27Ra KO OT-I cells on Day −1 and 1 μg i.m. injection of LNP-OVA on Day 0 (FIG. 6A). After 48 days, circulating CD8⁺ T cells were labeled with 3 μg of anti-CD8α antibody via I.V. injection followed by a 3 minute incubation prior to euthanasia. Tissues (PBL, spleen, draining LN, non-draining LN, lung, and liver) were harvested to assess distribution and phenotype of donor OT-I populations. The results show that loss of IL-27 signaling does not significantly alter OT-1 cell distribution within tissues (FIG. 6B). There is limited tissue-resident memory T cell generation in lung and liver and none in gut (FIG. 6C). Together, the results indicate that a first dose of LNP-mRNA induced IL-27 supports CD8⁺ T cell expansion, which may be less necessary following boost.

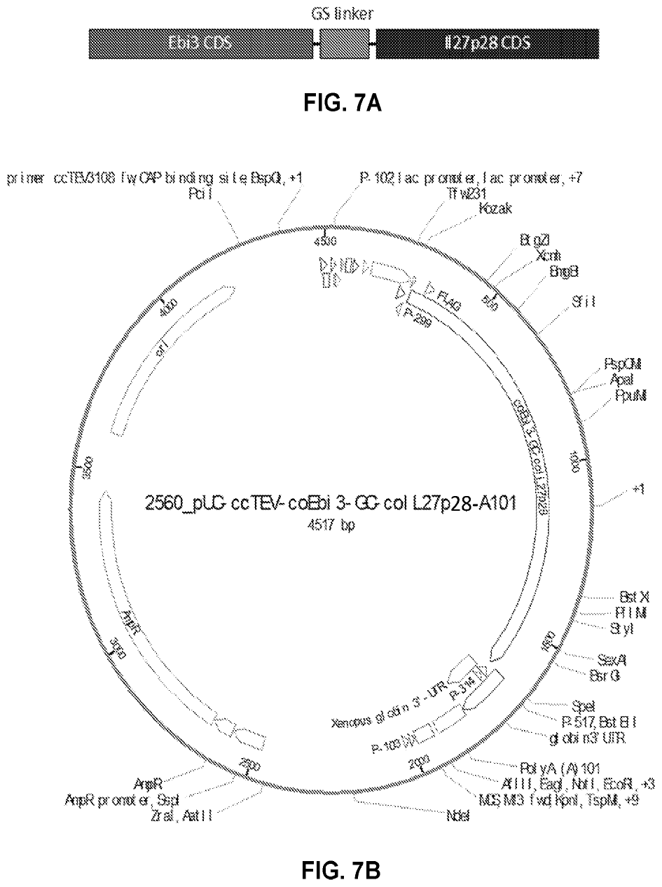

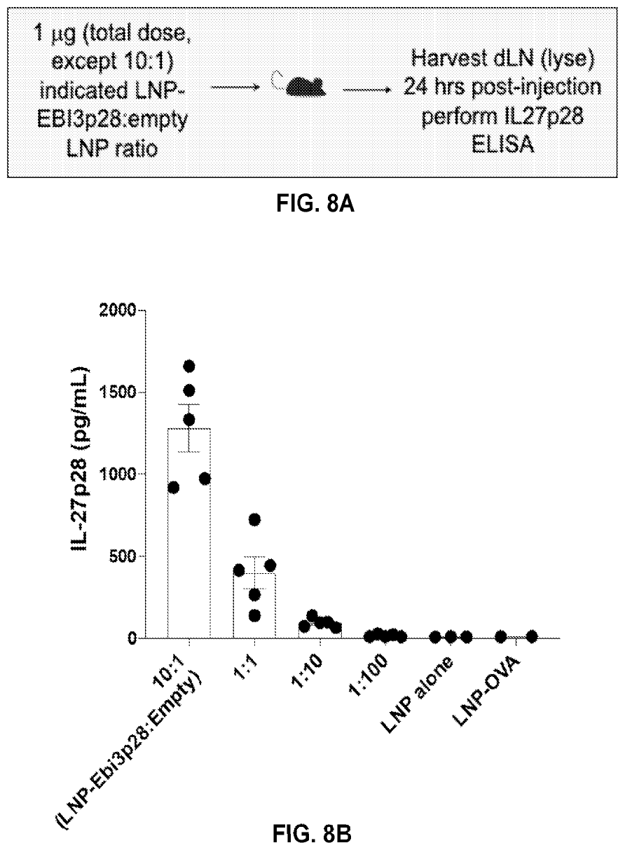

Next, LNP particles were designed and generated to comprise an N1-methyl-pseudouridine-modified codon-optimized mRNA that encodes both subunits of IL-27, Ebi3 and IL27p28, linked by a flexible glycine serine linker (FIG. 7A). This Ebi3p28 mRNA was based on a previously published recombinant IL-27 construct (Pflanz et al., 2002). The mRNA for the Ebi3-p28 IL-27 fusion is encoded on a pUC vector (FIG. 7B). The LNP-Ebi3p28 particles were used to test the in vivo dose response of the LNPs. A titration of the LNP-Ebi3p28 in mice was performed to find a preliminary dose for further studies. Mice received 1 g i.m. injection of an LNP mixture at various ratios of LNP-Ebi3p28 to empty LNPs, ranging from 10:1 to 1:100 (FIG. 8A). Draining lymph nodes were harvested and lysed as described above 24 h post-injection and analyzed for IL27p28 via ELISA which showed that as little as a single dose of 0.1 g of LNP-Ebi3p28 was sufficient to induce 10-fold increased expression of IL-27 detectable in whole tissue lysates of draining lymph nodes 24 hours after immunization (FIG. 8B).

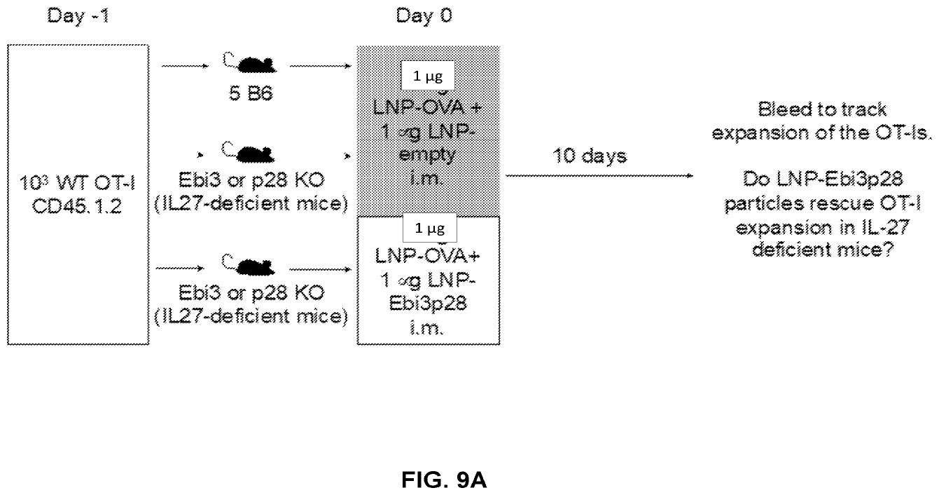

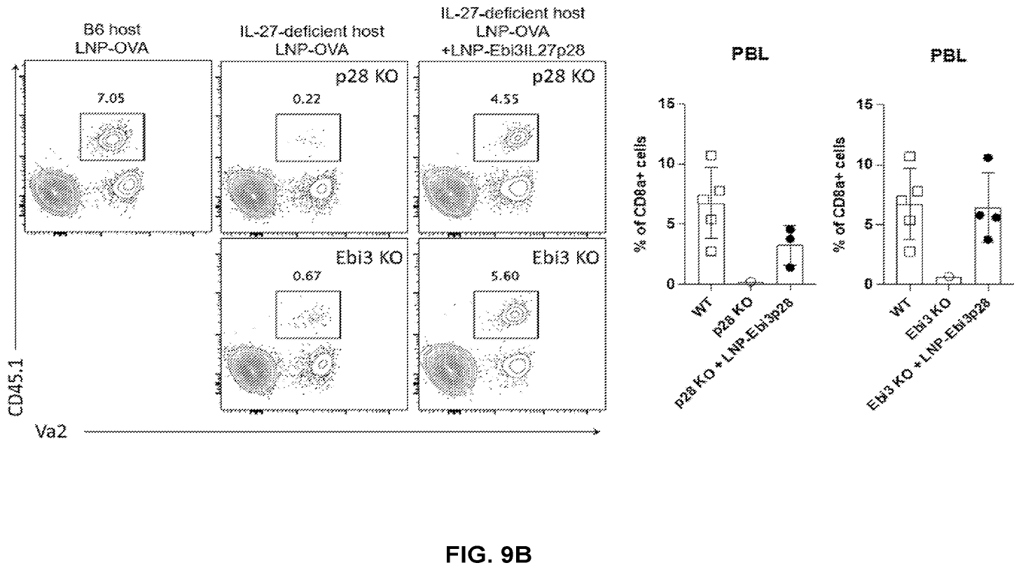

To determine whether the IL-27 produced by LNP-Ebi3p28 particles is functional, the ability of LNP-Ebi3p28 particles to rescue OT-I CD8⁺ T cells expansion in IL-27-deficient mice was tested (FIG. 9A). Mice deficient in either IL-27 subunit, Ebi3 (Ebi3 KO) or IL27p28 (p28 KO), were used as both KO mice have defective CD8⁺ T cell responses to LNP-mRNA immunization. Mice were injected with a 1 g dose of a mixture of LNP-OVA and LNP-Ebi3p28 particles, which at 24 hours post-immunization equates to a ˜40-fold increase in IL-27 levels. The results indicate that antigen-specific CD8⁺ T cell expansion is largely rescued at day 10 post-immunization (FIG. 9B). These data show that cytokine produced via LNP-Ebi3p28 immunization is biologically active and can induce alterations in the immune response in vivo.

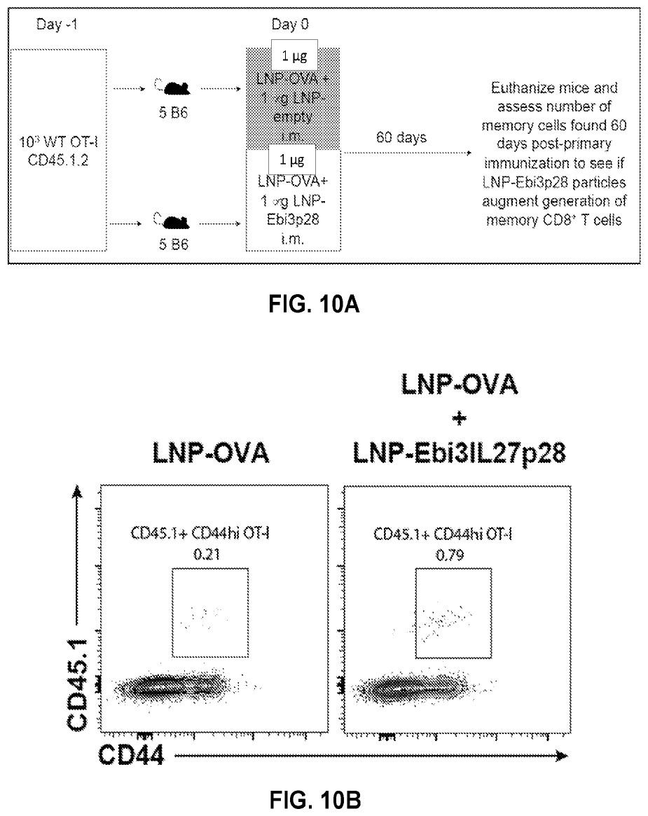

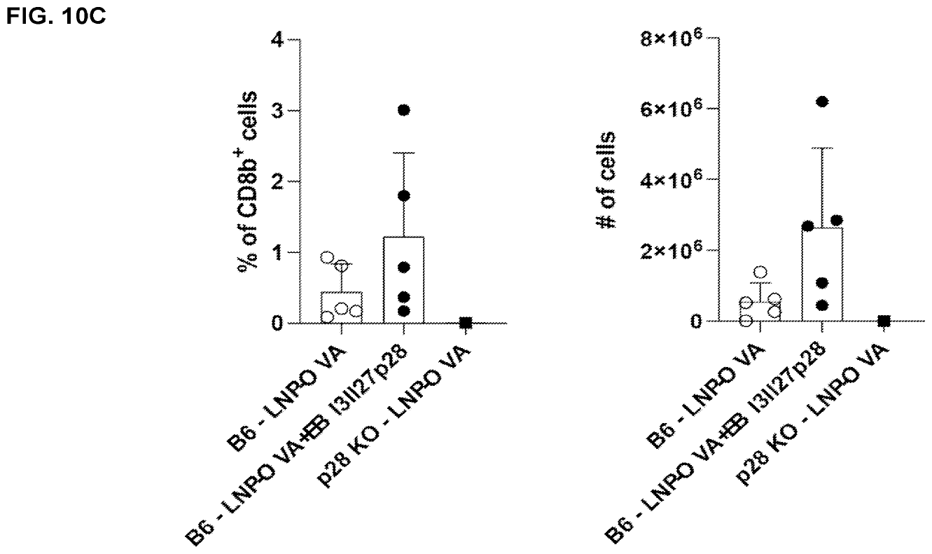

These promising data suggested that LNP-Ebi3p28 particles could be used to modify endogenous immune responses during vaccination. Therefore, whether addition of LNP-Ebi3p28 particles would augment the generation of long-lived memory CD8⁺ T cells following LNP-mRNA immunization was assessed. First, 10³ congenically distinct OT-I CD8⁺ T cells were adoptively transferred into wildtype mice, followed by intramuscular immunization of the mice with LNP-OVA alone or with the equivalent dose of LNP-OVA mixed with LNP-Ebi3p28 particles (FIG. 10A). Mice were euthanized 60 days following a single dose of LNP-mRNA particles, and the number of memory cells were assessed. The results show that indeed addition of LNP-Ebi3p28 particles resulted in an increase in proportion and number of memory CD8⁺ T cells detectable in the spleens of wildtype host mice (FIGS. 10B-10C). Overall, these data support the use of LNPs that contain mRNA encoding IL-27 to enhance the generation of CD8⁺ T cell immunity of LNP vaccines.

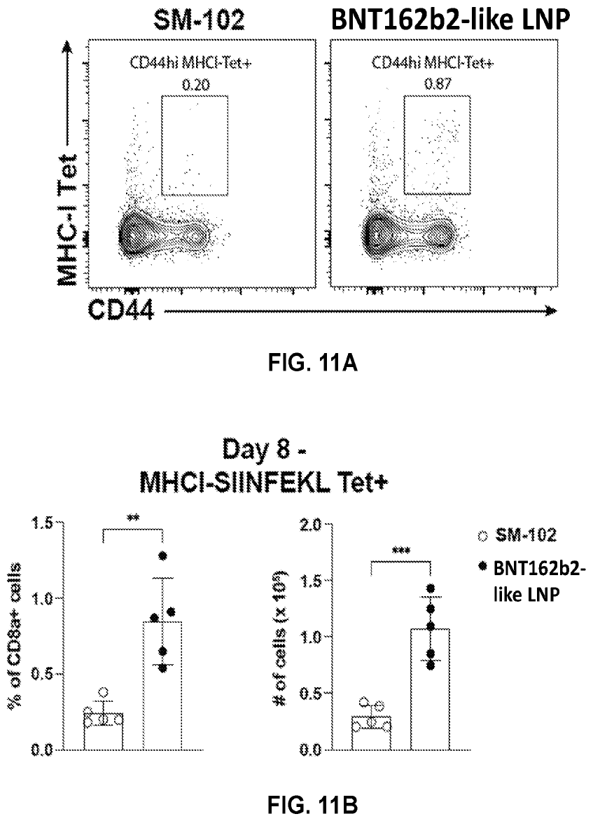

Similar to the observed differences in CD8⁺ T cell responses in patients receiving BNT162b2 or mRNA-1273 mice immunized with 1 μg of LNP-OVA particles formulated with SM-102 or a lipid similar to that in BNT162b2 illustrated a significant reduction in the capability of SM-102 formulated LNPs to drive expansion of antigen-specific CD8⁺ T cells (FIGS. 11A-11B). It is contemplated herein that this reduced capacity to induce CD8⁺ T cell responses by SM-102 containing LNPs could be augmented with the introduction of mRNA encoding IL-27 as additional cargo in LNP based vaccines formulated with SM-102.

In sum, it is demonstrated herein that differential capability of mRNA-1273 and BNT162b2 vaccines to induce CD8⁺ T cell responses may hinge on the induction of IL-27 by the specific ionizable lipid packaged within the formulation. Further, the results disclosed herein indicate that the capacity of an LNP vaccine to induce CD8⁺ T cell responses could be acquired via the introduction of mRNA cargo encoding IL-27 (or possibly other cytokines, e.g., IL-2, IL-12, IL-15, and/or TGF-β) in addition to the mRNA encoding antigen, thus providing a means for optimizing future LNP-mRNA vaccines for specific immunological outcomes. Indeed, as demonstrated herein for the case of LNPs comprising mRNA encoding IL-27 and mRNA encoding OVA antigen, LNP-mRNA vaccines provide an optimal platform for the addition of cytokine-encoding mRNAs to tailor future LNP-mRNA vaccines.

IL-27 is Uniquely Necessary for the Expansion of CD8⁺ T Cells after Primary LNP-mRNA Vaccine Dose

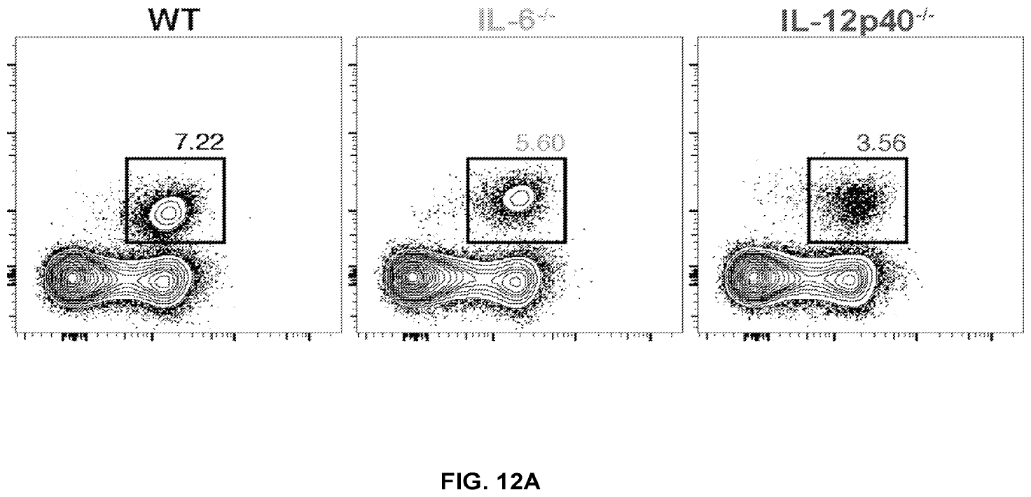

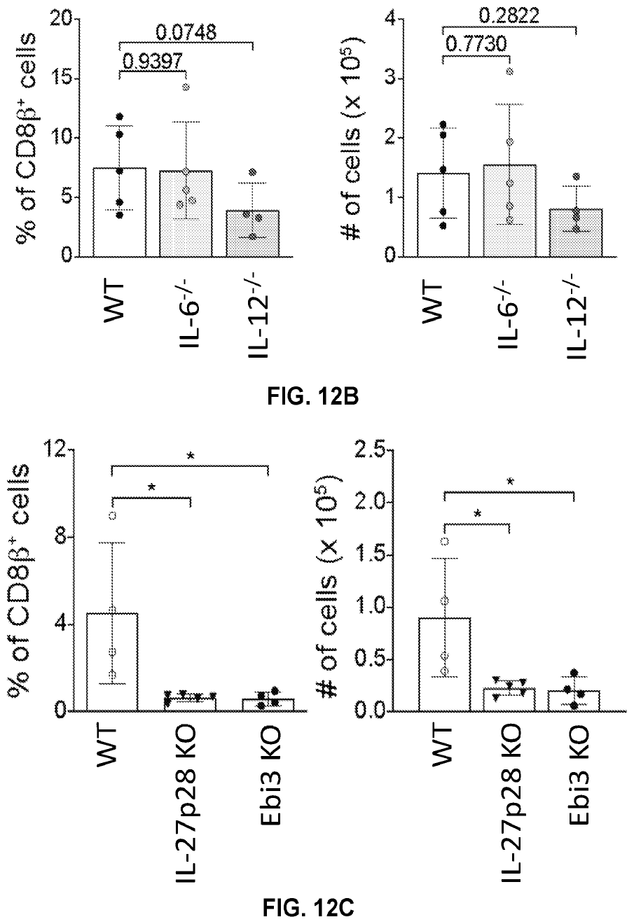

As disclosed above, IL-27 is necessary for the expansion of antigen-specific CD8⁺ T cells in response to LNP-mRNA vaccines, although whether this effect was specific to IL-27, or broadly applicable to other IL-6/IL-12 family members, remained unknown. Thus, whether mice that were defective in IL-6 (IL-6^{-/-}) or IL-12 (IL-12p40^{-/-}) exhibited defects in their antigen-specific CD8⁺ T cell responses following immunization with LNP-OVA vaccines was examined. Despite IL-6's importance in supporting the differentiation of T follicular helper (T_{FH}) CD4⁺ T cells following LNP-mRNA immunization, loss of IL-6 unexpectedly does not have an impact on the expansion of antigen-specific CD8 T cells (FIGS. 12A-12B). Loss of IL-12p40 resulted in a small decrease in the proportion and number of responding CD8⁺ T cells, but this was not comparable to the severe defect observed in mice deficient for either subunit of IL-27 (FIG. 12C). These data demonstrate that IL-27 exhibits a unique role in supporting the antigen-specific CD8⁺ T cell response to LNP-mRNA immunization.

Including mRNA Encoding IL-27 with Alternative Lipid Formulations Improves CD8⁺ T Cell Response to Primary mRNA Vaccine Dose

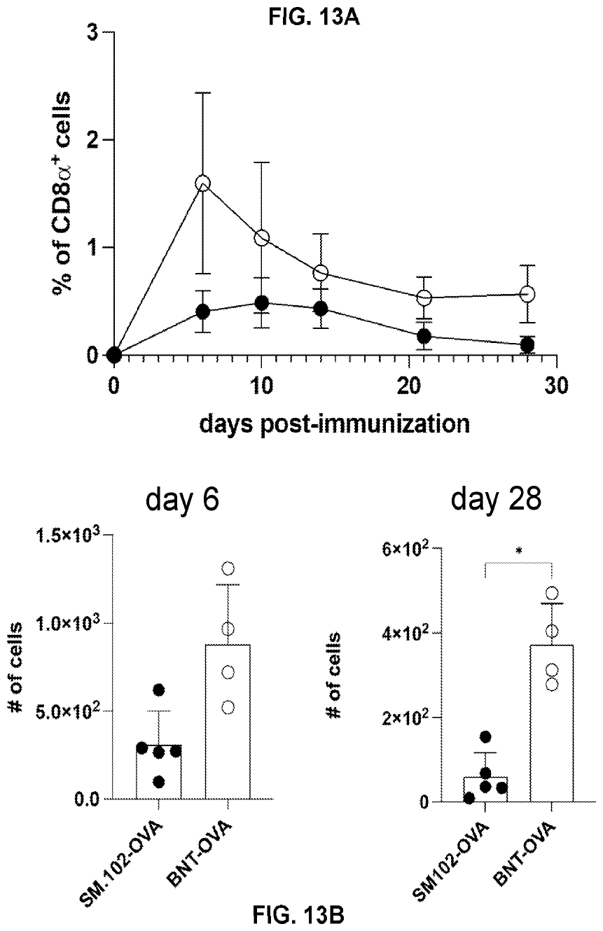

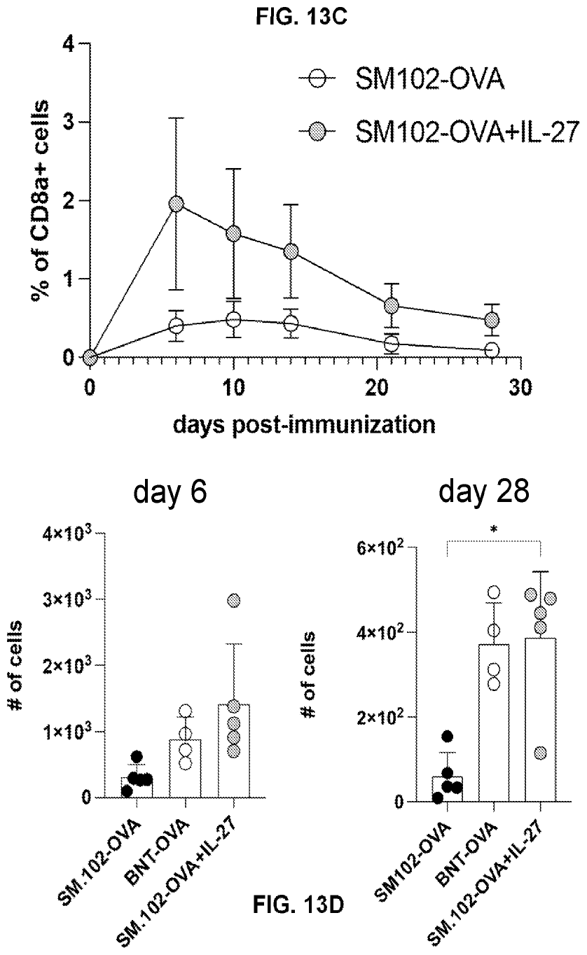

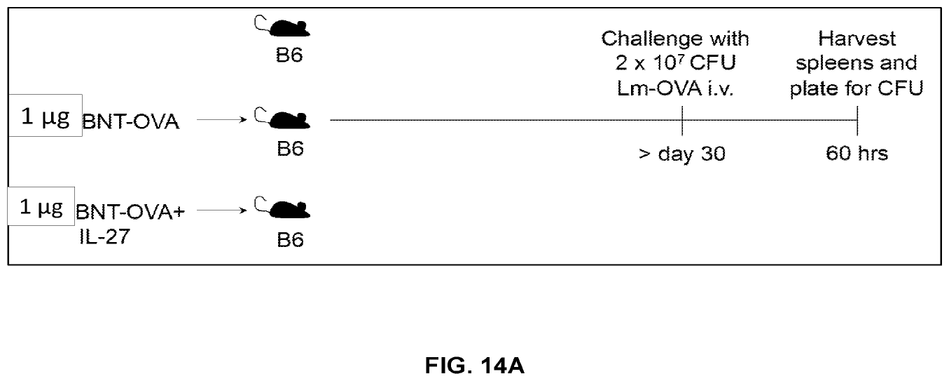

LNPs require an ionizable lipid for the delivery of mRNA cargo into cells for expression. These lipids vary in structure and inflammatory properties that may underly the differences in responses to current SARS-COV2 vaccines. Differences in the SARS-COV2-specific CD8⁺ T cell response have been reported in longitudinal studies of vaccinated individuals between the Pfizer-BioNtech (BNT162b2) and Moderna (mRNA-1273) vaccines. Therefore, whether a difference in CD8⁺ T cell responses was replicable in mice was examined by formulating particles that contained Ovalbumin mRNA but are similar to the Pfizer-BioNtech (BNT-OVA) and Moderna (SM102-OVA) vaccines. The results show that when administering the same dose (1 g) of BNT-OVA or SM102-OVA, there is a defect in the CD8⁺ T cell response observable in the blood to SM102-OVA at the peak (10 days post-immunization) of the response that becomes more significant following contraction (28 days post-immunization) (FIGS. 13A-13B). Given the unique role IL-27 plays in supporting the CD8⁺ T cell response following LNP-mRNA immunization, it was hypothesized that including IL-27 mRNA could improve the CD8⁺ T cell response to SM102-OVA. To test this, SM102-OVA and SM102-IL-27 particles were co-administered and the antigen-specific CD8⁺ T cell response was tracked via peripheral blood. The results show that co-administering particles that encode for IL-27 along with SM102-OVA significantly improved the antigen-specific CD8⁺ T cell response induced by the SM-102 formulation (FIGS. 13C-13D). This equated in a significantly increased number of long-lived antigen-specific CD8⁺ T cells observed in peripheral blood 28 days post-immunization (FIG. 13D). It is contemplated herein that this would result in improved protective capacity of the SM102-OVA elicited CD8⁺ T cells. In support of this, the data in the following section supports that providing IL-27 mRNA to BNT-OVA improves protection upon Listeria monocytogenes-OVA (LM-OVA) challenge.

Addition of IL-27 mRNA Improves Protection Against LM-OVA Challenge Following a Single Dose

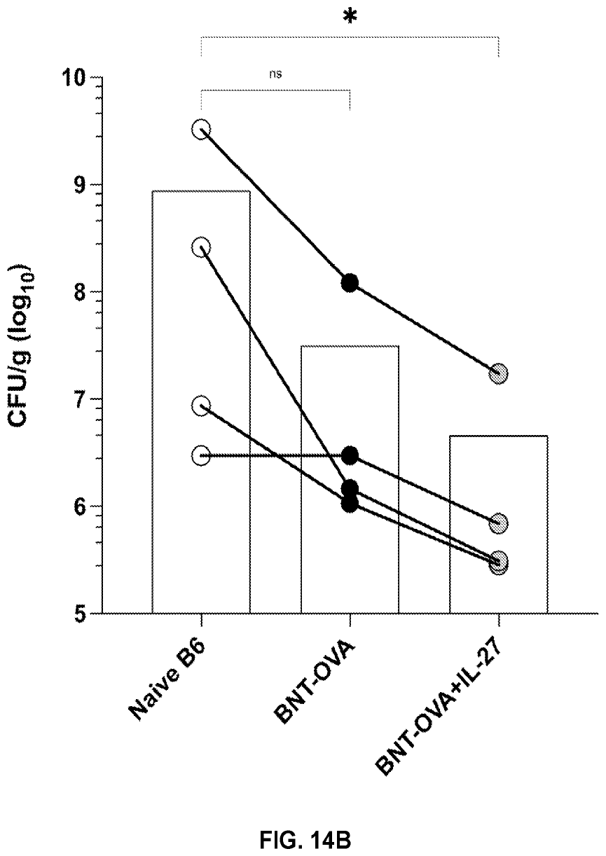

To assess the protective impact of improving the CD8⁺ T cell response by including IL-27 mRNA within an LNP, a Listeria monocytogenes (LM) challenge model was utilized. In mice, protection from challenge is largely antibody independent and requires CD8⁺ T cells. Therefore, it was hypothesized that improving the pool of antigen-specific CD8⁺ T cells would also improve protection from LM-OVA challenge following vaccination. Thus, B6 mice were immunized with either BNT-OVA or BNT-OVA co-administered with BNT-IL-27 (BNT-OVA+IL-27) particles at a 1 mg dose (FIG. 14A). 30 days following immunization, the mice were challenged with 2×10⁷ CFU of LM-OVA intravenously. 60 hrs later, spleens were harvested and processed to assess CFU of bacteria. Following rechallenge, the results indicate that both BNT-OVA and BNT-OVA+IL-27 provide protection in comparison to naive mice that were challenge with the equivalent dose in B6 mice (FIG. 14B). Mice that received BNT-OVA+IL-27 had significantly reduced bacterial burdens indicating that increasing the CD8⁺ T cell response via administration of IL-27 mRNA can provide a protective benefit in an infectious disease setting. It is contemplated herein that optimization of the dosing and ratio of antigen to cytokine mRNA at vaccination would likely result in an even greater protective benefit.

### IL-27 Signaling is Necessary for Tfh Differentiation Following LNP-mRNA Immunization

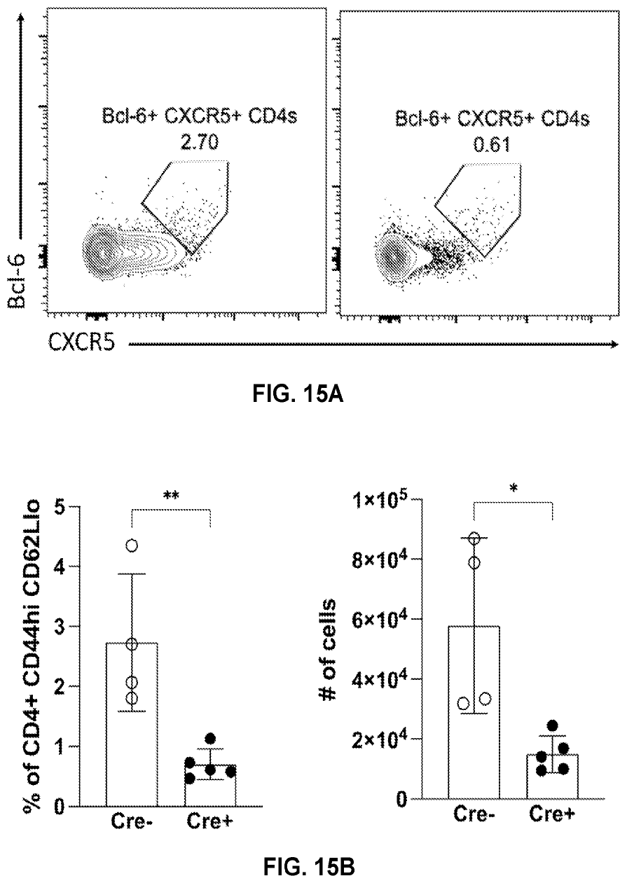

The analyses disclosed herein have thus far focused on CD8⁺ T cells. Next, whether the loss of IL-27 signaling on CD4′ T cells also impacts the generation of T follicular helper (T_{FH}) cells was examined. T_{FH} are critical for helping B cell responses to antigen. Prior studies have demonstrated that IL-6 is critical for LNP-mRNA vaccine induced T_{FH} responses, so whether T cell-specific loss of IL-27 signaling would also impair T_{FH} development was assessed. Mice with T cell-specific deletion of the IL27 Receptor alpha chain (IL27Ra^{fl/fl} CD4Cre⁺) were immunized with a 1 μg dose of BNT-OVA i.m. and then assessed for the T_{FH} response via flow cytometry 10 days post-immunization (FIGS. 15A-15B). The results indicate that the proportion and number of polyclonal T_{FH} generated in mice with T cell-specific loss of IL-27 signaling was significantly decreased. It is contemplated herein that this loss should correlate with a reduction in neutralizing antibody. Total germinal center B cells are also reduced (data not shown) correlating with the reduction in T_{FH}.

### Additional Cytokines can be Utilized for Modulating the CD8⁺ T Cell Response—Impacts of Each are not Obvious

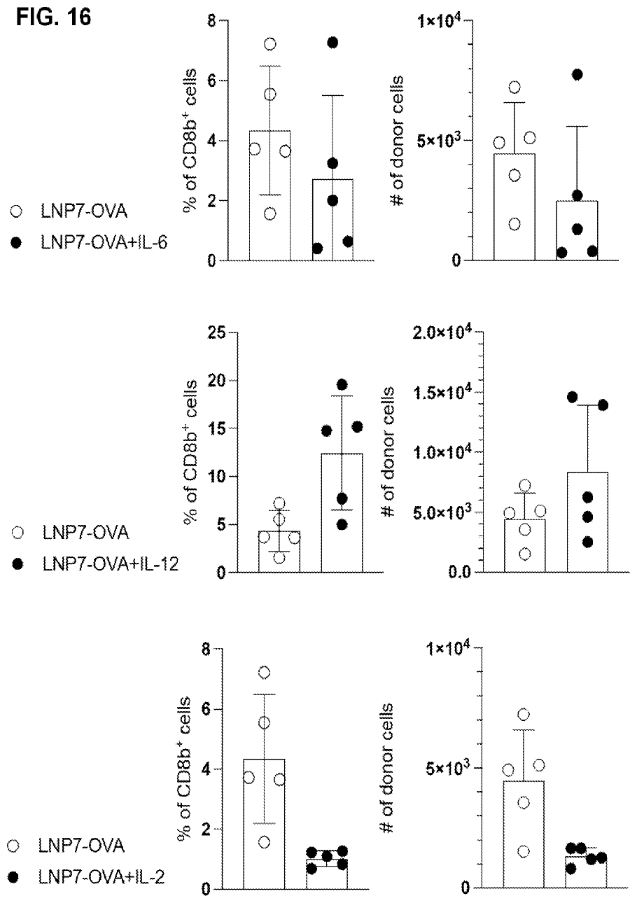

The promising data disclosed herein generated from including IL-27 mRNA within vaccine formulations to bolster the CD8⁺ T cell response demonstrates that this is a viable approach for improving the immune response to vaccine antigen. Thus, the effects of additional cytokines that could be relevant for various vaccines or therapies (i.e., IL-6, IL-12, and IL-2) were assessed (FIG. 16). Mice were vaccinated with LNP-OVA and co-administered LNPs formulated with IL-6, IL-12, or IL-2 and the expansion of donor CD8⁺ T cells was tracked as a simple readout of the cytokine's impact. The addition of IL-6 did not significantly alter the expansion of CD8⁺ T cells 8 days post-immunization (FIG. 16). In contrast, IL-12 significantly boosted the expansion of antigen-specific CD8⁺ T cells found in blood as early as day 8 post-immunization (FIG. 16). Surprisingly, IL-2, which was expected to support increased expansion of antigen-specific CD8⁺ T cells, instead suppressed the donor CD8⁺ T cell response (FIG. 16). These data highlight the potential that including specific cytokine mRNA within LNP-mRNA therapies holds in altering the immune response, and thus it is contemplated herein that cytokines can be chosen for specific therapies or outcomes. For example, therapeutic vaccines for cancer may be a distinct use case for the inclusion of IL-12 or IL-27 mRNA to enhance CD8⁺ T cell responses to tumors.

### Therapeutic Use of LNP-Ebi3IL27p28 Particles for Treatment of Tumors

The addition of IL-27 mRNA to LNP vaccines improves the protective capacity of LNP formulations to LM-OVA challenge by promoting the CD8⁺ T cell response. In line with this, it was anticipated that LNP-Ebi3IL27p28 particles would be efficacious for treatment of diseases where CD8⁺ T cell responses correlate with improved outcomes, such as cancer. Indeed, recent advances in the understanding of immune responses to malignancy has allowed immunotherapy to become a frontline treatment. This is largely due to the ability of checkpoint blockade inhibitors to unlock T cell responses to patient tumors. Thus, it is contemplated herein that the enhanced CD8⁺ T cell responses induced by the addition of LNP-Ebi3IL27p28 particles would be beneficial for the treatment of tumors.

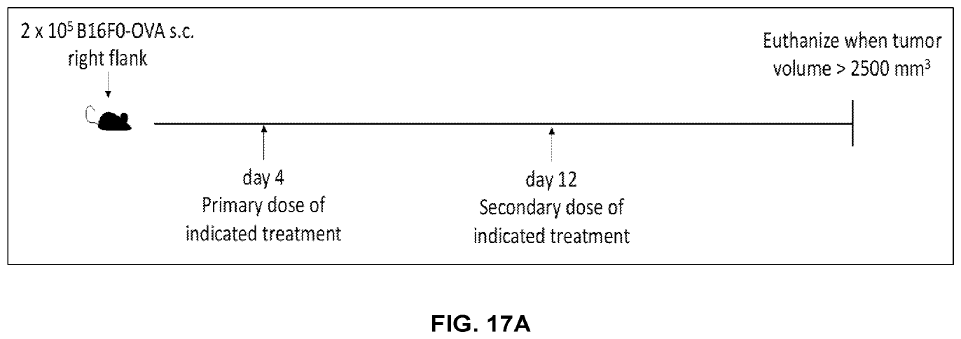

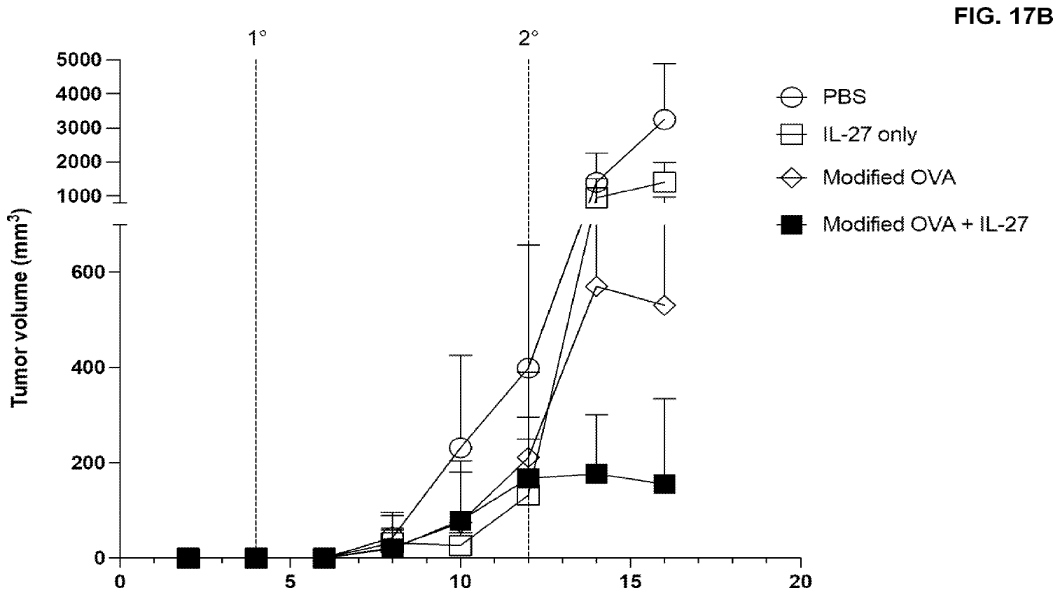

Thus, the effectiveness of a therapeutic vaccine was assessed using B16F0, an orthotopic model of melanoma that was engineered to express ovalbumin (B16F0-OVA). The experimental schema is detailed in FIG. 17A. In short, 2×10⁵ B16F0-OVA tumor cells were implanted subcutaneously in the right flank of wildtype mice 4 days prior to the initial dose of therapies. Mice were then randomized into treatment groups and administration of vaccines was performed in a single blinded fashion to limit bias during the measurement of tumor size throughout the course of the experiment. Cohorts of mice were divided into untreated (PBS), LNP-Ebi3IL27p28 (IL-27 only) alone, modified OVA (LNP-OVA), and modified OVA+IL27 (LNP-OVA+LNP-Ebi3IL27p28) (FIG. 17B). Mice that were untreated were the negative control and, as expected, tumors grew rapidly in this group. Mice that received LNPs encoding for IL-27 alone, with no antigen, failed to be protected, highlighting that IL-27 signaling supports the expansion of antigen-specific CD8⁺ T cells, but does not drive expansion independent of an antigen source. Administration of modified OVA LNPs did slow tumor growth somewhat in comparison to untreated mice, but was not significantly improved. Remarkably, mice that received modified OVA+IL-27 LNPs exhibited significantly reduced tumor growth in comparison to untreated mice (FIG. 17B). It is contemplated herein that optimization of the dosing and ratio (e.g., titrating the mix of IL-27:OVA containing particles) at vaccination would likely result in an even greater benefit. These data highlight the positive impact that delivery of cytokine encoding mRNAs via LNPs can have on prophylactic and therapeutic vaccines, in particular vaccines for cancer treatment, and highlight the potential to lower vaccine doses while maintaining efficacy of induction of protective responses.

### Example 2: IL-12 Amplifies Expansion of Antigen-Specific CD8⁺ T Cells

As demonstrated herein, lipid nanoparticles (LNPs) containing mRNA that encodes for cytokine drive expression of functional cytokine protein that can augment CD8⁺ T cell responses to immunization. Interleukin-12 (IL-12) has long been of interest as a vaccine adjuvant due to its ability to support antigen-specific humoral and cellular immunity during infection and immunization. IL-12 acts on CD8⁺ and CD4⁺ T cells to simulate proliferation, production of IFNγ, and cytolytic activity (Schmidt & Mescher, 1999, Journal of Immunology, 163(5), 2561-2567). However, the therapeutic administration of recombinant IL-12 protein has proven difficult, due to the protein's short half-life and potent toxicity (Leonard et al., 1997, Blood, 90(7), 2541-2548). Thus, it is contemplated herein that administration of IL-12 via IL-12 encoding mRNA-LNPs may circumvent these challenges by providing transient expression that scales with the dose of particles. Studies have shown that vaccine mRNA persists for several days following vaccination, and the cells that take up the LNPs are macrophages and dendritic cells, major producers of IL-12 (Fertig et al., 2022, Biomedicines, 10(7), 1538; Lenart et al., 2022, Molecular Therapy—Methods & Clinical Development, 27, 309-323). Thus, it is contemplated herein that by titrating the dose of IL-12 encoding mRNA-LNPs, one can induce immunologically relevant cell types to produce a sustained IL-12 signal during T cell priming.

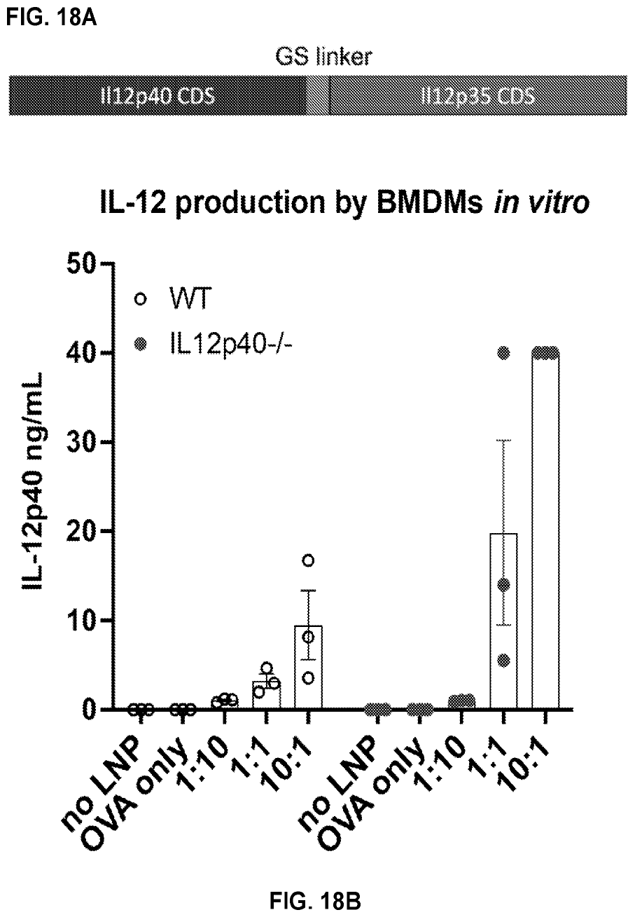

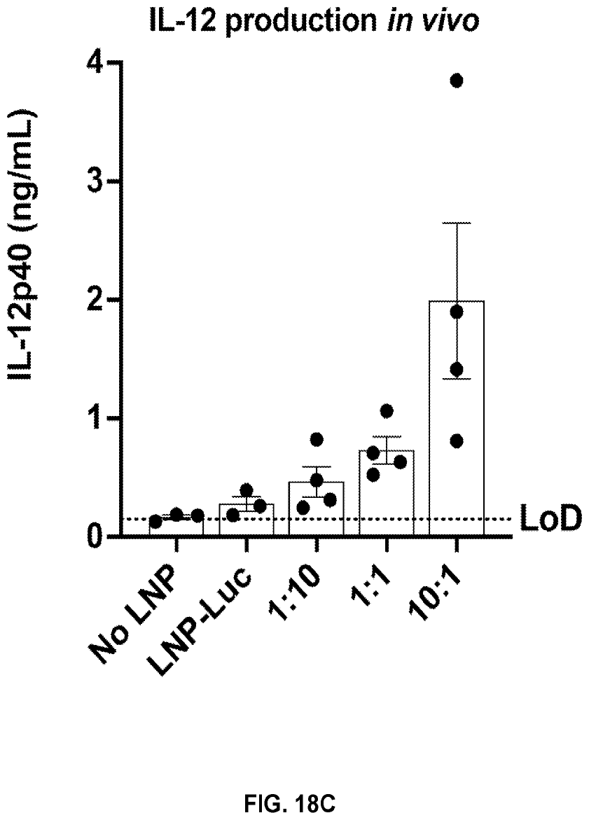

To assess the influence of incorporating IL-12 into mRNA LNP vaccination, a particle was designed that encapsulates N1-methyl-pseudouridine-modified codon-optimized mRNA that encodes for the coding sequence (CDS) of the two subunits of IL-12, p40 and p35 (FIG. 18A). These coding sequences are linked by a flexible glycine-serine linker based on a previously published recombinant IL-12 construct (Lieschke et al., 1997, Nature Biotechnology, 15(1), 35-40).

To confirm that LNPs containing the p40p35 construct (LNP-p40p35) induce IL-12 protein production, wild-type (WT) and IL-12 deficient (IL12p40−/−) bone-marrow derived macrophages (BMDMs) were incubated with increasing doses of LNP-p40p35 particles. To account for any potential adjuvant effect of the lipid nanoparticles, LNP-p40p35 particles were mixed with an LNP containing an unrelated mRNA construct (LNP-OVA) in varying ratios to give a consistent total dose of LNP. The data indicate a dose-dependent translation of IL-12p40 protein in response to LNP-p40p35 particles in both WT and IL12p40−/− BMDMs (FIG. 18B).

To determine if LNP-p40p35 particles induce production of IL-12 protein in vivo, mice were immunized intramuscularly with LNP-p40p35 in their hind legs 24 hours prior to sacrifice. Following euthanasia, draining lymph nodes were harvested and mechanically lysed. An ELISA specific for the detection of the p40 subunit of IL-12 was used to determine the amount of protein generated in response to varying doses of LNP-p40p35 construct. Again, LNP-p40p35 particles were mixed with an LNP containing an unrelated mRNA construct (LNP-OVA) in varying ratios to give a consistent total dose of LNP. This data indicated that there is dose dependent translation of IL-12 protein in response to LNP-p40p35 particles (FIG. 18C). In the following studies, a 1:1 dose of LNP-p40p35 and LNP-OVA was administered, which corresponds to a concentration of IL-12 that is well within limits of tolerated IL-12 (Portielje et al., 1999, Clinical Cancer Research, 5(12), 3983-3989).

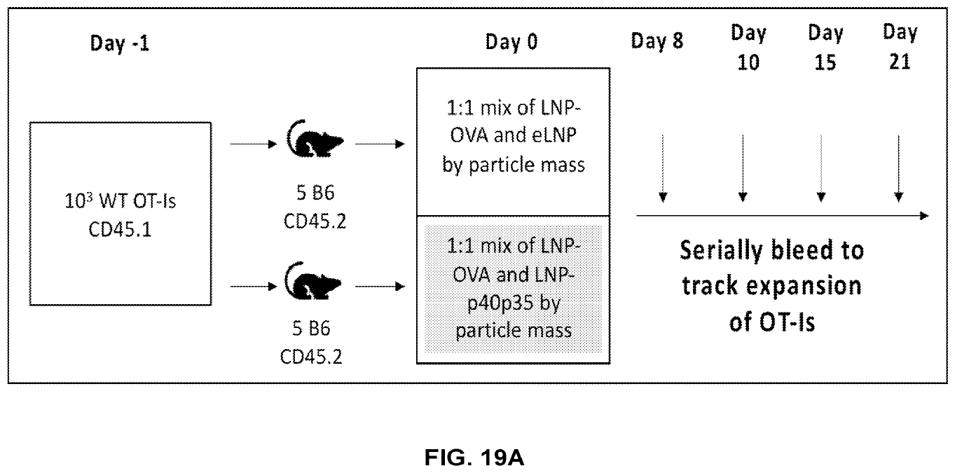

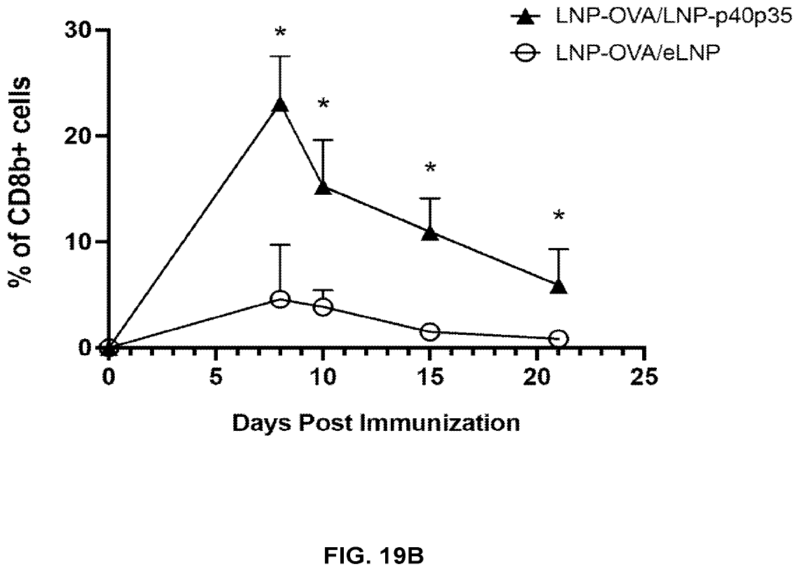

Next, the effect of IL-12 on the expansion of antigen-specific CD8⁺ T cells in response to immunization was examined. To accomplish this, congenically distinct wild-type (WT) OT-I CD8⁺ T cells (OT-Is) were adoptively transferred into a cohort of 10 WT mice. OT-Is are engineered to express a T cell receptor (TCR) that specifically recognizes an 8 amino-acid segment of the ovalbumin (OVA) protein, SIINFEKL. The following day, the mice were immunized intramuscularly with a mixture of LNPs containing either OVA mRNA (LNP-OVA) and empty LNPs (eLNPs, LNP-OVA/eLNP) or LNP-OVA and LNP-p40p35 (LNP-OVA/LNP-p40p35) (FIG. 19A). The number of OVA-mRNA copies was kept consistent between groups as well as the total dose of lipid (1 μg) due to its known role as an adjuvant. The mice were serially bled on days 8, 10, 15, and 21 post-immunization to track the kinetics of OT-I expansion and contraction. At every time point, mice that received LNP-OVA/LNP-p40p35 had at least a 4-fold greater frequency of OT-Is in the blood than those that received LNP-OVA/eLNP (FIG. 19B). These data indicate LNP-p40p35 particles augment the magnitude of the CD8⁺ T cell response to vaccination in vivo.

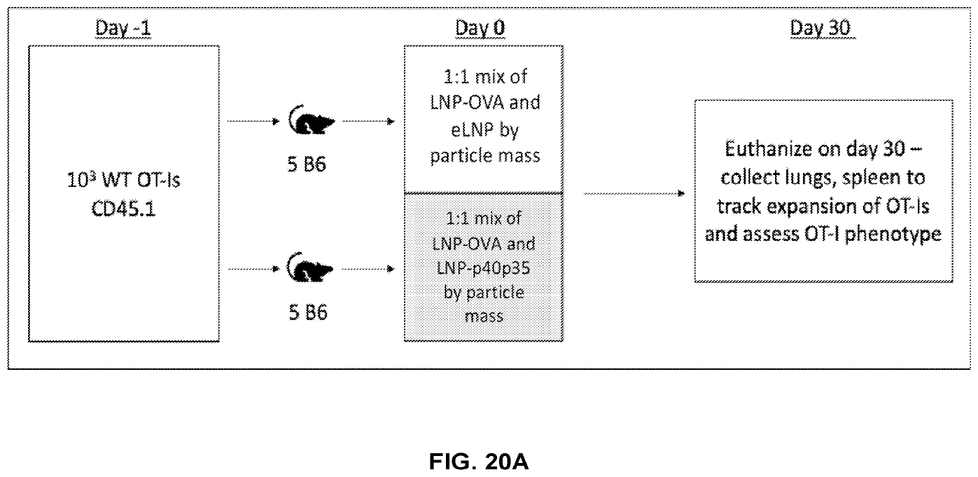

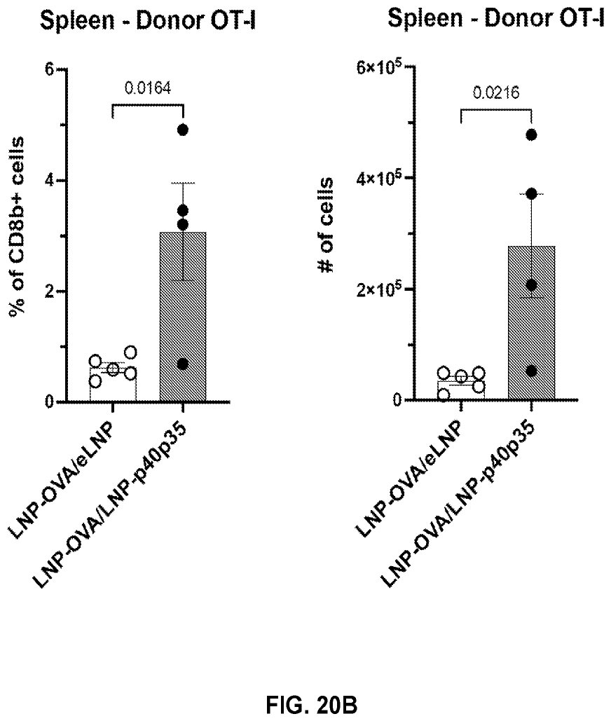

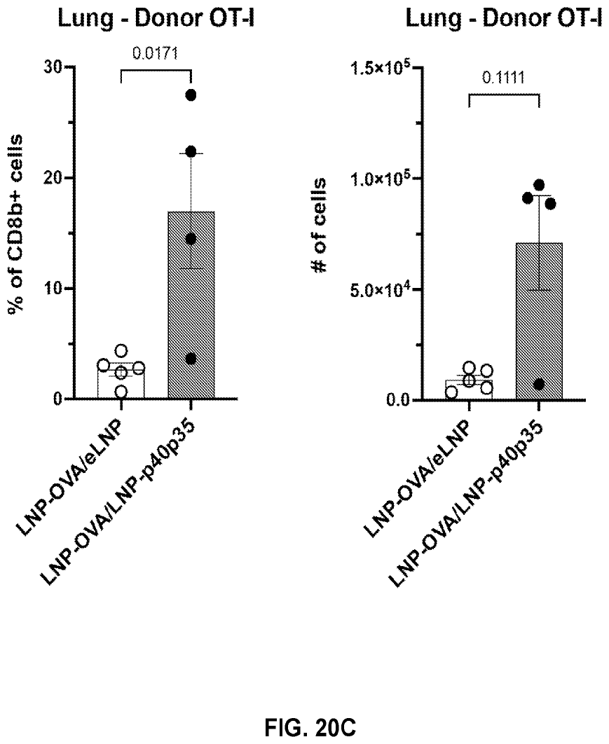

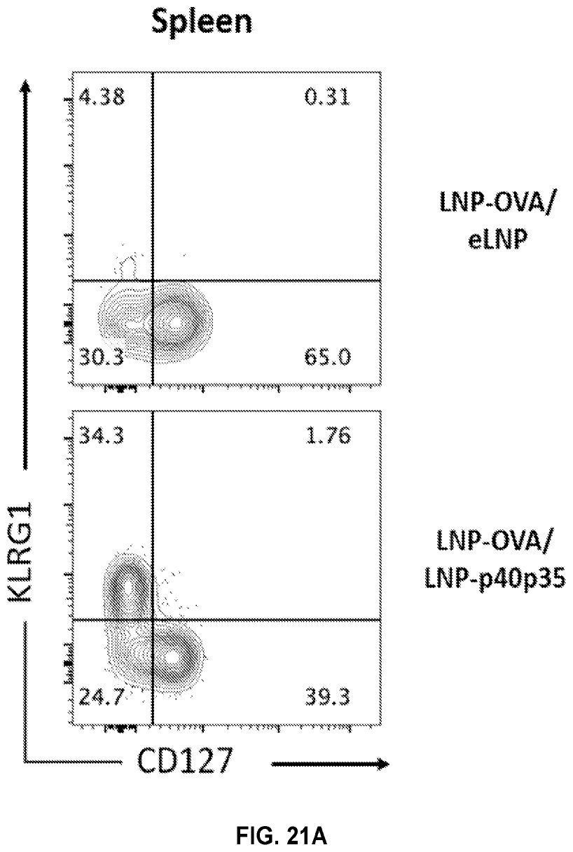

To further characterize the effect of LNP-derived IL-12 on the magnitude of the CD8⁺ T cell response, the mice were euthanized 30 days post-immunization and the lungs and spleens were collected (FIG. 20A). The frequency and total number of OT-Is found in spleen and lung were examined to determine if the improved expansion of OT-Is observed in the blood resulted in increased frequency and number of OT-Is in secondary lymphoid tissues (spleen) and peripheral tissues (lung). At day 30, both the spleen and the lung of LNP-OVA/LNP-p40p35 immunized mice showed a significantly higher frequency and total number of OT-Is compared to LNP-OVA/eLNP immunized mice (FIGS. 20B-20C). When considered with the PBL analysis, these data indicate that addition of LNP-p40p35 particles enhance the initial expansion of antigen-specific CD8⁺ T cells globally (FIGS. 19A-19B and FIGS. 20A-20C). Because contraction of the CD8⁺ T cell pool is relatively invariant, this also indicates that the memory pool of mice given LNP-p40p35 will be larger than those given LNP-OVA/eLNP (Badovinac et al., 2004, Nature Immunology, 5(8), 809-817).

### LNP-p40p35 Alters Differentiation and Function of CD8⁺ T Cells

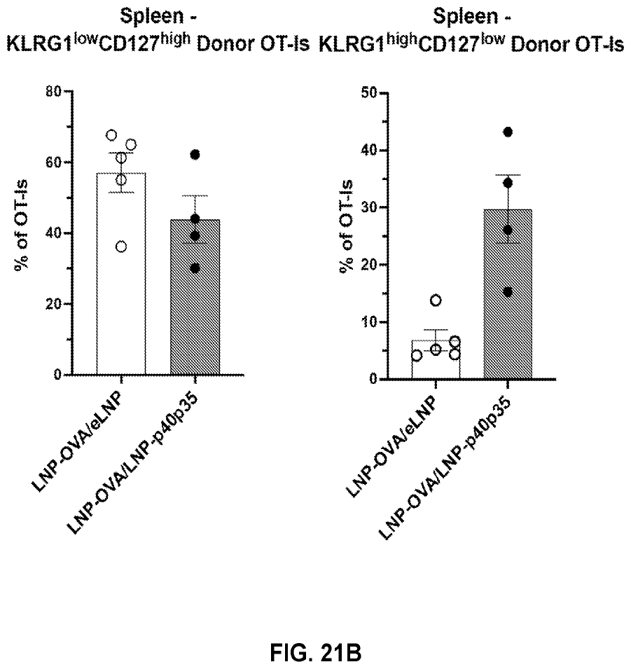

In addition to its role in enhancing T cell proliferation, IL-12 can also influence the phenotype and functionality of CD8⁺ T cells. Therefore, the phenotypic and functional changes resulting from immunization with LNP-OVA/LNP-p40p35 were evaluated. Several cell surface markers are commonly used to assess the differentiation state of antigen specific T cells generated in response to immunization or infection. Killer cell lectin type receptor G1 (KLRG1) and the alpha chain of the IL-7 receptor (CD127) are two such markers. KLRG1^{high}CD127^{low} T cells are more differentiated effectors that help to mediate pathogen clearance, while KLRG1^{low}CD127^{high} cells are longer-lived, less differentiated CD8⁺ T cells. IL-12 promotes the expression of KLRG1 in activated CD8⁺ T cells following infection with Toxoplasma gondii (Wilson et al., 2008, J Immunology, 180(9):5935-45). Therefore, whether providing LNP-p40p35 particles during immunization would skew the CD8⁺ T cell response away from a predominantly KLRG1^{low}CD127^{high} pool and towards more differentiated effector cells was evaluated. In mice immunized with LNP-OVA/LNP-p40p35, there was a higher frequency of KLRG1^{high}CD127^{low} OT-Is in both the spleen and lung at day 8 and 30 post immunization, indicating that providing LNP-p40p35 during immunization results in a greater frequency of these cells (FIGS. 21A-21B). The increased frequency of this effector phenotype also corresponded to a greater total number of KLRG1^{high}CD127^{low} OT-Is in the spleen and lung. At day 30 post immunization there was no significant difference in the frequency of KLRG1^{low}CD127^{high} OT-Is when comparing LNP-OVA/LNP-p40p35 to LNP-OVA/eLNP immunized mice (FIG. 21B). Due to the expanded population of OT-Is in LNP-p40p35 treated mice, the total number of KLRG1^{low}CD127^{high} OT-Is in this group was significantly greater than in mice immunized with LNP-OVA/eLNP (FIG. 21B).

Therefore, mice that received LNP-OVA/LNP-p40p35 during immunization generated a larger pool of KLRG1^{low}CD127^{high} cells than those that received LNP-OVA/eLNP. While providing IL-12 during immunization resulted in a skewing of the OT-I phenotype towards a greater frequency and total number of differentiated effector cells, the enhanced expansion of the OT-I population in LNP-p40p35 treated mice led to a greater total number of OT-Is of both phenotypes. This result demonstrates that providing LNP-p40p35 particles during immunization may be beneficial in prophylactic and therapeutic settings through induction of strong acute effector responses and a robust CD8 memory T cell population.

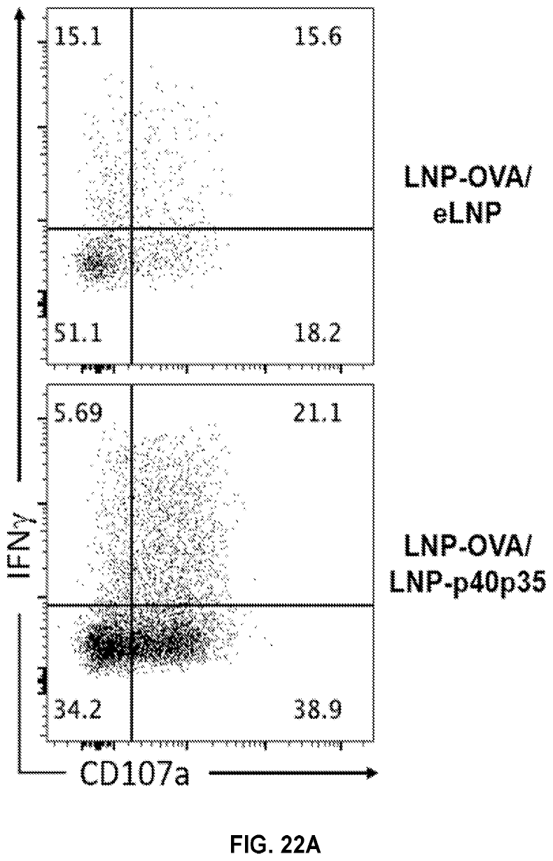

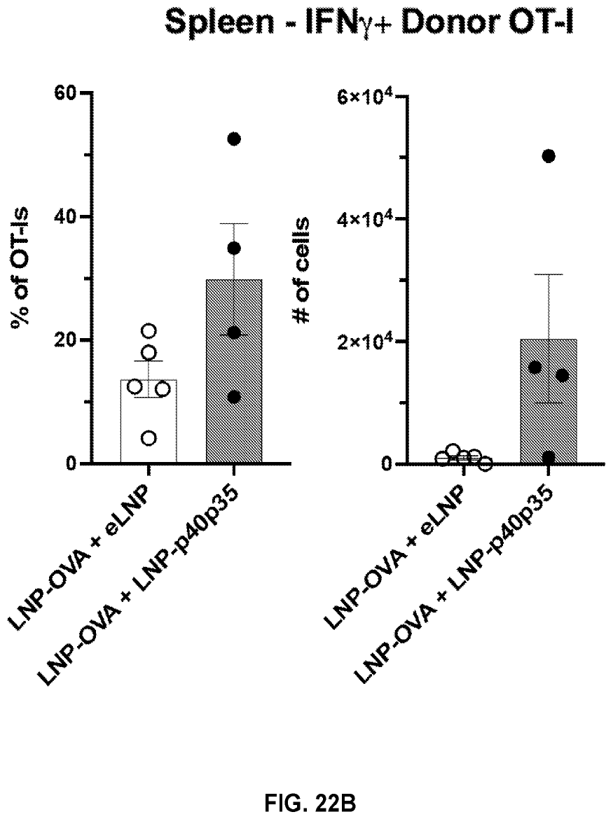

Because IL-12 induces production of the cytokine Interferon-gamma (IFNγ), whether LNP-OVA/LNP-p40p35 particles augmented the ability of antigen-specific CD8⁺ T cells to produce IFNγ in response to TCR stimulation was tested. To evaluate this, splenocytes were collected at day 30 post-immunization from the previously described experiment and restimulated with SIINFEKL (SEQ ID NO: 1), the cognate peptide of OT-Is. The frequency and total number of OT-Is that produced IFNγ was analyzed via flow cytometry after 4 hours (FIGS. 22A-22B). In response to TCR stimulation, a greater frequency and total number of OT-Is from LNP-OVA/LNP-p40p35 immunized mice produced IFNγ (FIGS. 22A-22B). These data indicate that LNP-OVA/LNP-p40p35-derived IL-12 leads to long-lasting effects on the functionality of antigen-specific CD8⁺ T cells. Taken together with the phenotypic data, these results show that immunization with LNP-OVA/LNP-p40p35 results in a larger pool of memory precursor CD8⁺ T cells and highly differentiated effector CD8⁺ T cells.

### LNP-p40p35 Particles Enhance Antibody Production in Aged Mice

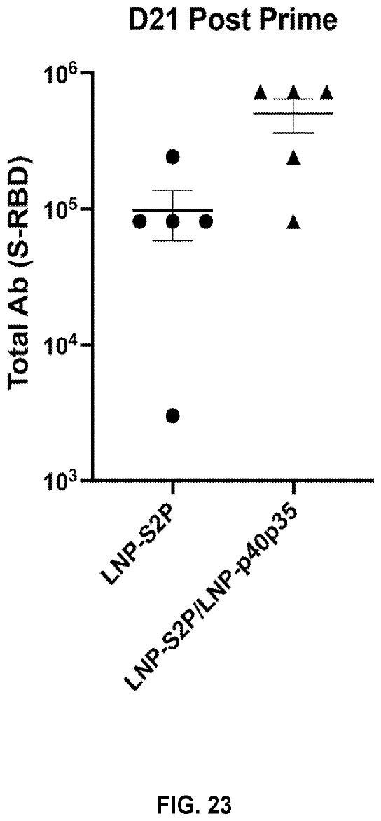

Although mRNA vaccines induce strong antibody responses, antibody generation is not as robust in elderly age groups (Palacios-Pedrero et al., 2022, Nature Aging, 2(10), 896-905). IL-12 activates antigen-specific CD4⁺ T cells which are important for the development of optimal humoral responses, and exogenous IL-12 during immunization has been shown to enhance B cell class-switching due to IFNγ production by T cells and NK cells (Metzger et al., 1996, Annals of the New York Academy of Sciences, 795 (1 Interleukin 1), 100-115). Thus, experiments were conducted in order to understand if administration of LNP-p40p35 during vaccination could enhance antibody production in aged mice. Specifically, aged mice were vaccinated with LNPs containing mRNA encoding for the SARS-CoV-2 spike protein (LNP-S2P), with or without LNP-p40p35. Antibody titers in serum were assessed for total antibody production 21 days following immunization. Serum levels of antibodies against the Spike protein receptor binding domain (S-RBD) were elevated in aged mice that received LNP-p4p35 during immunization compared to those that did not (FIG. 23).

### LNP-p40p35 Particles Enhance Protection Against Listeria Monocytogenes

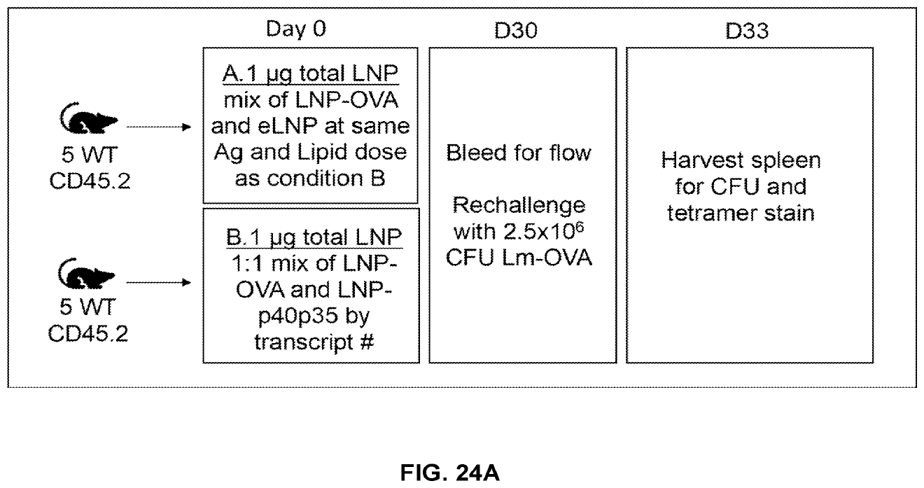

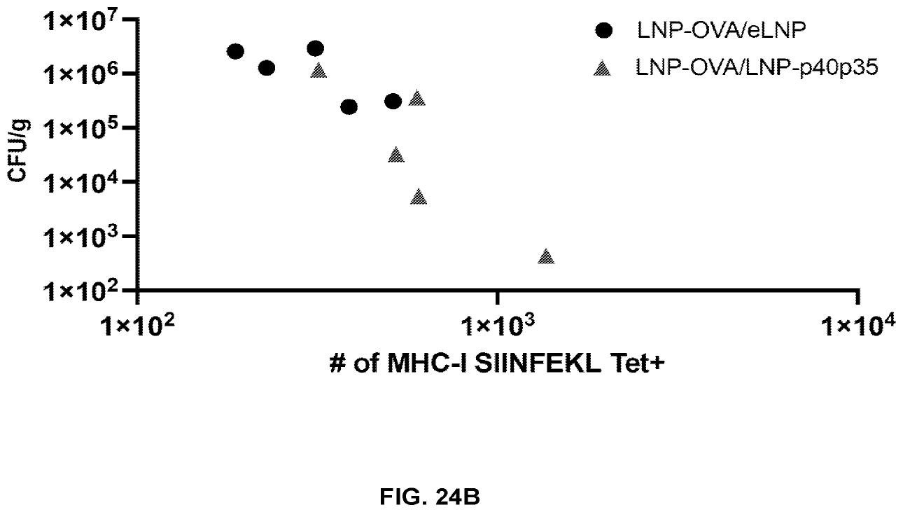

To understand if incorporation of IL-12 into mRNA vaccination enhances CD8⁺ T cell mediated protection against infection, a challenge experiment using recombinant Listeria monocytogenes (Lm) was performed. WT mice were immunized with LNP-OVA/eLNP or LNP-OVA/LNP-p40p35. Mice received a challenge with Lm that was engineered to express OVA (Lm-OVA) 30 days post immunization (FIG. 24A). Lm was selected for this challenge study because CD8⁺ T cells, and their production of IFNγ, is critical for control of this infection (Messingham et al., 2007, The Journal of Immunology, 179(4), 2457-2466). The results show that mice that received a single dose of IL-12 during vaccination (LNP-OVA/LNP-p40p35) cleared the infection more effectively than those that received LNP-OVA alone (FIG. 24B). This experiment highlights that IL-12's ability to enhance antigen-specific T cell expansion and IFNγ production enhances resistance to subsequent Lm challenge (FIG. 24B).

Therapeutic Vaccination with LNP-p40p35 Particles Enhances Clearance of Melanoma

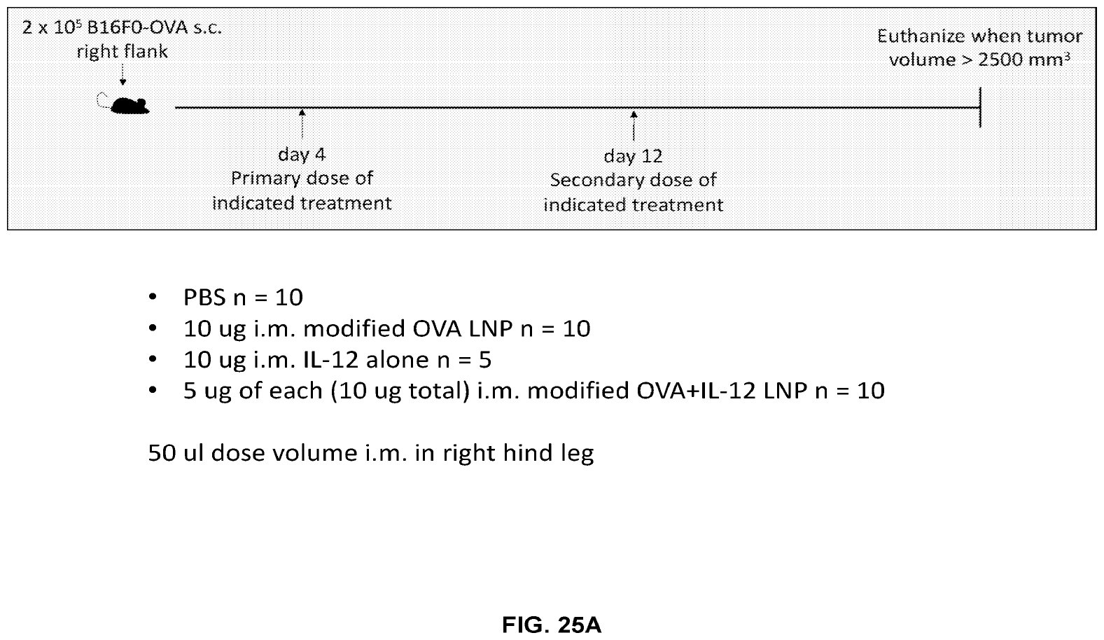

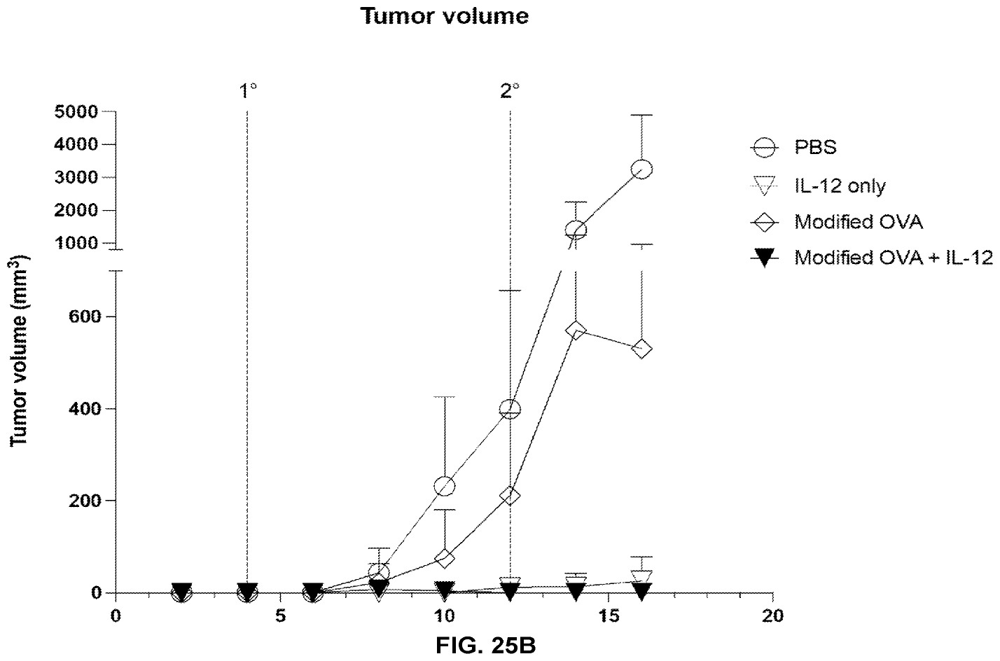

Because LNP-OVA/LNP-p40p35 vaccination enhanced protection to Lm challenge following a single dose, the potential therapeutic efficacy of LNP-p40p35 particles for the treatment of malignancy was examined. And because CD8⁺ T cells are crucial for clearance of tumors, the impact of LNP-p40p35 particles on clearance of a mouse model of melanoma was assessed. B16F0-OVA tumor cells were implanted subcutaneously into the right flank of mice prior to vaccine administration. On day 4 and day 12 post implantation, mice were dosed with LNP-OVA/eLNP, LNP-OVA/LNP-p40p35, LNP-p40p35 alone, or LNP-OVA containing unmodified OVA mRNA (FIG. 25A). Tumor volume was measured every other day. Remarkably, mice that were given LNP-OVA/p40p35 did not develop tumors at all (FIG. 25B). In comparison, mice that received LNP-OVA/eLNP developed tumors that progressed rapidly until the secondary dose was administered which slowed tumor growth, but did not result in clearance. Mice that received LNP-p40p35 alone largely did not develop tumors, though some did have palpable tumors by endpoint. These data indicates that LNP-p40p35, when combined with LNP-OVA, is more protective than vaccination with LNP-OVA/eLNP alone.

### Therapeutic Implications

Administration of LNP-p40p35 particles during mRNA vaccination results in greater expansion of antigen-specific CD8⁺ T cells, altered effector differentiation, and enhanced production of IFNγ. This impact on the CD8⁺ T cell response ultimately enhances protection against Listeria monocytogenes challenge following prophylactic immunization. Additionally, administration of LNP-OVA/LNP-p40p35 robustly induces the generation of differentiated effector CD8⁺ T cells, which was harnessed in a therapeutic vaccine against the orthotopic model of melanoma, B16F0-OVA. These results highlight the utility of LNP-p40p35 particles as adjuvants for both therapeutic and prophylactic vaccines.

Vaccination that includes LNP-p40p35 particles also enhances antibody production in aged animals. These data indicate that LNP-p40p35 particles can be particularly useful in enhancing immune responses of elderly adults and prove to be a novel, effective application of mRNA delivered via LNPs. Based on the unexpected results disclosed herein, it is contemplated herein that further engineering of the IL-12 mRNA, e.g., to leverage existing variants that have altered affinity to receptors, may prove useful for further optimizing the effects demonstrated herein. These IL-12 variants and their amino acid sequences are listed in Table 4.

Finally, it is contemplated herein that inclusion of LNP-p40p35 particles in vaccination may provide an avenue for lowering vaccine dosages while maintaining efficacy, which would be expected to lessen adverse events following vaccination.

TABLE 4
RNA and amino acid sequences of various
cytokines of the present disclosure

SEQ ID NO: 2 Murine IL-2 RNA
AGCATAAAAGTCTCAACACAACATATACAAAACAAACGAATCTCAAGCAATCAAGCATTCTACTTC
TATTGCAGCAATTTAAATCATTTCTTTTAAAGCAAAAGCAATTTTCTGAAAATTTTCACCATTTAC
GAACGATAGCGCTGCCACCATGTACTCCATGCAGCTGGCCTCCTGCGTGACCCTGACCCTGGTGCT
GCTGGTGAACTCCGCCCCCACCTCCTCCTCCACCTCCTCCTCCACCGCCGAGGCCCAGCAGCAGCA
GCAGCAGCAGCAGCAGCAGCAGCAGCACCTGGAGCAGCTGCTGATGGACCTGCAGGAGCTGCTGTC
CCGCATGGAGAACTACCGCAACCTGAAGCTGCCCCGCATGCTGACCTTCAAGTTCTACCTGCCCAA
GCAGGCCACCGAGCTGAAGGACCTGCAGTGCCTGGAGGACGAGCTGGGCCCCCTGCGCCACGTGCT
GGACCTGACCCAGTCCAAGTCCTTCCAGCTGGAGGACGCCGAGAACTTCATCTCCAACATCCGCGT
GACCGTGGTGAAGCTGAAGGGCTCCGACAACACCTTCGAGTGCCAGTTCGACGACGAGTCCGCCAC
CGTGGTGGACTTCCTGCGCCGCTGGATCGCCTTCTGCCAGTCCATCATCTCCACCTCCCCCCAGTA
ATAAACTAGTAGTGACTGACTAGGATCTGGTTACCACTAAACCAGCCTCAAGAACACCCGAATGGA
GTCTCTAAGCTACATAATACCAACTTACACTTACAAAATGTTGTCCCCCAAAATGTAGCCATTCGT
ATCTGCTCCTAATAAAAAGAAAGTTTCTTCACATTCTAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAA
AAAAAA
SEQ ID NO: 3 Murine IL-6 RNA
AGCATAAAAGTCTCAACACAACATATACAAAACAAACGAATCTCAAGCAATCAAGCATTCTACTTC
TATTGCAGCAATTTAAATCATTTCTTTTAAAGCAAAAGCAATTTTCTGAAAATTTTCACCATTTAC
GAACGATAGCGCTGCCACCATGAAGTTCCTGTCCGCCCGCGACTTCCACCCCGTGGCCTTCCTGGG
CCTGATGCTGGTGACCACCACCGCCTTCCCCACCTCCCAGGTGCGCCGCGGCGACTTCACCGAGGA
CACCACCCCCAACCGCCCCGTGTACACCACCTCCCAGGTGGGCGGCCTGATCACCCACGTGCTGTG
GGAGATCGTGGAGATGCGCAAGGAGCTGTGCAACGGCAACTCCGACTGCATGAACAACGACGACGC
CCTGGCCGAGAACAACCTGAAGCTGCCCGAGATCCAGCGCAACGACGGCTGCTACCAGACCGGCTA
CAACCAGGAGATCTGCCTGCTGAAGATCTCCTCCGGCCTGCTGGAGTACCACTCCTACCTGGAGTA
CATGAAGAACAACCTGAAGGACAACAAGAAGGACAAGGCCCGCGTGCTGCAGCGCGACACCGAGAC
CCTGATCCACATCTTCAACCAGGAGGTGAAGGACCTGCACAAGATCGTGCTGCCCACCCCCATCTC
CAACGCCCTGCTGACCGACAAGCTGGAGTCCCAGAAGGAGTGGCTGCGCACCAAGACCATCCAGTT
CATCCTGAAGTCCCTGGAGGAGTTCCTGAAGGTGACCCTGCGCTCCACCCGCCAGACCTAATAAAC
TAGTAGTGACTGACTAGGATCTGGTTACCACTAAACCAGCCTCAAGAACACCCGAATGGAGTCTCT
AAGCTACATAATACCAACTTACACTTACAAAATGTTGTCCCCCAAAATGTAGCCATTCGTATCTGC
TCCTAATAAAAAGAAAGTTTCTTCACATTCTAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAA
SEQ ID NO: 4 Murine IL-12 RNA
AGCATAAAAGTCTCAACACAACATATACAAAACAAACGAATCTCAAGCAATCAAGCATTCTACTTC
TATTGCAGCAATTTAAATCATTTCTTTTAAAGCAAAAGCAATTTTCTGAAAATTTTCACCATTTAC
GAACGATAGCGCTGCCACCATGTGCCCCCAGAAGCTGACCATCTCCTGGTTCGCCATCGTGCTGCT
GGTGTCCCCCCTGATGGCCATGTGGGAGCTGGAGAAGGACGTGTACGTGGTGGAGGTGGACTGGAC
CCCCGACGCCCCCGGCGAGACCGTGAACCTGACCTGCGACACCCCCGAGGAGGACGACATCACCTG
GACCTCCGACCAGCGCCACGGCGTGATCGGCTCCGGCAAGACCCTGACCATCACCGTGAAGGAGTT
CCTGGACGCCGGCCAGTACACCTGCCACAAGGGCGGCGAGACCCTGTCCCACTCCCACCTGCTGCT
GCACAAGAAGGAGAACGGCATCTGGTCCACCGAGATCCTGAAGAACTTCAAGAACAAGACCTTCCT
GAAGTGCGAGGCCCCCAACTACTCCGGCCGCTTCACCTGCTCCTGGCTGGTGCAGCGCAACATGGA
CCTGAAGTTCAACATCAAGTCCTCCTCCTCCTCCCCCGACTCCCGCGCCGTGACCTGCGGCATGGC
CTCCCTGTCCGCCGAGAAGGTGACCCTGGACCAGCGCGACTACGAGAAGTACTCCGTGTCCTGCCA
GGAGGACGTGACCTGCCCCACCGCCGAGGAGACCCTGCCCATCGAGCTGGCCCTGGAGGCCCGCCA
GCAGAACAAGTACGAGAACTACTCCACCTCCTTCTTCATCCGCGACATCATCAAGCCCGACCCCCC
CAAGAACCTGCAGATGAAGCCCCTGAAGAACTCCCAGGTGGAGGTGTCCTGGGAGTACCCCGACTC
CTGGTCCACCCCCCACTCCTACTTCTCCCTGAAGTTCTTCGTGCGCATCCAGCGCAAGAAGGAGAA
GATGAAGGAGACCGAGGAGGGCTGCAACCAGAAGGGCGCCTTCCTGGTGGAGAAGACCTCCACCGA
GGTGCAGTGCAAGGGCGGCAACGTGTGCGTGCAGGCCCAGGACCGCTACTACAACTCCTCCTGCTC
CAAGTGGGCCTGCGTGCCCTGCCGCGTGCGCTCCTCCGGCGGCGGCGGCTCCGGCGGCGGCGGCTC
CGGCGGCGGCGGCTCCCCCGTGTCCGTGCCCACCGCCTCCCCCTCCGCCTCCTCCTCCTCCTCCCA
GTGCCGCTCCTCCATGTGCCAGTCCCGCTACCTGCTGTTCCTGGCCACCCTGGCCCTGCTGAACCA
CCTGTCCCTGGCCCGCGTGATCCCCGTGTCCGGCCCCGCCCGCTGCCTGTCCCAGTCCCGCAACCT
GCTGAAGACCACCGACGACATGGTGAAGACCGCCCGCGAGAAGCTGAAGCACTACTCCTGCACCGC
CGAGGACATCGACCACGAGGACATCACCCGCGACCAGACCTCCACCCTGAAGACCTGCCTGCCCCT
GGAGCTGCACAAGAACGAGTCCTGCCTGGCCACCCGCGAGACCTCCTCCACCACCCGCGGCTCCTG
CCTGCCCCCCCAGAAGACCTCCCTGATGATGACCCTGTGCCTGGGCTCCATCTACGAGGACCTGAA
GATGTACCAGACCGAGTTCCAGGCCATCAACGCCGCCCTGCAGAACCACAACCACCAGCAGATCAT
CCTGGACAAGGGCATGCTGGTGGCCATCGACGAGCTGATGCAGTCCCTGAACCACAACGGCGAGAC
CCTGCGCCAGAAGCCCCCCGTGGGCGAGGCCGACCCCTACCGCGTGAAGATGAAGCTGTGCATCCT
GCTGCACGCCTTCTCCACCCGCGTGGTGACCATCAACCGCGTGATGGGCTACCTGTCCTCCGCCTG
AACTAGTAGTGACTGACTAGGATCTGGTTACCACTAAACCAGCCTCAAGAACACCCGAATGGAGTC
TCTAAGCTACATAATACCAACTTACACTTACAAAATGTTGTCCCCCAAAATGTAGCCATTCGTATC
TGCTCCTAATAAAAAGAAAGTTTCTTCACATTCTAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAA
AAA
SEQ ID NO: 5 murine IL-12 - IL-2 signal peptide modification RNA
AGCATAAAAGTCTCAACACAACATATACAAAACAAACGAATCTCAAGCAATCAAGCATTCTACTTC
TATTGCAGCAATTTAAATCATTTCTTTTAAAGCAAAAGCAATTTTCTGAAAATTTTCACCATTTAC
GAACGATAGCGCTGCCACCATGCGCATGCAGCTGCTGCTGCTGATCGCCCTGTCCCTGGCCCTGGT
GACCAACTCCATGTGGGAGCTGGAGAAGGACGTGTACGTGGTGGAGGTGGACTGGACCCCCGACGC
CCCCGGCGAGACCGTGAACCTGACCTGCGACACCCCCGAGGAGGACGACATCACCTGGACCTCCGA
CCAGCGCCACGGCGTGATCGGCTCCGGCAAGACCCTGACCATCACCGTGAAGGAGTTCCTGGACGC
CGGCCAGTACACCTGCCACAAGGGCGGCGAGACCCTGTCCCACTCCCACCTGCTGCTGCACAAGAA
GGAGAACGGCATCTGGTCCACCGAGATCCTGAAGAACTTCAAGAACAAGACCTTCCTGAAGTGCGA
GGCCCCCAACTACTCCGGCCGCTTCACCTGCTCCTGGCTGGTGCAGCGCAACATGGACCTGAAGTT
CAACATCAAGTCCTCCTCCTCCTCCCCCGACTCCCGCGCCGTGACCTGCGGCATGGCCTCCCTGTC
CGCCGAGAAGGTGACCCTGGACCAGCGCGACTACGAGAAGTACTCCGTGTCCTGCCAGGAGGACGT
GACCTGCCCCACCGCCGAGGAGACCCTGCCCATCGAGCTGGCCCTGGAGGCCCGCCAGCAGAACAA
GTACGAGAACTACTCCACCTCCTTCTTCATCCGCGACATCATCAAGCCCGACCCCCCCAAGAACCT
GCAGATGAAGCCCCTGAAGAACTCCCAGGTGGAGGTGTCCTGGGAGTACCCCGACTCCTGGTCCAC
CCCCCACTCCTACTTCTCCCTGAAGTTCTTCGTGCGCATCCAGCGCAAGAAGGAGAAGATGAAGGA
GACCGAGGAGGGCTGCAACCAGAAGGGCGCCTTCCTGGTGGAGAAGACCTCCACCGAGGTGCAGTG
CAAGGGCGGCAACGTGTGCGTGCAGGCCCAGGACCGCTACTACAACTCCTCCTGCTCCAAGTGGGC
CTGCGTGCCCTGCCGCGTGCGCTCCTCCGGCGGCGGCGGCTCCGGCGGCGGCGGCTCCGGCGGCGG
CGGCTCCCCCGTGTCCGTGCCCACCGCCTCCCCCTCCGCCTCCTCCTCCTCCTCCCAGTGCCGCTC
CTCCATGTGCCAGTCCCGCTACCTGCTGTTCCTGGCCACCCTGGCCCTGCTGAACCACCTGTCCCT
GGCCCGCGTGATCCCCGTGTCCGGCCCCGCCCGCTGCCTGTCCCAGTCCCGCAACCTGCTGAAGAC
CACCGACGACATGGTGAAGACCGCCCGCGAGAAGCTGAAGCACTACTCCTGCACCGCCGAGGACAT
CGACCACGAGGACATCACCCGCGACCAGACCTCCACCCTGAAGACCTGCCTGCCCCTGGAGCTGCA
CAAGAACGAGTCCTGCCTGGCCACCCGCGAGACCTCCTCCACCACCCGCGGCTCCTGCCTGCCCCC
CCAGAAGACCTCCCTGATGATGACCCTGTGCCTGGGCTCCATCTACGAGGACCTGAAGATGTACCA
GACCGAGTTCCAGGCCATCAACGCCGCCCTGCAGAACCACAACCACCAGCAGATCATCCTGGACAA
GGGCATGCTGGTGGCCATCGACGAGCTGATGCAGTCCCTGAACCACAACGGCGAGACCCTGCGCCA
GAAGCCCCCCGTGGGCGAGGCCGACCCCTACCGCGTGAAGATGAAGCTGTGCATCCTGCTGCACGC
CTTCTCCACCCGCGTGGTGACCATCAACCGCGTGATGGGCTACCTGTCCTCCGCCTGAACTAGTAG
TGACTGACTAGGATCTGGTTACCACTAAACCAGCCTCAAGAACACCCGAATGGAGTCTCTAAGCTA
CATAATACCAACTTACACTTACAAAATGTTGTCCCCCAAAATGTAGCCATTCGTATCTGCTCCTAA
TAAAAAGAAAGTTTCTTCACATTCTAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAA
SEQ ID NO: 6 murine IL-12 - 2X Alanine mutant RNA
ATGTGCCCCCAGAAGCTGACCATCTCCTGGTTCGCCATCGTGCTGCTGGTGTCCCCCCTGATGGCC
ATGTGGGAGCTGGAGAAGGACGTGTACGTGGTGGAGGTGGACTGGACCCCCGACGCCCCCGGCGAG
ACCGTGAACCTGACCTGCGACACCCCCGAGGAGGACGACATCACCTGGACCTCCGACCAGCGCCAC
GGCGTGATCGGCTCCGGCAAGACCCTGACCATCACCGTGAAGGCCGCCCTGGACGCCGGCCAGTAC
ACCTGCCACAAGGGCGGCGAGACCCTGTCCCACTCCCACCTGCTGCTGCACAAGAAGGAGAACGGC
ATCTGGTCCACCGAGATCCTGAAGAACTTCAAGAACAAGACCTTCCTGAAGTGCGAGGCCCCCAAC
TACTCCGGCCGCTTCACCTGCTCCTGGCTGGTGCAGCGCAACATGGACCTGAAGTTCAACATCAAG
TCCTCCTCCTCCTCCCCCGACTCCCGCGCCGTGACCTGCGGCATGGCCTCCCTGTCCGCCGAGAAG
GTGACCCTGGACCAGCGCGACTACGAGAAGTACTCCGTGTCCTGCCAGGAGGACGTGACCTGCCCC
ACCGCCGAGGAGACCCTGCCCATCGAGCTGGCCCTGGAGGCCCGCCAGCAGAACAAGTACGAGAAC
TACTCCACCTCCTTCTTCATCCGCGACATCATCAAGCCCGACCCCCCCAAGAACCTGCAGATGAAG
CCCCTGAAGAACTCCCAGGTGGAGGTGTCCTGGGAGTACCCCGACTCCTGGTCCACCCCCCACTCC
TACTTCTCCCTGAAGTTCTTCGTGCGCATCCAGCGCAAGAAGGAGAAGATGAAGGAGACCGAGGAG
GGCTGCAACCAGAAGGGCGCCTTCCTGGTGGAGAAGACCTCCACCGAGGTGCAGTGCAAGGGCGGC
AACGTGTGCGTGCAGGCCCAGGACCGCTACTACAACTCCTCCTGCTCCAAGTGGGCCTGCGTGCCC
TGCCGCGTGCGCTCC
SEQ ID NO: 7 murine IL-12 - 3X Alanine mutant RNA
ATGTGCCCCCAGAAGCTGACCATCTCCTGGTTCGCCATCGTGCTGCTGGTGTCCCCCCTGATGGCC
ATGTGGGAGCTGGAGAAGGACGTGTACGTGGTGGAGGTGGACTGGACCCCCGACGCCCCCGGCGAG
ACCGTGAACCTGACCTGCGACACCCCCGAGGAGGACGACATCACCTGGACCTCCGACCAGCGCCAC
GGCGTGATCGGCTCCGGCAAGACCCTGACCATCACCGTGAAGGCCGCCCTGGACGCCGGCCAGTAC
ACCTGCCACAAGGGCGGCGAGACCCTGTCCCACTCCCACCTGCTGCTGCACGCCAAGGAGAACGGC
ATCTGGTCCACCGAGATCCTGAAGAACTTCAAGAACAAGACCTTCCTGAAGTGCGAGGCCCCCAAC
TACTCCGGCCGCTTCACCTGCTCCTGGCTGGTGCAGCGCAACATGGACCTGAAGTTCAACATCAAG
TCCTCCTCCTCCTCCCCCGACTCCCGCGCCGTGACCTGCGGCATGGCCTCCCTGTCCGCCGAGAAG
GTGACCCTGGACCAGCGCGACTACGAGAAGTACTCCGTGTCCTGCCAGGAGGACGTGACCTGCCCC
ACCGCCGAGGAGACCCTGCCCATCGAGCTGGCCCTGGAGGCCCGCCAGCAGAACAAGTACGAGAAC
TACTCCACCTCCTTCTTCATCCGCGACATCATCAAGCCCGACCCCCCCAAGAACCTGCAGATGAAG
CCCCTGAAGAACTCCCAGGTGGAGGTGTCCTGGGAGTACCCCGACTCCTGGTCCACCCCCCACTCC
TACTTCTCCCTGAAGTTCTTCGTGCGCATCCAGCGCAAGAAGGAGAAGATGAAGGAGACCGAGGAG
GGCTGCAACCAGAAGGGCGCCTTCCTGGTGGAGAAGACCTCCACCGAGGTGCAGTGCAAGGGCGGC
AACGTGTGCGTGCAGGCCCAGGACCGCTACTACAACTCCTCCTGCTCCAAGTGGGCCTGCGTGCCC
TGCCGCGTGCGCTCC
SEQ ID NO: 8 murine IL-12 - 4X Alanine mutant RNA
ATGTGCCCCCAGAAGCTGACCATCTCCTGGTTCGCCATCGTGCTGCTGGTGTCCCCCCTGATGGCC
ATGTGGGAGCTGGAGAAGGACGTGTACGTGGTGGAGGTGGACTGGACCCCCGACGCCCCCGGCGAG
ACCGTGAACCTGACCTGCGACACCCCCGAGGAGGACGACATCACCTGGACCTCCGACCAGCGCCAC
GGCGTGATCGGCTCCGGCAAGACCCTGACCATCACCGTGAAGGCCGCCCTGGACGCCGGCCAGTAC
ACCTGCCACAAGGGCGGCGAGACCCTGTCCCACTCCCACCTGCTGCTGCACGCCAAGGAGAACGGC
ATCTGGTCCACCGAGATCCTGAAGAACTTCAAGAACAAGACCTTCCTGAAGTGCGAGGCCCCCAAC
TACTCCGGCCGCTTCACCTGCTCCTGGCTGGTGCAGCGCAACATGGACCTGAAGTTCAACATCAAG
TCCTCCTCCTCCTCCCCCGACTCCCGCGCCGTGACCTGCGGCATGGCCTCCCTGTCCGCCGAGAAG
GTGACCCTGGACCAGCGCGACTACGAGAAGTACTCCGTGTCCTGCCAGGAGGACGTGACCTGCCCC
ACCGCCGAGGAGACCCTGCCCATCGAGCTGGCCCTGGAGGCCCGCCAGCAGAACGCCTACGAGAAC
TACTCCACCTCCTTCTTCATCCGCGACATCATCAAGCCCGACCCCCCCAAGAACCTGCAGATGAAG
CCCCTGAAGAACTCCCAGGTGGAGGTGTCCTGGGAGTACCCCGACTCCTGGTCCACCCCCCACTCC
TACTTCTCCCTGAAGTTCTTCGTGCGCATCCAGCGCAAGAAGGAGAAGATGAAGGAGACCGAGGAG
GGCTGCAACCAGAAGGGCGCCTTCCTGGTGGAGAAGACCTCCACCGAGGTGCAGTGCAAGGGCGGC
AACGTGTGCGTGCAGGCCCAGGACCGCTACTACAACTCCTCCTGCTCCAAGTGGGCCTGCGTGCCC
TGCCGCGTGCGCTCC
SEQ ID NO: 9 murine IL-27 RNA
AGCATAAAAGTCTCAACACAACATATACAAAACAAACGAATCTCAAGCAATCAAGCATTCTACTTC
TATTGCAGCAATTTAAATCATTTCTTTTAAAGCAAAAGCAATTTTCTGAAAATTTTCACCATTTAC
GAACGATAGCGCTGCCACCATGTCCGCCCTGCTGATCCTGGCCCTGGTGGGCGCCGCCGTGGCCGA
CTACAAGGACGACGACGACAAGCTGGGCTACACCGAGACCGCCCTGGTGGCCCTGTCCCAGCCCCG
CGTGCAGTGCCACGCCTCCCGCTACCCCGTGGCCGTGGACTGCTCCTGGACCCCCCTGCAGGCCCC
CAACTCCACCCGCTCCACCTCCTTCATCGCCACCTACCGCCTGGGCGTGGCCACCCAGCAGCAGTC
CCAGCCCTGCCTGCAGCGCTCCCCCCAGGCCTCCCGCTGCACCATCCCCGACGTGCACCTGTTCTC
CACCGTGCCCTACATGCTGAACGTGACCGCCGTGCACCCCGGCGGCGCCTCCTCCTCCCTGCTGGC
CTTCGTGGCCGAGCGCATCATCAAGCCCGACCCCCCCGAGGGCGTGCGCCTGCGCACCGCCGGCCA
GCGCCTGCAGGTGCTGTGGCACCCCCCCGCCTCCTGGCCCTTCCCCGACATCTTCTCCCTGAAGTA
CCGCCTGCGCTACCGCCGCCGCGGCGCCTCCCACTTCCGCCAGGTGGGCCCCATCGAGGCCACCAC
CTTCACCCTGCGCAACTCCAAGCCCCACGCCAAGTACTGCATCCAGGTGTCCGCCCAGGACCTGAC
CGACTACGGCAAGCCCTCCGACTGGTCCCTGCCCGGCCAGGTGGAGTCCGCCCCCCACAAGCCCTC
CCGCGGCTCCGGCTCCGGCGGCTCCGGCGGCTCCGGCTCCGGCAAGCTGCCCACCGACCCCCTGTC
CCTGCAGGAGCTGCGCCGCGAGTTCACCGTGTCCCTGTACCTGGCCCGCAAGCTGCTGTCCGAGGT
GCAGGGCTACGTGCACTCCTTCGCCGAGTCCCGCCTGCCCGGCGTGAACCTGGACCTGCTGCCCCT
GGGCTACCACCTGCCCAACGTGTCCCTGACCTTCCAGGCCTGGCACCACCTGTCCGACTCCGAGCG
CCTGTGCTTCCTGGCCACCACCCTGCGCCCCTTCCCCGCCATGCTGGGCGGCCTGGGCACCCAGGG
CACCTGGACCTCCTCCGAGCGCGAGCAGCTGTGGGCCATGCGCCTGGACCTGCGCGACCTGCACCG
CCACCTGCGCTTCCAGGTGCTGGCCGCCGGCTTCAAGTGCTCCAAGGAGGAGGAGGACAAGGAGGA
GGAGGAGGAGGAGGAGGAGGAGGAGAAGAAGCTGCCCCTGGGCGCCCTGGGCGGCCCCAACCAGGT
GTCCTCCCAGGTGTCCTGGCCCCAGCTGCTGTACACCTACCAGCTGCTGCACTCCCTGGAGCTGGT
GCTGTCCCGCGCCGTGCGCGACCTGCTGCTGCTGTCCCTGCCCCGCCGCCCCGGCTCCGCCTGGGA
CTCCTTAATAAACTAGTAGTGACTGACTAGGATCTGGTTACCACTAAACCAGCCTCAAGAACACCC
GAATGGAGTCTCTAAGCTACATAATACCAACTTACACTTACAAAATGTTGTCCCCCAAAATGTAGC
CATTCGTATCTGCTCCTAATAAAAAGAAAGTTTCTTCACATTCTAAAAAAAAAAAAAAAAAAAAAA
AAAAAAAAAAAAAAAAAAAAAAAAAAAAAAAAAAAAAAAAAAAAAAAAAAAAAAAAAAAAAAAAAA
AAAAAAAAAAAAA
SEQ ID NO: 10 IL-12 2xAla variant
MCPQKLTISWFAIVLLVSPLMAMWELEKDVYVVEVDWTPDAPGETVNLTCDTPEEDDITWTSDQRH
GVIGSGKTLTITVKAALDAGQYTCHKGGETLSHSHLLLHKKENGIWSTEILKNFKNKTFLKCEAPN
YSGRFTCSWLVQRNMDLKFNIKSSSSSPDSRAVTCGMASLSAEKVTLDQRDYEKYSVSCQEDVTCP
TAEETLPIELALEARQQNKYENYSTSFFIRDIIKPDPPKNLQMKPLKNSQVEVSWEYPDSWSTPHS
YFSLKFFVRIQRKKEKMKETEEGCNQKGAFLVEKTSTEVQCKGGNVCVQAQDRYYNSSCSKWACVP
CRVRS
SEQ ID NO: 11 IL-12 3xAla variant
MCPQKLTISWFAIVLLVSPLMAMWELEKDVYVVEVDWTPDAPGETVNLTCDTPEEDDITWTSDQRH
GVIGSGKTLTITVKAALDAGQYTCHKGGETLSHSHLLLHAKENGIWSTEILKNEKNKTFLKCEAPN
YSGRFTCSWLVQRNMDLKFNIKSSSSSPDSRAVTCGMASLSAEKVTLDQRDYEKYSVSCQEDVTCP
TAEETLPIELALEARQQNKYENYSTSFFIRDIIKPDPPKNLQMKPLKNSQVEVSWEYPDSWSTPHS
YFSLKFFVRIQRKKEKMKETEEGCNQKGAFLVEKTSTEVQCKGGNVCVQAQDRYYNSSCSKWACVP
CRVRS
SEQ ID NO: 12 IL-12 4xAla variant
MCPQKLTISWFAIVLLVSPLMAMWELEKDVYVVEVDWTPDAPGETVNLICDTPEEDDITWTSDQRH
GVIGSGKTLTITVKAALDAGQYTCHKGGETLSHSHLLLHAKENGIWSTEILKNFKNKTFLKCEAPN
YSGRFTCSWLVQRNMDLKFNIKSSSSSPDSRAVTCGMASLSAEKVTLDQRDYEKYSVSCQEDVTCP
TAEETLPIELALEARQQNAYENYSTSFFIRDIIKPDPPKNLQMKPLKNSQVEVSWEYPDSWSTPHS
YESLKEEVRIQRKKERMKETEEGCNQKGAFLVEKTSTEVQCKGGNVCVQAQDRYYNSSCSKWACVP
CRVRS

### Enumerated Embodiments

The following exemplary embodiments are provided, the numbering of which is not to be construed as designating levels of importance:

Embodiment 1 provides a lipid nanoparticle (LNP), wherein the LNP comprises:
(a) at least one first nucleoside-modified ribonucleic acid (RNA), wherein the at least one first nucleoside-modified RNA encodes an antigen;
(b) at least one second nucleoside-modified RNA, wherein the at least one second nucleoside-modified RNA encodes a cytokine or immune receptor (such as but not limited to a cytokine receptor); and
(c) at least one ionizable lipid;
wherein the LNP is capable of eliciting a modulated immune response against the antigen in a subject.

Embodiment 2 provides the LNP of embodiment 1, wherein the modulated immune response comprises an enhanced immune response and/or a decreased immune response.

Embodiment 3 provides the LNP of embodiment 1 or 2, wherein the modulated immune response is tissue-specific.

Embodiment 4 provides the LNP of any one of the preceding embodiments, wherein the first nucleoside-modified RNA, the second nucleoside-modified RNA, or both, is/are messenger RNA (mRNA).

Embodiment 5 provides the LNP of any one of the preceding embodiments, wherein the first nucleoside-modified RNA, the second nucleoside-modified RNA, or both, independently comprise pseudouridines and/or 1-methyl-pseudouridines.

Embodiment 6 provides the LNP of any one of the preceding embodiments, wherein the first nucleoside-modified RNA, the second nucleoside-modified RNA, or both, is/are in vitro transcribed (IVT) RNA.

Embodiment 7 provides the e LNP of any one of the preceding embodiments, wherein the cytokine is selected from the group consisting of a chemokine, an interleukin (IL), an interferon (IFN), a tumor necrosis factor (TNF) family member, a transforming growth factor (TGF), and any combination thereof.

Embodiment 8 provides the LNP of any one of the preceding embodiments, wherein the cytokine is selected from the group consisting of IL-2, IL-6, IL-12, IL-15, IL-27, TGF-β, and any combination thereof.

Embodiment 9 provides the LNP of any one of the preceding embodiments, wherein the cytokine comprises IL-27.

Embodiment 10 provides the LNP of any one of the preceding embodiments, wherein the second nucleoside-modified RNA encodes an Epstein-Barr virus-induced gene 3 (Ebi3) subunit of IL-27 linked to an IL-27p28 subunit of IL-27 via a flexible linker, such as a flexible protein linker.

Embodiment 11 provides the LNP of any one of the preceding embodiments, wherein the cytokine comprises IL-12.

Embodiment 12 provides the LNP of any one of the preceding embodiments, wherein the second nucleoside-modified RNA encodes a p40 subunit of IL-12 linked to a p35 subunit of IL-12 via a flexible linker, such as a flexible protein linker.

Embodiment 13 provides the LNP of any one of the preceding embodiments, wherein the immune receptor (such as but not limited to a cytokine receptor) comprises a soluble immune receptor (such as but not limited to a cytokine receptor) or immune receptor (such as but not limited to a cytokine receptor) decoy.

Embodiment 14 provides the LNP of any one of the preceding embodiments, wherein the ionizable lipid encapsulates the first nucleoside-modified RNA and the second nucleoside-modified RNA.

Embodiment 15 provides the LNP of any one of the preceding embodiments, wherein the ionizable lipid is a cationic lipid.

Embodiment 16 provides the LNP of any one of the preceding embodiments, wherein the ionizable lipid is selected from those similar to that formulated in the BNT162b2 vaccine and SM-102.

Embodiment 17 provides the LNP of any one of the preceding embodiments, wherein the antigen is derived from a pathogen, and wherein the pathogen is selected from a virus, a bacterium, a fungus, or a parasite.

Embodiment 18 provides the LNP of any one of the preceding embodiments, wherein the pathogen is a severe acute respiratory syndrome coronavirus 2 (SARS-CoV-2) virus.

Embodiment 19 provides the LNP of any one of the preceding embodiments, wherein the SARS-CoV-2 is wild-type SARS-CoV-2 or a variant SARS-CoV-2.

Embodiment 20 provides the LNP of any one of the preceding embodiments, wherein the antigen is a SARS-CoV-2 antigen.

Embodiment 21 provides the LNP of any one of the preceding embodiments, wherein the antigen is a tumor antigen.

Embodiment 22 provides the LNP of any one of the preceding embodiments, wherein:
(i) the enhanced immune response in the subject comprises an augmented CD8+ T cell response in the subject compared to a CD8+ T cell response in a subject administered a control LNP lacking the second nucleoside-modified RNA encoding a cytokine or immune receptor (such as but not limited to a cytokine receptor); or
(ii) the reduced immune response in the subject comprises a decreased CD8+ T cell response in the subject compared to a CD8+ T cell response in a subject administered a control LNP lacking the second nucleoside-modified RNA encoding a cytokine or immune receptor (such as but not limited to a cytokine receptor).

Embodiment 23 provides a pharmaceutical composition comprising the LNP of any one of the preceding embodiments and at least one pharmaceutically acceptable carrier, diluent, and/or excipient.

Embodiment 24 provides the LNP of any one of the preceding embodiments, for use in a method of eliciting a modulated immune response against the antigen in the subject.

Embodiment 25 provides a method of eliciting a modulated immune response against an antigen in a subject, the method comprising administering to the subject an effective amount of a pharmaceutical composition comprising a lipid nanoparticle (LNP) and at least one pharmaceutically acceptable carrier, diluent, and/or excipient, wherein the LNP comprises:
(a) at least one first nucleoside-modified ribonucleic acid (RNA) encoding the antigen;
(b) at least one second nucleoside-modified RNA encoding a cytokine or immune receptor (such as but not limited to a cytokine receptor); and
(c) at least one ionizable lipid;
wherein the LNP elicits a modulated immune response against the antigen in the subject.

Embodiment 26 provides the method of any one of the preceding embodiments, wherein the modulated immune response comprises an enhanced immune response and/or a decreased immune response.

Embodiment 27 provides the method of any one of the preceding embodiments, wherein the modulated immune response is tissue-specific.

Embodiment 28 provides the method of any one of the preceding embodiments, wherein the first nucleoside-modified RNA, the second nucleoside-modified RNA, or both, is/are messenger RNA (mRNA).

Embodiment 29 provides the method of any one of the preceding embodiments, wherein the first nucleoside-modified RNA, the second nucleoside-modified RNA, or both, independently comprise pseudouridine and/or 1-methyl-pseudouridine.

Embodiment 30 provides the method of any one of the preceding embodiments, wherein the first nucleoside-modified RNA, the second nucleoside-modified RNA, or both, is/are in vitro transcribed (IVT) RNA.

Embodiment 31 provides the method of any one of the preceding embodiments, wherein the cytokine is selected from the group consisting of a chemokine, an interleukin (IL), an interferon (IFN), a tumor necrosis factor (TNF) family member, a transforming growth factor (TGF), and any combination thereof.

Embodiment 32 provides the method of any one of the preceding embodiments, wherein the cytokine is selected from the group consisting of IL-2, IL-6, IL-12, IL-15, IL-27, TGF-β, and any combination thereof.

Embodiment 33 provides the method of any one of the preceding embodiments, wherein the cytokine comprises IL-27.

Embodiment 34 provides the method of any one of the preceding embodiments, wherein the second nucleoside-modified RNA encodes an Epstein-Barr virus-induced gene 3 (Ebi3) subunit of IL-27 linked to an IL-27p28 subunit of IL-27 via a flexible linker, such as a flexible protein linker.

Embodiment 35 provides the method of any one of the preceding embodiments, wherein the cytokine comprises IL-12.

Embodiment 36 provides the method of any one of the preceding embodiments, wherein the second nucleoside-modified RNA encodes a p40 subunit of IL-12 linked to a p35 subunit of IL-12 via a flexible linker, such as a flexible protein linker.

Embodiment 37 provides the method of any one of the preceding embodiments, wherein the immune receptor (such as but not limited to a cytokine receptor) comprises a soluble immune receptor (such as but not limited to a cytokine receptor) or a immune receptor (such as but not limited to a cytokine receptor) decoy.

Embodiment 38 provides the method of any one of the preceding embodiments, wherein the ionizable lipid encapsulates the first nucleoside-modified RNA and the second nucleoside-modified RNA.

Embodiment 39 provides the method of any one of the preceding embodiments, wherein the ionizable lipid is a cationic lipid.

Embodiment 40 provides the method of any one of the preceding embodiments, wherein the ionizable lipid is selected from those similar to that formulated in the BNT162b2 vaccine and SM-102.

Embodiment 41 provides the method of any one of the preceding embodiments, wherein the antigen is derived from a pathogen, and wherein the pathogen is selected from a virus, a bacterium, a fungus, or a parasite.

Embodiment 42 provides the method of any one of the preceding embodiments, wherein the pathogen is a severe acute respiratory syndrome coronavirus 2 (SARS-CoV-2) virus.

Embodiment 43 provides the method of any one of the preceding embodiments, wherein the SARS-CoV-2 is wild-type SARS-CoV-2 or a variant SARS-CoV-2.

Embodiment 44 provides the method of any one of the preceding embodiments, wherein the antigen is a SARS-CoV-2 antigen.

Embodiment 45 provides the method of any one of the preceding embodiments, wherein the antigen is a tumor antigen.

Embodiment 46 provides the method of any one of the preceding embodiments, wherein:
(i) the enhanced immune response in the subject comprises an augmented CD8⁺ T cell response in the subject compared to a CD8⁺ T cell response in a subject administered a control composition lacking the second nucleoside-modified RNA encoding a cytokine or immune receptor (such as but not limited to a cytokine receptor); or
(ii) the reduced immune response in the subject comprises a decreased CD8⁺ T cell response in the subject compared to a CD8⁺ T cell response in a subject administered a control LNP lacking the second nucleoside-modified RNA encoding a cytokine or immune receptor (such as but not limited to a cytokine receptor).

Embodiment 47 provides the method of any one of the preceding embodiments, wherein the subject is a human.

Embodiment 48 provides the method of any one of the preceding embodiments, wherein the administering comprises intramuscular injection.

Embodiment 49 provides the method of any one of the preceding embodiments, wherein the administering comprises administering a first dose.

Embodiment 50 provides the method of any one of the preceding embodiments, wherein the administering further comprises administering at least one booster dose.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A lipid nanoparticle (LNP), wherein the LNP comprises:
(a) at least one first nucleoside-modified ribonucleic acid (RNA), wherein the at least one first nucleoside-modified RNA encodes an antigen;
(b) at least one second nucleoside-modified RNA, wherein the at least one second nucleoside-modified RNA encodes a cytokine or immune receptor, optionally wherein the immune receptor is a cytokine receptor; and
(c) at least one ionizable lipid;
wherein the LNP is capable of eliciting a modulated immune response against the antigen in a subject.

2. The LNP of claim 1, wherein the modulated immune response comprises an enhanced immune response and/or a decreased immune response, optionally wherein the modulated immune response is tissue-specific.

3. (canceled)

4. The LNP of claim 1, wherein at least one of the following applies:
(i) the first nucleoside-modified RNA, the second nucleoside-modified RNA, or both, is/are messenger RNA (mRNA),
(ii) the first nucleoside-modified RNA, the second nucleoside-modified RNA, or both, independently comprise pseudouridines or 1-methyl-pseudouridines;
(iii) at least one of the first nucleoside-modified RNA and the second nucleoside-modified RNA is in vitro transcribed (IVT) RNA.

5-6. (canceled)

7. The LNP of claim 1, wherein at least one of the following applies:
(i) the cytokine is selected from the group consisting of a chemokine, an interleukin (IL), an interferon (IFN), a tumor necrosis factor (TNF) family member, a transforming growth factor (TGF), and any combination thereof;
(ii) the cytokine is selected from the group consisting of IL-2, IL-6, IL-12, IL-15, IL-27, TGF-β, and any combination thereof;
(iii) the cytokine comprises IL-27:
(iv) the cytokine comprises IL-12;
(v) the immune receptor comprises a soluble immune receptor or an immune receptor decoy.

8-9. (canceled)

10. The LNP of claim 1, wherein:
(i) the second nucleoside-modified RNA encodes an Epstein-Barr virus-induced gene 3 (Ebi3) subunit of IL-27 linked to an IL-27p28 subunit of IL-27 via a flexible linker; or
(ii) the second nucleoside-modified RNA encodes a p40 subunit of IL-12 linked to a p35 subunit of IL-12 via a flexible linker.

11-13. (canceled)

14. The LNP of claim 1, wherein at least one of the following applies:
(i) the ionizable lipid encapsulates the first nucleoside-modified RNA and the second nucleoside-modified RNA,
(ii) the ionizable lipid is a cationic lipid;
(iii) the ionizable lipid is selected from those similar to that formulated in the BNT162b2 vaccine and SM-102.

15-16. (canceled)

17. The LNP of claim 1, wherein the antigen is derived from a pathogen, and further wherein at least one of the following applies:
(i) the pathogen is selected from the group consisting of a virus, a bacterium, a fungus, and a parasite,
(ii) the pathogen is a severe acute respiratory syndrome coronavirus 2 (SARS-CoV-2) virus, wherein the SARS-CoV-2 is wild-type SARS-CoV-2 or a variant SARS-CoV-2.

18-20. (canceled)

21. The LNP of claim 1, wherein the antigen is a tumor antigen.

22. The LNP of claim 1, wherein:
(i) the modulated immune response comprises an enhanced immune response comprising an augmented CD8⁺ T cell response in the subject compared to a CD8⁺ T cell response in a subject administered a control LNP lacking the second nucleoside-modified RNA encoding a cytokine or immune receptor, optionally wherein the immune receptor is a cytokine receptor; or
(ii) the modulated immune response comprises a reduced immune response comprising a decreased CD8⁺ T cell response in the subject compared to a CD8⁺ T cell response in a subject administered a control LNP lacking the second nucleoside-modified RNA encoding a cytokine or immune receptor, optionally wherein the immune receptor is a cytokine receptor.

23. A pharmaceutical composition comprising the LNP of claim 1 and at least one pharmaceutically acceptable carrier, diluent, or excipient.

24. (canceled)

25. A method of eliciting a modulated immune response against an antigen in a subject, the method comprising administering to the subject an effective amount of a pharmaceutical composition comprising a lipid nanoparticle (LNP) and at least one pharmaceutically acceptable carrier, diluent, or excipient, wherein the LNP comprises:
(a) at least one first nucleoside-modified ribonucleic acid (RNA) encoding the antigen;
(b) at least one second nucleoside-modified RNA encoding a cytokine or immune receptor, optionally wherein the immune receptor is a cytokine receptor; and
(c) at least one ionizable lipid;
wherein the LNP elicits a modulated immune response against the antigen in the subject.

26. The method of claim 25, wherein the modulated immune response comprises an enhanced immune response or a decreased immune response, optionally wherein the modulated immune response is tissue-specific.

27. (canceled)

28. The method of claim 25, wherein at least one of the following applies:
(i) the first nucleoside-modified RNA, the second nucleoside-modified RNA, or both, is/are messenger RNA (mRNA),
(ii) the first nucleoside-modified RNA, the second nucleoside-modified RNA, or both, independently comprise pseudouridines and/or 1-methyl-pseudouridines;
(iii) the first nucleoside-modified RNA, the second nucleoside-modified RNA, or both, is/are in vitro transcribed (IVT) RNA.

29-30. (canceled)

31. The method of claim 25, wherein at least one of the following applies:
(i) the cytokine is selected from the group consisting of a chemokine, an interleukin (IL), an interferon (IFN), a tumor necrosis factor (TNF) family member, a transforming growth factor (TGF), and any combination thereof;
(ii) the cytokine is selected from the group consisting of IL-2, IL-6, IL-12, IL-15, IL-27, TGF-β, and any combination thereof;
(iii) the cytokine comprises IL-27;
(iv) the cytokine comprises IL-12;
(v) the immune receptor comprises a soluble immune receptor or an immune receptor decoy.

32-33. (canceled)

34. The method of claim 25, wherein:
(i) the second nucleoside-modified RNA encodes an Epstein-Barr virus-induced gene 3 (Ebi3) subunit of IL-27 linked to an IL-27p28 subunit of IL-27 via a flexible linker; or
(ii) the second nucleoside-modified RNA encodes a p40 subunit of IL-12 linked to a p35 subunit of IL-12 via a flexible linker.

35-37. (canceled)

38. The method of claim 25, wherein at least one of the following applies:
(i) the ionizable lipid encapsulates the first nucleoside-modified RNA and the second nucleoside-modified RNA;
(ii) the ionizable lipid is a cationic lipid;
(iii) the ionizable lipid is selected from those similar to that formulated in the BNT162b2 vaccine and SM-102.

39-40. (canceled)

41. The method of claim 25, wherein the antigen is derived from a pathogen, and further wherein at least one of the following applies:
(i) the pathogen is selected from the group consisting of a virus, a bacterium, a fungus, and a parasite;
(ii) the pathogen is a severe acute respiratory syndrome coronavirus 2 (SARS-CoV-2) virus, wherein the SARS-CoV-2 is wild-type SARS-CoV-2 or a variant SARS-CoV-2.

42-44. (canceled)

45. The method of claim 25, wherein the antigen is a tumor antigen.

46. The method of claim 25, wherein:
(i) the modulated immune response comprises an enhanced immune response comprising an augmented CD8⁺ T cell response in the subject compared to a CD8⁺ T cell response in a subject administered a control LNP lacking the second nucleoside-modified RNA encoding a cytokine or immune receptor, optionally wherein the immune receptor is a cytokine receptor; or
(ii) the modulated immune response comprises a reduced immune response comprising a decreased CD8⁺ T cell response in the subject compared to a CD8⁺ T cell response in a subject administered a control LNP lacking the second nucleoside-modified RNA encoding a cytokine or immune receptor, optionally wherein the immune receptor is a cytokine receptor.

47. The method of claim 25, wherein at least one of the following applies:
(i) the subject is a human;
(ii) the administering comprises intramuscular injection;
(iii) the administering comprises administering a first dose, optionally wherein the administering further comprises administering at least one booster dose.

48-50. (canceled)
